(19) United States
(12) Patent Application Publication
(10) Pub. No.: US 2024/0191233 A1
(43) Pub. Date: Jun. 13, 2024

(54) COMPOUNDS AND METHODS FOR REDUCING NLRP3 EXPRESSION

(71) Applicant: IONIS PHARMACEUTICALS, INC., Carlsbad, CA (US)

(72) Inventors: Susan M. FREIER, Carlsbad, CA (US); Huynh-Hoa BUI, Carlsbad, CA (US)

(21) Appl. No.: 18/546,462

(22) PCT Filed: Feb. 17, 2022
(86) PCT No.: PCT/US2022/016822

§ 371 Date: Aug. 15, 2023

Related U.S. Application Data
(60) Provisional application No. 63/151,058, filed on Feb. 18, 2021.
(60) Provisional application No. 63/229,261, filed on Aug. 4, 2021.

(51) Int. Cl. C12N 15/113; A61P 1/16

(52) U.S. Cl. CPC C12N 15/113; A61P 1/16; C12N 2310/11; C12N 2310/315; C12N 2310/321; C12N 2310/3341

(57) ABSTRACT

Provided are compounds, methods, and pharmaceutical compositions for reducing the amount or activity of NLRP3 RNA in a cell or subject, and in certain instances reducing the amount of NLRP3 protein in a cell or subject. These compounds, methods, and pharmaceutical compositions are useful to ameliorate at least one symptom of a kidney injury or kidney disease, including acute kidney injury and chronic kidney disease. The compounds, methods, and pharmaceutical compositions are further useful in the treatment of a cardiac disorder or cardiac injury.

### FIELD

Provided herein are compounds, methods, and pharmaceutical compositions for reducing an amount of NLRP3 RNA in a cell or a subject, and in certain instances reducing the amount of NLRP3 protein in a cell or a subject. In certain embodiments, compounds, methods, and pharmaceutical compositions are useful ameliorating at least one symptom of a kidney disease or kidney injury. Symptoms of kidney diseases and kidney injuries include, but are not limited to, nausea, vomiting, loss of appetite, reduced urine output, elevated serum creatinine levels, muscle cramping, swelling, itching, chest pain, shortness of breath and elevated blood pressure. The compounds, methods, and pharmaceutical compositions are further useful in the treatment of a cardiac disorder or cardiac injury.

### BACKGROUND

Kidney diseases and kidney injuries can prevent kidneys from functioning properly. The kidney's main function is to filter and eliminate waste and fluids from the body. Symptoms of kidney diseases and injuries include, but are not limited to, nausea, vomiting, loss of appetite, reduced urine output, elevated serum creatinine levels, muscle cramping, swelling, itching, chest pain, shortness of breath and elevated blood pressure. Acute kidney injury (AKI) is an abrupt loss in kidney function. Individuals with diabetes, cancer, cardiovascular disease and human immunodeficiency virus (HIV) acquired immune deficiency syndrome (AIDS), or who have recently undergone a surgical procedure, are at risk for AKI. An individual with an increase in serum creatinine of at least 26.4 μmol/L (0.3 mg/dL), a percentage increase in serum creatinine of more than 50% from baseline, or a reduction in urine output (<0.5 mL/kg hourly for >6 h) may be diagnosed with AKI. Chronic kidney disease (CKD), also referred to as chronic kidney failure, is a gradual loss of kidney function. Kidney diseases and injuries may be treated with fluid replacement and dialysis. However, if not treated sufficiently, they may result in heart failure and/or death.

Cardiac disorders and cardiac injuries can prevent the heart from functioning properly. If not treated sufficiently, cardiac disorders and cardiac injuries may be fatal. Heart disease is a widespread and costly morbidity throughout the world, and the leading cause of death for men and women in the United States.

NLR family pyrin domain containing 3 (NLRP3) is a subunit of the inflammasome, a component of the innate immune system that functions as a pathogen- and damage-associated molecular pattern (PAMPs and DAMPs) recognition receptor. Many activators of the inflammasome have been linked to the pathology of disease. Among these are reactive oxygen species (ROS), advanced glycation end products (AGE), ATP, particulate/crystalline substances (e.g. monosodium urate, cholesterol, asbestos, etc.) and bacterial and viral pathogens. Hepatic NLRP3 inflammasome activation, which promotes caspase-1-dependent IL-1β production, occurs in patients with NASH, and evidence from knockout mouse models suggests activation of the inflammasome is important in NAFLD progression. NLRP3 has been implicated in cardiac disorders and diminished cardiac function, and in liver disorders.

### SUMMARY

Provided herein are compounds, methods and pharmaceutical compositions for reducing the amount of NLRP3 RNA, and in certain embodiments reducing the amount or activity of NLRP3 protein in a cell or a subject. In certain embodiments, the subject has or is at risk of having acute kidney injury (AKI) or chronic kidney disease (CKD). In certain embodiments, compounds useful for reducing the amount of NLRP3 RNA and/or NLRP3 protein are oligomeric compounds. In certain embodiments, oligomeric compounds comprise modified oligonucleotides.

Also provided are herein are methods useful for ameliorating at least one symptom of a kidney disease, e.g, CKD or kidney injury, e.g., AKI. In certain embodiments, the at least one symptom is nausea, vomiting, loss of appetite, reduced urine output, elevated serum creatinine levels, muscle cramping, swelling, itching, chest pain, shortness of breath and elevated blood pressure, or a combination thereof.

Also provided herein are methods useful for treating a cardiac disorder or cardiac injury. The cardiac disorder or cardiac injury may be heart failure, hypokalemia, a cardiomyopathy, or a cardiac arrhythmia. Symptoms of cardiac disorders and cardiac injuries include, but are not limited to, pain, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, and/or blood clots. In certain embodiments, the compounds, methods, and pharmaceutical compositions are useful in reducing a sign or a symptom of heart failure.

Also provided herein are methods useful for treating a liver disorder. The liver disorder may be non-alcoholic fatty liver disease (NAFLD) or non-alcoholic steatohepatitis (NASH). Symptoms of liver disorders include fatigue, ascites, pain in the upper right abdomen, bleeding and/or bruising easily, itchy skin, jaundice, loss of appetite, nausea, swelling in the legs, confusion, drowsiness, slurred speech, enlarged blood vessels, and red palms.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D configuration as found in naturally occurring deoxyribonucleic acids (DNA). In certain embodiments, a 2′-deoxynucleoside or nucleoside comprising an unmodified 2′-deoxyribosyl sugar moiety may comprise a modified nucleobase or may comprise an RNA nucleobase (uracil).

As used herein, “2′-MOE” or “2′-MOE sugar moiety” means a 2′-OCH₂CH₂OCH₃ group in place of the 2′-OH group of a ribosyl sugar moiety. “MOE” means methoxyethyl.

As used herein, “2′-MOE nucleoside” means a nucleoside comprising a 2′-MOE sugar moiety.

As used herein, “2′-OMe” or “2′-O-methyl sugar moiety” means a 2′-OCH₃ group in place of the 2′-OH group of a ribosyl sugar moiety.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “administering” means providing a pharmaceutical agent to a subject.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cleavable moiety” means a bond or group of atoms that is cleaved under physiological conditions, for example, inside a cell or a subject.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) with thymine (T), adenine (A) with uracil (U), cytosine (C) with guanine (G), and 5-methyl cytosine (mC) with guanine (G). Complementary oligonucleotides and/or nucleic acids need not have nucleobase complementarity at each nucleoside. Rather, some mismatches are tolerated. As used herein, “fully complementary” or “100% complementary” in reference to an oligonucleotide, or portion thereof, means that oligonucleotide, or portion thereof, is complementary to another oligonucleotide or nucleic acid at each nucleobase of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising cEt modified sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “double-stranded” refers to a region of hybridized nucleic acid(s). In certain embodiments, such double-strand results from hybridization of an oligonucleotide (or portion thereof) to a target region of a transcript. In certain embodiments, a double-strand results from hybridization of two oligonucleotides (or portions thereof) to one another. In certain embodiments, the hybridized regions are portions (including the entirety) of two separate molecules (e.g., no covalent bond connects the two complementary strands together). In certain embodiments, the hybridized regions are portions of the same molecule that have hybridized (e.g., a hairpin structure).

As used herein, “duplex” means a structure formed by two separate nucleic acid molecules at least a portion of which are complementary and that are hybridized to one another, but are not covalently bonded to one another.

As used herein, “gapmer” means a modified oligonucleotide comprising an internal region (gap segment) having at least 6 contiguous linked 2′-deoxynucleosides, a 5′ external region (5′ wing segment) having 1 to 7 linked nucleosides wherein at least 2 of the nucleosides comprise a modified sugar moiety, and a 3′ external region (3′ wing segment) having 1 to 7 linked nucleosides wherein at least 2 of the nucleosides comprise a modified sugar moiety.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” means the covalent linkage between contiguous nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in a free uptake assay in certain cell lines.

As used herein “prodrug” means a therapeutic agent in a form outside the body that is converted to a different form within a subject or cells thereof. Typically, conversion of a prodrug within the subject is facilitated by the action of an enzymes (e.g., endogenous or viral enzyme) or chemicals present in cells or tissues and/or by physiologic conditions.

As used herein, “kidney disease” means a condition of the kidney that reduces kidney function, such as urine production. Kidney diseases may be associated with or caused by a genetic mutation, diet, or a combination thereof. In certain embodiments, the kidney disease is chronic kidney disease (CKD).

As used herein, “kidney injury” means an injury of the kidney that reduces kidney function, such as urine production. Kidney injuries may be associated with or caused by a bodily impact, an infection, surgery, environmental toxin, or a combination thereof. In certain embodiment, the kidney injury is an acute kidney injury (AKI).

As used herein, “cardiac disorder” means a condition of the heart that reduces heart function, such as rhythm or circulation. Cardiac disorders may be associated with or caused by a genetic mutation, diet, or a combination thereof. In certain embodiments, the cardiac disorder is heart failure, hyperkalemia, a cardiomyopathy, or a cardiac arrhythmia.

As used herein, “cardiac injury” means damage to the heart that reduces heart function, such as rhythm or circulation. Cardiac injuries may be associated with or caused by a bodily impact, an infection, surgery, environmental toxin, or a combination thereof. The cardiac injury may be a myocardial infarction (MI).

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “reduced urine output” means an individual's a urine output is reduced relative to that of a healthy control subject that does not have a kidney injury or a kidney disease. In certain embodiments, administering an oligomeric compound disclosed herein or a pharmaceutical composition thereof to an individual with a kidney injury or kidney disease increases the individual's urine output relative to the individual's urine output before administering.

As used herein, “elevated serum creatinine levels” means an individual's serum creatinine levels are elevated relative to that of a healthy control subject that does not have a kidney injury or a kidney disease. In certain embodiments, administering an oligomeric compound disclosed herein or a pharmaceutical composition thereof to an individual with a kidney injury or kidney disease reduces the individual's serum creatinine levels relative to the individual's serum creatinine levels before administering.

As used herein, “elevated blood pressure” means a systolic blood pressure greater than 140 mmHg and/or a diastolic blood pressure greater than 90 mmHg. In certain embodiments, administering an oligomeric compound disclosed herein or a pharmaceutical composition thereof to an individual with a kidney injury or kidney disease reduces the individual's blood pressure relative to the individual's blood pressure before administering.

As used herein, “RNA” means an RNA transcript and includes pre-mRNA and mature mRNA unless otherwise specified.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “self-complementary” in reference to an oligonucleotide means an oligonucleotide that at least partially hybridizes to itself.

As used herein, “standard cell assay” means the assay described in Example 1 and reasonable variations thereof.

As used herein, “stereorandom” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “symptom” means any physical feature or test result that indicates the existence or extent of a disease or disorder. In certain embodiments, a symptom is apparent to a subject or to a medical professional examining or testing said subject.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to a subject. For example, a therapeutically effective amount improves a symptom of a disease.

### CERTAIN EMBODIMENTS

The present disclosure provides the following non-limiting numbered embodiments:
1. Embodiment 1. An oligomeric compound comprising a modified oligonucleotide consisting of 12 to 50, 12 to 45, 12 to 40, 12 to 35, 12 to 30, 12 to 25, or 12 to 20 linked nucleosides, wherein the nucleobase sequence of the modified oligonucleotide is at least 90% complementary to an equal length portion of a NLRP3 nucleic acid, and wherein the modified oligonucleotide comprises at least one modification selected from a modified sugar moiety and a modified internucleoside linkage.
2. Embodiment 2. An oligomeric compound comprising a modified oligonucleotide consisting of 12 to 30 linked nucleosides and having a nucleobase sequence comprising at least 8, 9, 10, 11, 12, 13, 14, 15, 16, 17, 18, 19, or 20 contiguous nucleobases complementary to an equal length portion of the nucleobase sequence of any one of the compounds of Tables 2-11.
3. Embodiment 3. An oligomeric compound comprising a modified oligonucleotide consisting of 12 to 50, 12 to 45, 12 to 40, 12 to 35, 12 to 30, 12 to 25, or 12 to 20 linked nucleosides and having a nucleobase sequence comprising at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases a hotspot region of Table 1.
4. Embodiment 4. The oligomeric compound of embodiment 3, wherein the modified oligonucleotide comprises at least 8, at least 9, at least 10, at least 11, at least 12, at least 13, at least 14, at least 15, at least 16, at least 17, at least 18, at least 19, or at least 20 contiguous nucleobases of the nucleobase sequence of an oligonucleotide of Table 1.
5. Embodiment 5. An oligomeric compound comprising a modified oligonucleotide consisting of 16 linked nucleosides, wherein the modified oligonucleotide has a nucleobase sequence of SEQ ID NO: 1454.
6. Embodiment 6. An oligomeric compound comprising a modified oligonucleotide consisting of 16 linked nucleosides, wherein the modified oligonucleotide has a nucleobase sequence of SEQ ID NO: 628.
7. Embodiment 7. An oligomeric compound comprising a modified oligonucleotide consisting of 16 linked nucleosides, wherein the modified oligonucleotide has a nucleobase sequence of SEQ ID NO: 178.
8. Embodiment 8. An oligomeric compound comprising a modified oligonucleotide consisting of 16 linked nucleosides, wherein the modified oligonucleotide has a nucleobase sequence of SEQ ID NO: 420.
9. Embodiment 9. The oligomeric compound of any one of embodiments 1-8, wherein the modified oligonucleotide has a nucleobase sequence that is at least 80%, 85%, 90%, 95%, or 100% complementary to an equal length portion of a nucleobase sequence selected from SEQ ID NO: 1 or SEQ ID NO: 2 when measured across the entire nucleobase sequence of the modified oligonucleotide.
10. Embodiment 10. The oligomeric compound of any one of embodiments 1-9, wherein at least one modified nucleoside comprises a modified sugar moiety.
11. Embodiment 11. The oligomeric compound of embodiment 10, wherein the modified sugar moiety comprises a bicyclic sugar moiety.
12. Embodiment 12. The oligomeric compound of embodiment 11, wherein the bicyclic sugar moiety comprises a 2′-4′ bridge selected from —O—CH₂—; and —O—CH(CH₃)—.
13. Embodiment 13. The oligomeric compound of embodiment 10, wherein the modified sugar moiety comprises a non-bicyclic modified sugar moiety.
14. Embodiment 14. The oligomeric compound of embodiment 13, wherein the non-bicyclic modified sugar moiety comprises a 2′-MOE sugar moiety or 2′-OMe sugar moiety.
15. Embodiment 15. The oligomeric compound of any one of embodiments 1-9, wherein at least one modified nucleoside comprises a sugar surrogate.
16. Embodiment 16. The oligomeric compound of embodiment 15, wherein the sugar surrogate is selected from morpholino and PNA.
17. Embodiment 17. The oligomeric compound of any of embodiments 1-16, wherein the modified oligonucleotide has a sugar motif comprising:
   a 5′ wing segment consisting of 1-5 linked nucleosides;
   a gap segment consisting of 6-10 linked nucleosides; and
   a 3′ wing segment consisting of 1-5 linked nucleosides,
   wherein each nucleoside of the 5′ wing segment and each nucleoside of the 3′ wing segment comprises a modified sugar moiety, and wherein each nucleoside of the gap segment comprises an unmodified 2′-deoxyribosyl sugar moiety.
18. Embodiment 18. The oligomeric compound of any one of embodiments 1-17, wherein the modified oligonucleotide comprises at least one modified internucleoside linkage.
19. Embodiment 19. The oligomeric compound of embodiment 18, wherein the modified internucleoside linkage is a phosphorothioate internucleoside linkage.
20. Embodiment 20. The oligomeric compound of embodiment 18, wherein each internucleoside linkage of the modified oligonucleotide is a modified internucleoside linkage.
21. Embodiment 21. The oligomeric compound of any one of embodiments 1-19, wherein the modified oligonucleotide comprises at least one phosphodiester internucleoside linkage.
22. Embodiment 22. The oligomeric compound of embodiment 20, wherein each internucleoside linkage is independently selected from a phosphodiester internucleoside linkage or a phosphorothioate internucleoside linkage.
23. Embodiment 23. The oligomeric compound of any of embodiments 1-22, wherein the modified oligonucleotide comprises at least one modified nucleobase.
24. Embodiment 24. The oligomeric compound of embodiment 23, wherein the modified nucleobase is a 5-methyl cytosine.
25. Embodiment 25. The oligomeric compound of any of embodiments 1-24, wherein the modified oligonucleotide consists of 12-30, 12-22, 12-20, 14-20, 15-25, 16-20, 18-22 or 18-20 linked nucleosides.
26. Embodiment 26. The oligomeric compound of any of embodiments 1-25, wherein the modified oligonucleotide consists of 16 linked nucleosides.
27. Embodiment 27. The oligomeric compound of embodiment 26, wherein each of nucleosides 1-3 and 14-16 (from 5′ to 3′) is a cEt nucleoside and each of nucleosides 4-13 is a 2′-deoxynucleoside.
28. Embodiment 28. The oligomeric compound of embodiment 26, wherein each of nucleosides 1, 3, 15 and 16 (from 5′ to 3′) is a cEt nucleoside, nucleosides 2, 13 and 14 is a 2′-MOE nucleoside, and each of nucleosides 4-12 is a 2′-deoxynucleoside.
29. Embodiment 29. The oligomeric compound of any of embodiments 1-28, consisting of the modified oligonucleotide.
30. Embodiment 30. The oligomeric compound of any of embodiments 1-28, comprising a conjugate group comprising a conjugate moiety and a conjugate linker.
31. Embodiment 31. The oligomeric compound of embodiment 30, wherein the conjugate group comprises 1-3 N-Acetylgalactosamine (GalNAc) groups.
32. Embodiment 32. The oligomeric compound of embodiments 30 or 31, wherein the conjugate linker consists of a single bond.
33. Embodiment 33. The oligomeric compound of embodiment 32, wherein the conjugate linker is cleavable.
34. Embodiment 34. The oligomeric compound of embodiment 33, wherein the conjugate linker comprises 1-3 linker-nucleosides.
35. Embodiment 35. The oligomeric compound of any of embodiments 30-34, wherein the conjugate group is attached to the modified oligonucleotide at the 5′-end of the modified oligonucleotide.
36. Embodiment 36. The oligomeric compound of any of embodiments 30-35, wherein the conjugate group is attached to the modified oligonucleotide at the 3′-end of the modified oligonucleotide.
37. Embodiment 37. The oligomeric compound of any of embodiments 1-36 comprising a terminal group.
38. Embodiment 38. The oligomeric compound of any of embodiments 1-37 wherein the oligomeric compound is a single-stranded oligomeric compound.
39. Embodiment 39. The oligomeric compound of any of embodiments 1-33 or 35-38, wherein the oligomeric compound does not comprise linker-nucleosides.
40. Embodiment 40. An oligomeric duplex comprising an oligomeric compound of any of embodiments 1-37 or 39.
41. Embodiment 41. An antisense compound comprising or consisting of an oligomeric compound of any of embodiments 1-39 or an oligomeric duplex of embodiment 40.
42. Embodiment 42. A modified oligonucleotide according to the following chemical structure:

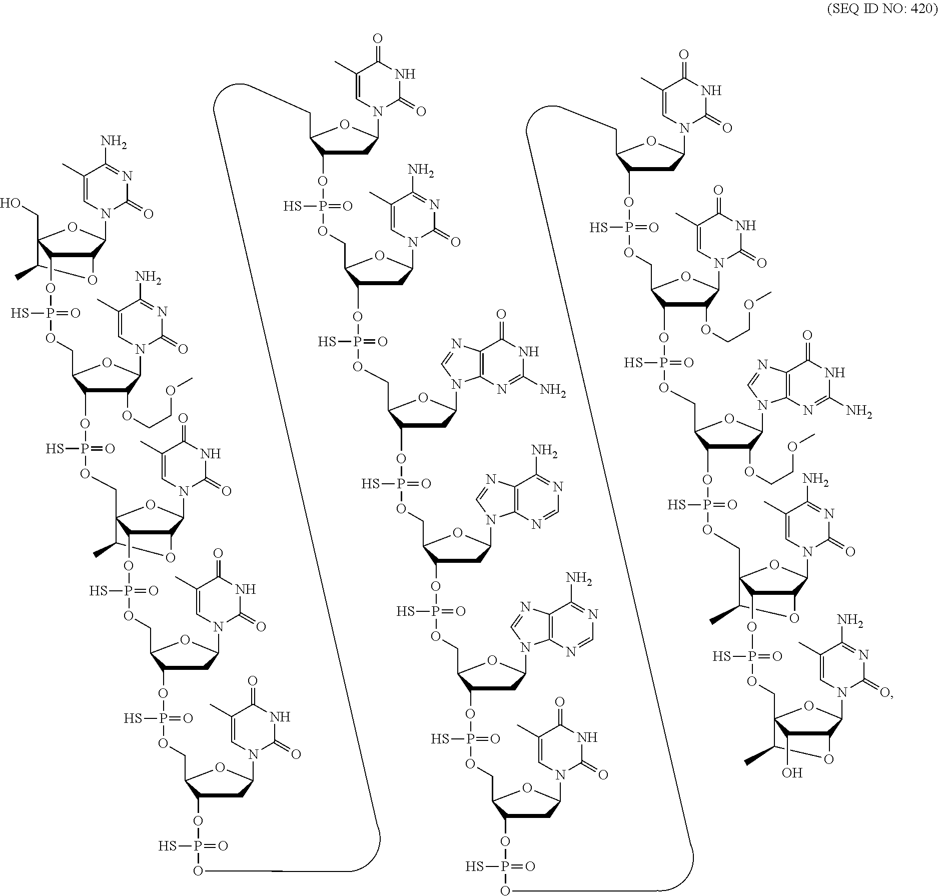

or a salt thereof.
43. Embodiment 43. The modified oligonucleotide of embodiment 42, which is a sodium salt or a potassium salt.
44. Embodiment 44. A modified oligonucleotide according to the following chemical structure:

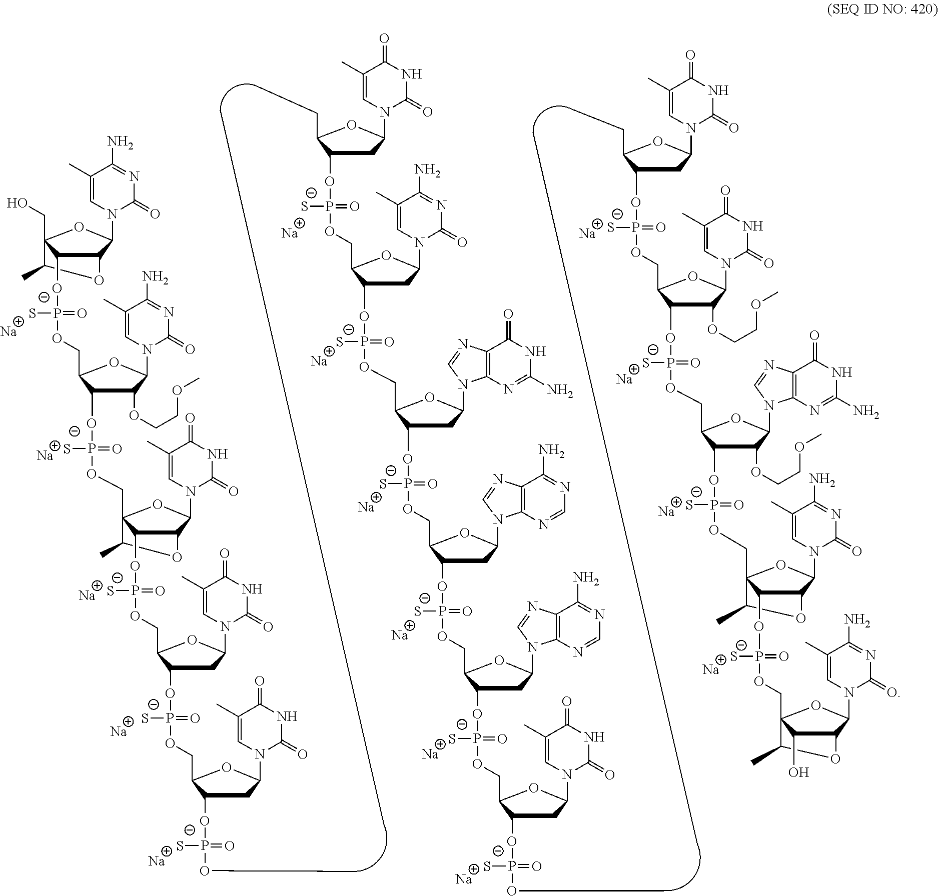

45. Embodiment 45. A modified oligonucleotide according to the following chemical notation:
   ^{m}Cₖₛ^{m}CₑₛTₖₛT_{ds}T_{ds}T_{ds}^{m}C_{ds}G_{ds}A_{ds}A_{ds}T_{ds}T_{ds}TₑₛGₑₛ^{m}Cₖₛ^{m}Cₖ (SEQ ID NO: 420); wherein
      A=an adenine nucleobase,
      mC=a 5-methyl cytosine nucleobase,
      G=a guanine nucleobase,
      T=a thymine nucleobase,
      k=a cEt modified sugar moiety,
      e=a 2′-MOE modified sugar moiety,
      d=a 2′-deoxyribose sugar, and
      s=a phosphorothioate internucleoside linkage.
46. Embodiment 46. A pharmaceutical composition comprising the oligomeric compound of any of embodiments 1-39, the oligomeric duplex of embodiment 40, the antisense compound of embodiment 41, or the modified oligonucleotides of any one of embodiments 42-45; and a pharmaceutically acceptable carrier or diluent.
47. Embodiment 47. The pharmaceutical composition of embodiment 46, wherein the pharmaceutically acceptable carrier or diluent comprises phosphate buffered saline.
48. Embodiment 48. The pharmaceutical composition of embodiment 47, consisting essentially of the oligomeric compound, antisense compound or oligomeric duplex, and phosphate buffered saline.
49. Embodiment 49. A method comprising administering to a subject the oligomeric compound of any of embodiments 1-39, the oligomeric duplex of embodiment 40, the antisense compound of embodiment 41, or the modified oligonucleotides of any one of embodiments 42-45, or the pharmaceutical composition of any of embodiments 46-48.
50. Embodiment 50. A method of treating a kidney disease or kidney injury comprising administering to a subject having or at risk for developing a kidney disease or kidney injury a therapeutically effective amount of the oligomeric compound of any of embodiments 1-39, the oligomeric duplex of embodiment 40, the antisense compound of embodiment 41, or the modified oligonucleotides of any one of embodiments 42-45, or the pharmaceutical composition of any of embodiments 46-48, thereby treating the kidney disease or kidney injury.
51. Embodiment 51. A method of reducing NLRP3 RNA or NLRP3 protein in a kidney, liver or heart of a subject having or at risk for developing a kidney disease or kidney injury comprising administering a therapeutically effective amount of the compound of any of embodiments 1-39, the oligomeric duplex of embodiment 40, the antisense compound of embodiment 41, or the modified oligonucleotides of any one of embodiments 42-45, or the pharmaceutical composition of any of embodiments 46-48, thereby reducing NLRP3 RNA or NLRP3 protein in the kidney, liver or heart.
52. Embodiment 52. The method of embodiment 50 or 51, wherein the kidney disease is chronic kidney disease (CKD).
53. Embodiment 53. The method of embodiment 50 or 51, wherein the kidney injury is acute kidney injury (AKI).
54. Embodiment 54. The method of any one of embodiments 50-53, wherein at least one symptom of the kidney disease or kidney injury is ameliorated.
55. Embodiment 55. The method of embodiment 54, wherein the symptom is selected from nausea, vomiting, loss of appetite, reduced urine output, elevated serum creatinine levels, muscle cramping, swelling, itching, chest pain, shortness of breath and elevated blood pressure.
56. Embodiment 56. The method of any of embodiments 50-55, wherein the method prevents or slows progression of the kidney disease or kidney injury.
57. Embodiment 57. Use of the oligomeric compound of any of embodiments 1-39, the oligomeric duplex of embodiment 40, the antisense compound of embodiment 41, or the modified oligonucleotides of any one of embodiments 42-45, or the pharmaceutical composition of any of embodiments 46-48 for the treatment of a kidney disease or kidney injury.
58. Embodiment 58. The use of embodiment 57, wherein the kidney disease is CKD.
59. Embodiment 59. The use of embodiment 57, wherein the kidney injury is AKI.

I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non-bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O- 2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(R_{b})═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently selected from: H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), and sulfoxyl (S(═O)-J₁); and each J₁ and J₂ is, independently selected from: H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, and a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

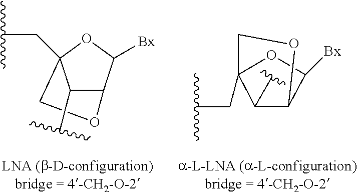

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

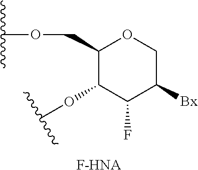

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

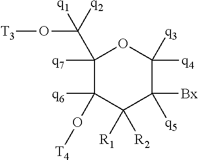

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

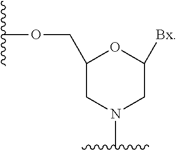

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

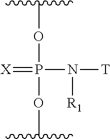

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.
   In certain embodiments, a modified internucleoside linkage comprises a mesyl phosphoramidate linking group having a formula:

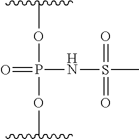 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

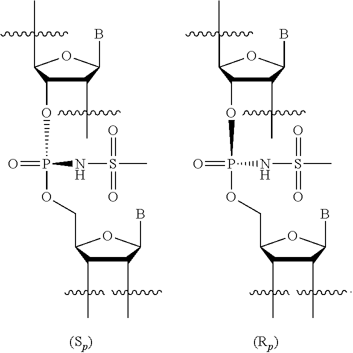

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration.

In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

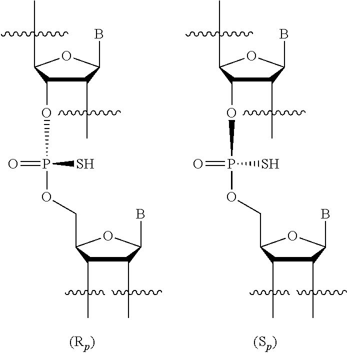

Unless otherwise indicated, chiral internucleoside linkages of modified oligonucleotides described herein can be stereorandom or in a particular stereochemical configuration.

In certain embodiments, populations of modified oligonucleotides comprise mesyl phosphoramidate internucleoside linkages wherein all of the mesyl phosphoramidate internucleoside linkages are stereorandom. In certain embodiments, populations of modified oligonucleotides comprise mesyl phosphoramidate internucleoside linkages wherein one or mesyl phosphoramidate internucleoside linkages is enriched for a particular configuration. In certain embodiments, populations of modified oligonucleotides comprise mesyl phosphoramidate internucleoside linkages wherein each mesyl phosphoramidate internucleoside linkage is enriched for a particular configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap segment (the 3′-most nucleoside of the 5′-wing and the 5′-most nucleoside of the 3′-wing) differ from the sugar moiety of the neighboring gap nucleosides, thus defining the boundary between the wings and the gap segment (i.e., the wing/gap junction). In certain embodiments, the sugar moieties within the gap are the same as one another. In certain embodiments, the gap segment includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap segment of a gapmer comprises 7-12 nucleosides. In certain embodiments, each nucleoside of the gap segment of a gapmer is an unmodified 2′-deoxynucleoside. In certain embodiments, at least one nucleoside of the gap segment of a gapmer is a modified nucleoside.

In certain embodiments, the gapmer is a deoxy gapmer. In certain embodiments, the nucleosides on the gap side of each wing/gap junction are unmodified 2′-deoxynucleosides and the nucleosides on the wing sides of each wing/gap junction are modified nucleosides. In certain embodiments, each nucleoside of the gap segment is an unmodified 2′-deoxynucleoside. In certain embodiments, each nucleoside of each wing of a gapmer is a modified nucleoside. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, one nucleoside of the gap comprises a modified sugar moiety and each remaining nucleoside of the gap comprises a 2′-deoxyribosyl sugar moiety. In certain embodiments, at least one nucleoside of the gap of a gapmer comprises a 2′-OMe sugar moiety.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing segment and the gap segment nucleosides comprise unmodified deoxynucleosides sugars. Thus, a 3-10-3 cEt gapmer consists of 3 linked cEt nucleosides in the 5′-wing, 10 linked deoxynucleosides in the gap, and 3 linked cEt nucleosides in the 3′-wing. Similarly, a 2-12-2 cEt gapmer consists of 2 linked cEt nucleosides in the 5′-wing, 12 linked deoxynucleosides in the gap, and 2 linked cEt nucleosides in the 3′-wing.

In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers. In certain embodiments, modified oligonucleotides are 2-12-2 BNA gapmers. In certain embodiments, modified oligonucleotides are 2-12-2 cEt gapmers. In certain embodiments, modified oligonucleotides are 2-12-2 LNA gapmers. In certain embodiments, modified oligonucleotides are 3-9-4 MOE/cEt gapmers. In certain embodiments, modified oligonucleotides are 2-9-5 MOE/cEt gapmers.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the gap segment of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate. In certain embodiments, the sugar motif of a modified oligonucleotide is a gapmer and the internucleoside linkages within the gap segment are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap segment comprises at least one Sp, Sp, Rp motif. In certain embodiments, populations of modified oligonucleotides are enriched for modified oligonucleotides comprising such internucleoside linkage motifs.

C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap segment of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

B. Certain Terminal Groups

In certain embodiments, oligomeric compounds comprise one or more terminal groups. In certain such embodiments, oligomeric compounds comprise a stabilized 5′-phophate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap segment of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap segment. In certain embodiments, the mismatch is at position 9, 8, 7, 6, 5, 4, 3, 2, 1 from the 3′-end of the gap segment. In certain embodiments, the mismatch is at position 1, 2, 3, or 4 from the 5′-end of the wing region. In certain embodiments, the mismatch is at position 4, 3, 2, or 1 from the 3′-end of the wing region.

B. NLRP3

In certain embodiments, oligomeric compounds comprise or consist of an oligonucleotide comprising a region that is complementary to a NLRP3 nucleic acid. In certain embodiments, the NLRP3 nucleic acid has the sequence set forth in SEQ ID NO: 1 (GENBANK Accession No. NC_000001.11, truncated from nucleosides 247413001 to 247454000), to SEQ ID NO: 2 (GENBANK Accession No. NM_004895.4), to SEQ ID NO: 3 (GENBANK Accession No. NM_183395.2), to SEQ ID NO: 4 (GENBANK Accession No. NM_001127461.2), to SEQ ID NO: 5 (GENBANK Accession No. NM_001079821.2).

In certain embodiments an oligomeric compound complementary to any one of SEQ ID NOs: 1-5 is capable of reducing an NLRP3 RNA in a cell. In certain embodiments an oligomeric compound complementary to any one of SEQ ID NOs: 1-5 is capable of reducing an NLRP3 protein in a cell. In certain embodiments, the cell is in vitro. In certain embodiments, the cell is in a subject. In certain embodiments, the oligomeric compound consists of a modified oligonucleotide.

In certain embodiments, an oligomeric compound complementary to any one of SEQ ID NOs: 1-5 is capable of ameliorating one or more symptoms of a kidney injury or kidney disease when it is administered to an individual in need thereof. In certain embodiments, the one or more symptoms are selected from nausea, vomiting, loss of appetite, reduced urine output, elevated serum creatinine levels, muscle cramping, swelling, itching, chest pain, shortness of breath and elevated blood pressure, or a combination thereof. In certain embodiments, the kidney injury or kidney disease is selected from acute kidney injury (AKI) and chronic kidney disease (CKD).

In certain embodiments, an oligomeric compound complementary to any one of SEQ ID NOs: 1-5 is capable of ameliorating one or more symptoms of a cardiac disorder or cardiac injury when it is administered to an individual in need thereof. In certain embodiments, the one or more symptoms are selected from pain, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, blood clots, or a combination thereof. In certain embodiments, the cardiac disorder or cardiac injury is heart failure, hyperkalemia, a cardiomyopathy, or a cardiac arrhythmia. The cardiac disorder may be associated with, or arise from, a cardiac injury. For example, the arrhythmia may be an MI-associated cardiac arrhythmia. The cardiomyopathy may be RBM20 cardiomyopathy. The cardiomyopathy may be associated with diabetes, long QT syndrome 2 (LQT2), catecholaminergic polymorphic ventricular tachycardia (CPVT), administration of doxorubicin, or acute beta-adrenergic stress.

The subject may have a cardiac disorder or cardiac injury. The cardiac disorder or cardiac injury may be heart failure, hypokalemia, a cardiomyopathy, or a cardiac arrhythmia. The cardiomyopathy may be, for example, hypertrophic cardiomyopathy, dilated cardiomyopathy, restrictive cardiomyopathy, arrhythmogenic right ventricular dysplasia, or Takotsubo cardiomyopathy (broken heart syndrome). The cardiomyopathy may be dilated cardiomyopathy. In certain embodiments, dilated cardiomyopathy is arrhythmogenic. In certain embodiments, dilated cardiomyopathy is genetic, in which the subject has a mutation in one or more of TTN, LMNA, RBM20, SCN5A, MYH7, TNNT2, and TPM1. The cardiomyopathy may be RBM20 cardiomyopathy. Cardiac arrhythmias may include, but are not limited to, atrial or ventricular arrhythmia, for example, atrial fibrillation (AF), ventricular fibrillation, or ventricular tachycardia (VT). The cardiac injury may be myocardial infarction (MI). The cardiac disorder may be an MI-associated arrhythmia, for example, MI-associated ventricular arrhythmia. Symptoms of cardiac disorders and cardiac injuries include, but are not limited to, pain, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, and/or blood clots. In some embodiments, the materials and methods provided herein improve one or more indices of heart function. Indices of heart function include cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, and heart rhythm in the subject. In certain embodiments, the compounds, methods, and pharmaceutical compositions are useful in reducing a progression of heart failure. Progression of heart failure may be classified according to a method known in the art or as provided herein.

Heart failure may be classified according to the New York Heart Association classification. The New York Heart Association classifies heart failure in four categories. In Class I heart failure, no symptoms are observed. In Class II heart failure, everyday activities can be performed without difficulty but a subject may experience dyspnea (shortness of breath), palpitation, or feel fatigued upon exertion. In Class III, completing everyday activities is difficult, and a subject may experience marked limitation of physical activity due to fatigue, palpitation, or dyspnea. and in Class IV, the most severe, a subject is unable to carry on any physical activity without discomfort, and the subject is short of breath even at rest.

Heart failure may be classified according to the American College of Cardiology/American Heart Association guidelines. In Class A, while a subject may have risk factors for heart failure, no objective evidence of cardiovascular disease is observed, and no limitation in ordinary physical activity is noted. In Class B, there is observed objective evidence of a minimal (structural) cardiovascular condition, including mild symptoms and slight limitation during ordinary activity, but the subject is comfortable at rest. In Class C, there is observed objective evidence of a moderately severe (structural) cardiovascular condition, including marked limitation in activity due to symptoms, even during less-than-ordinary activity, and the subject is comfortable only at rest. In Class D, there is observed objective evidence of a severe (structural) cardiovascular condition, and severe limitations, and the subject experiences symptoms even while at rest. Some specific structural cardiac conditions include mitral valve regurgitation or stenosis, aortic stenosis, coarctation of the aorta (mild to severe narrowing in the aorta), ventricular septal defects.

Examples of diseases associated with NLRP3 treatable with the oligomeric agents, oligomeric compounds, modified oligonucleotides, oligomeric duplexes, and methods provided herein include heart failure, hypokalemia, a cardiomyopathy, or a cardiac arrhythmia. In certain embodiments, cardiomyopathy is dilated cardiomyopathy. In certain embodiments, dilated cardiomyopathy is genetic, including TTN, LMNA, RBM20, SCN5A, MYH7, TNNT2, and TPM1 mutations. In certain embodiments, dilated cardiomyopathy is arrhythmogenic. In certain embodiments, cardiac arrhythmias is atrial fibrillation (AF), ventricular fibrillation, or ventricular tachycardia (VT). Further embodiment provide for improving an index of heart function selected from cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, and heart rhythm associated with heart failure, hypokalemia, a cardiomyopathy, or a cardiac arrhythmia.

Examples of conditions associated with NLRP3 treatable with the oligomeric agents, oligomeric compounds, modified oligonucleotides, oligomeric duplexes, and methods provided herein include a kidney injury or kidney disease, or a symptom thereof. In certain embodiments, the symptom is selected from nausea, vomiting, loss of appetite, reduced urine output, elevated serum creatinine levels, muscle cramping, swelling, itching, chest pain, shortness of breath and elevated blood pressure, or a combination thereof. In certain embodiments, the kidney injury or kidney disease is selected from acute kidney injury (AKI) and chronic kidney disease (CKD).

In certain embodiments, an oligomeric agent, oligomeric compound, modified oligonucleotide, or oligomeric duplex is for the manufacture or preparation of a medicament for treating or ameliorating a condition described herein.

In certain embodiments, an oligomeric compound complementary to any one of SEQ ID NOs: 1-5 is capable of reducing a detectable amount of an NLRP3 RNA in the plasma/serum, blood, kidney, liver or heart of a subject when the oligomeric compound is administered to the subject. In some instances, the oligomeric compound is administered subcutaneously. The detectable amount of the NLRP3 RNA may be reduced by at least 10%, at least 20%, at least 30%, at least 40%, at least 50%, at least 60%, at least 70%, at least 80%, or at least 90%. In certain embodiments, an oligomeric compound complementary to any one of SEQ ID NOs: 1-5 is capable of reducing a detectable amount of an NLRP3 protein in the plasma/serum blood, kidney, liver or heart of the subject when the oligomeric compound is administered to the subject. The detectable amount of the NLRP3 protein may be reduced by at least 10%, at least 20%, at least 30%, at least 40%, at least 50%, at least 60%, at least 70%, at least 80%, or at least 90%.

VI. Certain Hotspot Regions

In certain embodiments, the ranges described in Table 1 below comprise hotspot regions. Each hotspot region begins with the nucleobase of SEQ ID NO: 1 identified in the “Start Site SEQ ID NO: 1” column and ends with the nucleobase of SEQ ID NO: 1 identified in the “Stop Site SEQ ID NO: 1” column. In certain embodiments, oligomeric compounds comprise modified oligonucleotides that are complementary within any of the hotspot regions 1-11, as defined in Table 1 below. In certain embodiments, modified oligonucleotides are 16 nucleobases in length. In certain embodiments, modified oligonucleotides are 17 nucleobases in length. In certain embodiments, modified oligonucleotides are 18 nucleobases in length. In certain embodiments, modified oligonucleotides are 19 nucleobases in length. In certain embodiments, modified oligonucleotides are 20 nucleobases in length.

The nucleobase sequences of the compounds listed in the second to last column of Table 1 are complementary to SEQ ID NO: 1 within the specified hotspot regions defined by the provided Start Sites of SEQ ID NO: 1 (column 2) and Stop Sites of SEQ ID NO: 1 (column 3). The nucleobase sequences of these compounds are provided in the last column (“SEQ ID NOs in range”) in the same order as the compounds.

In certain embodiments, modified oligonucleotides complementary to nucleobases within the hotspot region achieve at least minimum % reduction, relative to untreated control cells, of NLRP3 RNA in vitro in the standard cell assay, (“Min.% Red. In vitro”), as indicated in Table 1. In certain embodiments, modified oligonucleotides complementary to nucleobases within the hotspot region achieve a maximum % reduction, relative to untreated control cells, of NLRP3 RNA in vitro in the standard cell assay, (“Max. % Red. In vitro”) as indicated in Table 1. In certain embodiments, modified oligonucleotides complementary to nucleobases within the hotspot region achieve an average % reduction, relative to untreated control cells, of NLRP3 RNA in vitro in the standard cell assay, (“Avg % Red. In vitro”) as indicated in Table 1.

TABLE 1
NLRP3 Hotspot Regions

| | Start Site | Stop Site | Min % | Max % | Avg % | |
|---|---|---|---|---|---|---|
| Hotspot | SEQ ID | SEQ ID | Red. In | Red. In | Red. In | |
| ID | NO: 1 | NO: 1 | vitro | vitro | vitro | Compound NOs in range |
| 1 | 12138 | 12155 | 41 | 78 | 61.9 | 1232844, 1232845, 1232846, |
| | | | | | | 1299765, 1299769, 1299773, |
| | | | | | | 1300531, 1300535, 1300539, |
| | | | | | | 1300983, 1300984, 1300987, |
| | | | | | | 1304716, 1304770, 1304822 |
| 2 | 14478 | 14523 | 21 | 88 | 71.9 | 1233082, 1233083, 1242439, |
| | | | | | | 1242440, 1242441, 1242442, |
| | | | | | | 1242443, 1242444, 1242445, |
| | | | | | | 1242446, 1242447, 1242448, |
| | | | | | | 1242449, 1242450, 1242451, |
| | | | | | | 1242452, 1242453, 1242454, |
| | | | | | | 1242455, 1242456, 1242457, |
| | | | | | | 1242458, 1242459, 1242460, |
| | | | | | | 1299631, 1299855, 1299865, |
| | | | | | | 1299877, 1300395, 1300623, |
| | | | | | | 1300635, 1300641, 1300850, |
| | | | | | | 1301074, 1301092, 1301098, |
| | | | | | | 1301199, 1304701, 1304704, |
| | | | | | | 1304707, 1304721, 1304723, |
| | | | | | | 1304735, 1304745, 1304763, |
| | | | | | | 1304766, 1304774, 1304803, |
| | | | | | | 1304812, 1304829 |
| 3 | 14578 | 14622 | 34 | 94 | 68.9 | 1242480, 1242481, 1242482, |
| | | | | | | 1242483, 1242484, 1242485, |
| | | | | | | 1242486, 1242487, 1242488, |
| | | | | | | 1242489, 1242490, 1242491, |
| | | | | | | 1242492, 1242493, 1242494, |
| | | | | | | 1242495, 1242496, 1242497, |
| | | | | | | 1242498, 1299635, 1299643, |
| | | | | | | 1300405, 1300406, 1300858, |
| | | | | | | 1300859, 1304689, 1304690, |
| | | | | | | 1304696, 1304711, 1304731, |
| | | | | | | 1304737, 1304746, 1304758, |
| | | | | | | 1304794, 1304798 |
| 4 | 14667 | 14693 | 43 | 93 | 75.8 | 1242499, 1242500, 1242501, |
| | | | | | | 1242502, 1242503, 1242504, |
| | | | | | | 1242505, 1242506, 1304698, |
| | | | | | | 1304709, 1304714, 1304728, |
| | | | | | | 1304739 |
| 5 | 15111 | 15130 | 61 | 90 | 77.1 | 1242516, 1242517, 1242518, |
| | | | | | | 1299656, 1299657, 1299659, |
| | | | | | | 1300421, 1300422, 1300424, |
| | | | | | | 1300874, 1300875, 1300876, |
| | | | | | | 1304724, 1304751 |
| 6 | 15182 | 15200 | 62 | 97 | 76.9 | 1242519, 1299658, 1299666, |
| | | | | | | 1299673, 1300431, 1300438, |
| | | | | | | 1300440, 1300883, 1300889, |
| | | | | | | 1300892, 1301213, 1304730 |
| 7 | 17668 | 17686 | 34 | 87 | 67.3 | 1233103, 1233104, 1242546, |
| | | | | | | 1242547, 1299676, 1299681, |
| | | | | | | 1299683, 1299684, 1300441, |
| | | | | | | 1300447, 1300449, 1300450, |
| | | | | | | 1300897, 1300898, 1300899, |
| | | | | | | 1300901, 1304749, 1304783, |
| | | | | | | 1304807 |
| 8 | 20272 | 20291 | 31 | 89 | 68.1 | 1242601, 1299796, 1299797, |
| | | | | | | 1299805, 1299809, 1299812, |
| | | | | | | 1300572, 1300575, 1300576, |
| | | | | | | 1300578, 1300580, 1301015, |
| | | | | | | 1301024, 1301026, 1301029, |
| | | | | | | 1301030, 1301277, 1301281, |
| | | | | | | 1301283, 1304702 |
| 9 | 20634 | 20657 | 51 | 89 | 69.7 | 1242626, 1242627, 1242628, |
| | | | | | | 1242629, 1242630, 1242631, |
| | | | | | | 1242632, 1304685, 1304686, |
| | | | | | | 1304699, 1304733 |
| 10 | 23475 | 23512 | 12 | 89 | 64.7 | 1233172, 1233173, 1233174, |
| | | | | | | 1233175, 1233176, 1233176, |
| | | | | | | 1233177, 1242724, 1299705, |
| | | | | | | 1299708, 1299709, 1299711, |
| | | | | | | 1299713, 1299715, 1299724, |
| | | | | | | 1300472, 1300474, 1300477, |
| | | | | | | 1300480, 1300481, 1300485, |
| | | | | | | 1300489, 1300924, 1300925, |
| | | | | | | 1300926, 1300929, 1300935, |
| | | | | | | 1300940, 1300941, 1301225, |
| | | | | | | 1301228, 1304712, 1304778, |
| | | | | | | 1304781, 1304821 |
| 11 | 25026 | 25049 | 47 | 84 | 70.1 | 1242821, 1242822, 1299857, |
| | | | | | | 1299858, 1300636, 1300640, |
| | | | | | | 1301075, 1301085, 1301314, |
| | | | | | | 1304688 |

VII. Certain Compounds

1. Compound No. 1233279

In certain embodiments, the oligomeric compound is Compound No. 1233279. In certain embodiments, Compound No. 1233279 is characterized as an oligomeric compound consisting of a modified oligonucleotide, wherein the modified oligonucleotide is a 3-10-3 cEt gapmer, having a sequence of (from 5′ to 3′) AACTATTAAGCAACGG (SEQ ID NO: 628), wherein each of nucleosides 1-3 (from 5′ to 3′) comprise a cEt modified sugar moiety, each of nucleosides 14-16 (from 5′ to 3′) comprise a cEt modified sugar moiety, and each of nucleosides 4-13 are 2′-deoxynucleosides, wherein the internucleoside linkages between all nucleosides are phosphorothioate linkages, and wherein each cytosine is a 5-methyl cytosine.

In certain embodiments, Compound 1233279 is characterized by the following chemical notation: AₖₛAₖₛ^{m} CₖₛT_{ds}A_{ds}T_{ds}T_{ds}A_{ds}A_{ds}G_{ds}^{m}C_{ds}A_{ds}A_{ds}^{m}CₖₛGₖₛGₖ (SEQ ID NO: 628); wherein
A=an adenine nucleobase,
mC=a 5-methyl cytosine nucleobase,
G=a guanine nucleobase,
T=a thymine nucleobase,
k=a cEt modified sugar moiety,
d=a 2′-β-D-deoxyribosyl sugar moiety, and
s=a phosphorothioate internucleoside linkage.

In certain embodiments, Compound No. 1233279 is represented by the following chemical structure:

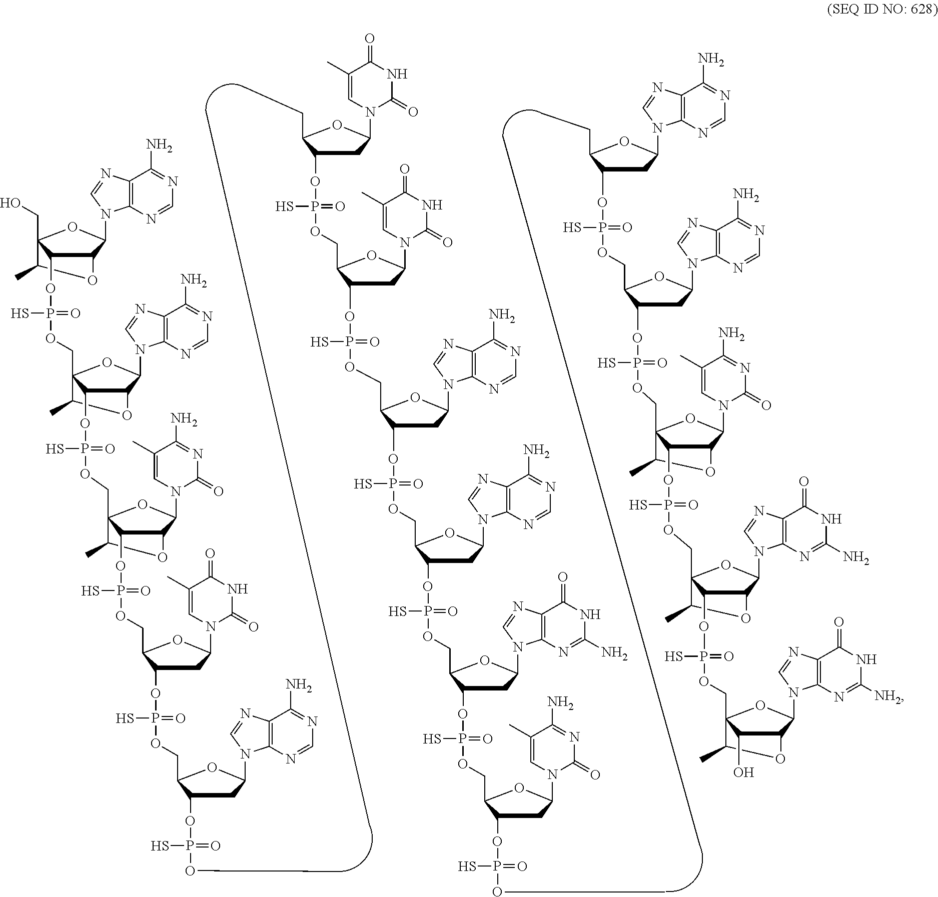

or a salt thereof. In certain embodiments, Compound No. 1233279 is in the form of an anion or a salt thereof. For example, the oligomeric compound may be in the form of a sodium salt. In certain embodiments, the oligomeric compound is in anionic form in a solution. In certain embodiments, Compound No. 1233279 is a sodium salt or a potassium salt.

In certain embodiments, Compound No. 1233279 is represented by the following chemical structure:

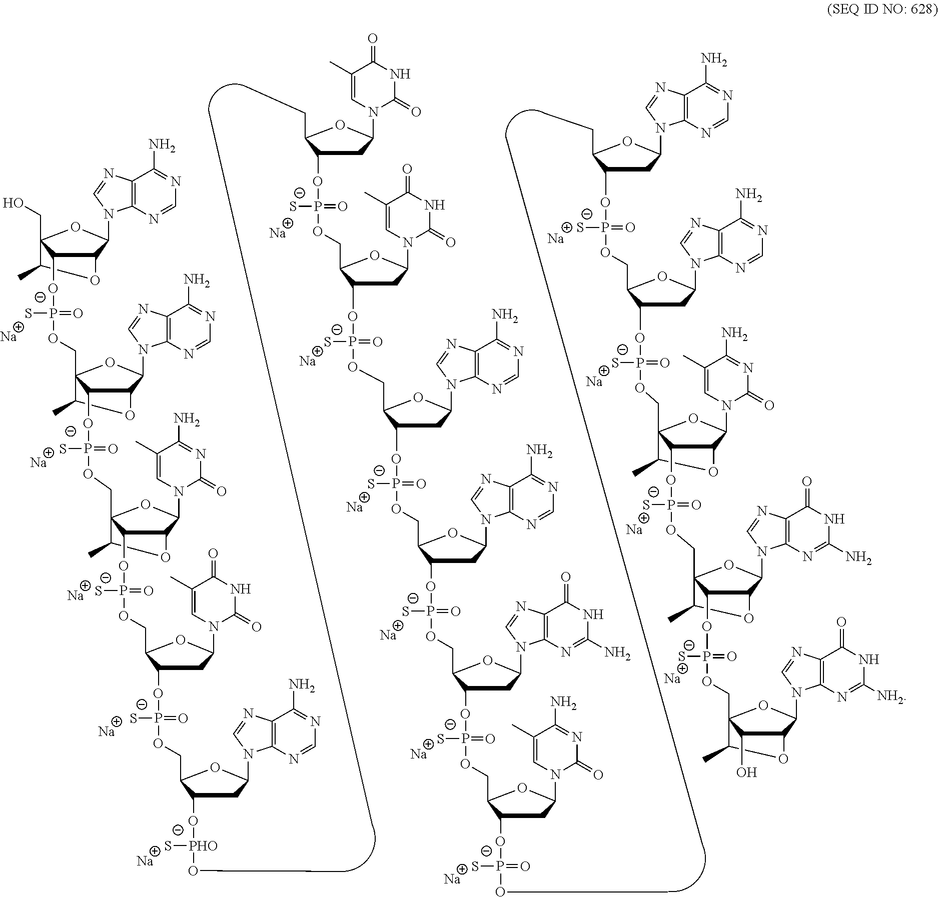

2. Compound No. 1242547

In certain embodiments, the oligomeric compound is Compound No. 1242547. In certain embodiments, Compound No. 1242547 is characterized as an oligomeric compound consisting of a modified oligonucleotide, wherein the modified oligonucleotide is a 3-10-3 cEt gapmer, having a sequence of (from 5′ to 3′) TGGAATATATCGAGCA (SEQ ID NO: 1454), wherein each of nucleosides 1-3 (from 5′ to 3′) comprise a cEt modified sugar moiety, each of nucleosides 14-16 (from 5′ to 3′) comprise a cEt modified sugar moiety, and each of nucleosides 4-13 are 2′-deoxynucleosides, wherein the internucleoside linkages between all nucleosides are phosphorothioate linkages, and wherein each cytosine is a 5-methyl cytosine.

In certain embodiments, Compound 1242547 is characterized by the following chemical notation: TₖₛGₖₛGₖₛA_{ds}A_{ds}T_{ds}A_{ds}T_{ds}A_{ds}T_{ds}^{m}C_{ds}G_{ds}A_{ds}Gₖₛ^{m}CₖₛAₖ (SEQ ID NO: 1454); wherein
A=an adenine nucleobase,
mC=a 5-methyl cytosine nucleobase,
G=a guanine nucleobase,
T=a thymine nucleobase,
k=a cEt modified sugar moiety,
d=a 2′-β-D-deoxyribosyl sugar moiety, and
s=a phosphorothioate internucleoside linkage.

In certain embodiments, Compound No. 1242547 is represented by the following chemical structure:

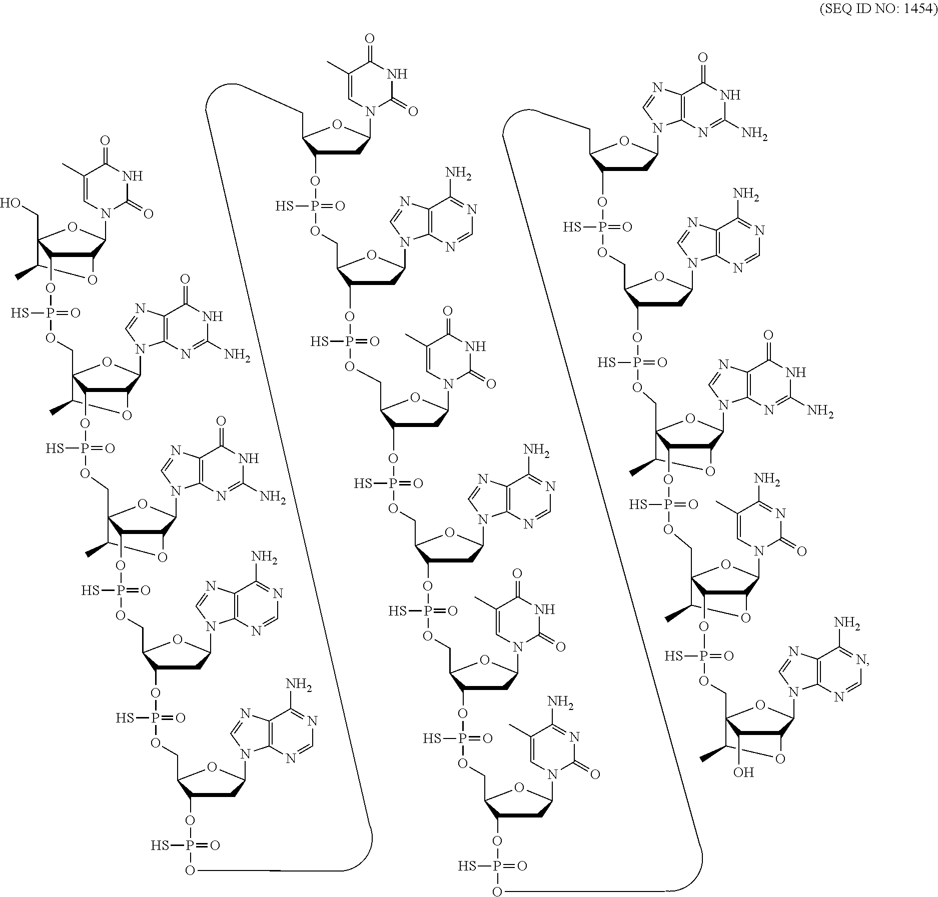

or a salt thereof. In certain embodiments, Compound No. 1242547 is in the form of an anion or a salt thereof. For example, the oligomeric compound may be in the form of a sodium salt. In certain embodiments, the oligomeric compound is in anionic form in a solution. In certain embodiments, Compound No. 1242547 is a sodium salt or a potassium salt.

In certain embodiments, Compound No. 1242547 is represented by the following chemical structure:

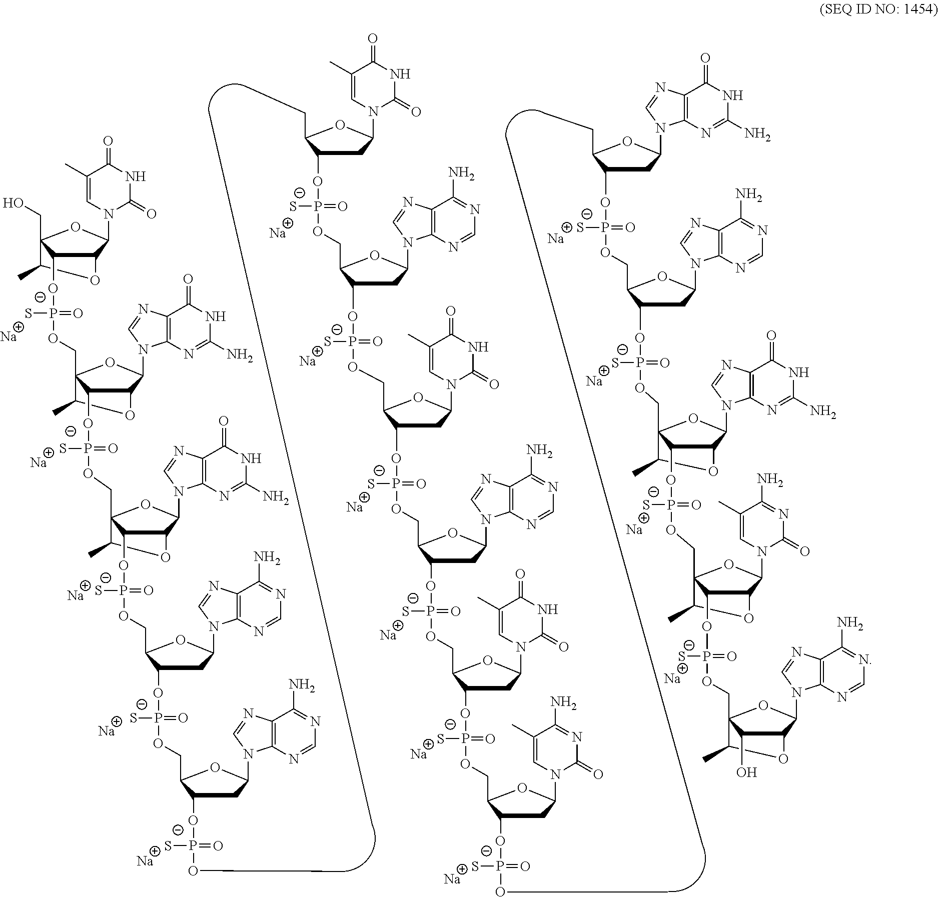

3. Compound No. 1299773

In certain embodiments, the oligomeric compound is Compound No. 1299773. In certain embodiments, Compound No. 1299773 is characterized as an oligomeric compound consisting of a modified oligonucleotide, wherein the modified oligonucleotide is a 3-9-4 MOE/cEt gapmer, having a sequence of (from 5′ to 3′) CCTTTTCGAATTTGCC (SEQ ID NO: 420), wherein each of nucleosides 1, 3, 15 and 16 (from 5′ to 3′) comprise a cEt modified sugar moiety, each of nucleosides 2, 13 and 14 (from 5′ to 3′) comprise a 2′-MOE modified sugar moiety, and each of nucleosides 4-12 are 2′-deoxynucleosides, wherein the internucleoside linkages between all nucleosides are phosphorothioate linkages, and wherein each cytosine is a 5-methyl cytosine.

In certain embodiments, Compound 1299773 is characterized by the following chemical notation: ^{m}Cₖₛ^{m}CₑₛTₖₛTₐₛTₐₛTₐₛ^{m}C_{ds}GₐₛA_{ds}A_{ds}TₐₛTₐₛTₑₛGₑₛ^{m}Cₖₛ^{m}Cₖ (SEQ ID NO: 420); wherein
A=an adenine nucleobase,
mC=a 5-methyl cytosine nucleobase,
G=a guanine nucleobase,
T=a thymine nucleobase,
k=a cEt modified sugar moiety,
e=a 2′-MOE β-D-ribosyl sugar moiety,
d=a 2′-β-D-deoxyribosyl sugar moiety, and
s=a phosphorothioate internucleoside linkage.

In certain embodiments, Compound No. 1299773 is represented by the following chemical structure:

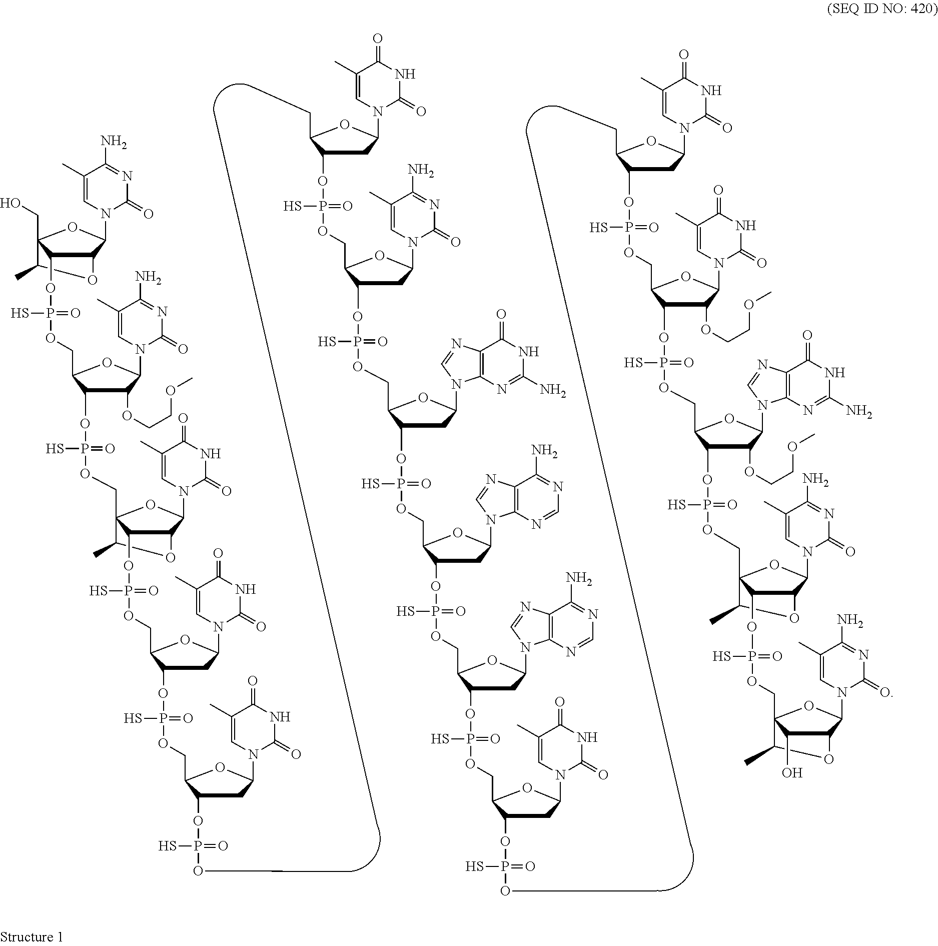

In certain embodiments, Compound No. 1299773 is in the form of an anion or a salt thereof. For example, the oligomeric compound may be in the form of a sodium salt. In certain embodiments, the oligomeric compound is in anionic form in a solution. In certain embodiments, Compound No. 1299773 is a sodium salt or a potassium salt.

In certain embodiments, Compound No. 1299773 is represented by the following chemical structure:

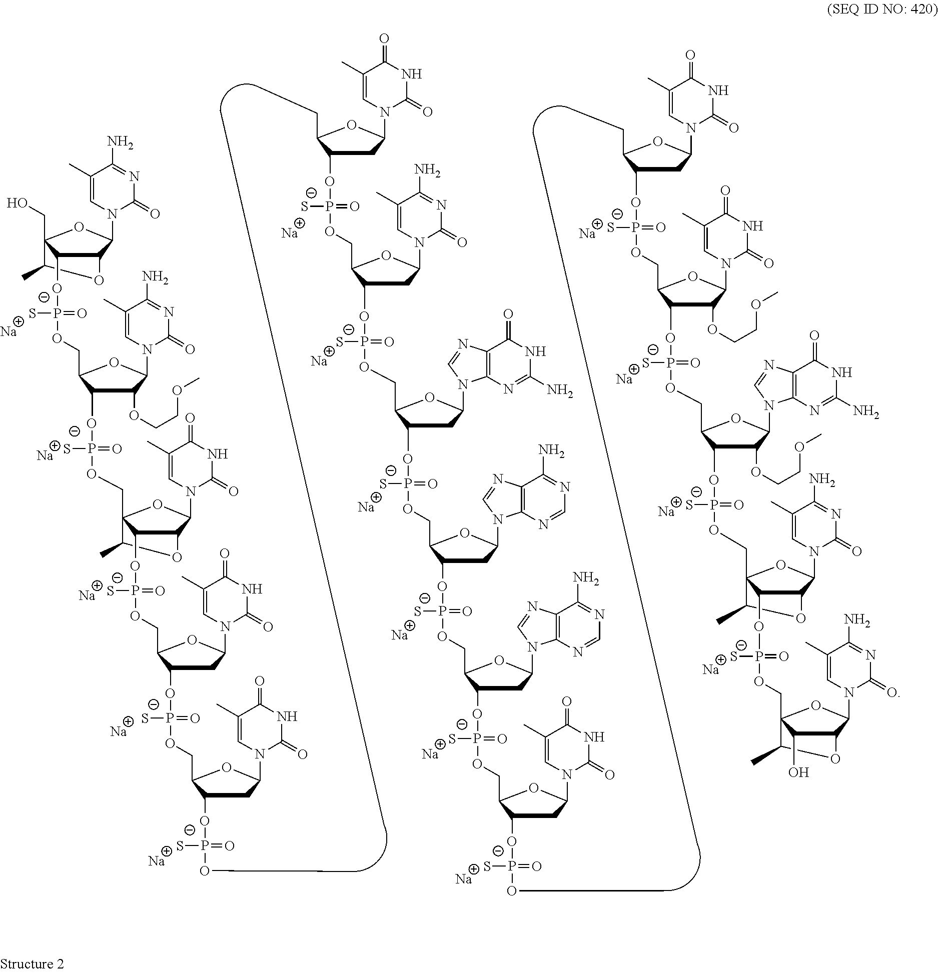

VIII. Certain Pharmaceutical Compositions & Delivery Systems

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Certain embodiments provide pharmaceutical compositions suitable for aerosolization and/or dispersal by a nebulizer or inhaler. In certain embodiments, the pharmaceutical composition is a solid comprising particles of compounds that are of respirable size. A solid particulate composition can optionally contain a dispersant which serves to facilitate the formation of an aerosol, e.g., lactose. Solid pharmaceutical compositions comprising an oligonucleotide can also be aerosolized using any solid particulate medicament aerosol generator known in the art, e.g., a dry powder inhaler. In certain embodiments, the powder employed in the inhaler consists of the compound comprising the active compound or of a powder blend comprising the active compound, a suitable powder diluent, and an optional surfactant. In certain embodiments, the pharmaceutical composition is a liquid. In certain such embodiments, the liquid is administered as an aerosol that is produced by any suitable means, such as with a nebulizer or inhaler. See, e.g., U.S. Pat. No. 4,501,729. In certain embodiments, the nebulizer is a device for producing a spray of liquid. Nebulizers are devices that transform solutions or suspensions into an aerosol mist and are well known in the art. Suitable nebulizers include jet nebulizers, ultrasonic nebulizers, electronic mesh nebulizers, and vibrating mesh nebulizers. In certain embodiments, the nebulizer is activated manually by squeezing a flexible bottle that contains the pharmaceutical composition. In certain embodiments, the aerosol is produced by a metered dose inhaler, which typically contains a suspension or solution formulation of the active compound in a liquefied propellant. Pharmaceutical compositions suitable for aerosolization can comprise propellants, surfactants, co-solvents, dispersants, preservatives, and/or other additives or excipients.

A compound described herein complementary to an NLRP3 nucleic acid can be utilized in pharmaceutical compositions by combining the compound with a suitable pharmaceutically acceptable diluent or carrier and/or additional components such that the pharmaceutical composition is suitable for aerosolization by a nebulizer or inhaler. In certain embodiments, a pharmaceutically acceptable diluent is phosphate buffered saline. Accordingly, in one embodiment, employed in the methods described herein is a pharmaceutical composition comprising a compound complementary to an NLRP3 nucleic acid and a pharmaceutically acceptable diluent. In certain embodiments, the pharmaceutically acceptable diluent is phosphate buffered saline. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts. A prodrug can include the incorporation of additional nucleosides at one or both ends of a compound which are cleaved by endogenous nucleases within the body, to form the active compound.

Under certain conditions, certain compounds disclosed herein are shown in the form of a free acid. Although such compounds may be drawn or described in protonated (free acid) form, aqueous solutions of such compounds may exist in equilibrium among an ionized (anion) form, and in association with a cation (salt form). For example, a phosphate linkage of an oligonucleotide in aqueous solution exists in equilibrium among free acid, anion, and salt forms. Unless otherwise indicated, compounds described herein are intended to include all such forms. Moreover, oligonucleotides have several such linkages, each of which is in equilibrium. Thus, oligonucleotides in solution exist in an ensemble of forms at multiple positions, all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salts thereof” expressly includes all such forms that may be fully or partially protonated/de-protonated/in association with a cation. In certain instances, one or more specific cation is identified.

In certain embodiments, oligomeric compounds disclosed herein are in a form of a sodium salt. In certain embodiments, oligomeric compounds disclosed herein are in a form of a potassium salt. In certain embodiments, oligomeric compounds disclosed herein are in aqueous solution with sodium. In certain embodiments, oligomeric compounds are in aqueous solution with potassium. In certain embodiments, oligomeric compounds are in PBS. In certain embodiments, oligomeric compounds are in water. In certain such embodiments, the pH of the solution is adjusted with NaOH and/or HCl to achieve a desired pH.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein mC indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or 13 such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of 3-10-3 cEt Uniform Phosphorothioate Modified Oligonucleotides on Human NLRP3 RNA In Vitro, Single Dose

Modified oligonucleotides complementary to human NLRP3 nucleic acid were designed and tested for their single dose effects on NLRP3 RNA in vitro. The modified oligonucleotides were tested in a series of experiments that had the same culture conditions.

The modified oligonucleotides in the tables below are 3-10-3 cEt modified oligonucleotides with uniform phosphorothioate internucleoside linkages. The modified oligonucleotides are 16 nucleosides in length, wherein the central gap segment consists of ten 2′-O-D-deoxynucleosides, and wherein the 5′ and 3′ wing segments each consist of three cEt modified nucleosides. The sugar motif for the modified oligonucleotides is (from 5′ to 3′): kkkddddddddddkkk; wherein each “d” represents a 2′-β-D-deoxyribosyl sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (GENBANK Accession No. NC_000001.11, truncated from nucleosides 247413001 to 247454000), to SEQ ID NO: 2 (GENBANK Accession No. NM_004895.4), to SEQ ID NO: 3 (GENBANK Accession No. NM_183395.2), to SEQ ID NO: 4 (GENBANK Accession No. NM_001127461.2), to SEQ ID NO: 5 (GENBANK Accession No. NM_001079821.2), or to any combination of these SEQ ID NOs. “N/A” indicates that the modified oligonucleotide is not 100% complementary to that particular target nucleic acid sequence.

Cultured THP-1 cells were treated with modified oligonucleotide at a concentration of 2000 nM by electroporation at a density of 300,000 cells per well. After a treatment period of approximately 24 hours, total RNA was isolated from the cells and NLRP3 RNA levels were measured by quantitative real-time RTPCR. NLRP3 RNA levels were measured by human primer-probe set RTS37509 (forward sequence GATGTTCTGTGAAGTGCTGAAAC, designated herein as SEQ ID NO: 10; reverse sequence AGCTCAGGCTTTTCTTCTTGA, designated herein as SEQ ID NO: 11; probe sequence ACCCCAGGTTCTGCAGGAGG, designated herein as SEQ ID NO: 12). NLRP3 RNA levels were normalized to total RNA content, as measured by RIBOGREEN®. Reduction of NLRP3 RNA is presented in the tables below as percent NLRP3 RNA relative to the amount of NLRP3 RNA in untreated control cells (% UTC). The values marked with a “T” indicate that the modified oligonucleotide is complementary to the amplicon region of the primer probe set. Additional assays may be used to measure the potency and efficacy of the modified oligonucleotides complementary to the amplicon region. “N.D.” in the tables below refers to instances where the value was Not Defined.

Each separate experimental analysis described in this example is identified by a letter ID in the table column below labeled “AID” (Analysis ID).

Compound No. 1232737 was used as a benchmark on multiple plates, and has a sugar motif of (from 5′ to 3′): kkkddddddddddkkk; wherein each “d” represents a 2′-β-D-deoxyribosyl sugar moiety, and each “k” represents a cEt sugar moiety, and an internucleoside linkage motif of (from 5′ to 3′): sssssssssssssss; wherein each “s” represents a phosphorothioate internucleoside linkage. Each cytosine residue of Compound No. 1232737 is a 5-methyl cytosine.

TABLE 2
Reduction of NLRP3 RNA by 3-10-3 cEt modified oligonucleotides with uniform
phosphorothioate internucleoside linkages at a concentration of 2000 nM in THP-1 cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | NLRP3 | | |
| Compound | Start | Stop | Start | Stop | | (% | | SEQ ID |
| Number | Site | Site | Site | Site | Sequence (5′ to 3′) | UTC) | AID | NO |
| 1232727 | 5089 | 5104 | 41 | 56 | GGCTATGACATTGGAC | 69 | A | 20 |
| 1232735 | 5133 | 5148 | 85 | 100 | ATCTTGACCCATCAGC | 46 | A | 21 |
| 1232737 | 5138 | 5153 | 90 | 105 | ATGCCATCTTGACCCA | 58 | A | 22 |
| 1232744 | 5159 | 5174 | 111 | 126 | ACGGTGAACAACCACT | 43 | A | 23 |
| 1232752 | 5168 | 5183 | 120 | 135 | TTACAGTTTACGGTGA | 72 | A | 24 |
| 1232760 | 5254 | 5269 | 206 | 221 | CGATCCAGGAGTGTGT | 68 | A | 25 |
| 1232768 | 5597 | 5612 | 549 | 564 | AAGTTAGCCAAGGCCG | 86 | A | 26 |
| 1232776 | 5760 | 5775 | 712 | 727 | CCTTAGGCTTCGGTCC | 54 | A | 27 |
| 1232784 | 5969 | 5984 | 921 | 936 | CCCCATTGAAGTCGAT | 77 | A | 28 |
| 1232792 | 6012 | 6027 | 964 | 979 | ATCGCAGCGAAGATCC | 45 | A | 29 |
| 1232800 | 10250 | 10265 | 1050 | 1065 | TCACAGTGGGATTCGA | 83 | A | 30 |
| 1232808 | 10309 | 10324 | 1109 | 1124 | TCTCGAAAGGTACTCC | 54 | A | 31 |
| 1232816 | 10953 | 10968 | 1256 | 1271 | CTTGATGAGACGCAGT | 51 | A | 32 |
| 1232824 | 11365 | 11380 | 1668 | 1683 | CGTCAAAGGCACCTTG | 55 | A | 33 |
| 1232832 | 11952 | 11967 | 2255 | 2270 | GAACAGGTTCATCCTC | 52 | A | 34 |
| 1232840 | 12115 | 12130 | 2418 | 2433 | CCAGAAGGACTGTCAC | 83 | A | 35 |
| 1232848 | 12145 | 12160 | 2448 | 2463 | AATACCCCTTTTCGAA | 65 | A | 36 |
| 1232856 | 12380 | 12395 | 2683 | 2698 | GGGAAATAGTCCATGG | 48 | A | 37 |
| 1232864 | 12540 | 12555 | 2843 | 2858 | CACCATATCAAGGTGT | 95 | A | 38 |
| 1232872 | 16618 | 16633 | 2936 | 2951 | TGAAAAGAGGCCCCGG | 56 | A | 39 |
| 1232880 | 16734 | 16749 | 3052 | 3067 | CTCCGAATGTTACAGC | 56 | A | 40 |
| 1232888 | 21115 | 21130 | 3086 | 3101 | CTCATGCGAGAGGCCA | 57 | A | 41 |
| 1232896 | 21138 | 21153 | 3109 | 3124 | ACCAAGGAGATGTCGA | 65 | A | 42 |
| 1232904 | 21207 | 21222 | 3178 | 3193 | CTGATTCCGAAGTCAC | 58 | A | 43 |
| 1232912 | 23078 | 23093 | 3353 | 3368 | AATTGCGACTCCTGAG | 58 | A | 44 |
| 1232920 | 23086 | 23101 | 3361 | 3376 | TCACATAAAATTGCGA | 63 | A | 45 |
| 1232928 | 31047 | 31062 | 3491 | 3506 | TCGCAGGTAAAGGTGC | 70 | A | 46 |
| 1232936 | 35436 | 35451 | 3789 | 3804 | TTTCTAACGCACTTTT | 35† | A | 47 |
| 1232944 | 35476 | 35491 | 3829 | 3844 | TCAAAGACGACGGTCA | 91† | A | 48 |
| 1232952 | 35532 | 35547 | 3885 | 3900 | AGGGACCGGAGAACAC | 93 | A | 49 |
| 1232960 | 35672 | 35687 | 4025 | 4040 | CGTAAAGGAGATGCCC | 70 | A | 50 |
| 1232968 | 35877 | 35892 | 4230 | 4245 | AATGATGATATGAGCA | 81 | A | 51 |
| 1232976 | 35888 | 35903 | 4241 | 4256 | AAGATAGCGGGAATGA | 85 | A | 52 |
| 1232984 | 3623 | 3638 | N/A | N/A | CGCATACCCTCCCAGC | 78 | A | 53 |
| | 3686 | 3701 | | | | | | |
| 1232992 | 3632 | 3647 | N/A | N/A | TATTAGCTCCGCATAC | 108 | A | 54 |
| | 3695 | 3710 | | | | | | |
| 1233000 | 3868 | 3883 | N/A | N/A | AAACGGTGGAGCTCTC | 89 | A | 55 |
| 1233008 | 4423 | 4438 | N/A | N/A | CAAAATTCGCAATTGT | 68 | A | 56 |
| 1233016 | 6625 | 6640 | N/A | N/A | AGTAGTAAGATTCACA | 44 | A | 57 |
| 1233024 | 7936 | 7951 | N/A | N/A | CAAAACATGTGGTCTG | 83 | A | 58 |
| 1233032 | 8651 | 8666 | N/A | N/A | TAAACCATGGGCCAGT | 63 | A | 59 |
| 1233040 | 9523 | 9538 | N/A | N/A | ATCCAAACAGTCACTA | 81 | A | 60 |
| 1233048 | 10165 | 10180 | N/A | N/A | ATGCAATTGAAACCTG | 90 | A | 61 |
| 1233056 | 10742 | 10757 | N/A | N/A | GTAACCGGAATGGAGA | 90 | A | 62 |
| 1233064 | 12708 | 12723 | N/A | N/A | GCATTATGATGTAGCT | 68 | A | 63 |
| 1233072 | 13046 | 13061 | N/A | N/A | TCCTTAAAGGAGGCTT | 54 | A | 64 |
| 1233080 | 14379 | 14394 | N/A | N/A | CTCTTAATGAGTAGTT | 71 | A | 65 |
| 1233088 | 15873 | 15888 | N/A | N/A | AAATCGCAAGCCCAGG | 68 | A | 66 |
| 1233096 | 17442 | 17457 | N/A | N/A | TAACATTAGGTCTTGT | 59 | A | 67 |
| 1233104 | 17669 | 17684 | N/A | N/A | GAATATATCGAGCATC | 30 | A | 68 |
| 1233112 | 18559 | 18574 | N/A | N/A | CAAACGCAAACCCCAT | 62 | A | 69 |
| 1233120 | 18739 | 18754 | N/A | N/A | GCACAATCGAATCAGA | 50 | A | 70 |
| 1233128 | 19002 | 19017 | N/A | N/A | AACTACTCGAAGAATT | 79 | A | 71 |
| 1233136 | 19798 | 19813 | N/A | N/A | CTATACTTTATCCCTG | 69 | A | 72 |
| 1233144 | 20583 | 20598 | N/A | N/A | GACCAGAGAGCTCCGG | 27 | A | 73 |
| | 20709 | 20724 | | | | | | |
| | 20751 | 20766 | | | | | | |
| | 20919 | 20934 | | | | | | |
| 1233152 | 20702 | 20717 | N/A | N/A | GAGCTCCGGAATAGAG | 90 | A | 74 |
| | 20744 | 20759 | | | | | | |
| | 20912 | 20927 | | | | | | |
| 1233160 | 22485 | 22500 | N/A | N/A | TCCTATAAGGACAATC | 76 | A | 75 |
| 1233168 | 23303 | 23318 | N/A | N/A | ATCTTATTAGCAGCAG | 23 | A | 76 |
| 1233176 | 23496 | 23511 | N/A | N/A | CCTAGTGTAGAGGCTG | 20 | A | 77 |
| | 23529 | 23544 | | | | | | |
| 1233184 | 23823 | 23838 | N/A | N/A | CATGATTATTAGTGGG | 66 | A | 78 |
| 1233192 | 24163 | 24178 | N/A | N/A | AAAGCTAAATTGGAGG | 53 | A | 79 |
| 1233200 | 24528 | 24543 | N/A | N/A | TTCTAAGGCGATTTGC | 59 | A | 80 |
| 1233208 | 25161 | 25176 | N/A | N/A | GACCTATAAGGACCCA | 29 | A | 81 |
| 1233216 | 26174 | 26189 | N/A | N/A | AATCACATAGGTCTTC | 82 | A | 82 |
| 1233224 | 26690 | 26705 | N/A | N/A | TTTCAATAGATAGGGT | 73 | A | 83 |
| 1233232 | 26940 | 26955 | N/A | N/A | ATAGATACCTATCTCA | 66 | A | 84 |
| 1233240 | 26973 | 26988 | N/A | N/A | ATGCATAAAAGTCTCG | N.D. | A | 85 |
| 1233248 | 27030 | 27045 | N/A | N/A | AACCCATAGGCTATAC | 57 | A | 86 |
| | 27066 | 27081 | | | | | | |
| 1233256 | 27364 | 27379 | N/A | N/A | ATTTAGGACCGTCCCC | 86 | A | 87 |
| 1233264 | 27772 | 27787 | N/A | N/A | AACTAATTCATAGCTG | 105 | A | 88 |
| 1233272 | 28687 | 28702 | N/A | N/A | AATCCTTGGATTAGAG | 61 | A | 89 |
| 1233280 | 28761 | 28776 | N/A | N/A | TAACTATTAAGCAACG | 73 | A | 90 |
| 1233288 | 29361 | 29376 | N/A | N/A | CAGGAATAGCGCAGGT | 87 | A | 91 |
| 1233296 | 31299 | 31314 | N/A | N/A | ATCTATATCATCTGGA | 70 | A | 92 |
| 1233304 | 31903 | 31918 | N/A | N/A | TGCAAATGAAGCGAGA | 71 | A | 93 |
| 1233312 | 32126 | 32141 | N/A | N/A | CCATAGACGAGTTCTA | 63 | A | 94 |
| 1233320 | 33802 | 33817 | N/A | N/A | TCATTAATATGGACAG | 53 | A | 95 |
| 1233328 | 34216 | 34231 | N/A | N/A | AGGAAGAGGACTCTCG | 57 | A | 96 |
| 1233336 | 34417 | 34432 | N/A | N/A | CCCTAATGTACGGTAC | 62 | A | 97 |
| 1232728 | 5104 | 5119 | 56 | 71 | TCCCGTTGATTACGGG | 109 | B | 98 |
| 1232736 | 5135 | 5150 | 87 | 102 | CCATCTTGACCCATCA | 47 | B | 99 |
| 1232737 | 5138 | 5153 | 90 | 105 | ATGCCATCTTGACCCA | 61 | B | 22 |
| 1232745 | 5160 | 5175 | 112 | 127 | TACGGTGAACAACCAC | 41 | B | 100 |
| 1232753 | 5170 | 5185 | 122 | 137 | TATTACAGTTTACGGT | 57 | B | 101 |
| 1232761 | 5256 | 5271 | 208 | 223 | CTCGATCCAGGAGTGT | 66 | B | 102 |
| 1232769 | 5714 | 5729 | 666 | 681 | CACGGATGAGTCTTTT | 63 | B | 103 |
| 1232777 | 5762 | 5777 | 714 | 729 | GTCCTTAGGCTTCGGT | 62 | B | 104 |
| 1232785 | 6000 | 6015 | 952 | 967 | ATCCACACGGCCATGG | 65 | B | 105 |
| 1232793 | 6013 | 6028 | 965 | 980 | GATCGCAGCGAAGATC | 94 | B | 106 |
| 1232801 | 10256 | 10271 | 1056 | 1071 | GGCATATCACAGTGGG | 53 | B | 107 |
| 1232809 | 10311 | 10326 | 1111 | 1126 | ATTCTCGAAAGGTACT | 77 | B | 108 |
| 1232817 | 11039 | 11054 | 1342 | 1357 | TTAATGGGACTCACGG | 46 | B | 109 |
| 1232825 | 11427 | 11442 | 1730 | 1745 | GCTCAGGAGAATGTCT | 86 | B | 110 |
| 1232833 | 12032 | 12047 | 2335 | 2350 | AGGTAGTACATGGCGG | 49 | B | 111 |
| 1232841 | 12132 | 12147 | 2435 | 2450 | GAATTTGCCATAGTTT | 39 | B | 112 |
| 1232849 | 12203 | 12218 | 2506 | 2521 | AAGTAGGAGGTCCTCT | 58 | B | 113 |
| 1232857 | 12400 | 12415 | 2703 | 2718 | AGAGATTGATCTCAAT | 78 | B | 114 |
| 1232865 | 16607 | 16622 | 2925 | 2940 | CCCGGCAAAAACTGGA | 100 | B | 115 |
| 1232873 | 16619 | 16634 | 2937 | 2952 | CTGAAAAGAGGCCCCG | 49 | B | 116 |
| 1232881 | 16735 | 16750 | 3053 | 3068 | TCTCCGAATGTTACAG | 66 | B | 117 |
| 1232889 | 21116 | 21131 | 3087 | 3102 | ACTCATGCGAGAGGCC | 76 | B | 118 |
| 1232897 | 21139 | 21154 | 3110 | 3125 | GACCAAGGAGATGTCG | 80 | B | 119 |
| 1232905 | 21240 | 21255 | 3211 | 3226 | CACAACAGGTGCTTCA | 77 | B | 120 |
| 1232913 | 23079 | 23094 | 3354 | 3369 | AAATTGCGACTCCTGA | 56 | B | 121 |
| 1232921 | 30973 | 30988 | 3417 | 3432 | GGCCAGAATTCACCAA | 71 | B | 122 |
| 1232929 | 31062 | 31077 | 3506 | 3521 | TCCGAGAGTGTTGCCT | 43 | B | 123 |
| 1232937 | 35437 | 35452 | 3790 | 3805 | GTTTCTAACGCACTTT | 16† | B | 124 |
| 1232945 | 35477 | 35492 | 3830 | 3845 | CTCAAAGACGACGGTC | 94 | B | 125 |
| 1232953 | 35536 | 35551 | 3889 | 3904 | CTGGAGGGACCGGAGA | 108 | B | 126 |
| 1232961 | 35712 | 35727 | 4065 | 4080 | ACCCGATGCTGTCATT | 70 | B | 127 |
| 1232969 | 35878 | 35893 | 4231 | 4246 | GAATGATGATATGAGC | 76 | B | 128 |
| 1232977 | 35889 | 35904 | 4242 | 4257 | AAAGATAGCGGGAATG | 99 | B | 129 |
| 1232985 | 3624 | 3639 | N/A | N/A | CCGCATACCCTCCCAG | 71 | B | 130 |
| | 3687 | 3702 | | | | | | |
| 1232993 | 3633 | 3648 | N/A | N/A | CTATTAGCTCCGCATA | 74 | B | 131 |
| | 3696 | 3711 | | | | | | |
| 1233001 | 3869 | 3884 | N/A | N/A | AAAACGGTGGAGCTCT | 80 | B | 132 |
| 1233009 | 6446 | 6461 | N/A | N/A | TTATTACGGCCGGGCG | 65 | B | 133 |
| 1233017 | 6626 | 6641 | N/A | N/A | GAGTAGTAAGATTCAC | 49 | B | 134 |
| 1233025 | 8120 | 8135 | N/A | N/A | AAAGAACCTTATCCCC | 90 | B | 135 |
| 1233033 | 8733 | 8748 | N/A | N/A | AAAGACGGTGTAGTGG | 42 | B | 136 |
| 1233041 | 9576 | 9591 | N/A | N/A | CATACTAAAAGCTCCC | 89 | B | 137 |
| 1233049 | 10180 | 10195 | N/A | N/A | CTATTATCACAGAGGA | 101 | B | 138 |
| 1233057 | 10743 | 10758 | N/A | N/A | GGTAACCGGAATGGAG | 79 | B | 139 |
| 1233065 | 12756 | 12771 | N/A | N/A | CCATACATCAATCTTT | 63 | B | 140 |
| 1233073 | 13928 | 13943 | N/A | N/A | GTAGAAACCGTACCCT | 69 | B | 141 |
| 1233081 | 14415 | 14430 | N/A | N/A | CATAAGGTCTCCCTGC | 72 | B | 142 |
| 1233089 | 15874 | 15889 | N/A | N/A | AAAATCGCAAGCCCAG | 81 | B | 143 |
| 1233097 | 17445 | 17460 | N/A | N/A | GATTAACATTAGGTCT | 52 | B | 144 |
| 1233105 | 17672 | 17687 | N/A | N/A | ATGGAATATATCGAGC | 29 | B | 145 |
| 1233113 | 18560 | 18575 | N/A | N/A | ACAAACGCAAACCCCA | 65 | B | 146 |
| 1233121 | 18751 | 18766 | N/A | N/A | AGCCACCGAACAGCAC | 50 | B | 147 |
| 1233129 | 19302 | 19317 | N/A | N/A | ACAAAGGGTAACATCC | 44 | B | 148 |
| 1233137 | 19800 | 19815 | N/A | N/A | AACTATACTTTATCCC | 96 | B | 149 |
| 1233145 | 20584 | 20599 | N/A | N/A | TGACCAGAGAGCTCCG | 43 | B | 150 |
| | 20710 | 20725 | | | | | | |
| | 20752 | 20767 | | | | | | |
| | 20920 | 20935 | | | | | | |
| 1233153 | 21596 | 21611 | N/A | N/A | AATATGAGGACCCAGG | 35 | B | 151 |
| 1233161 | 22667 | 22682 | N/A | N/A | ATTAATGGCCTTAAGC | 89 | B | 152 |
| 1233169 | 23347 | 23362 | N/A | N/A | ACACTAAGAAACTCTA | 67 | B | 153 |
| 1233177 | 23497 | 23512 | N/A | N/A | TCCTAGTGTAGAGGCT | 19 | B | 154 |
| | 23530 | 23545 | | | | | | |
| 1233185 | 23926 | 23941 | N/A | N/A | CCCTATAGCATCTTTC | 60 | B | 155 |
| 1233193 | 24272 | 24287 | N/A | N/A | AATGAAGACCTATCCA | 83 | B | 156 |
| 1233201 | 24531 | 24546 | N/A | N/A | ATATTCTAAGGCGATT | 59 | B | 157 |
| 1233209 | 25188 | 25203 | N/A | N/A | AAAGTAAGGACTGGGA | 71 | B | 158 |
| 1233217 | 26324 | 26339 | N/A | N/A | ATCTTAATCTCACACT | 56 | B | 159 |
| 1233225 | 26778 | 26793 | N/A | N/A | CGATAGATCTACACAA | 70 | B | 160 |
| 1233233 | 26941 | 26956 | N/A | N/A | TATAGATACCTATCTC | 75 | B | 161 |
| 1233241 | 27022 | 27037 | N/A | N/A | GGCTATACCTAACACA | 25 | B | 162 |
| | 27058 | 27073 | | | | | | |
| | 27112 | 27127 | | | | | | |
| 1233249 | 27031 | 27046 | N/A | N/A | TAACCCATAGGCTATA | 78 | B | 163 |
| | 27067 | 27082 | | | | | | |
| 1233257 | 27365 | 27380 | N/A | N/A | CATTTAGGACCGTCCC | 85 | B | 164 |
| 1233265 | 28559 | 28574 | N/A | N/A | TTATTAACTACCTGCC | 81 | B | 165 |
| 1233273 | 28709 | 28724 | N/A | N/A | AAACAACCATGTCCCA | 71 | B | 166 |
| 1233281 | 28824 | 28839 | N/A | N/A | AAATTGCAACAGACGG | 55 | B | 167 |
| 1233289 | 29461 | 29476 | N/A | N/A | GTCAAATTAAGCCCAT | 31 | B | 168 |
| 1233297 | 31515 | 31530 | N/A | N/A | ACTAACTAGCTTCAAG | 100 | B | 169 |
| 1233305 | 31921 | 31936 | N/A | N/A | AACCTAATCTTGAGTG | 96 | B | 170 |
| 1233313 | 32139 | 32154 | N/A | N/A | CAATTTGGATGACCCA | 46 | B | 171 |
| 1233321 | 33804 | 33819 | N/A | N/A | GATCATTAATATGGAC | 75 | B | 172 |
| 1233329 | 34319 | 34334 | N/A | N/A | CAGGAGAGATATCAGA | 72 | B | 173 |
| 1233337 | 34420 | 34435 | N/A | N/A | AACCCCTAATGTACGG | 65 | B | 174 |
| 1232729 | 5111 | 5126 | 63 | 78 | ATTTTTGTCCCGTTGA | 82 | C | 175 |
| 1232737 | 5138 | 5153 | 90 | 105 | ATGCCATCTTGACCCA | 62 | C | 22 |
| 1232738 | 5142 | 5157 | 94 | 109 | CACGATGCCATCTTGA | 51 | C | 176 |
| 1232746 | 5161 | 5176 | 113 | 128 | TTACGGTGAACAACCA | 57 | C | 177 |
| 1232754 | 5171 | 5186 | 123 | 138 | GTATTACAGTTTACGG | 41 | C | 178 |
| 1232762 | 5279 | 5294 | 231 | 246 | TTACCAGAAAGTTCTC | 54 | C | 179 |
| 1232770 | 5715 | 5730 | 667 | 682 | ACACGGATGAGTCTTT | 65 | C | 180 |
| 1232778 | 5805 | 5820 | 757 | 772 | TTGCAGCGGGTGCTTG | 79 | C | 181 |
| 1232786 | 6006 | 6021 | 958 | 973 | GCGAAGATCCACACGG | 47 | C | 182 |
| 1232794 | 6032 | 6047 | 984 | 999 | CATAAAGGTCTCTCCT | 81 | C | 183 |
| 1232802 | 10294 | 10309 | 1094 | 1109 | CAGTAAACCCATCCAC | 65 | C | 184 |
| 1232810 | 10312 | 10327 | 1112 | 1127 | GATTCTCGAAAGGTAC | 63 | C | 185 |
| 1232818 | 11040 | 11055 | 1343 | 1358 | CTTAATGGGACTCACG | 51 | C | 186 |
| 1232826 | 11527 | 11542 | 1830 | 1845 | GCCGAGGATGGTCCAG | 61 | C | 187 |
| 1232834 | 12080 | 12095 | 2383 | 2398 | AAACGACTCCCTGGAA | 80 | C | 188 |
| 1232842 | 12135 | 12150 | 2438 | 2453 | TTCGAATTTGCCATAG | 44 | C | 189 |
| 1232850 | 12204 | 12219 | 2507 | 2522 | CAAGTAGGAGGTCCTC | 51 | C | 190 |
| 1232858 | 12404 | 12419 | 2707 | 2722 | GTGGAGAGATTGATCT | 53 | C | 191 |
| 1232866 | 16608 | 16623 | 2926 | 2941 | CCCCGGCAAAAACTGG | 71 | C | 192 |
| 1232874 | 16651 | 16666 | 2969 | 2984 | CAATTCAGTTAGACTC | 63 | C | 193 |
| 1232882 | 16736 | 16751 | 3054 | 3069 | ATCTCCGAATGTTACA | 61 | C | 194 |
| 1232890 | 21126 | 21141 | 3097 | 3112 | TCGAAGCAGCACTCAT | 94 | C | 195 |
| 1232898 | 21201 | 21216 | 3172 | 3187 | CCGAAGTCACCGAGGG | 66 | C | 196 |
| 1232906 | 21250 | 21265 | 3221 | 3236 | CTTCAGATTGCACAAC | 91 | C | 197 |
| 1232914 | 23080 | 23095 | 3355 | 3370 | AAAATTGCGACTCCTG | 48 | C | 198 |
| 1232922 | 30978 | 30993 | 3422 | 3437 | CGTAAGGCCAGAATTC | 87 | C | 199 |
| 1232930 | 31063 | 31078 | 3507 | 3522 | CTCCGAGAGTGTTGCC | 66 | C | 200 |
| 1232938 | 35470 | 35485 | 3823 | 3838 | ACGACGGTCAGCTCAG | 85† | C | 201 |
| 1232946 | 35478 | 35493 | 3831 | 3846 | GCTCAAAGACGACGGT | 83 | C | 202 |
| 1232954 | 35571 | 35586 | 3924 | 3939 | ATGGATCGCAGCTCTC | 54 | C | 203 |
| 1232962 | 35713 | 35728 | 4066 | 4081 | CACCCGATGCTGTCAT | 69 | C | 204 |
| 1232970 | 35881 | 35896 | 4234 | 4249 | CGGGAATGATGATATG | 94 | C | 205 |
| 1232978 | 35890 | 35905 | 4243 | 4258 | GAAAGATAGCGGGAAT | 101 | C | 206 |
| 1232986 | 3626 | 3641 | N/A | N/A | CTCCGCATACCCTCCC | 86 | C | 207 |
| | 3689 | 3704 | | | | | | |
| 1232994 | 3634 | 3649 | N/A | N/A | CCTATTAGCTCCGCAT | 80 | C | 208 |
| | 3697 | 3712 | | | | | | |
| 1233002 | 3872 | 3887 | N/A | N/A | TGGAAAACGGTGGAGC | 79 | C | 209 |
| 1233010 | 6447 | 6462 | N/A | N/A | ATTATTACGGCCGGGC | 78 | C | 210 |
| 1233018 | 6718 | 6733 | N/A | N/A | ACATAGTTGAACCCGG | 60 | C | 211 |
| 1233026 | 8478 | 8493 | N/A | N/A | TATAAGTACTGACACA | 73 | C | 212 |
| 1233034 | 8734 | 8749 | N/A | N/A | CAAAGACGGTGTAGTG | 59 | C | 213 |
| 1233042 | 9735 | 9750 | N/A | N/A | CAAGAGTGAGAACGAC | 33 | C | 214 |
| 1233050 | 10431 | 10446 | N/A | N/A | ACCTATAGTATGGCCA | 65 | C | 215 |
| 1233058 | 10754 | 10769 | N/A | N/A | CGGAAGCGAGTGGTAA | 95 | C | 216 |
| 1233066 | 12758 | 12773 | N/A | N/A | TACCATACATCAATCT | 85 | C | 217 |
| 1233074 | 13933 | 13948 | N/A | N/A | CTGCCGTAGAAACCGT | 60 | C | 218 |
| 1233082 | 14498 | 14513 | N/A | N/A | TGCTATCTAGAGAAAT | 79 | C | 219 |
| | 14644 | 14659 | | | | | | |
| | 14717 | 14732 | | | | | | |
| | 14790 | 14805 | | | | | | |
| | 14936 | 14951 | | | | | | |
| | 15009 | 15024 | | | | | | |
| | 15082 | 15097 | | | | | | |
| 1233090 | 15875 | 15890 | N/A | N/A | GAAAATCGCAAGCCCA | 68 | C | 220 |
| 1233098 | 17450 | 17465 | N/A | N/A | GACTAGATTAACATTA | 65 | C | 221 |
| 1233106 | 17673 | 17688 | V/A | N/A | AATGGAATATATCGAG | 41 | C | 222 |
| 1233114 | 18561 | 18576 | N/A | N/A | AACAAACGCAAACCCC | 74 | C | 223 |
| 1233122 | 18757 | 18772 | N/A | N/A | CATTTAAGCCACCGAA | 77 | C | 224 |
| 1233130 | 19497 | 19512 | N/A | N/A | ACGATTTGAAATTTAC | 72 | C | 225 |
| 1233138 | 20485 | 20500 | N/A | N/A | CCGTAAACAGTGCCGG | 96 | C | 226 |
| 1233146 | 20593 | 20608 | N/A | N/A | GAATACACCTGACCAG | 69 | C | 227 |
| 1233154 | 21599 | 21614 | N/A | N/A | TACAATATGAGGACCC | 32 | C | 228 |
| 1233162 | 22790 | 22805 | N/A | N/A | AACAAGTAAGCATTCT | 68 | C | 229 |
| 1233170 | 23472 | 23487 | N/A | N/A | ATGCGATAACTTGCAT | 110 | C | 230 |
| 1233178 | 23597 | 23612 | N/A | N/A | CTACATAGGTGAAGGT | 68 | C | 231 |
| 1233186 | 23927 | 23942 | N/A | N/A | ACCCTATAGCATCTTT | 93 | C | 232 |
| 1233194 | 24363 | 24378 | N/A | N/A | GATTAGTGGATTACTC | 87 | C | 233 |
| 1233202 | 24532 | 24547 | N/A | N/A | AATATTCTAAGGCGAT | 73 | C | 234 |
| 1233210 | 25194 | 25209 | N/A | N/A | TCAACTAAAGTAAGGA | 65 | C | 235 |
| 1233218 | 26517 | 26532 | N/A | N/A | GAAACTTGGGACATGG | 71 | C | 236 |
| 1233226 | 26780 | 26795 | N/A | N/A | ATCGATAGATCTACAC | 55 | C | 237 |
| 1233234 | 26943 | 26958 | N/A | N/A | CTTATAGATACCTATC | 78 | C | 238 |
| 1233242 | 27024 | 27039 | N/A | N/A | TAGGCTATACCTAACA | 39 | C | 239 |
| | 27060 | 27075 | | | | | | |
| | 27096 | 27111 | | | | | | |
| | 27114 | 27129 | | | | | | |
| 1233250 | 27032 | 27047 | N/A | N/A | CTAACCCATAGGCTAT | 56 | C | 240 |
| | 27068 | 27083 | | | | | | |
| 1233258 | 27366 | 27381 | N/A | N/A | GCATTTAGGACCGTCC | 59 | C | 241 |
| 1233266 | 28584 | 28599 | N/A | N/A | AAGTTAATTGGTTGGG | 45 | C | 242 |
| 1233274 | 28753 | 28768 | N/A | N/A | AAGCAACGGTTACACT | 85 | C | 243 |
| 1233282 | 29060 | 29075 | N/A | N/A | CCCCGGTAAGAATTCT | 73 | C | 244 |
| 1233290 | 29462 | 29477 | N/A | N/A | AGTCAAATTAAGCCCA | 42 | C | 245 |
| 1233298 | 31516 | 31531 | N/A | N/A | GACTAACTAGCTTCAA | 105 | C | 246 |
| 1233306 | 31922 | 31937 | N/A | N/A | CAACCTAATCTTGAGT | 82 | C | 247 |
| 1233314 | 32154 | 32169 | N/A | N/A | ATGTAAAGACCCTTCC | 79 | C | 248 |
| 1233322 | 33917 | 33932 | N/A | N/A | GAATATTGGCTTGTCC | 78 | C | 249 |
| 1233330 | 34371 | 34386 | N/A | N/A | AGTTAGATGAAGTTGT | 70 | C | 250 |
| 1233338 | 34683 | 34698 | N/A | N/A | GCATTTACGCTTCCTC | 38 | C | 251 |
| 1232730 | 5112 | 5127 | 64 | 79 | AATTTTTGTCCCGTTG | 96 | D | 252 |
| 1232737 | 5138 | 5153 | 90 | 105 | ATGCCATCTTGACCCA | 47 | D | 22 |
| 1232739 | 5145 | 5160 | 97 | 112 | CTTCACGATGCCATCT | 55 | D | 253 |
| 1232747 | 5162 | 5177 | 114 | 129 | TTTACGGTGAACAACC | 56 | D | 254 |
| 1232755 | 5172 | 5187 | 124 | 139 | TGTATTACAGTTTACG | 57 | D | 255 |
| 1232763 | 5280 | 5295 | 232 | 247 | CTTACCAGAAAGTTCT | 53 | D | 256 |
| 1232771 | 5719 | 5734 | 671 | 686 | CGGCACACGGATGAGT | 70 | D | 257 |
| 1232779 | 5961 | 5976 | 913 | 928 | AAGTCGATCATTAGCG | 57 | D | 258 |
| 1232787 | 6007 | 6022 | 959 | 974 | AGCGAAGATCCACACG | 56 | D | 259 |
| 1232795 | 6033 | 6048 | 985 | 1000 | TCATAAAGGTCTCTCC | 54 | D | 260 |
| 1232803 | 10304 | 10319 | 1104 | 1119 | AAAGGTACTCCAGTAA | 68 | D | 261 |
| 1232811 | 10313 | 10328 | 1113 | 1128 | AGATTCTCGAAAGGTA | 75 | D | 262 |
| 1232819 | 11041 | 11056 | 1344 | 1359 | TCTTAATGGGACTCAC | 49 | D | 263 |
| 1232827 | 11528 | 11543 | 1831 | 1846 | TGCCGAGGATGGTCCA | 61 | D | 264 |
| 1232835 | 12081 | 12096 | 2384 | 2399 | CAAACGACTCCCTGGA | 67 | D | 265 |
| 1232843 | 12136 | 12151 | 2439 | 2454 | TTTCGAATTTGCCATA | 38 | D | 266 |
| 1232851 | 12235 | 12250 | 2538 | 2553 | GCTGAGAGATCTTGCA | 62 | D | 267 |
| 1232859 | 12454 | 12469 | 2757 | 2772 | CCCGATGACAGTTCTC | 40 | D | 268 |
| 1232867 | 16609 | 16624 | 2927 | 2942 | GCCCCGGCAAAAACTG | 94 | D | 269 |
| 1232875 | 16653 | 16668 | 2971 | 2986 | TCCAATTCAGTTAGAC | 45 | D | 270 |
| 1232883 | 16737 | 16752 | 3055 | 3070 | AATCTCCGAATGTTAC | 82 | D | 271 |
| 1232891 | 21127 | 21142 | 3098 | 3113 | GTCGAAGCAGCACTCA | 42 | D | 272 |
| 1232899 | 21202 | 21217 | 3173 | 3188 | TCCGAAGTCACCGAGG | 62 | D | 273 |
| 1232907 | 23026 | 23041 | 3301 | 3316 | AGGGAATGGCTGGTGC | 68 | D | 274 |
| 1232915 | 23081 | 23096 | 3356 | 3371 | TAAAATTGCGACTCCT | 80 | D | 275 |
| 1232923 | 31008 | 31023 | 3452 | 3467 | TACCGAGGACAAAGCT | 83 | D | 276 |
| 1232931 | 31064 | 31079 | 3508 | 3523 | TCTCCGAGAGTGTTGC | 46 | D | 277 |
| 1232939 | 35471 | 35486 | 3824 | 3839 | GACGACGGTCAGCTCA | 89† | D | 278 |
| 1232947 | 35487 | 35502 | 3840 | 3855 | ACCAAGAAGGCTCAAA | 78 | D | 279 |
| 1232955 | 35575 | 35590 | 3928 | 3943 | CTGGATGGATCGCAGC | 85 | D | 280 |
| 1232963 | 35715 | 35730 | 4068 | 4083 | AACACCCGATGCTGTC | 76 | D | 281 |
| 1232971 | 35882 | 35897 | 4235 | 4250 | GCGGGAATGATGATAT | 85 | D | 282 |
| 1232979 | 35923 | 35938 | 4276 | 4291 | AGTCAACTAGTTCTGT | 73 | D | 283 |
| 1232987 | 3627 | 3642 | N/A | N/A | GCTCCGCATACCCTCC | 98 | D | 284 |
| | 3690 | 3705 | | | | | | |
| 1232995 | 3635 | 3650 | N/A | N/A | CCCTATTAGCTCCGCA | 60 | D | 285 |
| | 3698 | 3713 | | | | | | |
| 1233003 | 3873 | 3888 | N/A | N/A | CTGGAAAACGGTGGAG | 94 | D | 286 |
| 1233011 | 6448 | 6463 | N/A | N/A | AATTATTACGGCCGGG | 66 | D | 287 |
| 1233019 | 6738 | 6753 | N/A | N/A | GAATTCCGACACATGC | 50 | D | 288 |
| 1233027 | 8481 | 8496 | N/A | N/A | GTATATAAGTACTGAC | 66 | D | 289 |
| 1233035 | 8735 | 8750 | N/A | N/A | GCAAAGACGGTGTAGT | 57 | D | 290 |
| 1233043 | 9823 | 9838 | N/A | N/A | CTAAGAAAGGTCACCC | 74 | D | 291 |
| 1233051 | 10432 | 10447 | N/A | N/A | AACCTATAGTATGGCC | 82 | D | 292 |
| 1233059 | 10831 | 10846 | N/A | N/A | CTACGGCAAAGACAGG | 92 | D | 293 |
| 1233067 | 12826 | 12841 | N/A | N/A | CTAAACTGGCATTCCC | 59 | D | 294 |
| 1233075 | 14024 | 14039 | N/A | N/A | TGCTTATAAGCCAACC | 33 | D | 295 |
| 1233083 | 14502 | 14517 | N/A | N/A | AAGGTGCTATCTAGAG | 16 | D | 296 |
| | 14648 | 14663 | | | | | | |
| | 14721 | 14736 | | | | | | |
| | 14794 | 14809 | | | | | | |
| | 14940 | 14955 | | | | | | |
| | 15013 | 15028 | | | | | | |
| | 15086 | 15101 | | | | | | |
| 1233091 | 15877 | 15892 | N/A | N/A | AAGAAAATCGCAAGCC | 79 | D | 297 |
| 1233099 | 17509 | 17524 | N/A | N/A | CCCTAATCCTCAAGAC | 108 | D | 298 |
| 1233107 | 18420 | 18435 | N/A | N/A | TGATAAGACTGGTGAC | 48 | D | 299 |
| 1233115 | 18604 | 18619 | N/A | N/A | ACTAAGATGTCTGTCC | 50 | D | 300 |
| 1233123 | 18922 | 18937 | N/A | N/A | AACACGGCGAGAGTCC | 62 | D | 301 |
| 1233131 | 19498 | 19513 | N/A | N/A | TACGATTTGAAATTTA | 81 | D | 302 |
| 1233139 | 20568 | 20583 | N/A | N/A | GAATACAGCACAAGGC | 59 | D | 303 |
| 1233147 | 20697 | 20712 | N/A | N/A | CCGGAATAGAGAAAGC | 50 | D | 304 |
| | 20739 | 20754 | | | | | | |
| | 20907 | 20922 | | | | | | |
| | 20991 | 21006 | | | | | | |
| 1233155 | 21604 | 21619 | N/A | N/A | TCTTATACAATATGAG | 69 | D | 305 |
| 1233163 | 23138 | 23153 | N/A | N/A | CATGAAGACTTACCCC | 80 | D | 306 |
| 1233171 | 23473 | 23488 | N/A | N/A | TATGCGATAACTTGCA | 96 | D | 307 |
| 1233179 | 23688 | 23703 | N/A | N/A | GGTAAAACATCCACCA | 109 | D | 308 |
| 1233187 | 24112 | 24127 | N/A | N/A | CTGTAGAGAGCCCATT | 75 | D | 309 |
| 1233195 | 24500 | 24515 | N/A | N/A | ATACCCTAAGGCGAGG | 53 | D | 310 |
| 1233203 | 24533 | 24548 | N/A | N/A | TAATATTCTAAGGCGA | 54 | D | 311 |
| 1233211 | 25294 | 25309 | N/A | N/A | AAGCTATAACATTAGC | 80 | D | 312 |
| 1233219 | 26554 | 26569 | N/A | N/A | CGTTACAAATCTAACA | 68 | D | 313 |
| 1233227 | 26781 | 26796 | N/A | N/A | AATCGATAGATCTACA | 64 | D | 314 |
| 1233235 | 26944 | 26959 | N/A | N/A | ACTTATAGATACCTAT | 95 | D | 315 |
| 1233243 | 27025 | 27040 | N/A | N/A | ATAGGCTATACCTAAC | 46 | D | 316 |
| | 27061 | 27076 | | | | | | |
| | 27097 | 27112 | | | | | | |
| | 27115 | 27130 | | | | | | |
| 1233251 | 27033 | 27048 | N/A | N/A | CCTAACCCATAGGCTA | 30 | D | 317 |
| | 27069 | 27084 | | | | | | |
| 1233259 | 27378 | 27393 | N/A | N/A | ACCGAAAGCAGAGCAT | 71 | D | 318 |
| 1233267 | 28585 | 28600 | N/A | N/A | TAAGTTAATTGGTTGG | 54 | D | 319 |
| 1233275 | 28754 | 28769 | N/A | N/A | TAAGCAACGGTTACAC | 59 | D | 320 |
| 1233283 | 29140 | 29155 | N/A | N/A | TAAGACTAATAGGCAA | 102 | D | 321 |
| 1233291 | 29498 | 29513 | N/A | N/A | CTCGATGGAATTAAGA | 62 | D | 322 |
| 1233299 | 31898 | 31913 | N/A | N/A | ATGAAGCGAGAGCCAT | 67 | D | 323 |
| 1233307 | 31949 | 31964 | N/A | N/A | AAATATCCACCACAAC | 104 | D | 324 |
| 1233315 | 32155 | 32170 | N/A | N/A | TATGTAAAGACCCTTC | 90 | D | 325 |
| 1233323 | 34083 | 34098 | N/A | N/A | AAACGGGTTGATTGCA | 48 | D | 326 |
| 1233331 | 34379 | 34394 | N/A | N/A | GTAGTTAGAGTTAGAT | 54 | D | 327 |
| 1233339 | 34874 | 34889 | N/A | N/A | CAATTAAACAGGTCAT | 77 | D | 328 |
| 1232731 | 5113 | 5128 | 65 | 80 | AAATTTTTGTCCCGTT | 85 | E | 329 |
| 1232737 | 5138 | 5153 | 90 | 105 | ATGCCATCTTGACCCA | 65 | E | 22 |
| 1232740 | 5147 | 5162 | 99 | 114 | CACTTCACGATGCCAT | 39 | E | 330 |
| 1232748 | 5163 | 5178 | 115 | 130 | GTTTACGGTGAACAAC | 77 | E | 331 |
| 1232756 | 5177 | 5192 | 129 | 144 | AGGATTGTATTACAGT | 70 | E | 332 |
| 1232764 | 5288 | 5303 | 240 | 255 | GCCAAATGCTTACCAG | 35 | E | 333 |
| 1232772 | 5723 | 5738 | 675 | 690 | AACACGGCACACGGAT | 50 | E | 334 |
| 1232780 | 5964 | 5979 | 916 | 931 | TTGAAGTCGATCATTA | 70 | E | 335 |
| 1232788 | 6008 | 6023 | 960 | 975 | CAGCGAAGATCCACAC | 54 | E | 336 |
| 1232796 | 6034 | 6049 | 986 | 1001 | CTCATAAAGGTCTCTC | 41 | E | 337 |
| 1232804 | 10305 | 10320 | 1105 | 1120 | GAAAGGTACTCCAGTA | 58 | E | 338 |
| 1232812 | 10317 | 10332 | 1117 | 1132 | ATAGAGATTCTCGAAA | 87 | E | 339 |
| 1232820 | 11042 | 11057 | 1345 | 1360 | ATCTTAATGGGACTCA | 47 | E | 340 |
| 1232828 | 11529 | 11544 | 1832 | 1847 | ATGCCGAGGATGGTCC | 58 | E | 341 |
| 1232836 | 12082 | 12097 | 2385 | 2400 | TCAAACGACTCCCTGG | 40 | E | 342 |
| 1232844 | 12138 | 12153 | 2441 | 2456 | CTTTTCGAATTTGCCA | 35 | E | 343 |
| 1232852 | 12357 | 12372 | 2660 | 2675 | CACGAAGTCCTCCTCC | 69 | E | 344 |
| 1232860 | 12456 | 12471 | 2759 | 2774 | CACCCGATGACAGTTC | 67 | E | 345 |
| 1232868 | 16611 | 16626 | 2929 | 2944 | AGGCCCCGGCAAAAAC | 88 | E | 346 |
| 1232876 | 16654 | 16669 | 2972 | 2987 | GTCCAATTCAGTTAGA | 51 | E | 347 |
| 1232884 | 16738 | 16753 | 3056 | 3071 | CAATCTCCGAATGTTA | 75 | E | 348 |
| 1232892 | 21129 | 21144 | 3100 | 3115 | ATGTCGAAGCAGCACT | 91 | E | 349 |
| 1232900 | 21203 | 21218 | 3174 | 3189 | TTCCGAAGTCACCGAG | 69 | E | 350 |
| 1232908 | 23039 | 23054 | 3314 | 3329 | ATAGAGTCTGGTCAGG | 52 | E | 351 |
| 1232916 | 23082 | 23097 | 3357 | 3372 | ATAAAATTGCGACTCC | 55 | E | 352 |
| 1232924 | 31009 | 31024 | 3453 | 3468 | GTACCGAGGACAAAGC | 66 | E | 353 |
| 1232932 | 31124 | 31139 | 3568 | 3583 | AACACCTGAAGCTTGC | 48 | E | 354 |
| 1232940 | 35472 | 35487 | 3825 | 3840 | AGACGACGGTCAGCTC | 77† | E | 355 |
| 1232948 | 35527 | 35542 | 3880 | 3895 | CCGGAGAACACTGGCG | 69 | E | 356 |
| 1232956 | 35605 | 35620 | 3958 | 3973 | CCGGAAGGATCACAGA | 85 | E | 357 |
| 1232964 | 35716 | 35731 | 4069 | 4084 | CAACACCCGATGCTGT | 74 | E | 358 |
| 1232972 | 35884 | 35899 | 4237 | 4252 | TAGCGGGAATGATGAT | 82 | E | 359 |
| 1232980 | 35926 | 35941 | 4279 | 4294 | TATAGTCAACTAGTTC | 56 | E | 360 |
| 1232988 | 3628 | 3643 | N/A | N/A | AGCTCCGCATACCCTC | 60 | E | 361 |
| | 3691 | 3706 | | | | | | |
| 1232996 | 3636 | 3651 | N/A | N/A | GCCCTATTAGCTCCGC | 78 | E | 362 |
| | 3699 | 3714 | | | | | | |
| 1233004 | 4024 | 4039 | N/A | N/A | ATCTTTAGATCTTCCA | 54 | E | 363 |
| 1233012 | 6449 | 6464 | N/A | N/A | AAATTATTACGGCCGG | 75 | E | 364 |
| 1233020 | 6831 | 6846 | N/A | N/A | GTAGAAGTAATCCAGG | 46 | E | 365 |
| 1233028 | 8483 | 8498 | N/A | N/A | GAGTATATAAGTACTG | 75 | E | 366 |
| 1233036 | 9083 | 9098 | N/A | N/A | CCCATAAAAGACCTCT | 64 | E | 367 |
| 1233044 | 9936 | 9951 | N/A | N/A | AAACATGCGAAGCAGG | 66 | E | 368 |
| 1233052 | 10433 | 10448 | N/A | N/A | GAACCTATAGTATGGC | 77 | E | 369 |
| 1233060 | 10834 | 10849 | N/A | N/A | AATCTACGGCAAAGAC | 98 | E | 370 |
| 1233068 | 12828 | 12843 | N/A | N/A | TGCTAAACTGGCATTC | 81 | E | 371 |
| 1233076 | 14045 | 14060 | N/A | N/A | CCGTAAGGTGAACCTT | 50 | E | 372 |
| 1233084 | 14547 | 14562 | N/A | N/A | AGCCCCCTACCAGGGC | 89 | E | 373 |
| | 15202 | 15217 | | | | | | |
| | 15275 | 15290 | | | | | | |
| 1233092 | 16541 | 16556 | N/A | N/A | AAGAAATCAGCCTCGA | 87 | E | 374 |
| 1233100 | 17665 | 17680 | N/A | N/A | ATATCGAGCATCATGG | 34 | E | 375 |
| 1233108 | 18422 | 18437 | N/A | N/A | ATTGATAAGACTGGTG | 45 | E | 376 |
| 1233116 | 18605 | 18620 | N/A | N/A | TACTAAGATGTCTGTC | 49 | E | 377 |
| 1233124 | 18997 | 19012 | N/A | N/A | CTCGAAGAATTGCTTG | 61 | E | 378 |
| 1233132 | 19500 | 19515 | N/A | N/A | ACTACGATTTGAAATT | 72 | E | 379 |
| 1233140 | 20576 | 20591 | N/A | N/A | GAGCTCCGGAATACAG | 79 | E | 380 |
| | 20954 | 20969 | | | | | | |
| 1233148 | 20698 | 20713 | N/A | N/A | TCCGGAATAGAGAAAG | 83 | E | 381 |
| | 20740 | 20755 | | | | | | |
| | 20908 | 20923 | | | | | | |
| | 20992 | 21007 | | | | | | |
| 1233156 | 21640 | 21655 | N/A | N/A | CAACATCGAGGGTCTG | 54 | E | 382 |
| 1233164 | 23210 | 23225 | N/A | N/A | CCAAAGTAACCCCCAT | 62 | E | 383 |
| 1233172 | 23475 | 23490 | N/A | N/A | ACTATGCGATAACTTG | 31 | E | 384 |
| 1233180 | 23689 | 23704 | N/A | N/A | AGGTAAAACATCCACC | 76 | E | 385 |
| 1233188 | 24157 | 24172 | N/A | N/A | AAATTGGAGGTCCCAC | 80 | E | 386 |
| 1233196 | 24501 | 24516 | N/A | N/A | AATACCCTAAGGCGAG | 50 | E | 387 |
| 1233204 | 24534 | 24549 | N/A | N/A | GTAATATTCTAAGGCG | 40 | E | 388 |
| 1233212 | 25671 | 25686 | N/A | N/A | AACCAGGCGGGTGTGT | 88 | E | 389 |
| 1233220 | 26568 | 26583 | N/A | N/A | AGTTAAGATCAGGTCG | 36 | E | 390 |
| 1233228 | 26782 | 26797 | N/A | N/A | CAATCGATAGATCTAC | 70 | E | 391 |
| 1233236 | 26945 | 26960 | N/A | N/A | CACTTATAGATACCTA | 68 | E | 392 |
| 1233244 | 27026 | 27041 | N/A | N/A | CATAGGCTATACCTAA | 29 | E | 393 |
| | 27062 | 27077 | | | | | | |
| | 27098 | 27113 | | | | | | |
| | 27116 | 27131 | | | | | | |
| 1233252 | 27099 | 27114 | N/A | N/A | ACATAGGCTATACCTA | 57 | E | 394 |
| | 27117 | 27132 | | | | | | |
| 1233260 | 27380 | 27395 | N/A | N/A | GAACCGAAAGCAGAGC | 58 | E | 395 |
| 1233268 | 28610 | 28625 | N/A | N/A | ACGGATATCTTGCCAT | 50 | E | 396 |
| 1233276 | 28756 | 28771 | N/A | N/A | ATTAAGCAACGGTTAC | 77 | E | 397 |
| 1233284 | 29142 | 29157 | N/A | N/A | AGTAAGACTAATAGGC | 81 | E | 398 |
| 1233292 | 29534 | 29549 | N/A | N/A | GCCTTTTAGAGCATAA | 60 | E | 399 |
| 1233300 | 31899 | 31914 | N/A | N/A | AATGAAGCGAGAGCCA | 61 | E | 400 |
| 1233308 | 32111 | 32126 | N/A | N/A | ACGAAGTTCGGTTCCT | 60 | E | 401 |
| 1233316 | 32156 | 32171 | N/A | N/A | CTATGTAAAGACCCTT | 63 | E | 402 |
| 1233324 | 34084 | 34099 | N/A | N/A | AAAACGGGTTGATTGC | 48 | E | 403 |
| 1233332 | 34381 | 34396 | N/A | N/A | AAGTAGTTAGAGTTAG | 53 | E | 404 |
| 1233340 | 34877 | 34892 | N/A | N/A | CGACAATTAAACAGGT | 68 | E | 405 |
| 1232732 | 5114 | 5129 | 66 | 81 | AAAATTTTTGTCCCGT | 78 | F | 406 |
| 1232737 | 5138 | 5153 | 90 | 105 | ATGCCATCTTGACCCA | 60 | F | 22 |
| 1232741 | 5148 | 5163 | 100 | 115 | CCACTTCACGATGCCA | 23 | F | 407 |
| 1232749 | 5164 | 5179 | 116 | 131 | AGTTTACGGTGAACAA | 62 | F | 408 |
| 1232757 | 5179 | 5194 | 131 | 146 | ACAGGATTGTATTACA | 81 | F | 409 |
| 1232765 | 5293 | 5308 | 245 | 260 | AGTTAGCCAAATGCTT | 79 | F | 410 |
| 1232773 | 5726 | 5741 | 678 | 693 | GTGAACACGGCACACG | 60 | F | 411 |
| 1232781 | 5965 | 5980 | 917 | 932 | ATTGAAGTCGATCATT | 87 | F | 412 |
| 1232789 | 6009 | 6024 | 961 | 976 | GCAGCGAAGATCCACA | 36 | F | 413 |
| 1232797 | 6035 | 6050 | 987 | 1002 | TCTCATAAAGGTCTCT | 42 | F | 414 |
| 1232805 | 10306 | 10321 | 1106 | 1121 | CGAAAGGTACTCCAGT | 38 | F | 415 |
| 1232813 | 10318 | 10333 | 1118 | 1133 | AATAGAGATTCTCGAA | 74 | F | 416 |
| 1232821 | 11043 | 11058 | 1346 | 1361 | CATCTTAATGGGACTC | 23 | F | 417 |
| 1232829 | 11530 | 11545 | 1833 | 1848 | CATGCCGAGGATGGTC | 58 | F | 418 |
| 1232837 | 12083 | 12098 | 2386 | 2401 | TTCAAACGACTCCCTG | 65 | F | 419 |
| 1232845 | 12139 | 12154 | 2442 | 2457 | CCTTTTCGAATTTGCC | 22 | F | 420 |
| 1232853 | 12358 | 12373 | 2661 | 2676 | GCACGAAGTCCTCCTC | 42 | F | 421 |
| 1232861 | 12458 | 12473 | 2761 | 2776 | TCCACCCGATGACAGT | 63 | F | 422 |
| 1232869 | 16615 | 16630 | 2933 | 2948 | AAAGAGGCCCCGGCAA | 73 | F | 423 |
| 1232877 | 16699 | 16714 | 3017 | 3032 | ACACAACACTCTCATC | 67 | F | 424 |
| 1232885 | 21111 | 21126 | 3082 | 3097 | TGCGAGAGGCCACAGC | 74 | F | 425 |
| 1232893 | 21131 | 21146 | 3102 | 3117 | AGATGTCGAAGCAGCA | 58 | F | 426 |
| 1232901 | 21204 | 21219 | 3175 | 3190 | ATTCCGAAGTCACCGA | 67 | F | 427 |
| 1232909 | 23040 | 23055 | 3315 | 3330 | CATAGAGTCTGGTCAG | 67 | F | 428 |
| 1232917 | 23083 | 23098 | 3358 | 3373 | CATAAAATTGCGACTC | 72 | F | 429 |
| 1232925 | 31010 | 31025 | 3454 | 3469 | AGTACCGAGGACAAAG | 62 | F | 430 |
| 1232933 | 31765 | 31780 | 3701 | 3716 | ACAGAACATCATGACC | 76† | F | 431 |
| 1232941 | 35473 | 35488 | 3826 | 3841 | AAGACGACGGTCAGCT | 91† | F | 432 |
| 1232949 | 35528 | 35543 | 3881 | 3896 | ACCGGAGAACACTGGC | 79 | F | 433 |
| 1232957 | 35608 | 35623 | 3961 | 3976 | CCACCGGAAGGATCAC | 79 | F | 434 |
| 1232965 | 35719 | 35734 | 4072 | 4087 | CAACAACACCCGATGC | 85 | F | 435 |
| 1232973 | 35885 | 35900 | 4238 | 4253 | ATAGCGGGAATGATGA | 65 | F | 436 |
| 1232981 | 3422 | 3437 | N/A | N/A | AAACTATGGTTGCTGC | 75 | F | 437 |
| 1232989 | 3629 | 3644 | N/A | N/A | TAGCTCCGCATACCCT | 60 | F | 438 |
| | 3692 | 3707 | | | | | | |
| 1232997 | 3637 | 3652 | N/A | N/A | AGCCCTATTAGCTCCG | 74 | F | 439 |
| | 3700 | 3715 | | | | | | |
| 1233005 | 4044 | 4059 | N/A | N/A | AACTAGCAATCACCTG | 78 | F | 440 |
| 1233013 | 6450 | 6465 | N/A | N/A | AAAATTATTACGGCCG | 87 | F | 441 |
| 1233021 | 6946 | 6961 | N/A | N/A | TACCGTATGATTCAGA | 41 | F | 442 |
| 1233029 | 8532 | 8547 | N/A | N/A | AGCAAAGTACTTCTAC | 61 | F | 443 |
| 1233037 | 9457 | 9472 | N/A | N/A | AACAAGAGTTAGTCAC | 79 | F | 444 |
| 1233045 | 9937 | 9952 | N/A | N/A | CAAACATGCGAAGCAG | 85 | F | 445 |
| 1233053 | 10544 | 10559 | N/A | N/A | CGCTTAATCACCGCCG | 49 | F | 446 |
| 1233061 | 10835 | 10850 | N/A | N/A | TAATCTACGGCAAAGA | 80 | F | 447 |
| 1233069 | 12841 | 12856 | N/A | N/A | ACTTAATACCTTGTGC | 82 | F | 448 |
| 1233077 | 14297 | 14312 | N/A | N/A | CAAAATCCATACGCTG | 67 | F | 449 |
| 1233085 | 14571 | 14586 | N/A | N/A | TGCCATCTAGAGGGAT | 34 | F | 450 |
| | 14863 | 14878 | | | | | | |
| | 15153 | 15168 | | | | | | |
| | 15226 | 15241 | | | | | | |
| 1233093 | 16573 | 16588 | N/A | N/A | CAATCTAGGAATTAGA | 99 | F | 451 |
| 1233101 | 17666 | 17681 | N/A | N/A | TATATCGAGCATCATG | 44 | F | 452 |
| 1233109 | 18423 | 18438 | N/A | N/A | AATTGATAAGACTGGT | 50 | F | 453 |
| 1233117 | 18606 | 18621 | N/A | N/A | GTACTAAGATGTCTGT | 31 | F | 454 |
| 1233125 | 18998 | 19013 | N/A | N/A | ACTCGAAGAATTGCTT | 50 | F | 455 |
| 1233133 | 19535 | 19550 | N/A | N/A | AAGGTTAAATGTCCCA | 46 | F | 456 |
| 1233141 | 20577 | 20592 | N/A | N/A | AGAGCTCCGGAATACA | 55 | F | 457 |
| | 20955 | 20970 | | | | | | |
| 1233149 | 20699 | 20714 | N/A | N/A | CTCCGGAATAGAGAAA | 63 | F | 458 |
| | 20741 | 20756 | | | | | | |
| | 20909 | 20924 | | | | | | |
| | 20993 | 21008 | | | | | | |
| 1233157 | 21641 | 21656 | N/A | N/A | ACAACATCGAGGGTCT | 28 | F | 459 |
| 1233165 | 23211 | 23226 | N/A | N/A | ACCAAAGTAACCCCCA | 55 | F | 460 |
| 1233173 | 23476 | 23491 | N/A | N/A | CACTATGCGATAACTT | 58 | F | 461 |
| 1233181 | 23817 | 23832 | N/A | N/A | TATTAGTGGGTATTCC | 65 | F | 462 |
| 1233189 | 24160 | 24175 | N/A | N/A | GCTAAATTGGAGGTCC | 51 | F | 463 |
| 1233197 | 24502 | 24517 | N/A | N/A | TAATACCCTAAGGCGA | 76 | F | 464 |
| 1233205 | 25020 | 25035 | N/A | N/A | AGTAACTACATAGAGC | 46 | F | 465 |
| 1233213 | 25673 | 25688 | N/A | N/A | TAAACCAGGCGGGTGT | 86 | F | 466 |
| 1233221 | 26683 | 26698 | N/A | N/A | AGATAGGGTGTTATGG | 36 | F | 467 |
| 1233229 | 26784 | 26799 | N/A | N/A | CCCAATCGATAGATCT | 30 | F | 468 |
| 1233237 | 26967 | 26982 | N/A | N/A | AAAAGTCTCGACTGAT | 53 | F | 469 |
| 1233245 | 27027 | 27042 | N/A | N/A | CCATAGGCTATACCTA | 51 | F | 470 |
| | 27063 | 27078 | | | | | | |
| 1233253 | 27136 | 27151 | N/A | N/A | AAATGTAGCTATACCT | 91 | F | 471 |
| 1233261 | 27498 | 27513 | N/A | N/A | AGCTAAGATGTCACTC | 75 | F | 472 |
| 1233269 | 28614 | 28629 | N/A | N/A | AAGCACGGATATCTTG | 68 | F | 473 |
| 1233277 | 28758 | 28773 | N/A | N/A | CTATTAAGCAACGGTT | 55 | F | 474 |
| 1233285 | 29143 | 29158 | N/A | N/A | AAGTAAGACTAATAGG | 81 | F | 475 |
| 1233293 | 30599 | 30614 | N/A | N/A | AATCCCCGTAACACTG | 80 | F | 476 |
| 1233301 | 31900 | 31915 | N/A | N/A | AAATGAAGCGAGAGCC | 80 | F | 477 |
| 1233309 | 32112 | 32127 | N/A | N/A | TACGAAGTTCGGTTCC | 53 | F | 478 |
| 1233317 | 32914 | 32929 | N/A | N/A | TATAATCTGGAGGCCA | 79 | F | 479 |
| 1233325 | 34085 | 34100 | N/A | N/A | GAAAACGGGTTGATTG | 71 | F | 480 |
| 1233333 | 34413 | 34428 | N/A | N/A | AATGTACGGTACTTAA | 69 | F | 481 |
| 1233341 | 34910 | 34925 | N/A | N/A | TATAATGAACCCCCTG | 77 | F | 482 |
| 1232733 | 5129 | 5144 | 81 | 96 | TGACCCATCAGCAAGA | 74 | G | 483 |
| 1232737 | 5138 | 5153 | 90 | 105 | ATGCCATCTTGACCCA | 72 | G | 22 |
| 1232742 | 5152 | 5167 | 104 | 119 | ACAACCACTTCACGAT | 70 | G | 484 |
| 1232750 | 5165 | 5180 | 117 | 132 | CAGTTTACGGTGAACA | 57 | G | 485 |
| 1232758 | 5184 | 5199 | 136 | 151 | CATAAACAGGATTGTA | 78 | G | 486 |
| 1232766 | 5294 | 5309 | 246 | 261 | AAGTTAGCCAAATGCT | 62 | G | 487 |
| 1232774 | 5727 | 5742 | 679 | 694 | AGTGAACACGGCACAC | 64 | G | 488 |
| 1232782 | 5966 | 5981 | 918 | 933 | CATTGAAGTCGATCAT | 84 | G | 489 |
| 1232790 | 6010 | 6025 | 962 | 977 | CGCAGCGAAGATCCAC | 45 | G | 490 |
| 1232798 | 10244 | 10259 | 1044 | 1059 | TGGGATTCGAAACACG | 84 | G | 491 |
| 1232806 | 10307 | 10322 | 1107 | 1122 | TCGAAAGGTACTCCAG | 41 | G | 492 |
| 1232814 | 10319 | 10334 | 1119 | 1134 | AAATAGAGATTCTCGA | 77 | G | 493 |
| 1232822 | 11213 | 11228 | 1516 | 1531 | CGACAGTGGATATAGA | 66 | G | 494 |
| 1232830 | 11531 | 11546 | 1834 | 1849 | ACATGCCGAGGATGGT | 66 | G | 495 |
| 1232838 | 12086 | 12101 | 2389 | 2404 | AGCTTCAAACGACTCC | 48 | G | 496 |
| 1232846 | 12140 | 12155 | 2443 | 2458 | CCCTTTTCGAATTTGC | 43 | G | 497 |
| 1232854 | 12377 | 12392 | 2680 | 2695 | AAATAGTCCATGGCCC | 69 | G | 498 |
| 1232862 | 12459 | 12474 | 2762 | 2777 | CTCCACCCGATGACAG | 62 | G | 499 |
| 1232870 | 16616 | 16631 | 2934 | 2949 | AAAAGAGGCCCCGGCA | 71 | G | 500 |
| 1232878 | 16732 | 16747 | 3050 | 3065 | CCGAATGTTACAGCCA | 44 | G | 501 |
| 1232886 | 21112 | 21127 | 3083 | 3098 | ATGCGAGAGGCCACAG | 68 | G | 502 |
| 1232894 | 21135 | 21150 | 3106 | 3121 | AAGGAGATGTCGAAGC | 70 | G | 503 |
| 1232902 | 21205 | 21220 | 3176 | 3191 | GATTCCGAAGTCACCG | 74 | G | 504 |
| 1232910 | 23041 | 23056 | 3316 | 3331 | ACATAGAGTCTGGTCA | 66 | G | 505 |
| 1232918 | 23084 | 23099 | 3359 | 3374 | ACATAAAATTGCGACT | 86 | G | 506 |
| 1232926 | 31011 | 31026 | 3455 | 3470 | GAGTACCGAGGACAAA | 79 | G | 507 |
| 1232934 | 35434 | 35449 | 3787 | 3802 | TCTAACGCACTTTTTG | 21† | G | 508 |
| 1232942 | 35474 | 35489 | 3827 | 3842 | AAAGACGACGGTCAGC | 80† | G | 509 |
| 1232950 | 35529 | 35544 | 3882 | 3897 | GACCGGAGAACACTGG | 73 | G | 510 |
| 1232958 | 35610 | 35625 | 3963 | 3978 | CTCCACCGGAAGGATC | 85 | G | 511 |
| 1232966 | 35724 | 35739 | 4077 | 4092 | GATGACAACAACACCC | 84 | G | 512 |
| 1232974 | 35886 | 35901 | 4239 | 4254 | GATAGCGGGAATGATG | 106 | G | 513 |
| 1232982 | 3621 | 3636 | N/A | N/A | CATACCCTCCCAGCTC | 81 | G | 514 |
| | 3684 | 3699 | | | | | | |
| 1232990 | 3630 | 3645 | N/A | N/A | TTAGCTCCGCATACCC | 82 | G | 515 |
| | 3693 | 3708 | | | | | | |
| 1232998 | 3638 | 3653 | N/A | N/A | CAGCCCTATTAGCTCC | 65 | G | 516 |
| | 3701 | 3716 | | | | | | |
| 1233006 | 4203 | 4218 | N/A | N/A | AGATATGAGACCATGT | 71 | G | 517 |
| 1233014 | 6451 | 6466 | N/A | N/A | AAAAATTATTACGGCC | 94 | G | 518 |
| 1233022 | 7764 | 7779 | N/A | N/A | CACTACCGTGAGAGGG | 60 | G | 519 |
| 1233030 | 8595 | 8610 | N/A | N/A | GATAATACCAGAAGCT | 74 | G | 520 |
| 1233038 | 9500 | 9515 | N/A | N/A | TATCTTAAGAAGGCAT | 74 | G | 521 |
| 1233046 | 10064 | 10079 | N/A | N/A | CAAAAGTCTGGTTGGG | 73 | G | 522 |
| 1233054 | 10581 | 10596 | N/A | N/A | TAAGTAATCTGCATCC | 75 | G | 523 |
| 1233062 | 10841 | 10856 | N/A | N/A | TTACGGTAATCTACGG | 76 | G | 524 |
| 1233070 | 12842 | 12857 | N/A | N/A | TACTTAATACCTTGTG | 89 | G | 525 |
| 1233078 | 14375 | 14390 | N/A | N/A | TAATGAGTAGTTCTGC | 78 | G | 526 |
| 1233086 | 14686 | 14701 | N/A | N/A | TGCCAGGGCTCCAATC | 86 | G | 527 |
| | 14832 | 14847 | | | | | | |
| | 14978 | 14993 | | | | | | |
| 1233094 | 17257 | 17272 | N/A | N/A | ATCTTAGACTTTCGGC | 60 | G | 528 |
| 1233102 | 17667 | 17682 | N/A | N/A | ATATATCGAGCATCAT | 46 | G | 529 |
| 1233110 | 18461 | 18476 | N/A | N/A | GACGAGGAAAAGTGGA | 71 | G | 530 |
| 1233118 | 18734 | 18749 | N/A | N/A | ATCGAATCAGAATTTT | 57 | G | 531 |
| 1233126 | 18999 | 19014 | N/A | N/A | TACTCGAAGAATTGCT | 55 | G | 532 |
| 1233134 | 19536 | 19551 | N/A | N/A | TAAGGTTAAATGTCCC | 74 | G | 533 |
| 1233142 | 20579 | 20594 | N/A | N/A | AGAGAGCTCCGGAATA | 59 | G | 534 |
| | 20705 | 20720 | | | | | | |
| | 20747 | 20762 | | | | | | |
| | 20915 | 20930 | | | | | | |
| | 20957 | 20972 | | | | | | |
| 1233150 | 20700 | 20715 | N/A | N/A | GCTCCGGAATAGAGAA | 65 | G | 535 |
| | 20742 | 20757 | | | | | | |
| | 20910 | 20925 | | | | | | |
| | 20994 | 21009 | | | | | | |
| 1233158 | 22431 | 22446 | N/A | N/A | GCATAATGTCCCCGAG | 52 | G | 536 |
| 1233166 | 23212 | 23227 | N/A | N/A | GACCAAAGTAACCCCC | 62 | G | 537 |
| 1233174 | 23494 | 23509 | N/A | N/A | TAGTGTAGAGGCTGCA | 22 | G | 538 |
| | 23527 | 23542 | | | | | | |
| 1233182 | 23819 | 23834 | N/A | N/A | ATTATTAGTGGGTATT | 80 | G | 539 |
| 1233190 | 24161 | 24176 | N/A | N/A | AGCTAAATTGGAGGTC | 79 | G | 540 |
| 1233198 | 24526 | 24541 | N/A | N/A | CTAAGGCGATTTGCTA | 64 | G | 541 |
| 1233206 | 25159 | 25174 | N/A | N/A | CCTATAAGGACCCAGC | 46 | G | 542 |
| 1233214 | 26089 | 26104 | N/A | N/A | ACCAATATATGTTCAC | 71 | G | 543 |
| 1233222 | 26686 | 26701 | N/A | N/A | AATAGATAGGGTGTTA | 68 | G | 544 |
| 1233230 | 26810 | 26825 | N/A | N/A | GTAAAGCACACTATGG | 64 | G | 545 |
| 1233238 | 26968 | 26983 | N/A | N/A | TAAAAGTCTCGACTGA | 56 | G | 546 |
| 1233246 | 27028 | 27043 | N/A | N/A | CCCATAGGCTATACCT | 34 | G | 547 |
| | 27064 | 27079 | | | | | | |
| 1233254 | 27154 | 27169 | N/A | N/A | CCAAAGTTCGAGAGAA | 79 | G | 548 |
| 1233262 | 27769 | 27784 | N/A | N/A | TAATTCATAGCTGGGC | 76 | G | 549 |
| 1233270 | 28621 | 28636 | N/A | N/A | ATTTACCAAGCACGGA | 45 | G | 550 |
| 1233278 | 28759 | 28774 | N/A | N/A | ACTATTAAGCAACGGT | 42 | G | 551 |
| 1233286 | 29147 | 29162 | N/A | N/A | CAGTAAGTAAGACTAA | 89 | G | 552 |
| 1233294 | 30605 | 30620 | N/A | N/A | ACTAATAATCCCCGTA | 91 | G | 553 |
| 1233302 | 31901 | 31916 | N/A | N/A | CAAATGAAGCGAGAGC | 66 | G | 554 |
| 1233310 | 32115 | 32130 | N/A | N/A | TTCTACGAAGTTCGGT | 62 | G | 555 |
| 1233318 | 33720 | 33735 | N/A | N/A | ACATAGCACATAAGAT | 79 | G | 556 |
| 1233326 | 34086 | 34101 | N/A | N/A | AGAAAACGGGTTGATT | 75 | G | 557 |
| 1233334 | 34414 | 34429 | N/A | N/A | TAATGTACGGTACTTA | 78 | G | 558 |
| 1233342 | 34961 | 34976 | N/A | N/A | TAACTATCTAGTTGGG | 65 | G | 559 |
| 1232734 | 5131 | 5146 | 83 | 98 | CTTGACCCATCAGCAA | 72 | H | 560 |
| 1232737 | 5138 | 5153 | 90 | 105 | ATGCCATCTTGACCCA | 59 | H | 22 |
| 1232743 | 5157 | 5172 | 109 | 124 | GGTGAACAACCACTTC | 85 | H | 561 |
| 1232751 | 5166 | 5181 | 118 | 133 | ACAGTTTACGGTGAAC | 58 | H | 562 |
| 1232759 | 5185 | 5200 | 137 | 152 | CCATAAACAGGATTGT | 85 | H | 563 |
| 1232767 | 5596 | 5611 | 548 | 563 | AGTTAGCCAAGGCCGG | 61 | H | 564 |
| 1232775 | 5735 | 5750 | 687 | 702 | TACCAGGCAGTGAACA | 70 | H | 565 |
| 1232783 | 5967 | 5982 | 919 | 934 | CCATTGAAGTCGATCA | 72 | H | 566 |
| 1232791 | 6011 | 6026 | 963 | 978 | TCGCAGCGAAGATCCA | 56 | H | 567 |
| 1232799 | 10245 | 10260 | 1045 | 1060 | GTGGGATTCGAAACAC | 96 | H | 568 |
| 1232807 | 10308 | 10323 | 1108 | 1123 | CTCGAAAGGTACTCCA | 44 | H | 569 |
| 1232815 | 10905 | 10920 | 1208 | 1223 | ACCCAGACGGGCATTC | 73 | H | 570 |
| 1232823 | 11303 | 11318 | 1606 | 1621 | CTCACGATCTTGTGGA | 69 | H | 571 |
| 1232831 | 11898 | 11913 | 2201 | 2216 | CCTGAGGTCGGACTCC | 42 | H | 572 |
| 1232839 | 12114 | 12129 | 2417 | 2432 | CAGAAGGACTGTCACG | 70 | H | 573 |
| 1232847 | 12141 | 12156 | 2444 | 2459 | CCCCTTTTCGAATTTG | 64 | H | 574 |
| 1232855 | 12378 | 12393 | 2681 | 2696 | GAAATAGTCCATGGCC | 72 | H | 575 |
| 1232863 | 12487 | 12502 | 2790 | 2805 | TGTTATGGAGAAACCC | 73 | H | 576 |
| 1232871 | 16617 | 16632 | 2935 | 2950 | GAAAAGAGGCCCCGGC | 55 | H | 577 |
| 1232879 | 16733 | 16748 | 3051 | 3066 | TCCGAATGTTACAGCC | 46 | H | 578 |
| 1232887 | 21113 | 21128 | 3084 | 3099 | CATGCGAGAGGCCACA | 82 | H | 579 |
| 1232895 | 21137 | 21152 | 3108 | 3123 | CCAAGGAGATGTCGAA | 76 | H | 580 |
| 1232903 | 21206 | 21221 | 3177 | 3192 | TGATTCCGAAGTCACC | 87 | H | 581 |
| 1232911 | 23043 | 23058 | 3318 | 3333 | CCACATAGAGTCTGGT | 91 | H | 582 |
| 1232919 | 23085 | 23100 | 3360 | 3375 | CACATAAAATTGCGAC | 61 | H | 583 |
| 1232927 | 31021 | 31036 | 3465 | 3480 | GATTAGTGCTGAGTAC | 66 | H | 584 |
| 1232935 | 35435 | 35450 | 3788 | 3803 | TTCTAACGCACTTTTT | 36† | H | 585 |
| 1232943 | 35475 | 35490 | 3828 | 3843 | CAAAGACGACGGTCAG | 104† | H | 586 |
| 1232951 | 35530 | 35545 | 3883 | 3898 | GGACCGGAGAACACTG | 94 | H | 587 |
| 1232959 | 35671 | 35686 | 4024 | 4039 | GTAAAGGAGATGCCCA | 80 | H | 588 |
| 1232967 | 35755 | 35770 | 4108 | 4123 | CAAGAGGAACATCCTC | 105 | H | 589 |
| 1232975 | 35887 | 35902 | 4240 | 4255 | AGATAGCGGGAATGAT | 83 | H | 590 |
| 1232983 | 3622 | 3637 | N/A | N/A | GCATACCCTCCCAGCT | 118 | H | 591 |
| | 3685 | 3700 | | | | | | |
| 1232991 | 3631 | 3646 | N/A | N/A | ATTAGCTCCGCATACC | 100 | H | 592 |
| | 3694 | 3709 | | | | | | |
| 1232999 | 3660 | 3675 | N/A | N/A | CCTGAGGTGACTGCTC | 56 | H | 593 |
| | 3723 | 3738 | | | | | | |
| 1233007 | 4264 | 4279 | N/A | N/A | GACTAGGAAGCCTGCC | 96 | H | 594 |
| 1233015 | 6452 | 6467 | N/A | N/A | TAAAAATTATTACGGC | 99 | H | 595 |
| 1233023 | 7861 | 7876 | N/A | N/A | GACTAATCATGGCAGG | 79 | H | 596 |
| 1233031 | 8596 | 8611 | N/A | N/A | GGATAATACCAGAAGC | 86 | H | 597 |
| 1233039 | 9512 | 9527 | N/A | N/A | CACTAGATAGCATATC | 81 | H | 598 |
| 1233047 | 10162 | 10177 | N/A | N/A | CAATTGAAACCTGGCC | 75 | H | 599 |
| 1233055 | 10697 | 10712 | N/A | N/A | TAGGAAAGGTGCCGAG | 85 | H | 600 |
| 1233063 | 10842 | 10857 | N/A | N/A | CTTACGGTAATCTACG | 95 | H | 601 |
| 1233071 | 12866 | 12881 | N/A | N/A | ATATTTGTAGGCAATC | 74 | H | 602 |
| 1233079 | 14378 | 14393 | N/A | N/A | TCTTAATGAGTAGTTC | 92 | H | 603 |
| 1233087 | 15661 | 15676 | N/A | N/A | AGCTATATGGATCTTT | 73 | H | 604 |
| 1233095 | 17440 | 17455 | N/A | N/A | ACATTAGGTCTTGTGT | 71 | H | 605 |
| 1233103 | 17668 | 17683 | N/A | N/A | AATATATCGAGCATCA | 39 | H | 606 |
| 1233111 | 18558 | 18573 | N/A | N/A | AAACGCAAACCCCATC | 82 | H | 607 |
| 1233119 | 18738 | 18753 | N/A | N/A | CACAATCGAATCAGAA | 60 | H | 608 |
| 1233127 | 19001 | 19016 | N/A | N/A | ACTACTCGAAGAATTG | 47 | H | 609 |
| 1233135 | 19743 | 19758 | N/A | N/A | CAATAGTTCATAGGTC | 88 | H | 610 |
| 1233143 | 20582 | 20597 | N/A | N/A | ACCAGAGAGCTCCGGA | 35 | H | 611 |
| | 20708 | 20723 | | | | | | |
| | 20750 | 20765 | | | | | | |
| | 20918 | 20933 | | | | | | |
| 1233151 | 20701 | 20716 | N/A | N/A | AGCTCCGGAATAGAGA | 67 | H | 612 |
| | 20743 | 20758 | | | | | | |
| | 20911 | 20926 | | | | | | |
| | 20995 | 21010 | | | | | | |
| 1233159 | 22482 | 22497 | N/A | N/A | TATAAGGACAATCAGA | 88 | H | 613 |
| 1233167 | 23284 | 23299 | N/A | N/A | ACATTATATGCCTCCA | 36 | H | 614 |
| 1233175 | 23495 | 23510 | N/A | N/A | CTAGTGTAGAGGCTGC | 18 | H | 615 |
| | 23528 | 23543 | | | | | | |
| 1233183 | 23820 | 23835 | N/A | N/A | GATTATTAGTGGGTAT | 53 | H | 616 |
| 1233191 | 24162 | 24177 | N/A | N/A | AAGCTAAATTGGAGGT | 83 | H | 617 |
| 1233199 | 24527 | 24542 | N/A | N/A | TCTAAGGCGATTTGCT | 67 | H | 618 |
| 1233207 | 25160 | 25175 | N/A | N/A | ACCTATAAGGACCCAG | 43 | H | 619 |
| 1233215 | 26091 | 26106 | N/A | N/A | CAACCAATATATGTTC | 86 | H | 620 |
| 1233223 | 26689 | 26704 | N/A | N/A | TTCAATAGATAGGGTG | 55 | H | 621 |
| 1233231 | 26814 | 26829 | N/A | N/A | TATGGTAAAGCACACT | 55 | H | 622 |
| 1233239 | 26971 | 26986 | N/A | N/A | GCATAAAAGTCTCGAC | 39 | H | 623 |
| 1233247 | 27029 | 27044 | N/A | N/A | ACCCATAGGCTATACC | 40 | H | 624 |
| | 27065 | 27080 | | | | | | |
| 1233255 | 27241 | 27256 | N/A | N/A | TAAACCAATGTTGCAA | 52 | H | 625 |
| 1233263 | 27771 | 27786 | N/A | N/A | ACTAATTCATAGCTGG | 78 | H | 626 |
| 1233271 | 28638 | 28653 | N/A | N/A | ACCTAACTGAGCTCTA | 75 | H | 627 |
| 1233279 | 28760 | 28775 | N/A | N/A | AACTATTAAGCAACGG | 35 | H | 628 |
| 1233287 | 29358 | 29373 | N/A | N/A | GAATAGCGCAGGTGAT | 76 | H | 629 |
| 1233295 | 31140 | 31155 | N/A | N/A | CCAAAGGACTTACTCC | 88 | H | 630 |
| 1233303 | 31902 | 31917 | N/A | N/A | GCAAATGAAGCGAGAG | 59 | H | 631 |
| 1233311 | 32125 | 32140 | N/A | N/A | CATAGACGAGTTCTAC | 78 | H | 632 |
| 1233319 | 33801 | 33816 | N/A | N/A | CATTAATATGGACAGA | 76 | H | 633 |
| 1233327 | 34088 | 34103 | N/A | N/A | TGAGAAAACGGGTTGA | 69 | H | 634 |
| 1233335 | 34415 | 34430 | N/A | N/A | CTAATGTACGGTACTT | 64 | H | 635 |
| 1233343 | 34962 | 34977 | N/A | N/A | TTAACTATCTAGTTGG | 82 | H | 636 |
| 1232737 | 5138 | 5153 | 90 | 105 | ATGCCATCTTGACCCA | 59 | I | 22 |
| 1241411 | 5061 | 5076 | 13 | 28 | ATTCCTCAACTTCAGT | 59 | I | 637 |
| 1241435 | 5144 | 5159 | 96 | 111 | TTCACGATGCCATCTT | 29 | I | 638 |
| 1241459 | 5251 | 5266 | 203 | 218 | TCCAGGAGTGTGTCCT | 20 | I | 639 |
| 1241483 | 5632 | 5647 | 584 | 599 | CATGACTGTAACAAGT | 41 | I | 640 |
| 1241507 | 5761 | 5776 | 713 | 728 | TCCTTAGGCTTCGGTC | 45 | I | 641 |
| 1241531 | 5896 | 5911 | 848 | 863 | GATGCAGCCCTTCTGG | 66 | I | 642 |
| 1241555 | 5971 | 5986 | 923 | 938 | CTCCCCATTGAAGTCG | 50 | I | 643 |
| 1241579 | 6021 | 6036 | 973 | 988 | CTCCTGTTGATCGCAG | 28 | I | 644 |
| 1241603 | 10249 | 10264 | 1049 | 1064 | CACAGTGGGATTCGAA | 54 | I | 645 |
| 1241627 | N/A | N/A | 1143 | 1158 | TACGGTAATCTTTCTT | 29 | I | 646 |
| 1241651 | 10889 | 10904 | 1192 | 1207 | CTGTCTTCAATGCACT | 44 | I | 647 |
| 1241675 | 11081 | 11096 | 1384 | 1399 | GGCTCAGAATGCTCAT | 36 | I | 648 |
| 1241699 | 11300 | 11315 | 1603 | 1618 | ACGATCTTGTGGATGG | 27 | I | 649 |
| 1241723 | 11425 | 11440 | 1728 | 1743 | TCAGGAGAATGTCTCC | 37 | I | 650 |
| 1241747 | 11735 | 11750 | 2038 | 2053 | GATGTCTGGGCAAGGC | 32 | i | 651 |
| 1241771 | 11947 | 11962 | 2250 | 2265 | GGTTCATCCTCAGGAA | 48 | I | 652 |
| 1241794 | 12146 | 12161 | 2449 | 2464 | AAATACCCCTTTTCGA | 100 | I | 653 |
| 1241818 | 12228 | 12243 | 2531 | 2546 | GATCTTGCAACTTAAT | 33 | I | 654 |
| 1241841 | 12361 | 12376 | 2664 | 2679 | TTTGCACGAAGTCCTC | 23 | I | 655 |
| 1241865 | 12466 | 12481 | 2769 | 2784 | ACAGTGACTCCACCCG | 11 | I | 656 |
| 1241889 | 12541 | 12556 | 2844 | 2859 | GCACCATATCAAGGTG | 91 | I | 657 |
| 1241913 | 16586 | 16601 | 2904 | 2919 | GGTGGCTGTTCACCAA | 48 | I | 658 |
| 1241937 | 16740 | 16755 | 3058 | 3073 | CACAATCTCCGAATGT | 69 | I | 659 |
| 1241961 | 21249 | 21264 | 3220 | 3235 | TTCAGATTGCACAACA | 36 | I | 660 |
| 1241985 | 23006 | 23021 | 3281 | 3296 | TACTGATGCAAGATCC | 26 | I | 661 |
| 1242009 | 23054 | 23069 | 3329 | 3344 | GGCATTCTCCCCCACA | 21 | I | 662 |
| 1242033 | 23117 | 23132 | 3392 | 3407 | CTGCAGGTTACACTGT | 90 | I | 663 |
| 1242057 | 31013 | 31028 | 3457 | 3472 | CTGAGTACCGAGGACA | 37 | I | 664 |
| 1242081 | 31057 | 31072 | 3501 | 3516 | GAGTGTTGCCTCGCAG | 19 | I | 665 |
| 1242105 | 31123 | 31138 | 3567 | 3582 | ACACCTGAAGCTTGCA | 27 | I | 666 |
| 1242129 | 31738 | 31753 | 3674 | 3689 | CAGGTCATTGTTGCCC | 72 | I | 667 |
| 1242153 | 35492 | 35507 | 3845 | 3860 | CTCCTACCAAGAAGGC | 104 | I | 668 |
| 1242177 | 35668 | 35683 | 4021 | 4036 | AAGGAGATGCCCAAGC | 56 | I | 669 |
| 1242201 | 35774 | 35789 | 4127 | 4142 | GCTAATTACATGAGGT | 12 | I | 670 |
| 1242225 | 35912 | 35927 | 4265 | 4280 | TCTGTGTTATGGTCAG | 72 | I | 671 |
| 1242249 | N/A | N/A | N/A | N/A | CACCAACCAGAGCTTC | 72 | I | 672 |
| 1242273 | 6741 | 6756 | N/A | N/A | AGGGAATTCCGACACA | 17 | I | 673 |
| 1242297 | 7859 | 7874 | N/A | N/A | CTAATCATGGCAGGTG | 49 | I | 674 |
| 1242321 | 8594 | 8609 | N/A | N/A | ATAATACCAGAAGCTG | 58 | I | 675 |
| 1242345 | 9502 | 9517 | N/A | N/A | CATATCTTAAGAAGGC | 36 | I | 676 |
| 1242369 | 10228 | 10243 | N/A | N/A | TGCATTATCTGAACCT | 55 | I | 677 |
| 1242393 | 12704 | 12719 | N/A | N/A | TATGATGTAGCTGTGG | 22 | I | 678 |
| 1242417 | 13107 | 13122 | N/A | N/A | CATACCAGTCTTCAGT | 62 | I | 679 |
| 1242441 | 14480 | 14495 | N/A | N/A | GTGCTGAGCCTCCTAC | 15 | I | 680 |
| | 14626 | 14641 | | | | | | |
| | 14772 | 14787 | | | | | | |
| | 15064 | 15079 | | | | | | |
| 1242465 | 14545 | 14560 | N/A | N/A | CCCCCTACCAGGGCTC | 63 | I | 681 |
| | 15200 | 15215 | | | | | | |
| | 15273 | 15288 | | | | | | |
| 1242489 | 14589 | 14604 | N/A | N/A | TCCTCAGAGTAAGGCA | 21 | I | 682 |
| | 14881 | 14896 | | | | | | |
| | 15171 | 15186 | | | | | | |
| | 15244 | 15259 | | | | | | |
| 1242513 | 14687 | 14702 | N/A | N/A | CTGCCAGGGCTCCAAT | 45 | I | 683 |
| | 14833 | 14848 | | | | | | |
| | 14979 | 14994 | | | | | | |
| 1242537 | 17441 | 17456 | N/A | N/A | AACATTAGGTCTTGTG | 22 | I | 684 |
| 1242561 | 18372 | 18387 | N/A | N/A | GCAAAATGTCCTAGAG | 45 | I | 685 |
| 1242585 | 19664 | 19679 | N/A | N/A | CAAATGGATCTCTACT | 52 | I | 686 |
| 1242609 | 20570 | 20585 | N/A | N/A | CGGAATACAGCACAAG | 32 | I | 687 |
| 1242633 | 20696 | 20711 | N/A | N/A | CGGAATAGAGAAAGCA | 9 | I | 688 |
| | 20738 | 20753 | | | | | | |
| | 20906 | 20921 | | | | | | |
| | 20990 | 21005 | | | | | | |
| 1242657 | 21600 | 21615 | N/A | N/A | ATACAATATGAGGACC | 55 | I | 689 |
| 1242680 | 22670 | 22685 | N/A | N/A | ACAATTAATGGCCTTA | 39 | I | 690 |
| 1242704 | 23285 | 23300 | N/A | N/A | TACATTATATGCCTCC | 11 | I | 691 |
| 1242728 | 23594 | 23609 | N/A | N/A | CATAGGTGAAGGTGCT | 26 | I | 692 |
| 1242752 | 23925 | 23940 | N/A | N/A | CCTATAGCATCTTTCT | 33 | I | 693 |
| 1242776 | 24181 | 24196 | N/A | N/A | GGTAAGATGAGGTTTA | 74 | I | 694 |
| 1242800 | 24507 | 24522 | N/A | N/A | AAGGGTAATACCCTAA | 78 | I | 695 |
| 1242824 | 25083 | 25098 | N/A | N/A | GTTTAGGCCGCTCTGT | 36 | I | 696 |
| 1242848 | 26000 | 26015 | N/A | N/A | ACGGATGGAAAATAGA | 49 | I | 697 |
| 1242872 | 26462 | 26477 | N/A | N/A | GATAAAGGCAATGGTG | 66 | I | 698 |
| 1242896 | 26796 | 26811 | N/A | N/A | GGGATATTATGCCCCA | 76 | I | 699 |
| 1242920 | 27179 | 27194 | N/A | N/A | ACAATATGCAGCCAGA | 35 | I | 700 |
| 1242944 | 27680 | 27695 | N/A | N/A | GATGAGATCAGGCACT | 44 | I | 701 |
| 1242968 | 28656 | 28671 | N/A | N/A | CTCGATTTCAACCAAT | 26 | I | 702 |
| 1242992 | 28914 | 28929 | N/A | N/A | TAAAGTTATGCAGGCT | 24 | I | 703 |
| 1243016 | 29240 | 29255 | N/A | N/A | GTAAAGCATGAACAGA | 36 | I | 704 |
| 1243040 | 29603 | 29618 | N/A | N/A | ACCTTTTAGTCTCATC | 29 | I | 705 |
| 1243064 | 31223 | 31238 | N/A | N/A | CTAAGATACAGATTAT | 59 | I | 706 |
| 1243088 | 31601 | 31616 | N/A | N/A | CTTAACCATCCCCCTA | 137 | I | 707 |
| 1243112 | 33369 | 33384 | N/A | N/A | AGTGATAAGAAGACGA | 63 | I | 708 |
| 1243136 | 33920 | 33935 | N/A | N/A | TGGGAATATTGGCTTG | 27 | I | 709 |
| 1243160 | 34243 | 34258 | N/A | N/A | TAACAAGAAGGTAGCC | 41 | I | 710 |
| 1243184 | 34430 | 34445 | N/A | N/A | ATGAAGCCCGAACCCC | 87 | I | 711 |
| 1243208 | 34637 | 34652 | N/A | N/A | TCATTTAGTCACTAAC | 86 | I | 712 |
| 1243232 | 7790 | 7805 | N/A | N/A | AACAGTTTGCCTCCAC | 50 | I | 713 |
| 1232737 | 5138 | 5153 | 90 | 105 | ATGCCATCTTGACCCA | 65 | J | 22 |
| 1241412 | 5062 | 5077 | 14 | 29 | TATTCCTCAACTTCAG | 57 | J | 714 |
| 1241436 | 5146 | 5161 | 98 | 113 | ACTTCACGATGCCATC | 45 | J | 715 |
| 1241460 | 5252 | 5267 | 204 | 219 | ATCCAGGAGTGTGTCC | 73 | J | 716 |
| 1241484 | 5689 | 5704 | 641 | 656 | CCACTTTGAGAGATAT | 59 | J | 717 |
| 1241508 | 5763 | 5778 | 715 | 730 | GGTCCTTAGGCTTCGG | 50 | J | 718 |
| 1241532 | 5897 | 5912 | 849 | 864 | GGATGCAGCCCTTCTG | 50 | J | 719 |
| 1241556 | 5972 | 5987 | 924 | 939 | CCTCCCCATTGAAGTC | 75 | J | 720 |
| 1241580 | 6022 | 6037 | 974 | 989 | TCTCCTGTTGATCGCA | 34 | J | 721 |
| 1241604 | 10251 | 10266 | 1051 | 1066 | ATCACAGTGGGATTCG | 50 | J | 722 |
| 1241628 | N/A | N/A | 1144 | 1159 | TTACGGTAATCTTTCT | 54 | J | 723 |
| 1241652 | 10890 | 10905 | 1193 | 1208 | CCTGTCTTCAATGCAC | 50 | J | 724 |
| 1241676 | 11097 | 11112 | 1400 | 1415 | CACCACGGTGTGCACA | 57 | J | 725 |
| 1241700 | 11301 | 11316 | 1604 | 1619 | CACGATCTTGTGGATG | 55 | J | 726 |
| 1241724 | 11438 | 11453 | 1741 | 1756 | CTGATGAGGCTGCTCA | 58 | J | 727 |
| 1241748 | 11736 | 11751 | 2039 | 2054 | GGATGTCTGGGCAAGG | 37 | J | 728 |
| 1241772 | 11948 | 11963 | 2251 | 2266 | AGGTTCATCCTCAGGA | 62 | J | 729 |
| 1241795 | 12147 | 12162 | 2450 | 2465 | CAAATACCCCTTTTCG | 41 | J | 730 |
| 1241819 | 12230 | 12245 | 2533 | 2548 | GAGATCTTGCAACTTA | 42 | J | 731 |
| 1241842 | 12362 | 12377 | 2665 | 2680 | CTTTGCACGAAGTCCT | 47 | J | 732 |
| 1241866 | 12467 | 12482 | 2770 | 2785 | GACAGTGACTCCACCC | 20 | J | 733 |
| 1241890 | 12542 | 12557 | 2845 | 2860 | TGCACCATATCAAGGT | 82 | J | 734 |
| 1241914 | 16589 | 16604 | 2907 | 2922 | TGAGGTGGCTGTTCAC | 56 | J | 735 |
| 1241938 | 16741 | 16756 | 3059 | 3074 | CCACAATCTCCGAATG | 69 | J | 736 |
| 1241962 | N/A | N/A | 3233 | 3248 | CAACCAGAGCTTCTTC | 83 | J | 737 |
| 1241986 | 23007 | 23022 | 3282 | 3297 | ATACTGATGCAAGATC | 53 | J | 738 |
| 1242010 | 23055 | 23070 | 3330 | 3345 | AGGCATTCTCCCCCAC | 45 | J | 739 |
| 1242034 | 23120 | 23135 | 3395 | 3410 | TTTCTGCAGGTTACAC | 67 | J | 740 |
| 1242058 | 31014 | 31029 | 3458 | 3473 | GCTGAGTACCGAGGAC | 38 | J | 741 |
| 1242082 | 31058 | 31073 | 3502 | 3517 | AGAGTGTTGCCTCGCA | 43 | J | 742 |
| 1242106 | 31125 | 31140 | 3569 | 3584 | CAACACCTGAAGCTTG | 77 | J | 743 |
| 1242130 | 31739 | 31754 | 3675 | 3690 | CCAGGTCATTGTTGCC | 61 | J | 744 |
| 1242154 | 35494 | 35509 | 3847 | 3862 | CACTCCTACCAAGAAG | 103 | J | 745 |
| 1242178 | 35697 | 35712 | 4050 | 4065 | TGTCCTGGTGTCTTCC | 57 | J | 746 |
| 1242202 | 35775 | 35790 | 4128 | 4143 | AGCTAATTACATGAGG | 37 | J | 747 |
| 1242226 | 35913 | 35928 | 4266 | 4281 | TTCTGTGTTATGGTCA | 77 | J | 748 |
| 1242250 | N/A | N/A | N/A | N/A | ATTCACCAACCAGAGC | 76 | J | 749 |
| 1242274 | 6769 | 6784 | N/A | N/A | GAGTATTTATACAGTC | 32 | J | 750 |
| 1242298 | 7874 | 7889 | N/A | N/A | TCAGATAAAGAGGGAC | 56 | J | 751 |
| 1242322 | 8599 | 8614 | N/A | N/A | GAGGGATAATACCAGA | 75 | J | 752 |
| 1242346 | 9503 | 9518 | N/A | N/A | GCATATCTTAAGAAGG | 68 | J | 753 |
| 1242370 | 10364 | 10379 | N/A | N/A | TCAACTAGAAGCACCA | 87 | J | 754 |
| 1242394 | 12707 | 12722 | N/A | N/A | CATTATGATGTAGCTG | 56 | J | 755 |
| 1242418 | 13119 | 13134 | N/A | N/A | CATGATTTGGCTCATA | 84 | J | 756 |
| 1242442 | 14481 | 14496 | N/A | N/A | GGTGCTGAGCCTCCTA | 19 | J | 757 |
| | 14627 | 14642 | | | | | | |
| | 14773 | 14788 | | | | | | |
| | 15065 | 15080 | | | | | | |
| 1242466 | 14546 | 14561 | N/A | N/A | GCCCCCTACCAGGGCT | 83 | J | 758 |
| | 15201 | 15216 | | | | | | |
| | 15274 | 15289 | | | | | | |
| 1242490 | 14590 | 14605 | N/A | N/A | GTCCTCAGAGTAAGGC | 44 | J | 759 |
| | 14882 | 14897 | | | | | | |
| | 15172 | 15187 | | | | | | |
| | 15245 | 15260 | | | | | | |
| 1242514 | 14704 | 14719 | N/A | N/A | AATGGGTGCTGAGCCC | 30 | J | 760 |
| | 14996 | 15011 | | | | | | |
| 1242538 | 17446 | 17461 | N/A | N/A | AGATTAACATTAGGTC | 30 | J | 761 |
| 1242562 | 18553 | 18568 | N/A | N/A | CAAACCCCATCCCCGG | 72 | J | 762 |
| 1242586 | 19665 | 19680 | N/A | N/A | ACAAATGGATCTCTAC | 50 | J | 763 |
| 1242610 | 20571 | 20586 | N/A | N/A | CCGGAATACAGCACAA | 65 | J | 764 |
| 1242634 | 20703 | 20718 | N/A | N/A | AGAGCTCCGGAATAGA | 60 | J | 765 |
| | 20745 | 20760 | | | | | | |
| | 20913 | 20928 | | | | | | |
| 1242658 | 21601 | 21616 | N/A | N/A | TATACAATATGAGGAC | 79 | J | 766 |
| 1242681 | 22672 | 22687 | N/A | N/A | GCACAATTAATGGCCT | 50 | J | 767 |
| 1242705 | 23288 | 23303 | N/A | N/A | GTATACATTATATGCC | 56 | J | 768 |
| 1242729 | 23595 | 23610 | N/A | N/A | ACATAGGTGAAGGTGC | 63 | J | 769 |
| 1242753 | 23928 | 23943 | N/A | N/A | TACCCTATAGCATCTT | 73 | J | 770 |
| 1242777 | 24183 | 24198 | N/A | N/A | CAGGTAAGATGAGGTT | 55 | J | 771 |
| 1242801 | 24508 | 24523 | N/A | N/A | GAAGGGTAATACCCTA | 77 | J | 772 |
| 1242825 | 25100 | 25115 | N/A | N/A | GGCAATAAACTCTTGT | 42 | J | 773 |
| 1242849 | 26065 | 26080 | N/A | N/A | GATACTCCCTTGGATG | 93 | J | 774 |
| 1242873 | 26544 | 26559 | N/A | N/A | CTAACATGACTTGTGT | 61 | J | 775 |
| 1242897 | 26798 | 26813 | N/A | N/A | ATGGGATATTATGCCC | 57 | J | 776 |
| 1242921 | 27180 | 27195 | N/A | N/A | CACAATATGCAGCCAG | 53 | J | 777 |
| 1242945 | 27770 | 27785 | N/A | N/A | CTAATTCATAGCTGGG | 80 | J | 778 |
| 1242969 | 28674 | 28689 | N/A | N/A | GAGGAATGTGATTGAC | 34 | J | 779 |
| 1242993 | 28915 | 28930 | N/A | N/A | TTAAAGTTATGCAGGC | 42 | J | 780 |
| 1243017 | 29241 | 29256 | N/A | N/A | AGTAAAGCATGAACAG | 75 | J | 781 |
| 1243041 | 29604 | 29619 | N/A | N/A | CACCTTTTAGTCTCAT | 42 | J | 782 |
| 1243065 | 31241 | 31256 | N/A | N/A | TAAGAAGACACCTCAC | 80 | J | 783 |
| 1243089 | 31602 | 31617 | N/A | N/A | CCTTAACCATCCCCCT | 93 | J | 784 |
| 1243113 | 33395 | 33410 | N/A | N/A | CATGAGTTTGTTGCTC | 52 | J | 785 |
| 1243137 | 33957 | 33972 | N/A | N/A | ACCAATCCTTTGTTTG | 72 | J | 786 |
| 1243161 | 34244 | 34259 | N/A | N/A | ATAACAAGAAGGTAGC | 48 | J | 787 |
| 1243185 | 34431 | 34446 | N/A | N/A | GATGAAGCCCGAACCC | 53 | J | 788 |
| 1243209 | 34639 | 34654 | N/A | N/A | GTTCATTTAGTCACTA | 36 | J | 789 |
| 1243233 | 32160 | 32175 | N/A | N/A | CCCACTATGTAAAGAC | 74 | J | 790 |
| 1232737 | 5138 | 5153 | 90 | 105 | ATGCCATCTTGACCCA | 56 | K | 22 |
| 1241413 | 5067 | 5082 | 19 | 34 | TTCACTATTCCTCAAC | 77 | K | 791 |
| 1241437 | 5149 | 5164 | 101 | 116 | ACCACTTCACGATGCC | 52 | K | 792 |
| 1241461 | 5253 | 5268 | 205 | 220 | GATCCAGGAGTGTGTC | 72 | K | 793 |
| 1241485 | 5690 | 5705 | 642 | 657 | TCCACTTTGAGAGATA | 69 | K | 794 |
| 1241509 | 5765 | 5780 | 717 | 732 | AGGGTCCTTAGGCTTC | 72 | K | 795 |
| 1241533 | 5898 | 5913 | 850 | 865 | GGGATGCAGCCCTTCT | 71 | K | 796 |
| 1241557 | 5973 | 5988 | 925 | 940 | TCCTCCCCATTGAAGT | 96 | K | 797 |
| 1241581 | 6023 | 6038 | 975 | 990 | CTCTCCTGTTGATCGC | 47 | K | 798 |
| 1241605 | 10254 | 10269 | 1054 | 1069 | CATATCACAGTGGGAT | 63 | K | 799 |
| 1241629 | N/A | N/A | 1145 | 1160 | CTTACGGTAATCTTTC | 54 | K | 800 |
| 1241653 | 10891 | 10906 | 1194 | 1209 | TCCTGTCTTCAATGCA | 48 | K | 801 |
| 1241677 | 11103 | 11118 | 1406 | 1421 | CTGGAACACCACGGTG | 76 | K | 802 |
| 1241701 | 11302 | 11317 | 1605 | 1620 | TCACGATCTTGTGGAT | 50 | K | 803 |
| 1241725 | 11469 | 11484 | 1772 | 1787 | GAGCAGAGAGGCCTCG | 70 | K | 804 |
| 1241749 | 11737 | 11752 | 2040 | 2055 | TGGATGTCTGGGCAAG | 40 | K | 805 |
| 1241773 | 11950 | 11965 | 2253 | 2268 | ACAGGTTCATCCTCAG | 36 | K | 806 |
| 1241796 | 12149 | 12164 | 2452 | 2467 | ATCAAATACCCCTTTT | 60 | K | 807 |
| 1241820 | 12231 | 12246 | 2534 | 2549 | AGAGATCTTGCAACTT | 40 | K | 808 |
| 1241843 | 12363 | 12378 | 2666 | 2681 | CCTTTGCACGAAGTCC | 30 | K | 809 |
| 1241867 | 12475 | 12490 | 2778 | 2793 | ACCCCAGGGACAGTGA | 37 | K | 810 |
| 1241891 | 12543 | 12558 | 2846 | 2861 | CTGCACCATATCAAGG | 85 | K | 811 |
| 1241915 | 16592 | 16607 | 2910 | 2925 | AAGTGAGGTGGCTGTT | 70 | K | 812 |
| 1241939 | 16742 | 16757 | 3060 | 3075 | ACCACAATCTCCGAAT | 92 | K | 813 |
| 1241963 | N/A | N/A | 3236 | 3251 | GACCAACCAGAGCTTC | 69 | K | 814 |
| 1241987 | 23012 | 23027 | 3287 | 3302 | GCTCAATACTGATGCA | 89 | K | 815 |
| 1242011 | 23057 | 23072 | 3332 | 3347 | CAAGGCATTCTCCCCC | 42 | K | 816 |
| 1242035 | 23121 | 23136 | 3396 | 3411 | GTTTCTGCAGGTTACA | 72 | K | 817 |
| 1242059 | 31015 | 31030 | 3459 | 3474 | TGCTGAGTACCGAGGA | 41 | K | 818 |
| 1242083 | 31060 | 31075 | 3504 | 3519 | CGAGAGTGTTGCCTCG | 103 | K | 819 |
| 1242107 | 31126 | 31141 | 3570 | 3585 | CCAACACCTGAAGCTT | 53 | K | 820 |
| 1242131 | 31740 | 31755 | 3676 | 3691 | CCCAGGTCATTGTTGC | 62 | K | 821 |
| 1242155 | 35500 | 35515 | 3853 | 3868 | CGTTTCCACTCCTACC | 75 | K | 822 |
| 1242179 | 35702 | 35717 | 4055 | 4070 | GTCATTGTCCTGGTGT | 45 | K | 823 |
| 1242203 | 35776 | 35791 | 4129 | 4144 | GAGCTAATTACATGAG | 56 | K | 824 |
| 1242227 | 35914 | 35929 | 4267 | 4282 | GTTCTGTGTTATGGTC | 80 | K | 825 |
| 1242251 | N/A | N/A | N/A | N/A | AATTCACCAACCAGAG | 91 | K | 826 |
| 1242275 | 6782 | 6797 | N/A | N/A | ATATGGTACCATGGAG | 56 | K | 827 |
| 1242299 | 8091 | 8106 | N/A | N/A | GGATTAAGAGAATTTC | 107 | K | 828 |
| 1242323 | 8638 | 8653 | N/A | N/A | AGTTAGGATCTCTTCC | 66 | K | 829 |
| 1242347 | 9507 | 9522 | N/A | N/A | GATAGCATATCTTAAG | 66 | K | 830 |
| 1242371 | 10428 | 10443 | N/A | N/A | TATAGTATGGCCAAGT | 82 | K | 831 |
| 1242395 | 12709 | 12724 | N/A | N/A | GGCATTATGATGTAGC | 55 | K | 832 |
| 1242419 | 13178 | 13193 | N/A | N/A | CCAAGGAAACTGTGTC | 36 | K | 833 |
| 1242443 | 14484 | 14499 | N/A | N/A | ATGGGTGCTGAGCCTC | 19 | K | 834 |
| | 14630 | 14645 | | | | | | |
| | 14776 | 14791 | | | | | | |
| | 15068 | 15083 | | | | | | |
| 1242467 | 14548 | 14563 | N/A | N/A | GAGCCCCCTACCAGGG | 70 | K | 835 |
| | 15130 | 15145 | | | | | | |
| | 15203 | 15218 | | | | | | |
| | 15276 | 15291 | | | | | | |
| 1242491 | 14591 | 14606 | N/A | N/A | TGTCCTCAGAGTAAGG | 33 | K | 836 |
| | 14883 | 14898 | | | | | | |
| | 15173 | 15188 | | | | | | |
| | 15246 | 15261 | | | | | | |
| 1242515 | 14924 | 14939 | N/A | N/A | AAATGGTTGCTGAGCC | 59 | K | 837 |
| 1242539 | 17447 | 17462 | N/A | N/A | TAGATTAACATTAGGT | 45 | K | 838 |
| 1242563 | 18555 | 18570 | N/A | N/A | CGCAAACCCCATCCCC | 69 | K | 839 |
| 1242587 | 19666 | 19681 | N/A | N/A | GACAAATGGATCTCTA | 38 | K | 840 |
| 1242611 | 20578 | 20593 | N/A | N/A | GAGAGCTCCGGAATAC | 50 | K | 841 |
| | 20956 | 20971 | | | | | | |
| 1242635 | 20704 | 20719 | N/A | N/A | GAGAGCTCCGGAATAG | 55 | K | 842 |
| | 20746 | 20761 | | | | | | |
| | 20914 | 20929 | | | | | | |
| 1242659 | 21603 | 21618 | N/A | N/A | CTTATACAATATGAGG | 45 | K | 843 |
| 1242682 | 22760 | 22775 | N/A | N/A | ATACTACATCTTCCCA | 68 | K | 844 |
| 1242706 | 23301 | 23316 | N/A | N/A | CTTATTAGCAGCAGTA | 35 | K | 845 |
| 1242730 | 23596 | 23611 | N/A | N/A | TACATAGGTGAAGGTG | 74 | K | 846 |
| 1242754 | 23987 | 24002 | N/A | N/A | AAGTTATGAGGCTCCC | 68 | K | 847 |
| 1242778 | 24214 | 24229 | N/A | N/A | TTCAAAACTGCCTAGT | 92 | K | 848 |
| 1242802 | 24510 | 24525 | N/A | N/A | AGGAAGGGTAATACCC | 63 | K | 849 |
| 1242826 | 25101 | 25116 | N/A | N/A | AGGCAATAAACTCTTG | 72 | K | 850 |
| 1242850 | 26088 | 26103 | N/A | N/A | CCAATATATGTTCACA | 57 | K | 851 |
| 1242874 | 26547 | 26562 | N/A | N/A | AATCTAACATGACTTG | 73 | K | 852 |
| 1242898 | 26813 | 26828 | N/A | N/A | ATGGTAAAGCACACTA | 46 | K | 853 |
| 1242922 | 27198 | 27213 | N/A | N/A | GTATTGAGACAGGCAG | 36 | K | 854 |
| 1242946 | 27788 | 27803 | N/A | N/A | CACCAAGATGTTTCAA | 57 | K | 855 |
| 1242970 | 28688 | 28703 | N/A | N/A | CAATCCTTGGATTAGA | 71 | K | 856 |
| 1242994 | 28916 | 28931 | N/A | N/A | ATTAAAGTTATGCAGG | 61 | K | 857 |
| 1243018 | 29243 | 29258 | N/A | N/A | ACAGTAAAGCATGAAC | 78 | K | 858 |
| 1243042 | 29690 | 29705 | N/A | N/A | GAAATCTAAGGCTTAT | 64 | K | 859 |
| 1243066 | 31242 | 31257 | N/A | N/A | CTAAGAAGACACCTCA | 71 | K | 860 |
| 1243090 | 31878 | 31893 | N/A | N/A | GATTTTGATAACAGTC | 49 | K | 861 |
| 1243114 | 33471 | 33486 | N/A | N/A | CAGAATAAGCAAGGGT | 92 | K | 862 |
| 1243138 | 33958 | 33973 | N/A | N/A | GACCAATCCTTTGTTT | 97 | K | 863 |
| 1243162 | 34245 | 34260 | N/A | N/A | CATAACAAGAAGGTAG | 70 | K | 864 |
| 1243186 | 34434 | 34449 | N/A | N/A | CATGATGAAGCCCGAA | 85 | K | 865 |
| 1243210 | 34682 | 34697 | N/A | N/A | CATTTACGCTTCCTCA | 67 | K | 866 |
| 1243234 | 26441 | 26456 | N/A | N/A | GAGGCATCTACAGGTC | 61 | K | 867 |
| 1004826 | 11973 | 11988 | 2276 | 2291 | CTCGCAGTCCACTTCC | 68 | L | 868 |
| 1004847 | 12233 | 12248 | 2536 | 2551 | TGAGAGATCTTGCAAC | 69 | L | 869 |
| 1232737 | 5138 | 5153 | 90 | 105 | ATGCCATCTTGACCCA | 57 | L | 22 |
| 1241414 | 5072 | 5087 | 24 | 39 | AACTCTTCACTATTCC | 53 | L | 870 |
| 1241438 | 5150 | 5165 | 102 | 117 | AACCACTTCACGATGC | 60 | L | 871 |
| 1241462 | 5255 | 5270 | 207 | 222 | TCGATCCAGGAGTGTG | 70 | L | 872 |
| 1241486 | 5691 | 5706 | 643 | 658 | CTCCACTTTGAGAGAT | 65 | L | 873 |
| 1241510 | 5766 | 5781 | 718 | 733 | CAGGGTCCTTAGGCTT | 64 | L | 874 |
| 1241534 | 5914 | 5929 | 866 | 881 | CTGACCCCTCGGGAGG | 74 | L | 875 |
| 1241558 | 5975 | 5990 | 927 | 942 | TCTCCTCCCCATTGAA | 70 | L | 876 |
| 1241582 | 6024 | 6039 | 976 | 991 | TCTCTCCTGTTGATCG | 42 | L | 877 |
| 1241606 | 10255 | 10270 | 1055 | 1070 | GCATATCACAGTGGGA | 59 | L | 878 |
| 1241630 | N/A | N/A | 1146 | 1161 | TCTTACGGTAATCTTT | 58 | L | 879 |
| 1241654 | 10899 | 10914 | 1202 | 1217 | ACGGGCATTCCTGTCT | 67 | L | 880 |
| 1241678 | 11106 | 11121 | 1409 | 1424 | CCCCTGGAACACCACG | 62 | L | 881 |
| 1241702 | 11304 | 11319 | 1607 | 1622 | TCTCACGATCTTGTGG | 55 | L | 882 |
| 1241726 | 11493 | 11508 | 1796 | 1811 | CAGGGCCACAGGTCTC | 56 | L | 883 |
| 1241750 | 11817 | 11832 | 2120 | 2135 | GCAGAGGCCGTGCTCC | 56 | L | 884 |
| 1241797 | 12150 | 12165 | 2453 | 2468 | AATCAAATACCCCTTT | 65 | L | 885 |
| 1241844 | 12369 | 12384 | 2672 | 2687 | CATGGCCCTTTGCACG | 50 | L | 886 |
| 1241868 | 12486 | 12501 | 2789 | 2804 | GTTATGGAGAAACCCC | 30 | L | 887 |
| 1241892 | 12545 | 12560 | 2848 | 2863 | CACTGCACCATATCAA | 73 | L | 888 |
| 1241916 | 16593 | 16608 | 2911 | 2926 | GAAGTGAGGTGGCTGT | 56 | L | 889 |
| 1241940 | N/A | N/A | 3061 | 3076 | AACCACAATCTCCGAA | 72 | L | 890 |
| 1241964 | 22970 | 22985 | 3245 | 3260 | GCAGCAGCTGACCAAC | 65 | L | 891 |
| 1241988 | 23013 | 23028 | 3288 | 3303 | TGCTCAATACTGATGC | 57 | L | 892 |
| 1242012 | 23061 | 23076 | 3336 | 3351 | CTCCCAAGGCATTCTC | 72 | L | 893 |
| 1242036 | 23122 | 23137 | 3397 | 3412 | AGTTTCTGCAGGTTAC | 59 | L | 894 |
| 1242060 | 31016 | 31031 | 3460 | 3475 | GTGCTGAGTACCGAGG | 39 | L | 895 |
| 1242084 | 31061 | 31076 | 3505 | 3520 | CCGAGAGTGTTGCCTC | 49 | L | 896 |
| 1242108 | N/A | N/A | 3572 | 3587 | TTCCAACACCTGAAGC | 63 | L | 897 |
| 1242132 | 31741 | 31756 | 3677 | 3692 | GCCCAGGTCATTGTTG | 87 | L | 898 |
| 1242156 | 35501 | 35516 | 3854 | 3869 | CCGTTTCCACTCCTAC | 48 | L | 899 |
| 1242180 | 35703 | 35718 | 4056 | 4071 | TGTCATTGTCCTGGTG | 51 | L | 900 |
| 1242204 | 35787 | 35802 | 4140 | 4155 | CTTTATTGAATGAGCT | 43 | L | 901 |
| 1242228 | 35915 | 35930 | 4268 | 4283 | AGTTCTGTGTTATGGT | 76 | L | 902 |
| 1242252 | N/A | N/A | N/A | N/A | GAATTCACCAACCAGA | 63 | L | 903 |
| 1242276 | 6783 | 6798 | N/A | N/A | TATATGGTACCATGGA | 59 | L | 904 |
| 1242300 | 8092 | 8107 | N/A | N/A | GGGATTAAGAGAATTT | 95 | L | 905 |
| 1242324 | 8640 | 8655 | N/A | N/A | CCAGTTAGGATCTCTT | 46 | L | 906 |
| 1242348 | 9510 | 9525 | N/A | N/A | CTAGATAGCATATCTT | 75 | L | 907 |
| 1242372 | 10429 | 10444 | N/A | N/A | CTATAGTATGGCCAAG | 74 | L | 908 |
| 1242396 | 12710 | 12725 | N/A | N/A | TGGCATTATGATGTAG | 59 | L | 909 |
| 1242420 | 13215 | 13230 | N/A | N/A | CTCTATTTCCCTTGAT | 72 | L | 910 |
| 1242444 | 14485 | 14500 | N/A | N/A | AATGGGTGCTGAGCCT | 24 | L | 911 |
| | 14631 | 14646 | | | | | | |
| | 14777 | 14792 | | | | | | |
| | 15069 | 15084 | | | | | | |
| 1242468 | 14549 | 14564 | N/A | N/A | TGAGCCCCCTACCAGG | 55 | L | 912 |
| | 15131 | 15146 | | | | | | |
| | 15204 | 15219 | | | | | | |
| | 15277 | 15292 | | | | | | |
| 1242492 | 14592 | 14607 | N/A | N/A | CTGTCCTCAGAGTAAG | 42 | L | 913 |
| | 14884 | 14899 | | | | | | |
| | 15174 | 15189 | | | | | | |
| | 15247 | 15262 | | | | | | |
| 1242516 | 15111 | 15126 | N/A | N/A | CAGTCAGAGTCTGTCC | 13 | L | 914 |
| | 15257 | 15272 | | | | | | |
| 1242540 | 17448 | 17463 | N/A | N/A | CTAGATTAACATTAGG | 58 | L | 915 |
| 1242564 | 18568 | 18583 | N/A | N/A | CAAGACAAACAAACGC | 40 | L | 916 |
| 1242588 | 19721 | 19736 | N/A | N/A | CCTGTTAGACCACCTT | 28 | L | 917 |
| 1242612 | 20580 | 20595 | N/A | N/A | CAGAGAGCTCCGGAAT | 58 | L | 918 |
| | 20706 | 20721 | | | | | | |
| | 20748 | 20763 | | | | | | |
| | 20916 | 20931 | | | | | | |
| | 20958 | 20973 | | | | | | |
| 1242636 | 20775 | 20790 | N/A | N/A | TAGAGAATGCATCAGG | 52 | L | 919 |
| 1242660 | 21636 | 21651 | N/A | N/A | ATCGAGGGTCTGCATT | 65 | L | 920 |
| 1242683 | 22762 | 22777 | N/A | N/A | CAATACTACATCTTCC | 77 | L | 921 |
| 1242707 | 23305 | 23320 | N/A | N/A | TTATCTTATTAGCAGC | 27 | L | 922 |
| 1242731 | 23598 | 23613 | N/A | N/A | GCTACATAGGTGAAGG | 56 | L | 923 |
| 1242755 | 23988 | 24003 | N/A | N/A | AAAGTTATGAGGCTCC | 54 | L | 924 |
| 1242779 | 24216 | 24231 | N/A | N/A | GATTCAAAACTGCCTA | 60 | L | 925 |
| 1242803 | 24535 | 24550 | N/A | N/A | AGTAATATTCTAAGGC | 60 | L | 926 |
| 1242827 | 25189 | 25204 | N/A | N/A | TAAAGTAAGGACTGGG | 62 | L | 927 |
| 1242851 | 26100 | 26115 | N/A | N/A | CATAATTAGCAACCAA | 70 | L | 928 |
| 1242875 | 26549 | 26564 | N/A | N/A | CAAATCTAACATGACT | 63 | L | 929 |
| 1242899 | 26815 | 26830 | N/A | N/A | TTATGGTAAAGCACAC | 51 | L | 930 |
| 1242923 | 27201 | 27216 | N/A | N/A | GCAGTATTGAGACAGG | 30 | L | 931 |
| 1242947 | 27800 | 27815 | N/A | N/A | GATTAAAAGAACCACC | 72 | L | 932 |
| 1242971 | 28690 | 28705 | N/A | N/A | ACCAATCCTTGGATTA | 76 | L | 933 |
| 1242995 | 28978 | 28993 | N/A | N/A | ATTCTAGTAGAATGGG | 70 | L | 934 |
| 1243019 | 29360 | 29375 | N/A | N/A | AGGAATAGCGCAGGTG | 54 | L | 935 |
| 1243043 | 29723 | 29738 | N/A | N/A | CAGCTAAAACTATTAC | 80 | L | 936 |
| 1243067 | 31243 | 31258 | N/A | N/A | ACTAAGAAGACACCTC | 85 | L | 937 |
| 1243091 | 31879 | 31894 | N/A | N/A | GGATTTTGATAACAGT | 45 | L | 938 |
| 1243115 | 33472 | 33487 | N/A | N/A | GCAGAATAAGCAAGGG | 79 | L | 939 |
| 1243139 | 33985 | 34000 | N/A | N/A | ATGTAGCAGTGTCCCC | 53 | L | 940 |
| 1243163 | 34312 | 34327 | N/A | N/A | GATATCAGAGAGTTTG | 63 | L | 941 |
| 1243187 | 34435 | 34450 | N/A | N/A | CCATGATGAAGCCCGA | 47 | L | 942 |
| 1243211 | 34684 | 34699 | N/A | N/A | AGCATTTACGCTTCCT | 55 | L | 943 |
| 1243235 | 29510 | 29525 | N/A | N/A | AAATGCATCAATCTCG | 53 | L | 944 |
| 1232737 | 5138 | 5153 | 90 | 105 | ATGCCATCTTGACCCA | 85 | M | 22 |
| 1241415 | 5076 | 5091 | 28 | 43 | GACAAACTCTTCACTA | 64 | M | 945 |
| 1241439 | 5151 | 5166 | 103 | 118 | CAACCACTTCACGATG | 56 | M | 946 |
| 1241463 | 5261 | 5276 | 213 | 228 | GTTGGCTCGATCCAGG | 72 | M | 947 |
| 1241487 | 5695 | 5710 | 647 | 662 | AAGTCTCCACTTTGAG | 65 | M | 948 |
| 1241511 | 5769 | 5784 | 721 | 736 | TTTCAGGGTCCTTAGG | 62 | M | 949 |
| 1241535 | 5915 | 5930 | 867 | 882 | TCTGACCCCTCGGGAG | 77 | M | 950 |
| 1241559 | 5985 | 6000 | 937 | 952 | GCCCACGCCTTCTCCT | 67 | M | 951 |
| 1241583 | 6028 | 6043 | 980 | 995 | AAGGTCTCTCCTGTTG | 56 | M | 952 |
| 1241607 | 10257 | 10272 | 1057 | 1072 | TGGCATATCACAGTGG | 40 | M | 953 |
| 1241631 | N/A | N/A | 1147 | 1162 | TTCTTACGGTAATCTT | 38 | M | 954 |
| 1241655 | 10901 | 10916 | 1204 | 1219 | AGACGGGCATTCCTGT | 53 | M | 955 |
| 1241679 | 11107 | 11122 | 1410 | 1425 | CCCCCTGGAACACCAC | 47 | M | 956 |
| 1241703 | 11307 | 11322 | 1610 | 1625 | TTTTCTCACGATCTTG | 32 | M | 957 |
| 1241727 | 11511 | 11526 | 1814 | 1829 | CAAGTGCTGCAGTTTC | 76 | M | 958 |
| 1241751 | 11818 | 11833 | 2121 | 2136 | CGCAGAGGCCGTGCTC | 70 | M | 959 |
| 1241774 | 11977 | 11992 | 2280 | 2295 | ACTTCTCGCAGTCCAC | 31 | M | 960 |
| 1241798 | 12161 | 12176 | 2464 | 2479 | CGTACAACAAAAATCA | 52 | M | 961 |
| 1241821 | 12234 | 12249 | 2537 | 2552 | CTGAGAGATCTTGCAA | 43 | M | 962 |
| 1241845 | 12372 | 12387 | 2675 | 2690 | GTCCATGGCCCTTTGC | 49 | M | 963 |
| 1241869 | 12488 | 12503 | 2791 | 2806 | ATGTTATGGAGAAACC | 59 | M | 964 |
| 1241893 | 12546 | 12561 | 2849 | 2864 | ACACTGCACCATATCA | 56 | M | 965 |
| 1241917 | 16595 | 16610 | 2913 | 2928 | TGGAAGTGAGGTGGCT | 56 | M | 966 |
| 1241941 | N/A | N/A | 3062 | 3077 | CAACCACAATCTCCGA | 62 | M | 967 |
| 1241965 | 22971 | 22986 | 3246 | 3261 | GGCAGCAGCTGACCAA | 46 | M | 968 |
| 1241989 | 23014 | 23029 | 3289 | 3304 | GTGCTCAATACTGATG | 44 | M | 969 |
| 1242013 | 23063 | 23078 | 3338 | 3353 | GTCTCCCAAGGCATTC | 46 | M | 970 |
| 1242037 | 23123 | 23138 | 3398 | 3413 | CAGTTTCTGCAGGTTA | 34 | M | 971 |
| 1242061 | 31017 | 31032 | 3461 | 3476 | AGTGCTGAGTACCGAG | 33 | M | 972 |
| 1242085 | 31065 | 31080 | 3509 | 3524 | GTCTCCGAGAGTGTTG | 46 | M | 973 |
| 1242109 | N/A | N/A | 3578 | 3593 | GTCTAATTCCAACACC | 41 | M | 974 |
| 1242133 | 31742 | 31757 | 3678 | 3693 | CGCCCAGGTCATTGTT | 80 | M | 975 |
| 1242157 | 35531 | 35546 | 3884 | 3899 | GGGACCGGAGAACACT | 82 | M | 976 |
| 1242181 | 35705 | 35720 | 4058 | 4073 | GCTGTCATTGTCCTGG | 48 | M | 977 |
| 1242205 | 35788 | 35803 | 4141 | 4156 | GCTTTATTGAATGAGC | 65 | M | 978 |
| 1242229 | 35916 | 35931 | 4269 | 4284 | TAGTTCTGTGTTATGG | 72 | M | 979 |
| 1242253 | 3156 | 3171 | N/A | N/A | GCTAGAGAGGAAATGC | 106 | M | 980 |
| 1242277 | 6784 | 6799 | N/A | N/A | GTATATGGTACCATGG | 58 | M | 981 |
| 1242301 | 8121 | 8136 | N/A | N/A | GAAAGAACCTTATCCC | 63 | M | 982 |
| 1242325 | 8652 | 8667 | N/A | N/A | ATAAACCATGGGCCAG | 61 | M | 983 |
| 1242349 | 9511 | 9526 | N/A | N/A | ACTAGATAGCATATCT | 63 | M | 984 |
| 1242373 | 10430 | 10445 | N/A | N/A | CCTATAGTATGGCCAA | 63 | M | 985 |
| 1242397 | 12754 | 12769 | N/A | N/A | ATACATCAATCTTTGC | 51 | M | 986 |
| 1242421 | 13226 | 13241 | N/A | N/A | CCACATTTCTGCTCTA | 48 | M | 987 |
| 1242445 | 14491 | 14506 | N/A | N/A | TAGAGAAATGGGTGCT | 18 | M | 988 |
| | 14637 | 14652 | | | | | | |
| | 14710 | 14725 | | | | | | |
| | 14783 | 14798 | | | | | | |
| | 15002 | 15017 | | | | | | |
| | 15075 | 15090 | | | | | | |
| 1242469 | 14550 | 14565 | N/A | N/A | CTGAGCCCCCTACCAG | 58 | M | 989 |
| | 15132 | 15147 | | | | | | |
| | 15205 | 15220 | | | | | | |
| | 15278 | 15293 | | | | | | |
| 1242493 | 14594 | 14609 | N/A | N/A | GTCTGTCCTCAGAGTA | 12 | M | 990 |
| | 14886 | 14901 | | | | | | |
| | 15176 | 15191 | | | | | | |
| | 15249 | 15264 | | | | | | |
| 1242517 | 15112 | 15127 | N/A | N/A | CCAGTCAGAGTCTGTC | 16 | M | 991 |
| | 15258 | 15273 | | | | | | |
| 1242541 | 17449 | 17464 | N/A | N/A | ACTAGATTAACATTAG | 49 | M | 992 |
| 1242565 | 18603 | 18618 | N/A | N/A | CTAAGATGTCTGTCCA | 42 | M | 993 |
| 1242589 | 19738 | 19753 | N/A | N/A | GTTCATAGGTCCTGGA | 37 | M | 994 |
| 1242613 | 20581 | 20596 | N/A | N/A | CCAGAGAGCTCCGGAA | 38 | M | 995 |
| | 20707 | 20722 | | | | | | |
| | 20749 | 20764 | | | | | | |
| | 20917 | 20932 | | | | | | |
| 1242637 | 20776 | 20791 | N/A | N/A | ATAGAGAATGCATCAG | 55 | M | 996 |
| 1242661 | 21643 | 21658 | N/A | N/A | ATACAACATCGAGGGT | 39 | M | 997 |
| 1242684 | 22763 | 22778 | N/A | N/A | CCAATACTACATCTTC | 80 | M | 998 |
| 1242708 | 23336 | 23351 | N/A | N/A | CTCTAGTTCTATAATC | 73 | M | 999 |
| 1242732 | 23632 | 23647 | N/A | N/A | CTAACAAACCAGCCTG | 64 | M | 1000 |
| 1242756 | 23989 | 24004 | N/A | N/A | CAAAGTTATGAGGCTC | 60 | M | 1001 |
| 1242780 | 24236 | 24251 | N/A | N/A | GATATTATTGTCAGAA | 36 | M | 1002 |
| 1242804 | 24553 | 24568 | N/A | N/A | GTCAAATTGCCAAGGA | 28 | M | 1003 |
| 1242828 | 25190 | 25205 | N/A | N/A | CTAAAGTAAGGACTGG | 43 | M | 1004 |
| 1242852 | 26101 | 26116 | N/A | N/A | CCATAATTAGCAACCA | 49 | M | 1005 |
| 1242876 | 26550 | 26565 | N/A | N/A | ACAAATCTAACATGAC | 78 | M | 1006 |
| 1242900 | 26817 | 26832 | N/A | N/A | AATTATGGTAAAGCAC | 80 | M | 1007 |
| 1242924 | 27251 | 27266 | N/A | N/A | GTATGATAATTAAACC | 101 | M | 1008 |
| 1242948 | 27801 | 27816 | N/A | N/A | AGATTAAAAGAACCAC | 80 | M | 1009 |
| 1242972 | 28710 | 28725 | N/A | N/A | CAAACAACCATGTCCC | 37 | M | 1010 |
| 1242996 | 29010 | 29025 | N/A | N/A | GATTAGTAGCATTCCT | 98 | M | 1011 |
| 1243020 | 29362 | 29377 | N/A | N/A | CCAGGAATAGCGCAGG | 47 | M | 1012 |
| 1243044 | 29750 | 29765 | N/A | N/A | TTATATCAACCTGGCA | 47 | M | 1013 |
| 1243068 | 31244 | 31259 | N/A | N/A | CACTAAGAAGACACCT | 86 | M | 1014 |
| 1243092 | 31919 | 31934 | N/A | N/A | CCTAATCTTGAGTGGC | 73 | M | 1015 |
| 1243116 | 33588 | 33603 | N/A | N/A | GGAATAATGCAGGTGG | 76 | M | 1016 |
| 1243140 | 33986 | 34001 | N/A | N/A | CATGTAGCAGTGTCCC | 45 | M | 1017 |
| 1243164 | 34321 | 34336 | N/A | N/A | TACAGGAGAGATATCA | 90 | M | 1018 |
| 1243188 | 34441 | 34456 | N/A | N/A | CAATTCCCATGATGAA | 71 | M | 1019 |
| 1243212 | 34685 | 34700 | N/A | N/A | CAGCATTTACGCTTCC | 52 | M | 1020 |
| 1243236 | 17707 | 17722 | N/A | N/A | TCCAACTCTCATCTAT | 61 | M | 1021 |
| 1232737 | 5138 | 5153 | 90 | 105 | ATGCCATCTTGACCCA | 59 | N | 22 |
| 1241416 | 5077 | 5092 | 29 | 44 | GGACAAACTCTTCACT | 48 | N | 1022 |
| 1241440 | 5154 | 5169 | 106 | 121 | GAACAACCACTTCACG | 76 | N | 1023 |
| 1241464 | 5262 | 5277 | 214 | 229 | TGTTGGCTCGATCCAG | 77 | N | 1024 |
| 1241488 | 5712 | 5727 | 664 | 679 | CGGATGAGTCTTTTTT | 38 | N | 1025 |
| 1241512 | 5771 | 5786 | 723 | 738 | GTTTTCAGGGTCCTTA | 62 | N | 1026 |
| 1241536 | 5916 | 5931 | 868 | 883 | GTCTGACCCCTCGGGA | 62 | N | 1027 |
| 1241560 | 5988 | 6003 | 940 | 955 | ATGGCCCACGCCTTCT | 62 | N | 1028 |
| 1241584 | 6030 | 6045 | 982 | 997 | TAAAGGTCTCTCCTGT | 61 | N | 1029 |
| 1241608 | 10262 | 10277 | 1062 | 1077 | CTTCCTGGCATATCAC | 37 | N | 1030 |
| 1241632 | 10845 | 10860 | 1148 | 1163 | CTTCTTACGGTAATCT | 45 | N | 1031 |
| 1241656 | 10902 | 10917 | 1205 | 1220 | CAGACGGGCATTCCTG | 51 | N | 1032 |
| 1241680 | 11108 | 11123 | 1411 | 1426 | GCCCCCTGGAACACCA | 50 | N | 1033 |
| 1241704 | 11310 | 11325 | 1613 | 1628 | GGGTTTTCTCACGATC | 36 | N | 1034 |
| 1241728 | 11512 | 11527 | 1815 | 1830 | GCAAGTGCTGCAGTTT | 58 | N | 1035 |
| 1241752 | 11819 | 11834 | 2122 | 2137 | GCGCAGAGGCCGTGCT | 96 | N | 1036 |
| 1241775 | 11978 | 11993 | 2281 | 2296 | AACTTCTCGCAGTCCA | 25 | N | 1037 |
| 1241799 | 12180 | 12195 | 2483 | 2498 | TACCAGGCCAAAGAGG | 84 | N | 1038 |
| 1241822 | 12244 | 12259 | 2547 | 2562 | GCCTGATTTGCTGAGA | 38 | N | 1039 |
| 1241846 | 12374 | 12389 | 2677 | 2692 | TAGTCCATGGCCCTTT | 47 | N | 1040 |
| 1241870 | 12489 | 12504 | 2792 | 2807 | CATGTTATGGAGAAAC | 77 | N | 1041 |
| 1241894 | 12547 | 12562 | 2850 | 2865 | CACACTGCACCATATC | 57 | N | 1042 |
| 1241918 | 16596 | 16611 | 2914 | 2929 | CTGGAAGTGAGGTGGC | 60 | N | 1043 |
| 1241942 | N/A | N/A | 3063 | 3078 | CCAACCACAATCTCCG | 83 | N | 1044 |
| 1241966 | 22982 | 22997 | 3257 | 3272 | TGCTGATGTGAGGCAG | 59 | N | 1045 |
| 1241990 | 23015 | 23030 | 3290 | 3305 | GGTGCTCAATACTGAT | 54 | N | 1046 |
| 1242014 | 23065 | 23080 | 3340 | 3355 | GAGTCTCCCAAGGCAT | 40 | N | 1047 |
| 1242038 | 23124 | 23139 | 3399 | 3414 | CCAGTTTCTGCAGGTT | 43 | N | 1048 |
| 1242062 | 31018 | 31033 | 3462 | 3477 | TAGTGCTGAGTACCGA | 36 | N | 1049 |
| 1242086 | 31066 | 31081 | 3510 | 3525 | TGTCTCCGAGAGTGTT | 39 | N | 1050 |
| 1242110 | 31651 | 31666 | 3587 | 3602 | GTTGCAGTTGTCTAAT | 61 | N | 1051 |
| 1242134 | 31756 | 31771 | 3692 | 3707 | CATGACCCCCAGGTCG | 59† | N | 1052 |
| 1242158 | 35538 | 35553 | 3891 | 3906 | AGCTGGAGGGACCGGA | 110 | N | 1053 |
| 1242182 | 35711 | 35726 | 4064 | 4079 | CCCGATGCTGTCATTG | 51 | N | 1054 |
| 1242206 | 35789 | 35804 | 4142 | 4157 | TGCTTTATTGAATGAG | 29 | N | 1055 |
| 1242230 | 35917 | 35932 | 4270 | 4285 | CTAGTTCTGTGTTATG | 62 | N | 1056 |
| 1242254 | 3161 | 3176 | N/A | N/A | GAACAGCTAGAGAGGA | 71 | N | 1057 |
| 1242278 | 6786 | 6801 | N/A | N/A | TGGTATATGGTACCAT | 85 | N | 1058 |
| 1242302 | 8189 | 8204 | N/A | N/A | CTACATCATCATCCTG | 45 | N | 1059 |
| 1242326 | 8655 | 8670 | N/A | N/A | GAAATAAACCATGGGC | 66 | N | 1060 |
| 1242350 | 9551 | 9566 | N/A | N/A | CAACATGCATCTGTGT | 59 | N | 1061 |
| 1242374 | 10477 | 10492 | N/A | N/A | GACAATGGATTTCCTG | 57 | N | 1062 |
| 1242398 | 12755 | 12770 | N/A | N/A | CATACATCAATCTTTG | 59 | N | 1063 |
| 1242422 | 13242 | 13257 | N/A | N/A | CCTAGAAAGAGGGTCT | 54 | N | 1064 |
| 1242446 | 14492 | 14507 | N/A | N/A | CTAGAGAAATGGGTGC | 25 | N | 1065 |
| | 14638 | 14653 | | | | | | |
| | 14711 | 14726 | | | | | | |
| | 14784 | 14799 | | | | | | |
| | 15003 | 15018 | | | | | | |
| | 15076 | 15091 | | | | | | |
| 1242470 | 14558 | 14573 | N/A | N/A | GATGGGTGCTGAGCCC | 19 | N | 1066 |
| | 14850 | 14865 | | | | | | |
| | 15140 | 15155 | | | | | | |
| | 15213 | 15228 | | | | | | |
| | 15286 | 15301 | | | | | | |
| 1242494 | 14603 | 14618 | N/A | N/A | TCAGTGAGAGTCTGTC | 20 | N | 1067 |
| | 14749 | 14764 | | | | | | |
| | 14895 | 14910 | | | | | | |
| | 15041 | 15056 | | | | | | |
| 1242518 | 15113 | 15128 | N/A | N/A | TCCAGTCAGAGTCTGT | 16 | N | 1068 |
| | 15259 | 15274 | | | | | | |
| 1242542 | 17463 | 17478 | N/A | N/A | TCTTTAAAGGAGTGAC | 40 | N | 1069 |
| 1242566 | 18693 | 18708 | N/A | N/A | AGAGAATGGTCGGGTG | 49 | N | 1070 |
| 1242590 | 19742 | 19757 | N/A | N/A | AATAGTTCATAGGTCC | 51 | N | 1071 |
| 1242614 | 20585 | 20600 | N/A | N/A | CTGACCAGAGAGCTCC | 29 | N | 1072 |
| | 20711 | 20726 | | | | | | |
| | 20753 | 20768 | | | | | | |
| | 20879 | 20894 | | | | | | |
| | 20921 | 20936 | | | | | | |
| | 21047 | 21062 | | | | | | |
| 1242638 | 20778 | 20793 | N/A | N/A | GAATAGAGAATGCATC | 105 | N | 1073 |
| 1242662 | 21644 | 21659 | N/A | N/A | CATACAACATCGAGGG | 32 | N | 1074 |
| 1242685 | 22764 | 22779 | N/A | N/A | ACCAATACTACATCTT | 76 | N | 1075 |
| 1242709 | 23340 | 23355 | N/A | N/A | GAAACTCTAGTTCTAT | 35 | N | 1076 |
| 1242733 | 23633 | 23648 | N/A | N/A | ACTAACAAACCAGCCT | 56 | N | 1077 |
| 1242757 | 23991 | 24006 | N/A | N/A | GACAAAGTTATGAGGC | 76 | N | 1078 |
| 1242781 | 24238 | 24253 | N/A | N/A | TAGATATTATTGTCAG | 40 | N | 1079 |
| 1242805 | 24723 | 24738 | N/A | N/A | GTTTAGCATGTCTATT | 27 | N | 1080 |
| 1242829 | 25271 | 25286 | N/A | N/A | ACTAAGTAGTGACCTA | 55 | N | 1081 |
| 1242853 | 26102 | 26117 | N/A | N/A | TCCATAATTAGCAACC | 73 | N | 1082 |
| 1242877 | 26553 | 26568 | N/A | N/A | GTTACAAATCTAACAT | 76 | N | 1083 |
| 1242901 | 26856 | 26871 | N/A | N/A | AAAGAACAGTTATCCC | 37 | N | 1084 |
| 1242925 | 27313 | 27328 | N/A | N/A | CTATTCAATGAGGCAG | 83 | N | 1085 |
| 1242949 | 27868 | 27883 | N/A | N/A | GATAGTAGAAAGTGGG | 69 | N | 1086 |
| 1242973 | 28711 | 28726 | N/A | N/A | TCAAACAACCATGTCC | 62 | N | 1087 |
| 1242997 | 29011 | 29026 | N/A | N/A | GGATTAGTAGCATTCC | 73 | N | 1088 |
| 1243021 | 29375 | 29390 | N/A | N/A | CTATTCCATGAAGCCA | 70 | N | 1089 |
| 1243045 | 29751 | 29766 | N/A | N/A | ATTATATCAACCTGGC | 37 | N | 1090 |
| 1243069 | 31245 | 31260 | N/A | N/A | ACACTAAGAAGACACC | 67 | N | 1091 |
| 1243093 | 31920 | 31935 | N/A | N/A | ACCTAATCTTGAGTGG | 83 | N | 1092 |
| 1243117 | 33589 | 33604 | N/A | N/A | GGGAATAATGCAGGTG | 95 | N | 1093 |
| 1243141 | 33989 | 34004 | N/A | N/A | CTACATGTAGCAGTGT | 57 | N | 1094 |
| 1243165 | 34323 | 34338 | N/A | N/A | CTTACAGGAGAGATAT | 88 | N | 1095 |
| 1243189 | 34444 | 34459 | N/A | N/A | TAACAATTCCCATGAT | 92 | N | 1096 |
| 1243213 | 34708 | 34723 | N/A | N/A | CTTAGTATCACTGTAT | 44 | N | 1097 |
| 1243237 | 9843 | 9858 | N/A | N/A | ATCCCCAACACGGCTG | 51 | N | 1098 |
| 1232737 | 5138 | 5153 | 90 | 105 | ATGCCATCTTGACCCA | 50 | O | 22 |
| 1241417 | 5078 | 5093 | 30 | 45 | TGGACAAACTCTTCAC | 52 | O | 1099 |
| 1241441 | 5156 | 5171 | 108 | 123 | GTGAACAACCACTTCA | 61 | O | 1100 |
| 1241465 | 5263 | 5278 | 215 | 230 | CTGTTGGCTCGATCCA | 78 | O | 1101 |
| 1241489 | 5717 | 5732 | 669 | 684 | GCACACGGATGAGTCT | 63 | O | 1102 |
| 1241513 | 5775 | 5790 | 727 | 742 | AGCTGTTTTCAGGGTC | 59 | O | 1103 |
| 1241537 | 5917 | 5932 | 869 | 884 | TGTCTGACCCCTCGGG | 49 | O | 1104 |
| 1241561 | 5990 | 6005 | 942 | 957 | CCATGGCCCACGCCTT | 44 | O | 1105 |
| 1241585 | 6031 | 6046 | 983 | 998 | ATAAAGGTCTCTCCTG | 60 | O | 1106 |
| 1241609 | 10263 | 10278 | 1063 | 1078 | TCTTCCTGGCATATCA | 44 | O | 1107 |
| 1241633 | 10846 | 10861 | 1149 | 1164 | ACTTCTTACGGTAATC | 44 | O | 1108 |
| 1241657 | 10903 | 10918 | 1206 | 1221 | CCAGACGGGCATTCCT | 50 | O | 1109 |
| 1241681 | 11177 | 11192 | 1480 | 1495 | TGGTAGAGTGTCCCCG | 41 | O | 1110 |
| 1241705 | 11319 | 11334 | 1622 | 1637 | GATTCTGGAGGGTTTT | 72 | O | 1111 |
| 1241729 | 11519 | 11534 | 1822 | 1837 | TGGTCCAGCAAGTGCT | 49 | O | 1112 |
| 1241753 | 11829 | 11844 | 2132 | 2147 | CCAGAGGTGGGCGCAG | 93 | O | 1113 |
| 1241776 | 11979 | 11994 | 2282 | 2297 | GAACTTCTCGCAGTCC | 31 | O | 1114 |
| 1241800 | 12181 | 12196 | 2484 | 2499 | TTACCAGGCCAAAGAG | 73 | O | 1115 |
| 1241823 | 12249 | 12264 | 2552 | 2567 | CTCCAGCCTGATTTGC | 96 | O | 1116 |
| 1241847 | 12375 | 12390 | 2678 | 2693 | ATAGTCCATGGCCCTT | 38 | O | 1117 |
| 1241871 | 12491 | 12506 | 2794 | 2809 | GGCATGTTATGGAGAA | 43 | O | 1118 |
| 1241895 | 12549 | 12564 | 2852 | 2867 | GACACACTGCACCATA | 38 | O | 1119 |
| 1241919 | 16620 | 16635 | 2938 | 2953 | ACTGAAAAGAGGCCCC | 41 | O | 1120 |
| 1241943 | N/A | N/A | 3064 | 3079 | CCCAACCACAATCTCC | 73 | O | 1121 |
| 1241967 | 22983 | 22998 | 3258 | 3273 | ATGCTGATGTGAGGCA | 68 | O | 1122 |
| 1241991 | 23016 | 23031 | 3291 | 3306 | TGGTGCTCAATACTGA | 49 | O | 1123 |
| 1242015 | 23066 | 23081 | 3341 | 3356 | TGAGTCTCCCAAGGCA | 31 | O | 1124 |
| 1242039 | N/A | N/A | 3410 | 3425 | ATTCACCAACCCCAGT | 68 | O | 1125 |
| 1242063 | 31019 | 31034 | 3463 | 3478 | TTAGTGCTGAGTACCG | 31 | O | 1126 |
| 1242087 | 31067 | 31082 | 3511 | 3526 | TTGTCTCCGAGAGTGT | 46 | O | 1127 |
| 1242111 | 31652 | 31667 | 3588 | 3603 | GGTTGCAGTTGTCTAA | 43 | O | 1128 |
| 1242135 | 31758 | 31773 | 3694 | 3709 | ATCATGACCCCCAGGT | 65† | O | 1129 |
| 1242159 | 35540 | 35555 | 3893 | 3908 | CCAGCTGGAGGGACCG | 121 | O | 1130 |
| 1242183 | 35717 | 35732 | 4070 | 4085 | ACAACACCCGATGCTG | 55 | O | 1131 |
| 1242207 | 35790 | 35805 | 4143 | 4158 | GTGCTTTATTGAATGA | 34 | O | 1132 |
| 1242231 | 35918 | 35933 | 4271 | 4286 | ACTAGTTCTGTGTTAT | 69 | O | 1133 |
| 1242255 | 3165 | 3180 | N/A | N/A | TCAGGAACAGCTAGAG | 81 | O | 1134 |
| 1242279 | 6826 | 6841 | N/A | N/A | AGTAATCCAGGTGTTC | 53 | O | 1135 |
| 1242303 | 8222 | 8237 | N/A | N/A | CATTTGTACTCCTCCT | 64 | O | 1136 |
| 1242327 | 8718 | 8733 | N/A | N/A | GTTAAGAGCCAGGCTT | 77 | O | 1137 |
| 1242351 | 9574 | 9589 | N/A | N/A | TACTAAAAGCTCCCAG | 81 | O | 1138 |
| 1242375 | 10478 | 10493 | N/A | N/A | TGACAATGGATTTCCT | 58 | O | 1139 |
| 1242399 | 12812 | 12827 | N/A | N/A | CCCAAACATTTGTTCC | 62 | O | 1140 |
| 1242423 | 13498 | 13513 | N/A | N/A | CTTAATTTTGCCCCAC | 81 | O | 1141 |
| 1242447 | 14493 | 14508 | N/A | N/A | TCTAGAGAAATGGGTG | 21 | O | 1142 |
| | 14639 | 14654 | | | | | | |
| | 14712 | 14727 | | | | | | |
| | 14785 | 14800 | | | | | | |
| | 15004 | 15019 | | | | | | |
| | 15077 | 15092 | | | | | | |
| 1242471 | 14559 | 14574 | N/A | N/A | GGATGGGTGCTGAGCC | 21 | O | 1143 |
| | 14851 | 14866 | | | | | | |
| | 15141 | 15156 | | | | | | |
| | 15214 | 15229 | | | | | | |
| 1242495 | 14604 | 14619 | N/A | N/A | TTCAGTGAGAGTCTGT | 23 | O | 1144 |
| | 14750 | 14765 | | | | | | |
| | 14896 | 14911 | | | | | | |
| | 15042 | 15057 | | | | | | |
| 1242519 | 15182 | 15197 | N/A | N/A | GTCAGAGTCTGTCCTC | 9 | O | 1145 |
| | 15255 | 15270 | | | | | | |
| 1242543 | 17464 | 17479 | N/A | N/A | GTCTTTAAAGGAGTGA | 23 | O | 1146 |
| 1242567 | 18758 | 18773 | N/A | N/A | GCATTTAAGCCACCGA | 23 | O | 1147 |
| 1242591 | 19744 | 19759 | N/A | N/A | GCAATAGTTCATAGGT | 51 | O | 1148 |
| 1242615 | 20586 | 20601 | N/A | N/A | CCTGACCAGAGAGCTC | 42 | O | 1149 |
| | 20628 | 20643 | | | | | | |
| | 20670 | 20685 | | | | | | |
| | 20712 | 20727 | | | | | | |
| | 20754 | 20769 | | | | | | |
| | 20796 | 20811 | | | | | | |
| | 20922 | 20937 | | | | | | |
| | 21048 | 21063 | | | | | | |
| 1242639 | 20781 | 20796 | N/A | N/A | CTGGAATAGAGAATGC | 85 | O | 1150 |
| 1242663 | 21645 | 21660 | N/A | N/A | GCATACAACATCGAGG | 26 | O | 1151 |
| 1242686 | 22765 | 22780 | N/A | N/A | CACCAATACTACATCT | 100 | O | 1152 |
| 1242710 | 23344 | 23359 | N/A | N/A | CTAAGAAACTCTAGTT | 82 | O | 1153 |
| 1242734 | 23635 | 23650 | N/A | N/A | ACACTAACAAACCAGC | 46 | O | 1154 |
| 1242758 | 24069 | 24084 | N/A | N/A | GGAATTAAGGCAGTGG | 37 | O | 1155 |
| 1242782 | 24260 | 24275 | N/A | N/A | TCCAATTTGCTTATTG | 87 | O | 1156 |
| 1242806 | 24732 | 24747 | N/A | N/A | GGGTATATTGTTTAGC | 42 | O | 1157 |
| 1242830 | 25678 | 25693 | N/A | N/A | AAAGGTAAACCAGGCG | 66 | O | 1158 |
| 1242854 | 26103 | 26118 | N/A | N/A | GTCCATAATTAGCAAC | 87 | O | 1159 |
| 1242878 | 26601 | 26616 | N/A | N/A | TTATTTGGTGATACGG | 27 | O | 1160 |
| 1242902 | 26942 | 26957 | N/A | N/A | TTATAGATACCTATCT | 96 | O | 1161 |
| 1242926 | 27315 | 27330 | N/A | N/A | CACTATTCAATGAGGC | 98 | O | 1162 |
| 1242950 | 27890 | 27905 | N/A | N/A | ATAAGTAGGAGCTTCT | 47 | O | 1163 |
| 1242974 | 28712 | 28727 | N/A | N/A | ATCAAACAACCATGTC | 46 | O | 1164 |
| 1242998 | 29012 | 29027 | N/A | N/A | GGGATTAGTAGCATTC | 82 | O | 1165 |
| 1243022 | 29400 | 29415 | N/A | N/A | GTAATTCCTAGGAGCA | 43 | O | 1166 |
| 1243046 | 29752 | 29767 | N/A | N/A | TATTATATCAACCTGG | 64 | O | 1167 |
| 1243070 | 31266 | 31281 | N/A | N/A | CATGATCCTGTAACAA | 80 | O | 1168 |
| 1243094 | 31948 | 31963 | N/A | N/A | AATATCCACCACAACT | 86 | O | 1169 |
| 1243118 | 33590 | 33605 | N/A | N/A | AGGGAATAATGCAGGT | 112 | O | 1170 |
| 1243142 | 34005 | 34020 | N/A | N/A | GGCTAGGTCAGTGATG | 69 | O | 1171 |
| 1243166 | 34365 | 34380 | N/A | N/A | ATGAAGTTGTGAGGGT | 61 | O | 1172 |
| 1243190 | 34445 | 34460 | N/A | N/A | TTAACAATTCCCATGA | 85 | O | 1173 |
| 1243214 | 34709 | 34724 | N/A | N/A | TCTTAGTATCACTGTA | 66 | O | 1174 |
| 1243238 | 24855 | 24870 | N/A | N/A | GCCCATCCCACGCTTC | 73 | O | 1175 |
| 1232737 | 5138 | 5153 | 90 | 105 | ATGCCATCTTGACCCA | 56 | P | 22 |
| 1241418 | 5083 | 5098 | 35 | 50 | GACATTGGACAAACTC | 32 | P | 1176 |
| 1241442 | 5158 | 5173 | 110 | 125 | CGGTGAACAACCACTT | 66 | P | 1177 |
| 1241466 | 5264 | 5279 | 216 | 231 | CCTGTTGGCTCGATCC | 47 | P | 1178 |
| 1241490 | 5718 | 5733 | 670 | 685 | GGCACACGGATGAGTC | 58 | P | 1179 |
| 1241514 | 5796 | 5811 | 748 | 763 | GTGCTTGCCATCTTCA | 35 | P | 1180 |
| 1241538 | 5918 | 5933 | 870 | 885 | CTGTCTGACCCCTCGG | 35 | P | 1181 |
| 1241562 | 5995 | 6010 | 947 | 962 | CACGGCCATGGCCCAC | 56 | P | 1182 |
| 1241586 | 6036 | 6051 | 988 | 1003 | TTCTCATAAAGGTCTC | 31 | P | 1183 |
| 1241610 | 10264 | 10279 | 1064 | 1079 | GTCTTCCTGGCATATC | 49 | P | 1184 |
| 1241634 | 10848 | 10863 | 1151 | 1166 | GTACTTCTTACGGTAA | 68 | P | 1185 |
| 1241658 | 10904 | 10919 | 1207 | 1222 | CCCAGACGGGCATTCC | 60 | P | 1186 |
| 1241682 | 11180 | 11195 | 1483 | 1498 | TCTTGGTAGAGTGTCC | 48 | P | 1187 |
| 1241706 | 11320 | 11335 | 1623 | 1638 | GGATTCTGGAGGGTTT | 56 | P | 1188 |
| 1241730 | 11520 | 11535 | 1823 | 1838 | ATGGTCCAGCAAGTGC | 56 | P | 1189 |
| 1241754 | 11830 | 11845 | 2133 | 2148 | CCCAGAGGTGGGCGCA | 91 | P | 1190 |
| 1241777 | 12031 | 12046 | 2334 | 2349 | GGTAGTACATGGCGGC | 36 | P | 1191 |
| 1241801 | 12183 | 12198 | 2486 | 2501 | GTTTACCAGGCCAAAG | 78 | P | 1192 |
| 1241824 | 12265 | 12280 | 2568 | 2583 | CAATCCATTTCAGCAG | 52 | P | 1193 |
| 1241848 | 12376 | 12391 | 2679 | 2694 | AATAGTCCATGGCCCT | 33 | P | 1194 |
| 1241872 | 12498 | 12513 | 2801 | 2816 | CTCCTTGGGCATGTTA | 49 | P | 1195 |
| 1241896 | 12550 | 12565 | 2853 | 2868 | GGACACACTGCACCAT | 35 | P | 1196 |
| 1241920 | 16621 | 16636 | 2939 | 2954 | AACTGAAAAGAGGCCC | 88 | P | 1197 |
| 1241944 | N/A | N/A | 3067 | 3082 | CGCCCCAACCACAATC | 67 | P | 1198 |
| 1241968 | 22986 | 23001 | 3261 | 3276 | AACATGCTGATGTGAG | 34 | P | 1199 |
| 1241992 | 23017 | 23032 | 3292 | 3307 | CTGGTGCTCAATACTG | 62 | P | 1200 |
| 1242016 | 23072 | 23087 | 3347 | 3362 | GACTCCTGAGTCTCCC | 35 | P | 1201 |
| 1242040 | N/A | N/A | 3412 | 3427 | GAATTCACCAACCCCA | 75 | P | 1202 |
| 1242064 | 31020 | 31035 | 3464 | 3479 | ATTAGTGCTGAGTACC | 58 | P | 1203 |
| 1242088 | 31068 | 31083 | 3512 | 3527 | CTTGTCTCCGAGAGTG | 44 | P | 1204 |
| 1242112 | 31653 | 31668 | 3589 | 3604 | AGGTTGCAGTTGTCTA | 59 | P | 1205 |
| 1242136 | 31764 | 31779 | 3700 | 3715 | CAGAACATCATGACCC | 58† | P | 1206 |
| 1242160 | 35555 | 35570 | 3908 | 3923 | TCCACCTGAGGGCCCC | 70 | P | 1207 |
| 1242184 | 35718 | 35733 | 4071 | 4086 | AACAACACCCGATGCT | 89 | P | 1208 |
| 1242208 | 35791 | 35806 | 4144 | 4159 | AGTGCTTTATTGAATG | 49 | P | 1209 |
| 1242232 | 35920 | 35935 | 4273 | 4288 | CAACTAGTTCTGTGTT | 91 | P | 1210 |
| 1242256 | 3168 | 3183 | N/A | N/A | GCCTCAGGAACAGCTA | 96 | P | 1211 |
| 1242280 | 6827 | 6842 | N/A | N/A | AAGTAATCCAGGTGTT | 92 | P | 1212 |
| 1242304 | 8225 | 8240 | N/A | N/A | ACTCATTTGTACTCCT | 63 | P | |
| 1242328 | 8771 | 8786 | N/A | N/A | GTTATAACTTTTCTGC | 96 | P | 1214 |
| 1242352 | 9677 | 9692 | N/A | N/A | GACTTTAGAATCACCC | 86 | P | 1215 |
| 1242376 | 10542 | 10557 | N/A | N/A | CTTAATCACCGCCGCT | 48 | P | 1216 |
| 1242400 | 12825 | 12840 | N/A | N/A | TAAACTGGCATTCCCC | 51 | P | 1217 |
| 1242424 | 14186 | 14201 | N/A | N/A | TTAGGAAAGGGACCCC | 66 | P | 1218 |
| 1242448 | 14494 | 14509 | N/A | N/A | ATCTAGAGAAATGGGT | 35 | P | 1219 |
| | 14640 | 14655 | | | | | | |
| | 14713 | 14728 | | | | | | |
| | 14786 | 14801 | | | | | | |
| | 15005 | 15020 | | | | | | |
| | 15078 | 15093 | | | | | | |
| 1242472 | 14569 | 14584 | N/A | N/A | CCATCTAGAGGGATGG | 77 | P | 1220 |
| | 14861 | 14876 | | | | | | |
| | 15151 | 15166 | | | | | | |
| | 15224 | 15239 | | | | | | |
| 1242496 | 14605 | 14620 | N/A | N/A | CTTCAGTGAGAGTCTG | 24 | P | 1221 |
| | 14751 | 14766 | | | | | | |
| | 14897 | 14912 | | | | | | |
| | 15043 | 15058 | | | | | | |
| 1242520 | 15290 | 15305 | N/A | N/A | GAAAGATGGGTGCTGA | 86 | P | 1222 |
| 1242544 | 17492 | 17507 | N/A | N/A | TCATAATGTGGCTGTT | 51 | P | 1223 |
| 1242568 | 18759 | 18774 | N/A | N/A | AGCATTTAAGCCACCG | 43 | P | 1224 |
| 1242592 | 19770 | 19785 | N/A | N/A | GAACAACATTCTAGCT | 68 | P | 1225 |
| 1242616 | 20587 | 20602 | N/A | N/A | ACCTGACCAGAGAGCT | 31 | P | 1226 |
| | 20629 | 20644 | | | | | | |
| | 20671 | 20686 | | | | | | |
| | 20713 | 20728 | | | | | | |
| | 20755 | 20770 | | | | | | |
| | 20797 | 20812 | | | | | | |
| | 20923 | 20938 | | | | | | |
| | 21049 | 21064 | | | | | | |
| 1242640 | 20837 | 20852 | N/A | N/A | CTGACAAGAGAGCTCT | 76 | P | 1227 |
| 1242664 | 21728 | 21743 | N/A | N/A | GGTGAGAAATGGCCCT | 52 | P | 1228 |
| 1242687 | 22787 | 22802 | N/A | N/A | AAGTAAGCATTCTCCA | 53 | P | 1229 |
| 1242711 | 23345 | 23360 | N/A | N/A | ACTAAGAAACTCTAGT | 125 | P | 1230 |
| 1242735 | 23686 | 23701 | N/A | N/A | TAAAACATCCACCATG | 97 | P | 1231 |
| 1242759 | 24070 | 24085 | N/A | N/A | GGGAATTAAGGCAGTG | 87 | P | 1232 |
| 1242783 | 24265 | 24280 | N/A | N/A | ACCTATCCAATTTGCT | 93 | P | 1233 |
| 1242807 | 24743 | 24758 | N/A | N/A | TCTTATACACAGGGTA | 31 | P | 1234 |
| 1242831 | 25745 | 25760 | N/A | N/A | GTTGAAATTGAGAGGT | 31 | P | 1235 |
| 1242855 | 26114 | 26129 | N/A | N/A | GAAATCCATTTGTCCA | 68 | P | 1236 |
| 1242879 | 26602 | 26617 | N/A | N/A | CTTATTTGGTGATACG | 54 | P | 1237 |
| 1242903 | 26946 | 26961 | N/A | N/A | CCACTTATAGATACCT | 43 | P | 1238 |
| 1242927 | 27316 | 27331 | N/A | N/A | CCACTATTCAATGAGG | 85 | P | 1239 |
| 1242951 | 27891 | 27906 | N/A | N/A | AATAAGTAGGAGCTTC | 49 | P | 1240 |
| 1242975 | 28714 | 28729 | N/A | N/A | GAATCAAACAACCATG | 60 | P | 1241 |
| 1242999 | 29013 | 29028 | N/A | N/A | AGGGATTAGTAGCATT | 72 | P | 1242 |
| 1243023 | 29401 | 29416 | N/A | N/A | CGTAATTCCTAGGAGC | 57 | P | 1243 |
| 1243047 | 29755 | 29770 | N/A | N/A | GATTATTATATCAACC | 108 | P | 1244 |
| 1243071 | 31297 | 31312 | N/A | N/A | CTATATCATCTGGAAG | 94 | P | 1245 |
| 1243095 | 32070 | 32085 | N/A | N/A | CATGATCGCATGAGGG | 53 | P | 1246 |
| 1243119 | 33606 | 33621 | N/A | N/A | GAATTTAGGAAGGTAC | 113 | P | 1247 |
| 1243143 | 34050 | 34065 | N/A | N/A | CTATTTTGAGCCTGTC | 50 | P | 1248 |
| 1243167 | 34366 | 34381 | N/A | N/A | GATGAAGTTGTGAGGG | 73 | P | 1249 |
| 1243191 | 34446 | 34461 | N/A | N/A | CTTAACAATTCCCATG | 70 | P | 1250 |
| 1243215 | 34724 | 34739 | N/A | N/A | CGTTTAACAATAATTT | 105 | P | 1251 |
| 1243239 | 33636 | 33651 | N/A | N/A | ACAGCCTCGCAGAGAA | 82 | P | 1252 |
| 1232737 | 5138 | 5153 | 90 | 105 | ATGCCATCTTGACCCA | 60 | Q | 22 |
| 1241419 | 5088 | 5103 | 40 | 55 | GCTATGACATTGGACA | 54 | Q | 1253 |
| 1241443 | 5167 | 5182 | 119 | 134 | TACAGTTTACGGTGAA | 50 | Q | 1254 |
| 1241467 | 5265 | 5280 | 217 | 232 | TCCTGTTGGCTCGATC | 56 | Q | 1255 |
| 1241491 | 5728 | 5743 | 680 | 695 | CAGTGAACACGGCACA | 23 | Q | 1256 |
| 1241515 | 5797 | 5812 | 749 | 764 | GGTGCTTGCCATCTTC | 47 | Q | 1257 |
| 1241539 | 5919 | 5934 | 871 | 886 | TCTGTCTGACCCCTCG | 32 | Q | 1258 |
| 1241563 | 5996 | 6011 | 948 | 963 | ACACGGCCATGGCCCA | 33 | Q | 1259 |
| 1241587 | 6038 | 6053 | 990 | 1005 | CTTTCTCATAAAGGTC | 52 | Q | 1260 |
| 1241611 | 10265 | 10280 | 1065 | 1080 | TGTCTTCCTGGCATAT | 52 | Q | 1261 |
| 1241635 | 10849 | 10864 | 1152 | 1167 | TGTACTTCTTACGGTA | 39 | Q | 1262 |
| 1241659 | 10906 | 10921 | 1209 | 1224 | CACCCAGACGGGCATT | 57 | Q | 1263 |
| 1241683 | 11181 | 11196 | 1484 | 1499 | GTCTTGGTAGAGTGTC | 31 | Q | 1264 |
| 1241707 | 11321 | 11336 | 1624 | 1639 | AGGATTCTGGAGGGTT | 60 | Q | 1265 |
| 1241731 | 11521 | 11536 | 1824 | 1839 | GATGGTCCAGCAAGTG | 39 | Q | 1266 |
| 1241755 | 11831 | 11846 | 2134 | 2149 | CCCCAGAGGTGGGCGC | 65 | Q | 1267 |
| 1241778 | 12033 | 12048 | 2336 | 2351 | CAGGTAGTACATGGCG | 47 | Q | 1268 |
| 1241802 | 12184 | 12199 | 2487 | 2502 | GGTTTACCAGGCCAAA | 38 | Q | 1269 |
| 1241825 | 12274 | 12289 | 2577 | 2592 | CTTTCACTTCAATCCA | 36 | Q | 1270 |
| 1241849 | 12401 | 12416 | 2704 | 2719 | GAGAGATTGATCTCAA | 68 | Q | 1271 |
| 1241873 | 12499 | 12514 | 2802 | 2817 | CCTCCTTGGGCATGTT | 41 | Q | 1272 |
| 1241897 | 12551 | 12566 | 2854 | 2869 | AGGACACACTGCACCA | 38 | Q | 1273 |
| 1241921 | 16644 | 16659 | 2962 | 2977 | GTTAGACTCTGGCTGG | 29 | Q | 1274 |
| 1241945 | 21109 | 21124 | 3080 | 3095 | CGAGAGGCCACAGCGC | 66 | Q | 1275 |
| 1241969 | 22987 | 23002 | 3262 | 3277 | CAACATGCTGATGTGA | 54 | Q | 1276 |
| 1241993 | 23018 | 23033 | 3293 | 3308 | GCTGGTGCTCAATACT | 48 | Q | 1277 |
| 1242017 | 23073 | 23088 | 3348 | 3363 | CGACTCCTGAGTCTCC | 57 | Q | 1278 |
| 1242041 | N/A | N/A | 3413 | 3428 | AGAATTCACCAACCCC | 43 | Q | 1279 |
| 1242065 | 31022 | 31037 | 3466 | 3481 | TGATTAGTGCTGAGTA | 43 | Q | 1280 |
| 1242089 | 31069 | 31084 | 3513 | 3528 | CCTTGTCTCCGAGAGT | 37 | Q | 1281 |
| 1242113 | 31654 | 31669 | 3590 | 3605 | GAGGTTGCAGTTGTCT | 43 | Q | 1282 |
| 1242137 | 31766 | 31781 | 3702 | 3717 | CACAGAACATCATGAC | 63† | Q | 1283 |
| 1242161 | 35570 | 35585 | 3923 | 3938 | TGGATCGCAGCTCTCT | 33 | Q | 1284 |
| 1242185 | 35721 | 35736 | 4074 | 4089 | GACAACAACACCCGAT | 77 | Q | 1285 |
| 1242209 | 35872 | 35887 | 4225 | 4240 | TGATATGAGCAAAAGT | 78 | Q | 1286 |
| 1242233 | 35921 | 35936 | 4274 | 4289 | TCAACTAGTTCTGTGT | 62 | Q | 1287 |
| 1242257 | N/A | N/A | N/A | N/A | ATCCAGATGCCAGCCT | 75 | Q | 1288 |
| 1242281 | 6828 | 6843 | N/A | N/A | GAAGTAATCCAGGTGT | 49 | Q | 1289 |
| 1242305 | 8251 | 8266 | N/A | N/A | CTTAACATGTGTCTCC | 63 | O | 1290 |
| 1242329 | 8774 | 8789 | N/A | N/A | CTAGTTATAACTTTTC | 82 | Q | 1291 |
| 1242353 | 9691 | 9706 | N/A | N/A | TGGGAATCCTGGCTGA | 78 | Q | 1292 |
| 1242377 | 10543 | 10558 | N/A | N/A | GCTTAATCACCGCCGC | 52 | Q | 1293 |
| 1242401 | 12827 | 12842 | N/A | N/A | GCTAAACTGGCATTCC | 60 | Q | 1294 |
| 1242425 | 14187 | 14202 | N/A | N/A | CTTAGGAAAGGGACCC | 66 | Q | 1295 |
| 1242449 | 14495 | 14510 | N/A | N/A | TATCTAGAGAAATGGG | 32 | Q | 1296 |
| | 14641 | 14656 | | | | | | |
| | 14714 | 14729 | | | | | | |
| | 14787 | 14802 | | | | | | |
| | 15006 | 15021 | | | | | | |
| | 15079 | 15094 | | | | | | |
| 1242473 | 14570 | 14585 | N/A | N/A | GCCATCTAGAGGGATG | 53 | Q | 1297 |
| | 14862 | 14877 | | | | | | |
| | 15152 | 15167 | | | | | | |
| | 15225 | 15240 | | | | | | |
| 1242497 | 14606 | 14621 | N/A | N/A | GCTTCAGTGAGAGTCT | 24 | Q | 1298 |
| | 14752 | 14767 | | | | | | |
| | 14898 | 14913 | | | | | | |
| | 15044 | 15059 | | | | | | |
| 1242521 | 15292 | 15307 | N/A | N/A | TAGAAAGATGGGTGCT | 51 | Q | 1299 |
| 1242545 | 17657 | 17672 | N/A | N/A | CATCATGGTTGTTTCA | 31 | Q | 1300 |
| 1242569 | 18760 | 18775 | N/A | N/A | AAGCATTTAAGCCACC | 47 | Q | 1301 |
| 1242593 | 19793 | 19808 | N/A | N/A | CTTTATCCCTGTCATG | 73 | Q | 1302 |
| 1242617 | 20588 | 20603 | N/A | N/A | CACCTGACCAGAGAGC | 28 | Q | 1303 |
| | 20630 | 20645 | | | | | | |
| | 20672 | 20687 | | | | | | |
| | 20714 | 20729 | | | | | | |
| | 20756 | 20771 | | | | | | |
| | 20798 | 20813 | | | | | | |
| | 20924 | 20939 | | | | | | |
| | 21050 | 21065 | | | | | | |
| 1242641 | 20877 | 20892 | N/A | N/A | GACCAGAGAGCTCCAG | 33 | Q | 1304 |
| | 21045 | 21060 | | | | | | |
| 1242665 | 21874 | 21889 | N/A | N/A | TAGGAATTGCTGTGCT | 34 | Q | 1305 |
| 1242688 | 22788 | 22803 | N/A | N/A | CAAGTAAGCATTCTCC | 32 | Q | 1306 |
| 1242712 | 23346 | 23361 | N/A | N/A | CACTAAGAAACTCTAG | 77 | Q | 1307 |
| 1242736 | 23687 | 23702 | N/A | N/A | GTAAAACATCCACCAT | 66 | Q | 1308 |
| 1242760 | 24071 | 24086 | N/A | N/A | TGGGAATTAAGGCAGT | 63 | Q | 1309 |
| 1242784 | 24316 | 24331 | N/A | N/A | CTTGAATGCTGATCTG | 65 | Q | 1310 |
| 1242808 | 24744 | 24759 | N/A | N/A | CTCTTATACACAGGGT | 30 | Q | 1311 |
| 1242832 | 25773 | 25788 | N/A | N/A | ATGATATCTGGGTTTG | 35 | Q | 1312 |
| 1242856 | 26124 | 26139 | N/A | N/A | GCATATACAGGAAATC | 79 | Q | 1313 |
| 1242880 | 26603 | 26618 | N/A | N/A | CCTTATTTGGTGATAC | 36 | Q | 1314 |
| 1242904 | 26956 | 26971 | N/A | N/A | CTGATTAATGCCACTT | 22 | Q | 1315 |
| 1242928 | 27367 | 27382 | N/A | N/A | AGCATTTAGGACCGTC | 25 | Q | 1316 |
| 1242952 | 27919 | 27934 | N/A | N/A | GACAAAATGAGGATCA | 72 | Q | 1317 |
| 1242976 | 28750 | 28765 | N/A | N/A | CAACGGTTACACTGAA | 40 | Q | 1318 |
| 1243000 | 29014 | 29029 | N/A | N/A | AAGGGATTAGTAGCAT | 61 | Q | 1319 |
| 1243024 | 29415 | 29430 | N/A | N/A | AAAATCACTGGTGGCG | 40 | Q | 1320 |
| 1243048 | 29756 | 29771 | N/A | N/A | TGATTATTATATCAAC | 95 | Q | 1321 |
| 1243072 | 31298 | 31313 | N/A | N/A | TCTATATCATCTGGAA | 73 | Q | 1322 |
| 1243096 | 32087 | 32102 | N/A | N/A | ACAGAATCCTCCTTGG | 84 | Q | 1323 |
| 1243120 | 33712 | 33727 | N/A | N/A | CATAAGATGTGCTACA | 90 | Q | 1324 |
| 1243144 | 34052 | 34067 | N/A | N/A | ATCTATTTTGAGCCTG | 38 | Q | 1325 |
| 1243168 | 34369 | 34384 | N/A | N/A | TTAGATGAAGTTGTGA | 58 | Q | 1326 |
| 1243192 | 34482 | 34497 | N/A | N/A | CAATTTGGTGGTGTTA | 57 | Q | 1327 |
| 1243216 | 34750 | 34765 | N/A | N/A | TTACAATGTCACTATC | 58 | Q | 1328 |
| 1243240 | 19916 | 19931 | N/A | N/A | GCTGGGATTCCACATC | 55 | Q | 1329 |
| 1232737 | 5138 | 5153 | 90 | 105 | ATGCCATCTTGACCCA | 45 | R | 22 |
| 1241420 | 5090 | 5105 | 42 | 57 | GGGCTATGACATTGGA | 70 | R | 1330 |
| 1241444 | 5169 | 5184 | 121 | 136 | ATTACAGTTTACGGTG | 73 | R | 1331 |
| 1241468 | 5270 | 5285 | 222 | 237 | AGTTCTCCTGTTGGCT | 45 | R | 1332 |
| 1241492 | 5729 | 5744 | 681 | 696 | GCAGTGAACACGGCAC | 46 | R | 1333 |
| 1241516 | 5810 | 5825 | 762 | 777 | CCAGCTTGCAGCGGGT | 57 | R | 1334 |
| 1241540 | 5936 | 5951 | 888 | 903 | CCACATGGTCTGCCTT | 38 | R | 1335 |
| 1241564 | 5997 | 6012 | 949 | 964 | CACACGGCCATGGCCC | 37 | R | 1336 |
| 1241588 | 6039 | 6054 | 991 | 1006 | GCTTTCTCATAAAGGT | 49 | R | 1337 |
| 1241612 | 10266 | 10281 | 1066 | 1081 | CTGTCTTCCTGGCATA | 52 | R | 1338 |
| 1241636 | 10850 | 10865 | 1153 | 1168 | CTGTACTTCTTACGGT | 46 | R | 1339 |
| 1241660 | 10923 | 10938 | 1226 | 1241 | GTTGAGGCTCACACTC | 43 | R | 1340 |
| 1241684 | 11212 | 11227 | 1515 | 1530 | GACAGTGGATATAGAA | 54 | R | 1341 |
| 1241708 | 11323 | 11338 | 1626 | 1641 | AGAGGATTCTGGAGGG | 68 | R | 1342 |
| 1241732 | 11522 | 11537 | 1825 | 1840 | GGATGGTCCAGCAAGT | 42 | R | 1343 |
| 1241756 | 11843 | 11858 | 2146 | 2161 | AAAGAGCAGAGCCCCC | 91 | R | 1344 |
| 1241779 | 12034 | 12049 | 2337 | 2352 | GCAGGTAGTACATGGC | 51 | R | 1345 |
| 1241803 | 12185 | 12200 | 2488 | 2503 | TGGTTTACCAGGCCAA | 69 | R | 1346 |
| 1241826 | 12275 | 12290 | 2578 | 2593 | GCTTTCACTTCAATCC | 24 | R | 1347 |
| 1241850 | 12402 | 12417 | 2705 | 2720 | GGAGAGATTGATCTCA | 29 | R | 1348 |
| 1241874 | 12501 | 12516 | 2804 | 2819 | TTCCTCCTTGGGCATG | 33 | R | 1349 |
| 1241898 | 12552 | 12567 | 2855 | 2870 | GAGGACACACTGCACC | 25 | R | 1350 |
| 1241922 | 16645 | 16660 | 2963 | 2978 | AGTTAGACTCTGGCTG | 42 | R | 1351 |
| 1241946 | 21110 | 21125 | 3081 | 3096 | GCGAGAGGCCACAGCG | 61 | R | 1352 |
| 1241970 | 22988 | 23003 | 3263 | 3278 | ACAACATGCTGATGTG | 68 | R | 1353 |
| 1241994 | 23024 | 23039 | 3299 | 3314 | GGAATGGCTGGTGCTC | 33 | R | 1354 |
| 1242018 | 23074 | 23089 | 3349 | 3364 | GCGACTCCTGAGTCTC | 48 | R | 1355 |
| 1242042 | 30971 | 30986 | 3415 | 3430 | CCAGAATTCACCAACC | 62 | R | 1356 |
| 1242066 | 31023 | 31038 | 3467 | 3482 | CTGATTAGTGCTGAGT | 34 | R | 1357 |
| 1242090 | 31073 | 31088 | 3517 | 3532 | ATCCCCTTGTCTCCGA | 46 | R | 1358 |
| 1242114 | 31655 | 31670 | 3591 | 3606 | TGAGGTTGCAGTTGTC | 36 | R | 1359 |
| 1242138 | 31779 | 31794 | 3715 | 3730 | TGTTTCAGCACTTCAC | 28† | R | 1360 |
| 1242162 | 35572 | 35587 | 3925 | 3940 | GATGGATCGCAGCTCT | 67 | R | 1361 |
| 1242186 | 35722 | 35737 | 4075 | 4090 | TGACAACAACACCCGA | 77 | R | 1362 |
| 1242210 | 35876 | 35891 | 4229 | 4244 | ATGATGATATGAGCAA | 74 | R | 1363 |
| 1242234 | 35922 | 35937 | 4275 | 4290 | GTCAACTAGTTCTGTG | 42 | R | 1364 |
| 1242258 | N/A | N/A | N/A | N/A | TCCTCATCCAGATGCC | 74 | R | 1365 |
| 1242282 | 6829 | 6844 | N/A | N/A | AGAAGTAATCCAGGTG | 39 | R | 1366 |
| 1242306 | 8252 | 8267 | N/A | N/A | CCTTAACATGTGTCTC | 61 | R | 1367 |
| 1242330 | 8821 | 8836 | N/A | N/A | CTAAAACCTAACCCCA | 95 | R | 1368 |
| 1242354 | 9705 | 9720 | N/A | N/A | CCTAATCTAGTGGTTG | 64 | R | 1369 |
| 1242378 | 10557 | 10572 | N/A | N/A | ATCAAACTTGAAACGC | 35 | R | 1370 |
| 1242402 | 12839 | 12854 | N/A | N/A | TTAATACCTTGTGCTA | 48 | R | 1371 |
| 1242426 | 14230 | 14245 | N/A | N/A | GTTAAGTTATGAGGGT | 38 | R | 1372 |
| 1242450 | 14496 | 14511 | N/A | N/A | CTATCTAGAGAAATGG | 33 | R | 1373 |
| | 14642 | 14657 | | | | | | |
| | 14715 | 14730 | | | | | | |
| | 14788 | 14803 | | | | | | |
| | 14934 | 14949 | | | | | | |
| | 15007 | 15022 | | | | | | |
| | 15080 | 15095 | | | | | | |
| 1242474 | 14572 | 14587 | N/A | N/A | GTGCCATCTAGAGGGA | 36 | R | 1374 |
| | 14864 | 14879 | | | | | | |
| | 15154 | 15169 | | | | | | |
| | 15227 | 15242 | | | | | | |
| 1242498 | 14607 | 14622 | N/A | N/A | GGCTTCAGTGAGAGTC | 31 | R | 1375 |
| | 14753 | 14768 | | | | | | |
| | 14899 | 14914 | | | | | | |
| | 15045 | 15060 | | | | | | |
| 1242522 | 15294 | 15309 | N/A | N/A | TCTAGAAAGATGGGTG | 54 | R | 1376 |
| 1242546 | 17670 | 17685 | N/A | N/A | GGAATATATCGAGCAT | 13 | R | 1377 |
| 1242570 | 18852 | 18867 | N/A | N/A | GTAACATATCCATGAA | 28 | R | 1378 |
| 1242594 | 19795 | 19810 | N/A | N/A | TACTTTATCCCTGTCA | 57 | R | 1379 |
| 1242618 | 20589 | 20604 | N/A | N/A | ACACCTGACCAGAGAG | 23 | R | 1380 |
| | 20631 | 20646 | | | | | | |
| | 20673 | 20688 | | | | | | |
| | 20715 | 20730 | | | | | | |
| | 20757 | 20772 | | | | | | |
| | 20799 | 20814 | | | | | | |
| | 20925 | 20940 | | | | | | |
| | 21051 | 21066 | | | | | | |
| 1242642 | 20878 | 20893 | N/A | N/A | TGACCAGAGAGCTCCA | 32 | R | 1381 |
| | 21046 | 21061 | | | | | | |
| 1242666 | 22296 | 22311 | N/A | N/A | TCTAATAGTGTTGCTA | 48 | R | 1382 |
| 1242689 | 22789 | 22804 | N/A | N/A | ACAAGTAAGCATTCTC | 29 | R | 1383 |
| 1242713 | 23381 | 23396 | N/A | N/A | CAATTGGGCTGCACCA | 45 | R | 1384 |
| 1242737 | 23690 | 23705 | N/A | N/A | GAGGTAAAACATCCAC | 69 | R | 1385 |
| 1242761 | 24086 | 24101 | N/A | N/A | CCTAAGGCAATGCACT | 50 | R | 1386 |
| 1242785 | 24328 | 24343 | N/A | N/A | GCCAAATTTTGTCTTG | 59 | R | 1387 |
| 1242809 | 24745 | 24760 | N/A | N/A | GCTCTTATACACAGGG | 27 | R | 1388 |
| 1242833 | 25778 | 25793 | N/A | N/A | TGTGAATGATATCTGG | 24 | R | 1389 |
| 1242857 | 26129 | 26144 | N/A | N/A | GATCAGCATATACAGG | 83 | R | 1390 |
| 1242881 | 26648 | 26663 | N/A | N/A | GACTTTATATGTGGAA | 41 | R | 1391 |
| 1242905 | 26957 | 26972 | N/A | N/A | ACTGATTAATGCCACT | 22 | R | 1392 |
| 1242929 | 27368 | 27383 | N/A | N/A | GAGCATTTAGGACCGT | 23 | R | 1393 |
| 1242953 | 27995 | 28010 | N/A | N/A | ACTTTAAACAGGGTTA | 55 | R | 1394 |
| 1242977 | 28752 | 28767 | N/A | N/A | AGCAACGGTTACACTG | 43 | R | 1395 |
| 1243001 | 29030 | 29045 | N/A | N/A | GTTAATTAACATGCAG | 62 | R | 1396 |
| 1243025 | 29416 | 29431 | N/A | N/A | TAAAATCACTGGTGGC | 39 | R | 1397 |
| 1243049 | 30077 | 30092 | N/A | N/A | ATTAGTTAGGGTGGTG | 40 | R | 1398 |
| 1243073 | 31300 | 31315 | N/A | N/A | CATCTATATCATCTGG | 41 | R | 1399 |
| 1243097 | 32140 | 32155 | N/A | N/A | CCAATTTGGATGACCC | 37 | R | 1400 |
| 1243121 | 33713 | 33728 | N/A | N/A | ACATAAGATGTGCTAC | 71 | R | 1401 |
| 1243145 | 34054 | 34069 | N/A | N/A | CAATCTATTTTGAGCC | 55 | R | 1402 |
| 1243169 | 34370 | 34385 | N/A | N/A | GTTAGATGAAGTTGTG | 63 | R | 1403 |
| 1243193 | 34483 | 34498 | N/A | N/A | TCAATTTGGTGGTGTT | 55 | R | 1404 |
| 1243217 | 34800 | 34815 | N/A | N/A | CTGCATTTAATGCATC | 82 | R | 1405 |
| 1243241 | 22847 | 22862 | N/A | N/A | AGGGAGTGGACACAAG | 78 | R | 1406 |
| 1232737 | 5138 | 5153 | 90 | 105 | ATGCCATCTTGACCCA | 60 | S | 22 |
| 1241421 | 5106 | 5121 | 58 | 73 | TGTCCCGTTGATTACG | 76 | S | 1407 |
| 1241445 | 5176 | 5191 | 128 | 143 | GGATTGTATTACAGTT | 56 | S | 1408 |
| 1241469 | 5271 | 5286 | 223 | 238 | AAGTTCTCCTGTTGGC | 62 | S | 1409 |
| 1241493 | 5736 | 5751 | 688 | 703 | ATACCAGGCAGTGAAC | 83 | S | 1410 |
| 1241517 | 5819 | 5834 | 771 | 786 | GGTACCTGGCCAGCTT | 95 | S | 1411 |
| 1241541 | 5938 | 5953 | 890 | 905 | ATCCACATGGTCTGCC | 72 | S | 1412 |
| 1241565 | 5998 | 6013 | 950 | 965 | CCACACGGCCATGGCC | 51 | S | 1413 |
| 1241589 | 6053 | 6068 | 1005 | 1020 | GCTCATCTCTTTTTGC | 78 | S | 1414 |
| 1241613 | 10277 | 10292 | 1077 | 1092 | CCTCTTCAATGCTGTC | 33 | S | 1415 |
| 1241637 | 10851 | 10866 | 1154 | 1169 | TCTGTACTTCTTACGG | 46 | S | 1416 |
| 1241661 | 10928 | 10943 | 1231 | 1246 | CGTTTGTTGAGGCTCA | 25 | S | 1417 |
| 1241685 | 11233 | 11248 | 1536 | 1551 | GTGTCACAAGGCTCAC | 53 | S | 1418 |
| 1241709 | 11354 | 11369 | 1657 | 1672 | CCTTGCAGCTCATCGA | 44 | S | 1419 |
| 1241733 | 11523 | 11538 | 1826 | 1841 | AGGATGGTCCAGCAAG | 57 | S | 1420 |
| 1241757 | 11896 | 11911 | 2199 | 2214 | TGAGGTCGGACTCCTC | 108 | S | 1421 |
| 1241780 | 12084 | 12099 | 2387 | 2402 | CTTCAAACGACTCCCT | 66 | S | 1422 |
| 1241804 | 12186 | 12201 | 2489 | 2504 | CTGGTTTACCAGGCCA | 54 | S | 1423 |
| 1241827 | 12289 | 12304 | 2592 | 2607 | GCTTTTTAGCTTTGGC | 38 | S | 1424 |
| 1241851 | 12403 | 12418 | 2706 | 2721 | TGGAGAGATTGATCTC | 69 | S | 1425 |
| 1241875 | 12525 | 12540 | 2828 | 2843 | TCGGCCTTCCTTTTCC | 67 | S | 1426 |
| 1241899 | 12553 | 12568 | 2856 | 2871 | GGAGGACACACTGCAC | 55 | S | 1427 |
| 1241923 | 16646 | 16661 | 2964 | 2979 | CAGTTAGACTCTGGCT | 41 | S | 1428 |
| 1241947 | 21117 | 21132 | 3088 | 3103 | CACTCATGCGAGAGGC | 47 | S | 1429 |
| 1241971 | 22989 | 23004 | 3264 | 3279 | GACAACATGCTGATGT | 64 | S | 1430 |
| 1241995 | 23025 | 23040 | 3300 | 3315 | GGGAATGGCTGGTGCT | 39 | S | 1431 |
| 1242019 | 23076 | 23091 | 3351 | 3366 | TTGCGACTCCTGAGTC | 55 | S | 1432 |
| 1242043 | 30972 | 30987 | 3416 | 3431 | GCCAGAATTCACCAAC | 54 | S | 1433 |
| 1242067 | 31024 | 31039 | 3468 | 3483 | TCTGATTAGTGCTGAG | 37 | S | 1434 |
| 1242091 | 31074 | 31089 | 3518 | 3533 | GATCCCCTTGTCTCCG | 52 | S | 1435 |
| 1242115 | 31656 | 31671 | 3592 | 3607 | GTGAGGTTGCAGTTGT | 36 | S | 1436 |
| 1242139 | 31781 | 31796 | 3717 | 3732 | GCTGTTTCAGCACTTC | 8† | S | 1437 |
| 1242163 | 35573 | 35588 | 3926 | 3941 | GGATGGATCGCAGCTC | 36 | S | 1438 |
| 1242187 | 35723 | 35738 | 4076 | 4091 | ATGACAACAACACCCG | 68 | S | 1439 |
| 1242211 | 35879 | 35894 | 4232 | 4247 | GGAATGATGATATGAG | 89 | S | 1440 |
| 1242235 | 35924 | 35939 | 4277 | 4292 | TAGTCAACTAGTTCTG | 53 | S | 1441 |
| 1242259 | N/A | N/A | N/A | N/A | TTCCTCATCCAGATGC | 63 | S | 1442 |
| 1242283 | 6865 | 6880 | N/A | N/A | CAACACTCATAACAGC | 82 | S | 1443 |
| 1242307 | 8254 | 8269 | N/A | N/A | TGCCTTAACATGTGTC | 77 | S | 1444 |
| 1242331 | 8844 | 8859 | N/A | N/A | GTAAATTAGCCAAAGT | 74 | S | 1445 |
| 1242355 | 9764 | 9779 | N/A | N/A | CTATTGTCAAGTCTAG | 56 | S | 1446 |
| 1242379 | 10561 | 10576 | N/A | N/A | CCTTATCAAACTTGAA | 57 | S | 1447 |
| 1242403 | 12840 | 12855 | N/A | N/A | CTTAATACCTTGTGCT | 70 | S | 1448 |
| 1242427 | 14296 | 14311 | N/A | N/A | AAAATCCATACGCTGA | 75 | S | 1449 |
| 1242451 | 14497 | 14512 | N/A | N/A | GCTATCTAGAGAAATG | 32 | S | 1450 |
| | 14643 | 14658 | | | | | | |
| | 14716 | 14731 | | | | | | |
| | 14789 | 14804 | | | | | | |
| | 14935 | 14950 | | | | | | |
| | 15008 | 15023 | | | | | | |
| | 15081 | 15096 | | | | | | |
| 1242475 | 14573 | 14588 | N/A | N/A | GGTGCCATCTAGAGGG | 71 | S | 1451 |
| | 14865 | 14880 | | | | | | |
| | 15155 | 15170 | | | | | | |
| | 15228 | 15243 | | | | | | |
| 1242499 | 14667 | 14682 | N/A | N/A | GTCTGTCCTCAGAGGA | 8 | S | 1452 |
| | 14740 | 14755 | | | | | | |
| | 14813 | 14828 | | | | | | |
| | 14959 | 14974 | | | | | | |
| | 15032 | 15047 | | | | | | |
| 1242523 | 15312 | 15327 | N/A | N/A | AGAGTAAGGCAGCTGC | 30 | S | 1453 |
| 1242547 | 17671 | 17686 | N/A | N/A | TGGAATATATCGAGCA | 13 | S | 1454 |
| 1242571 | 18855 | 18870 | N/A | N/A | GCAGTAACATATCCAT | 15 | S | 1455 |
| 1242595 | 19797 | 19812 | N/A | N/A | TATACTTTATCCCTGT | 73 | S | 1456 |
| 1242619 | 20592 | 20607 | N/A | N/A | AATACACCTGACCAGA | 68 | S | 1457 |
| 1242643 | 20893 | 20908 | N/A | N/A | GCATCAGAACACATCT | 54 | S | 1458 |
| | 20977 | 20992 | | | | | | |
| 1242667 | 22297 | 22312 | N/A | N/A | TTCTAATAGTGTTGCT | 64 | S | 1459 |
| 1242690 | 22791 | 22806 | N/A | N/A | TAACAAGTAAGCATTC | 70 | S | 1460 |
| 1242714 | 23383 | 23398 | N/A | N/A | GGCAATTGGGCTGCAC | 49 | S | 1461 |
| 1242738 | 23691 | 23706 | N/A | N/A | GGAGGTAAAACATCCA | 71 | S | 1462 |
| 1242762 | 24087 | 24102 | N/A | N/A | TCCTAAGGCAATGCAC | 53 | S | 1463 |
| 1242786 | 24362 | 24377 | N/A | N/A | ATTAGTGGATTACTCC | 52 | S | 1464 |
| 1242810 | 24784 | 24799 | N/A | N/A | AGAGAAGCACCCCTCG | 71 | S | 1465 |
| 1242834 | 25819 | 25834 | N/A | N/A | ATGTTTACACCAAGCA | 24 | S | 1466 |
| 1242858 | 26143 | 26158 | N/A | N/A | ATAAATACCACCCAGA | 82 | S | 1467 |
| 1242882 | 26655 | 26670 | N/A | N/A | CCTCTAAGACTTTATA | 95 | S | 1468 |
| 1242906 | 26972 | 26987 | N/A | N/A | TGCATAAAAGTCTCGA | 40 | S | 1469 |
| 1242930 | 27377 | 27392 | N/A | N/A | CCGAAAGCAGAGCATT | 68 | S | 1470 |
| 1242954 | 27996 | 28011 | N/A | N/A | GACTTTAAACAGGGTT | 46 | S | 1471 |
| 1242978 | 28755 | 28770 | N/A | N/A | TTAAGCAACGGTTACA | 53 | S | 1472 |
| 1243002 | 29057 | 29072 | N/A | N/A | CGGTAAGAATTCTATC | 83 | S | 1473 |
| 1243026 | 29417 | 29432 | N/A | N/A | CTAAAATCACTGGTGG | 55 | S | 1474 |
| 1243050 | 30078 | 30093 | N/A | N/A | AATTAGTTAGGGTGGT | 48 | S | 1475 |
| 1243074 | 31313 | 31328 | N/A | N/A | ACTCAAGTTCCTACAT | 78 | S | 1476 |
| 1243098 | 32507 | 32522 | N/A | N/A | CCCATTTAGAAATCCT | 52 | S | 1477 |
| 1243122 | 33714 | 33729 | N/A | N/A | CACATAAGATGTGCTA | 101 | S | 1478 |
| 1243146 | 34077 | 34092 | N/A | N/A | GTTGATTGCATTCATG | 49 | S | 1479 |
| 1243170 | 34373 | 34388 | N/A | N/A | AGAGTTAGATGAAGTT | 49 | S | 1480 |
| 1243194 | 34484 | 34499 | N/A | N/A | CTCAATTTGGTGGTGT | 62 | S | 1481 |
| 1243218 | 34843 | 34858 | N/A | N/A | CTATTGCCCACCCACT | 107 | S | 1482 |
| 1243242 | 27679 | 27694 | N/A | N/A | ATGAGATCAGGCACTC | 86 | S | 1483 |
| 1232737 | 5138 | 5153 | 90 | 105 | ATGCCATCTTGACCCA | 48 | T | 22 |
| 1241422 | 5107 | 5122 | 59 | 74 | TTGTCCCGTTGATTAC | 63 | T | 1484 |
| 1241446 | 5180 | 5195 | 132 | 147 | AACAGGATTGTATTAC | 88 | T | 1485 |
| 1241470 | 5272 | 5287 | 224 | 239 | AAAGTTCTCCTGTTGG | 59 | T | 1486 |
| 1241494 | 5737 | 5752 | 689 | 704 | GATACCAGGCAGTGAA | 63 | T | 1487 |
| 1241518 | 5868 | 5883 | 820 | 835 | TCTAAGTGCATCTTAA | 43 | T | 1488 |
| 1241542 | 5939 | 5954 | 891 | 906 | GATCCACATGGTCTGC | 49 | T | 1489 |
| 1241566 | 5999 | 6014 | 951 | 966 | TCCACACGGCCATGGC | 48 | T | 1490 |
| 1241590 | 6057 | 6072 | 1009 | 1024 | TTCGGCTCATCTCTTT | 56 | T | 1491 |
| 1241614 | 10291 | 10306 | 1091 | 1106 | TAAACCCATCCACTCC | 43 | T | 1492 |
| 1241638 | 10852 | 10867 | 1155 | 1170 | TTCTGTACTTCTTACG | 33 | T | 1493 |
| 1241662 | 10952 | 10967 | 1255 | 1270 | TTGATGAGACGCAGTC | 40 | T | 1494 |
| 1241686 | 11234 | 11249 | 1537 | 1552 | TGTGTCACAAGGCTCA | 37 | T | 1495 |
| 1241710 | 11357 | 11372 | 1660 | 1675 | GCACCTTGCAGCTCAT | 41 | T | 1496 |
| 1241734 | 11526 | 11541 | 1829 | 1844 | CCGAGGATGGTCCAGC | 54 | T | 1497 |
| 1241758 | 11897 | 11912 | 2200 | 2215 | CTGAGGTCGGACTCCT | 51 | T | 1498 |
| 1241781 | 12085 | 12100 | 2388 | 2403 | GCTTCAAACGACTCCC | 56 | T | 1499 |
| 1241805 | 12187 | 12202 | 2490 | 2505 | CCTGGTTTACCAGGCC | 71 | T | 1500 |
| 1241828 | 12294 | 12309 | 2597 | 2612 | CTGCAGCTTTTTAGCT | 71 | T | 1501 |
| 1241852 | 12406 | 12421 | 2709 | 2724 | TGGTGGAGAGATTGAT | 68 | T | 1502 |
| 1241876 | 12526 | 12541 | 2829 | 2844 | GTCGGCCTTCCTTTTC | 67 | T | 1503 |
| 1241900 | 12555 | 12570 | 2858 | 2873 | TGGGAGGACACACTGC | 54 | T | 1504 |
| 1241924 | 16647 | 16662 | 2965 | 2980 | TCAGTTAGACTCTGGC | 42 | T | 1505 |
| 1241948 | 21118 | 21133 | 3089 | 3104 | GCACTCATGCGAGAGG | 55 | T | 1506 |
| 1241972 | 22990 | 23005 | 3265 | 3280 | TGACAACATGCTGATG | 67 | T | 1507 |
| 1241996 | 23029 | 23044 | 3304 | 3319 | GTCAGGGAATGGCTGG | 61 | T | 1508 |
| 1242020 | 23077 | 23092 | 3352 | 3367 | ATTGCGACTCCTGAGT | 48 | T | 1509 |
| 1242044 | 30975 | 30990 | 3419 | 3434 | AAGGCCAGAATTCACC | 59 | T | 1510 |
| 1242068 | 31025 | 31040 | 3469 | 3484 | TTCTGATTAGTGCTGA | 56 | T | 1511 |
| 1242092 | 31076 | 31091 | 3520 | 3535 | TTGATCCCCTTGTCTC | 72 | T | 1512 |
| 1242116 | 31657 | 31672 | 3593 | 3608 | CGTGAGGTTGCAGTTG | 49 | T | 1513 |
| 1242140 | N/A | N/A | 3753 | 3768 | TTTCAGACAACCCCAG | 22† | T | 1514 |
| 1242164 | 35576 | 35591 | 3929 | 3944 | CCTGGATGGATCGCAG | 75 | T | 1515 |
| 1242188 | 35728 | 35743 | 4081 | 4096 | CTGTGATGACAACAAC | 108 | T | 1516 |
| 1242212 | 35880 | 35895 | 4233 | 4248 | GGGAATGATGATATGA | 126 | T | 1517 |
| 1242236 | 35925 | 35940 | 4278 | 4293 | ATAGTCAACTAGTTCT | 66 | T | 1518 |
| 1242260 | N/A | N/A | N/A | N/A | AAGGTTTCCCCAGATG | 92 | T | 1519 |
| 1242284 | 6945 | 6960 | N/A | N/A | ACCGTATGATTCAGAA | 53 | T | 1520 |
| 1242308 | 8279 | 8294 | N/A | N/A | TGTCATTACACCCACA | 79 | T | 1521 |
| 1242332 | 8848 | 8863 | N/A | N/A | GACAGTAAATTAGCCA | 49 | T | 1522 |
| 1242356 | 9784 | 9799 | N/A | N/A | ATCCATTTTACACACG | 38 | T | 1523 |
| 1242380 | 10563 | 10578 | N/A | N/A | TGCCTTATCAAACTTG | 59 | T | 1524 |
| 1242404 | 12843 | 12858 | N/A | N/A | CTACTTAATACCTTGT | 73 | T | 1525 |
| 1242428 | 14298 | 14313 | N/A | N/A | CCAAAATCCATACGCT | 69 | T | 1526 |
| 1242452 | 14499 | 14514 | N/A | N/A | GTGCTATCTAGAGAAA | 33 | T | 1527 |
| | 14645 | 14660 | | | | | | |
| | 14718 | 14733 | | | | | | |
| | 14791 | 14806 | | | | | | |
| | 14937 | 14952 | | | | | | |
| | 15010 | 15025 | | | | | | |
| | 15083 | 15098 | | | | | | |
| 1242476 | 14574 | 14589 | N/A | N/A | AGGTGCCATCTAGAGG | 19 | T | 1528 |
| | 14866 | 14881 | | | | | | |
| | 15156 | 15171 | | | | | | |
| | 15229 | 15244 | | | | | | |
| 1242500 | 14668 | 14683 | N/A | N/A | AGTCTGTCCTCAGAGG | 12 | T | 1529 |
| | 14741 | 14756 | | | | | | |
| | 14814 | 14829 | | | | | | |
| | 14960 | 14975 | | | | | | |
| | 15033 | 15048 | | | | | | |
| 1242524 | 15336 | 15351 | N/A | N/A | CATACAAAGCAAGCTC | 49 | T | 1530 |
| 1242548 | 17674 | 17689 | N/A | N/A | AAATGGAATATATCGA | 82 | T | 1531 |
| 1242572 | 18856 | 18871 | N/A | N/A | TGCAGTAACATATCCA | 22 | T | 1532 |
| 1242596 | 19799 | 19814 | N/A | N/A | ACTATACTTTATCCCT | 61 | T | 1533 |
| 1242620 | 20596 | 20611 | N/A | N/A | TCAGAATACACCTGAC | 75 | T | 1534 |
| 1242644 | 20894 | 20909 | N/A | N/A | AGCATCAGAACACATC | 36 | T | 1535 |
| | 20978 | 20993 | | | | | | |
| 1242668 | 22375 | 22390 | N/A | N/A | GCTGAATGTACATATA | 42 | T | 1536 |
| 1242691 | 22795 | 22810 | N/A | N/A | GGAATAACAAGTAAGC | 41 | T | 1537 |
| 1242715 | 23384 | 23399 | N/A | N/A | AGGCAATTGGGCTGCA | 34 | T | 1538 |
| 1242739 | 23693 | 23708 | N/A | N/A | CAGGAGGTAAAACATC | 79 | T | 1539 |
| 1242763 | 24088 | 24103 | N/A | N/A | CTCCTAAGGCAATGCA | 46 | T | 1540 |
| 1242787 | 24365 | 24380 | N/A | N/A | TGGATTAGTGGATTAC | 56 | T | 1541 |
| 1242811 | 24786 | 24801 | N/A | N/A | GCAGAGAAGCACCCCT | 66 | T | 1542 |
| 1242835 | 25822 | 25837 | N/A | N/A | GATATGTTTACACCAA | 24 | T | 1543 |
| 1242859 | 26146 | 26161 | N/A | N/A | GATATAAATACCACCC | 93 | T | 1544 |
| 1242883 | 26685 | 26700 | N/A | N/A | ATAGATAGGGTGTTAT | 109 | T | 1545 |
| 1242907 | 27023 | 27038 | N/A | N/A | AGGCTATACCTAACAC | 21 | T | 1546 |
| | 27059 | 27074 | | | | | | |
| | 27095 | 27110 | | | | | | |
| | 27113 | 27128 | | | | | | |
| 1242931 | 27379 | 27394 | N/A | N/A | AACCGAAAGCAGAGCA | 72 | T | 1547 |
| 1242955 | 28557 | 28572 | N/A | N/A | ATTAACTACCTGCCTA | 121 | T | 1548 |
| 1242979 | 28757 | 28772 | N/A | N/A | TATTAAGCAACGGTTA | 113 | T | 1549 |
| 1243003 | 29074 | 29089 | N/A | N/A | TACTAAGCTCTCATCC | 86 | T | 1550 |
| 1243027 | 29418 | 29433 | N/A | N/A | TCTAAAATCACTGGTG | 65 | T | 1551 |
| 1243051 | 30284 | 30299 | N/A | N/A | CATGATTGCGCTGCCA | 50 | T | 1552 |
| 1243075 | 31323 | 31338 | N/A | N/A | GATGGTAAGGACTCAA | 43 | T | 1553 |
| 1243099 | 32574 | 32589 | N/A | N/A | GCAATATCCTGACACA | 58 | T | 1554 |
| 1243123 | 33719 | 33734 | N/A | N/A | CATAGCACATAAGATG | 91 | T | 1555 |
| 1243147 | 34087 | 34102 | N/A | N/A | GAGAAAACGGGTTGAT | 69 | T | 1556 |
| 1243171 | 34374 | 34389 | N/A | N/A | TAGAGTTAGATGAAGT | 56 | T | 1557 |
| 1243195 | 34485 | 34500 | N/A | N/A | TCTCAATTTGGTGGTG | 64 | T | 1558 |
| 1243219 | 34876 | 34891 | N/A | N/A | GACAATTAAACAGGTC | 81 | T | 1559 |
| 1243243 | 23380 | 23395 | N/A | N/A | AATTGGGCTGCACCAT | 54 | T | 1560 |
| 1232737 | 5138 | 5153 | 90 | 105 | ATGCCATCTTGACCCA | 60 | U | 22 |
| 1241423 | 5108 | 5123 | 60 | 75 | TTTGTCCCGTTGATTA | 70 | U | 1561 |
| 1241447 | 5186 | 5201 | 138 | 153 | TCCATAAACAGGATTG | 61 | U | 1562 |
| 1241471 | 5273 | 5288 | 225 | 240 | GAAAGTTCTCCTGTTG | 58 | U | 1563 |
| 1241495 | 5738 | 5753 | 690 | 705 | AGATACCAGGCAGTGA | 60 | U | 1564 |
| 1241519 | 5869 | 5884 | 821 | 836 | CTCTAAGTGCATCTTA | 51 | U | 1565 |
| 1241543 | 5940 | 5955 | 892 | 907 | AGATCCACATGGTCTG | 64 | U | 1566 |
| 1241567 | 6001 | 6016 | 953 | 968 | GATCCACACGGCCATG | 54 | U | 1567 |
| 1241591 | 6058 | 6073 | 1010 | 1025 | CTTCGGCTCATCTCTT | 51 | U | 1568 |
| 1241615 | 10292 | 10307 | 1092 | 1107 | GTAAACCCATCCACTC | 55 | U | 1569 |
| 1241639 | 10858 | 10873 | 1161 | 1176 | CGTACTTTCTGTACTT | 83 | U | 1570 |
| 1241663 | 10954 | 10969 | 1257 | 1272 | CCTTGATGAGACGCAG | 45 | U | 1571 |
| 1241687 | 11235 | 11250 | 1538 | 1553 | CTGTGTCACAAGGCTC | 26 | U | 1572 |
| 1241711 | 11364 | 11379 | 1667 | 1682 | GTCAAAGGCACCTTGC | 52 | U | 1573 |
| 1241735 | 11557 | 11572 | 1860 | 1875 | TGGCCTCGGAGAAACC | 91 | U | 1574 |
| 1241759 | 11902 | 11917 | 2205 | 2220 | GATTCCTGAGGTCGGA | 47 | U | 1575 |
| 1241782 | 12088 | 12103 | 2391 | 2406 | GAAGCTTCAAACGACT | 83 | U | 1576 |
| 1241806 | 12188 | 12203 | 2491 | 2506 | TCCTGGTTTACCAGGC | 98 | U | 1577 |
| 1241829 | 12295 | 12310 | 2598 | 2613 | TCTGCAGCTTTTTAGC | 64 | U | 1578 |
| 1241853 | 12408 | 12423 | 2711 | 2726 | TCTGGTGGAGAGATTG | 73 | U | 1579 |
| 1241877 | 12527 | 12542 | 2830 | 2845 | TGTCGGCCTTCCTTTT | 89 | U | 1580 |
| 1241901 | 12556 | 12571 | 2859 | 2874 | TTGGGAGGACACACTG | 48 | U | 1581 |
| 1241925 | 16648 | 16663 | 2966 | 2981 | TTCAGTTAGACTCTGG | 49 | U | 1582 |
| 1241949 | 21120 | 21135 | 3091 | 3106 | CAGCACTCATGCGAGA | 75 | U | 1583 |
| 1241973 | 22991 | 23006 | 3266 | 3281 | CTGACAACATGCTGAT | 51 | U | 1584 |
| 1241997 | 23032 | 23047 | 3307 | 3322 | CTGGTCAGGGAATGGC | 41 | U | 1585 |
| 1242021 | 23087 | 23102 | 3362 | 3377 | TTCACATAAAATTGCG | 49 | U | 1586 |
| 1242045 | 30976 | 30991 | 3420 | 3435 | TAAGGCCAGAATTCAC | 61 | U | 1587 |
| 1242069 | 31027 | 31042 | 3471 | 3486 | GATTCTGATTAGTGCT | 66 | U | 1588 |
| 1242093 | 31077 | 31092 | 3521 | 3536 | TTTGATCCCCTTGTCT | 90 | U | 1589 |
| 1242117 | 31683 | 31698 | 3619 | 3634 | GTGGAAAGATCCCAGC | 50 | U | 1590 |
| 1242141 | N/A | N/A | 3754 | 3769 | ATTTCAGACAACCCCA | 31† | U | 1591 |
| 1242165 | 35577 | 35592 | 3930 | 3945 | GCCTGGATGGATCGCA | 65 | U | 1592 |
| 1242189 | 35743 | 35758 | 4096 | 4111 | CCTCTAACTGAGGCGC | 57 | U | 1593 |
| 1242213 | 35883 | 35898 | 4236 | 4251 | AGCGGGAATGATGATA | 80 | U | 1594 |
| 1242237 | 35927 | 35942 | 4280 | 4295 | ATATAGTCAACTAGTT | 90 | U | 1595 |
| 1242261 | N/A | N/A | N/A | N/A | GAAGAAAGGTTTCCCC | 77 | U | 1596 |
| 1242285 | 6956 | 6971 | N/A | N/A | TTAACAGAATTACCGT | 50 | U | 1597 |
| 1242309 | 8477 | 8492 | N/A | N/A | ATAAGTACTGACACAA | 52 | U | 1598 |
| 1242333 | 9223 | 9238 | N/A | N/A | CCTAACTAATGTTTGC | 86 | U | 1599 |
| 1242357 | 9824 | 9839 | N/A | N/A | GCTAAGAAAGGTCACC | 64 | U | 1600 |
| 1242381 | 10580 | 10595 | N/A | N/A | AAGTAATCTGCATCCT | 71 | U | 1601 |
| 1242405 | 12844 | 12859 | N/A | N/A | CCTACTTAATACCTTG | 42 | U | 1602 |
| 1242429 | 14299 | 14314 | N/A | N/A | CCCAAAATCCATACGC | 58 | U | 1603 |
| 1242453 | 14500 | 14515 | N/A | N/A | GGTGCTATCTAGAGAA | 21 | U | 1604 |
| | 14646 | 14661 | | | | | | |
| | 14719 | 14734 | | | | | | |
| | 14792 | 14807 | | | | | | |
| | 14938 | 14953 | | | | | | |
| | 15011 | 15026 | | | | | | |
| | 15084 | 15099 | | | | | | |
| 1242477 | 14575 | 14590 | N/A | N/A | CAGGTGCCATCTAGAG | 25 | U | 1605 |
| | 14867 | 14882 | | | | | | |
| | 15157 | 15172 | | | | | | |
| | 15230 | 15245 | | | | | | |
| 1242501 | 14673 | 14688 | N/A | N/A | ATCAGAGTCTGTCCTC | 15 | U | 1606 |
| | 14819 | 14834 | | | | | | |
| | 14965 | 14980 | | | | | | |
| 1242525 | 15378 | 15393 | N/A | N/A | CTAATAGATGCTCAGG | 35 | U | 1607 |
| 1242549 | 17675 | 17690 | N/A | N/A | GAAATGGAATATATCG | 59 | U | 1608 |
| 1242573 | 18880 | 18895 | N/A | N/A | GTAAAAGCCCAGACAC | 69 | U | 1609 |
| 1242597 | 19880 | 19895 | N/A | N/A | GATTAGTTTCCCCGTC | 39 | U | 1610 |
| 1242621 | 20597 | 20612 | N/A | N/A | ATCAGAATACACCTGA | 68 | U | 1611 |
| 1242645 | 20928 | 20943 | N/A | N/A | AACACACCTGACCAGA | 29 | U | 1612 |
| | 21054 | 21069 | | | | | | |
| 1242669 | 22432 | 22447 | N/A | N/A | AGCATAATGTCCCCGA | 28 | U | 1613 |
| 1242692 | 22797 | 22812 | N/A | N/A | GTGGAATAACAAGTAA | 83 | U | 1614 |
| 1242716 | 23425 | 23440 | N/A | N/A | CTATTCATGTGAGTGT | 43 | U | 1615 |
| 1242740 | 23709 | 23724 | N/A | N/A | CCATAGTTCTCTGCAA | 57 | U | 1616 |
| 1242764 | 24101 | 24116 | N/A | N/A | CCATTAGCAACATCTC | 37 | U | 1617 |
| 1242788 | 24370 | 24385 | N/A | N/A | AATTATGGATTAGTGG | 72 | U | 1618 |
| 1242812 | 24787 | 24802 | N/A | N/A | AGCAGAGAAGCACCCC | 45 | U | 1619 |
| 1242836 | 25825 | 25840 | N/A | N/A | TATGATATGTTTACAC | 93 | U | 1620 |
| 1242860 | 26195 | 26210 | N/A | N/A | GTATTACAAATGCAAC | 89 | U | 1621 |
| 1242884 | 26687 | 26702 | N/A | N/A | CAATAGATAGGGTGTT | 48 | U | 1622 |
| 1242908 | 27100 | 27115 | N/A | N/A | CACATAGGCTATACCT | 40 | U | 1623 |
| | 27118 | 27133 | | | | | | |
| 1242932 | 27391 | 27406 | N/A | N/A | AGTGGTAGAGGGAACC | 76 | U | 1624 |
| 1242956 | 28558 | 28573 | N/A | N/A | TATTAACTACCTGCCT | 71 | U | 1625 |
| 1242980 | 28762 | 28777 | N/A | N/A | GTAACTATTAAGCAAC | 68 | U | 1626 |
| 1243004 | 29075 | 29090 | N/A | N/A | CTACTAAGCTCTCATC | 80 | U | 1627 |
| 1243028 | 29419 | 29434 | N/A | N/A | CTCTAAAATCACTGGT | 81 | U | 1628 |
| 1243052 | 30503 | 30518 | N/A | N/A | GCTGATTTAAACATCA | 77 | U | 1629 |
| 1243076 | 31332 | 31347 | N/A | N/A | GAAAACTGTGATGGTA | 51 | U | 1630 |
| 1243100 | 32575 | 32590 | N/A | N/A | GGCAATATCCTGACAC | 57 | U | 1631 |
| 1243124 | 33777 | 33792 | N/A | N/A | GACTAATGGAATTTAT | 87 | U | 1632 |
| 1243148 | 34089 | 34104 | N/A | N/A | CTGAGAAAACGGGTTG | 57 | U | 1633 |
| 1243172 | 34376 | 34391 | N/A | N/A | GTTAGAGTTAGATGAA | 60 | U | 1634 |
| 1243196 | 34525 | 34540 | N/A | N/A | CTAATAAATCCACCCA | 70 | U | 1635 |
| 1243220 | 34889 | 34904 | N/A | N/A | ATTACCCAAGTTCGAC | 56 | U | 1636 |
| 1243244 | 10356 | 10371 | N/A | N/A | AAGCACCACCCCAGTC | 88 | U | 1637 |
| 1232737 | 5138 | 5153 | 90 | 105 | ATGCCATCTTGACCCA | 51 | V | 22 |
| 1241424 | 5109 | 5124 | 61 | 76 | TTTTGTCCCGTTGATT | 62 | V | 1638 |
| 1241448 | 5187 | 5202 | 139 | 154 | ATCCATAAACAGGATT | 89 | V | 1639 |
| 1241472 | 5278 | 5293 | 230 | 245 | TACCAGAAAGTTCTCC | 57 | V | 1640 |
| 1241496 | 5739 | 5754 | 691 | 706 | AAGATACCAGGCAGTG | 49 | V | 1641 |
| 1241520 | 5870 | 5885 | 822 | 837 | CCTCTAAGTGCATCTT | 35 | V | 1642 |
| 1241544 | 5941 | 5956 | 893 | 908 | TAGATCCACATGGTCT | 78 | V | 1643 |
| 1241568 | 6002 | 6017 | 954 | 969 | AGATCCACACGGCCAT | 38 | V | 1644 |
| 1241592 | 6061 | 6076 | 1013 | 1028 | CCACTTCGGCTCATCT | 49 | V | 1645 |
| 1241616 | 10293 | 10308 | 1093 | 1108 | AGTAAACCCATCCACT | 78 | V | 1646 |
| 1241640 | 10872 | 10887 | 1175 | 1190 | GAATCTGCTTCTCACG | 36 | V | 1647 |
| 1241664 | 10955 | 10970 | 1258 | 1273 | TCCTTGATGAGACGCA | 38 | V | 1648 |
| 1241688 | 11236 | 11251 | 1539 | 1554 | TCTGTGTCACAAGGCT | 28 | V | 1649 |
| 1241712 | 11379 | 11394 | 1682 | 1697 | CGGTCCTATGTGCTCG | 56 | V | 1650 |
| 1241736 | 11565 | 11580 | 1868 | 1883 | TTTCCTTTTGGCCTCG | 59 | V | 1651 |
| 1241760 | 11904 | 11919 | 2207 | 2222 | ATGATTCCTGAGGTCG | 44 | V | 1652 |
| 1241783 | 12089 | 12104 | 2392 | 2407 | GGAAGCTTCAAACGAC | 71 | V | 1653 |
| 1241807 | 12189 | 12204 | 2492 | 2507 | CTCCTGGTTTACCAGG | 75 | V | 1654 |
| 1241830 | 12298 | 12313 | 2601 | 2616 | GGATCTGCAGCTTTTT | 43 | V | 1655 |
| 1241854 | 12413 | 12428 | 2716 | 2731 | TCCATTCTGGTGGAGA | 51 | V | 1656 |
| 1241878 | 12528 | 12543 | 2831 | 2846 | GTGTCGGCCTTCCTTT | 49 | V | 1657 |
| 1241902 | 12557 | 12572 | 2860 | 2875 | CTTGGGAGGACACACT | 73 | V | 1658 |
| 1241926 | 16649 | 16664 | 2967 | 2982 | ATTCAGTTAGACTCTG | 83 | V | 1659 |
| 1241950 | 21130 | 21145 | 3101 | 3116 | GATGTCGAAGCAGCAC | 52 | V | 1660 |
| 1241974 | 22994 | 23009 | 3269 | 3284 | ATCCTGACAACATGCT | 46 | V | 1661 |
| 1241998 | 23033 | 23048 | 3308 | 3323 | TCTGGTCAGGGAATGG | 36 | V | 1662 |
| 1242022 | 23103 | 23118 | 3378 | 3393 | GTGGATTCTTGGCTTT | 49 | V | 1663 |
| 1242046 | 30977 | 30992 | 3421 | 3436 | GTAAGGCCAGAATTCA | 41 | V | 1664 |
| 1242070 | 31028 | 31043 | 3472 | 3487 | AGATTCTGATTAGTGC | 51 | V | 1665 |
| 1242094 | 31078 | 31093 | 3522 | 3537 | GTTTGATCCCCTTGTC | 42 | V | 1666 |
| 1242118 | 31685 | 31700 | 3621 | 3636 | GTGTGGAAAGATCCCA | 32 | V | 1667 |
| 1242142 | N/A | N/A | 3755 | 3770 | CATTTCAGACAACCCC | 41† | V | 1668 |
| 1242166 | 35604 | 35619 | 3957 | 3972 | CGGAAGGATCACAGAG | 72 | V | 1669 |
| 1242190 | 35744 | 35759 | 4097 | 4112 | TCCTCTAACTGAGGCG | 56 | V | 1670 |
| 1242214 | 35892 | 35907 | 4245 | 4260 | TAGAAAGATAGCGGGA | 113 | V | 1671 |
| 1242238 | 35928 | 35943 | 4281 | 4296 | TATATAGTCAACTAGT | 55 | V | 1672 |
| 1242262 | 6124 | 6139 | N/A | N/A | GGAAAATTGTACACTA | 41 | V | 1673 |
| 1242286 | 6980 | 6995 | N/A | N/A | CAGTAGAGCAGTTCTT | 54 | V | 1674 |
| 1242310 | 8479 | 8494 | N/A | N/A | ATATAAGTACTGACAC | 55 | V | 1675 |
| 1242334 | 9405 | 9420 | N/A | N/A | CAGGAATCTACAAGTA | 70 | V | 1676 |
| 1242358 | 9847 | 9862 | N/A | N/A | CTATATCCCCAACACG | 78 | V | 1677 |
| 1242382 | 10582 | 10597 | N/A | N/A | TTAAGTAATCTGCATC | 78 | V | 1678 |
| 1242406 | 12858 | 12873 | N/A | N/A | AGGCAATCCTACATCC | 73 | V | 1679 |
| 1242430 | 14374 | 14389 | N/A | N/A | AATGAGTAGTTCTGCC | 55 | V | 1680 |
| 1242454 | 14501 | 14516 | N/A | N/A | AGGTGCTATCTAGAGA | 12 | V | 1681 |
| | 14647 | 14662 | | | | | | |
| | 14720 | 14735 | | | | | | |
| | 14793 | 14808 | | | | | | |
| | 14939 | 14954 | | | | | | |
| | 15012 | 15027 | | | | | | |
| | 15085 | 15100 | | | | | | |
| 1242478 | 14576 | 14591 | N/A | N/A | GCAGGTGCCATCTAGA | 33 | V | 1682 |
| | 14868 | 14883 | | | | | | |
| | 15158 | 15173 | | | | | | |
| | 15231 | 15246 | | | | | | |
| 1242502 | 14674 | 14689 | N/A | N/A | AATCAGAGTCTGTCCT | 42 | V | 1683 |
| | 14820 | 14835 | | | | | | |
| | 14966 | 14981 | | | | | | |
| 1242526 | 15381 | 15396 | N/A | N/A | TTTCTAATAGATGCTC | 42 | V | 1684 |
| 1242550 | 17694 | 17709 | N/A | N/A | TATGAACATGGCCCAT | 54 | V | 1685 |
| 1242574 | 19268 | 19283 | N/A | N/A | GACACTAGAGCCTCAG | 28 | V | 1686 |
| 1242598 | 19882 | 19897 | N/A | N/A | TTGATTAGTTTCCCCG | 18 | V | 1687 |
| 1242622 | 20598 | 20613 | N/A | N/A | CATCAGAATACACCTG | 66 | V | 1688 |
| 1242646 | 20929 | 20944 | N/A | N/A | GAACACACCTGACCAG | 37 | V | 1689 |
| | 21055 | 21070 | | | | | | |
| 1242670 | 22433 | 22448 | N/A | N/A | TAGCATAATGTCCCCG | 36 | V | 1690 |
| 1242693 | 22829 | 22844 | N/A | N/A | CTATGTACACTTCTGA | 45 | V | 1691 |
| 1242717 | 23427 | 23442 | N/A | N/A | GACTATTCATGTGAGT | 64 | V | 1692 |
| 1242741 | 23711 | 23726 | N/A | N/A | AACCATAGTTCTCTGC | 51 | V | 1693 |
| 1242765 | 24114 | 24129 | N/A | N/A | CCCTGTAGAGAGCCCA | 65 | V | 1694 |
| 1242789 | 24371 | 24386 | N/A | N/A | TAATTATGGATTAGTG | 79 | V | 1695 |
| 1242813 | 24788 | 24803 | N/A | N/A | GAGCAGAGAAGCACCC | 64 | V | 1696 |
| 1242837 | 25826 | 25841 | N/A | N/A | CTATGATATGTTTACA | 82 | V | 1697 |
| 1242861 | 26233 | 26248 | N/A | N/A | CTCTAACAGACTAGGC | 54 | V | 1698 |
| 1242885 | 26688 | 26703 | N/A | N/A | TCAATAGATAGGGTGT | 34 | V | 1699 |
| 1242909 | 27101 | 27116 | N/A | N/A | ACACATAGGCTATACC | 66 | V | 1700 |
| | 27119 | 27134 | | | | | | |
| 1242933 | 27454 | 27469 | N/A | N/A | TTTGAACAAAGGGTCA | 60 | V | 1701 |
| 1242957 | 28586 | 28601 | N/A | N/A | TTAAGTTAATTGGTTG | 65 | V | 1702 |
| 1242981 | 28787 | 28802 | N/A | N/A | TCATATCCAAATTGTG | 67 | V | 1703 |
| 1243005 | 29076 | 29091 | N/A | N/A | TCTACTAAGCTCTCAT | 54 | V | 1704 |
| 1243029 | 29420 | 29435 | N/A | N/A | GCTCTAAAATCACTGG | 59 | V | 1705 |
| 1243053 | 30606 | 30621 | N/A | N/A | AACTAATAATCCCCGT | 85 | V | 1706 |
| 1243077 | 31371 | 31386 | N/A | N/A | AATGTAAGTGTCATAC | 59 | V | 1707 |
| 1243101 | 32674 | 32689 | N/A | N/A | GAAAAGTGCAATCATC | 86 | V | 1708 |
| 1243125 | 33797 | 33812 | N/A | N/A | AATATGGACAGAAGTC | 67 | V | 1709 |
| 1243149 | 34090 | 34105 | N/A | N/A | CCTGAGAAAACGGGTT | 59 | V | 1710 |
| 1243173 | 34377 | 34392 | N/A | N/A | AGTTAGAGTTAGATGA | 64 | V | 1711 |
| 1243197 | 34526 | 34541 | N/A | N/A | ACTAATAAATCCACCC | 86 | V | 1712 |
| 1243221 | 34911 | 34926 | N/A | N/A | GTATAATGAACCCCCT | 69 | V | 1713 |
| 1243245 | 32944 | 32959 | N/A | N/A | CCAAGCACGGAGACAC | 84 | V | 1714 |
| 1232737 | 5138 | 5153 | 90 | 105 | ATGCCATCTTGACCCA | 44 | W | 22 |
| 1241425 | 5110 | 5125 | 62 | 77 | TTTTTGTCCCGTTGAT | 92 | W | 1715 |
| 1241449 | 5217 | 5232 | 169 | 184 | TTCCCTATGGAGGGAA | 113 | W | 1716 |
| 1241473 | 5281 | 5296 | 233 | 248 | GCTTACCAGAAAGTTC | 49 | W | 1717 |
| 1241497 | 5740 | 5755 | 692 | 707 | TAAGATACCAGGCAGT | 73 | W | 1718 |
| 1241521 | 5871 | 5886 | 823 | 838 | TCCTCTAAGTGCATCT | 36 | W | 1719 |
| 1241545 | 5942 | 5957 | 894 | 909 | CTAGATCCACATGGTC | 58 | W | 1720 |
| 1241569 | 6003 | 6018 | 955 | 970 | AAGATCCACACGGCCA | 56 | W | 1721 |
| 1241593 | 6062 | 6077 | 1014 | 1029 | CCCACTTCGGCTCATC | 61 | W | 1722 |
| 1241617 | 10295 | 10310 | 1095 | 1110 | CCAGTAAACCCATCCA | 38 | W | 1723 |
| 1241641 | 10879 | 10894 | 1182 | 1197 | TGCACTGGAATCTGCT | 67 | W | 1724 |
| 1241665 | 10956 | 10971 | 1259 | 1274 | CTCCTTGATGAGACGC | 35 | W | 1725 |
| 1241689 | 11237 | 11252 | 1540 | 1555 | CTCTGTGTCACAAGGC | 27 | W | 1726 |
| 1241713 | 11389 | 11404 | 1692 | 1707 | CAGTGCAGAGCGGTCC | 29 | W | 1727 |
| 1241737 | 11592 | 11607 | 1895 | 1910 | ATCAGAGAAGTACTTG | 54 | W | 1728 |
| 1241761 | 11911 | 11926 | 2214 | 2229 | GCAGTCCATGATTCCT | 32 | W | 1729 |
| 1241784 | 12090 | 12105 | 2393 | 2408 | GGGAAGCTTCAAACGA | 47 | W | 1730 |
| 1241808 | 12190 | 12205 | 2493 | 2508 | TCTCCTGGTTTACCAG | 49 | W | 1731 |
| 1241831 | 12299 | 12314 | 2602 | 2617 | TGGATCTGCAGCTTTT | 35 | W | 1732 |
| 1241855 | 12415 | 12430 | 2718 | 2733 | GGTCCATTCTGGTGGA | 48 | W | 1733 |
| 1241879 | 12529 | 12544 | 2832 | 2847 | GGTGTCGGCCTTCCTT | 51 | W | 1734 |
| 1241903 | 12560 | 12575 | 2863 | 2878 | GAGCTTGGGAGGACAC | 53 | W | 1735 |
| 1241927 | 16660 | 16675 | 2978 | 2993 | ACTGAGGTCCAATTCA | 56 | W | 1736 |
| 1241951 | 21134 | 21149 | 3105 | 3120 | AGGAGATGTCGAAGCA | 61 | W | 1737 |
| 1241975 | 22995 | 23010 | 3270 | 3285 | GATCCTGACAACATGC | 51 | W | 1738 |
| 1241999 | 23034 | 23049 | 3309 | 3324 | GTCTGGTCAGGGAATG | 42 | W | 1739 |
| 1242023 | 23104 | 23119 | 3379 | 3394 | TGTGGATTCTTGGCTT | 86 | W | 1740 |
| 1242047 | 30992 | 31007 | 3436 | 3451 | GAACAACAGACTGACG | 55 | W | 1741 |
| 1242071 | 31029 | 31044 | 3473 | 3488 | GAGATTCTGATTAGTG | 22 | W | 1742 |
| 1242095 | 31079 | 31094 | 3523 | 3538 | AGTTTGATCCCCTTGT | 49 | W | 1743 |
| 1242119 | 31686 | 31701 | 3622 | 3637 | AGTGTGGAAAGATCCC | 22 | W | 1744 |
| 1242143 | 35404 | 35419 | 3757 | 3772 | TACATTTCAGACAACC | 29† | W | 1745 |
| 1242167 | 35609 | 35624 | 3962 | 3977 | TCCACCGGAAGGATCA | 56 | W | 1746 |
| 1242191 | 35749 | 35764 | 4102 | 4117 | GAACATCCTCTAACTG | 51 | W | 1747 |
| 1242215 | 35893 | 35908 | 4246 | 4261 | ATAGAAAGATAGCGGG | 102 | W | 1748 |
| 1242239 | 36019 | 36034 | 4372 | 4387 | GCTACAAAAAGCATGG | 38 | W | 1749 |
| 1242263 | 6492 | 6507 | N/A | N/A | GCACAATGTGAGTGTA | 49 | W | 1750 |
| 1242287 | 6982 | 6997 | N/A | N/A | AACAGTAGAGCAGTTC | 53 | W | 1751 |
| 1242311 | 8480 | 8495 | N/A | N/A | TATATAAGTACTGACA | 69 | W | 1752 |
| 1242335 | 9421 | 9436 | N/A | N/A | GATAACTTCAGGTGGG | 50 | W | 1753 |
| 1242359 | 9939 | 9954 | N/A | N/A | AGCAAACATGCGAAGC | 56 | W | 1754 |
| 1242383 | 10585 | 10600 | N/A | N/A | CCTTTAAGTAATCTGC | 34 | W | 1755 |
| 1242407 | 12865 | 12880 | N/A | N/A | TATTTGTAGGCAATCC | 41 | W | 1756 |
| 1242431 | 14376 | 14391 | N/A | N/A | TTAATGAGTAGTTCTG | 61 | W | 1757 |
| 1242455 | 14503 | 14518 | N/A | N/A | GAAGGTGCTATCTAGA | 13 | W | 1758 |
| | 14649 | 14664 | | | | | | |
| | 14722 | 14737 | | | | | | |
| | 14795 | 14810 | | | | | | |
| | 14941 | 14956 | | | | | | |
| | 15014 | 15029 | | | | | | |
| | 15087 | 15102 | | | | | | |
| 1242479 | 14577 | 14592 | N/A | N/A | GGCAGGTGCCATCTAG | 56 | W | 1759 |
| | 14869 | 14884 | | | | | | |
| | 15159 | 15174 | | | | | | |
| | 15232 | 15247 | | | | | | |
| 1242503 | 14675 | 14690 | N/A | N/A | CAATCAGAGTCTGTCC | 27 | W | 1760 |
| | 14821 | 14836 | | | | | | |
| | 14967 | 14982 | | | | | | |
| 1242527 | 15405 | 15420 | N/A | N/A | TAAGATTGGGTCCACA | 36 | W | 1761 |
| 1242551 | 17695 | 17710 | N/A | N/A | CTATGAACATGGCCCA | 31 | W | 1762 |
| 1242575 | 19290 | 19305 | N/A | N/A | ATCCATAGAGCATGGA | 71 | W | 1763 |
| 1242599 | 19883 | 19898 | N/A | N/A | CTTGATTAGTTTCCCC | 26 | W | 1764 |
| 1242623 | 20610 | 20625 | N/A | N/A | GAATAGAGAAAGCATC | 46 | W | 1765 |
| | 20652 | 20667 | | | | | | |
| | 20694 | 20709 | | | | | | |
| | 20736 | 20751 | | | | | | |
| | 20820 | 20835 | | | | | | |
| | 20862 | 20877 | | | | | | |
| | 20904 | 20919 | | | | | | |
| | 20988 | 21003 | | | | | | |
| 1242647 | 20930 | 20945 | N/A | N/A | AGAACACACCTGACCA | 40 | W | 1766 |
| | 21056 | 21071 | | | | | | |
| 1242671 | 22446 | 22461 | N/A | N/A | AGCTTATCTTACTTAG | 80 | W | 1767 |
| 1242694 | 22831 | 22846 | N/A | N/A | CTCTATGTACACTTCT | 36 | W | 1768 |
| 1242718 | 23440 | 23455 | N/A | N/A | ACATAAAATGGGTGAC | 59 | W | 1769 |
| 1242742 | 23713 | 23728 | N/A | N/A | GAAACCATAGTTCTCT | 34 | W | 1770 |
| 1242766 | 24123 | 24138 | N/A | N/A | ATGAAGAAGCCCTGTA | 65 | W | 1771 |
| 1242790 | 24372 | 24387 | N/A | N/A | TTAATTATGGATTAGT | 83 | W | 1772 |
| 1242814 | 24827 | 24842 | N/A | N/A | CTCCAATGTCACCCGC | 44 | W | 1773 |
| 1242838 | 25827 | 25842 | N/A | N/A | ACTATGATATGTTTAC | 68 | W | 1774 |
| 1242862 | 26243 | 26258 | N/A | N/A | ATTACAGTAGCTCTAA | 45 | W | 1775 |
| 1242886 | 26691 | 26706 | N/A | N/A | GTTTCAATAGATAGGG | 32 | W | 1776 |
| 1242910 | 27102 | 27117 | N/A | N/A | AACACATAGGCTATAC | 78 | W | 1777 |
| | 27120 | 27135 | | | | | | |
| 1242934 | 27496 | 27511 | N/A | N/A | CTAAGATGTCACTCTG | 56 | W | 1778 |
| 1242958 | 28588 | 28603 | N/A | N/A | TATTAAGTTAATTGGT | 82 | W | 1779 |
| 1242982 | 28825 | 28840 | N/A | N/A | GAAATTGCAACAGACG | 42 | W | 1780 |
| 1243006 | 29091 | 29106 | N/A | N/A | AAGTTTTGAGTGGCTT | 42 | W | 1781 |
| 1243030 | 29429 | 29444 | N/A | N/A | AGCATAAGAGCTCTAA | 92 | W | 1782 |
| 1243054 | 30608 | 30623 | N/A | N/A | TCAACTAATAATCCCC | 49 | W | 1783 |
| 1243078 | 31406 | 31421 | N/A | N/A | TACCAAATTCTGTGCC | 108 | W | 1784 |
| 1243102 | 32757 | 32772 | N/A | N/A | CTGAGTAGAAGGATCT | 41 | W | 1785 |
| 1243126 | 33874 | 33889 | N/A | N/A | CACAAGGATGCAAGGC | 43 | W | 1786 |
| 1243150 | 34103 | 34118 | N/A | N/A | CTTGTTATCGCAGCCT | 41 | W | 1787 |
| 1243174 | 34380 | 34395 | N/A | N/A | AGTAGTTAGAGTTAGA | 36 | W | 1788 |
| 1243198 | 34530 | 34545 | N/A | N/A | CTTAACTAATAAATCC | 76 | W | 1789 |
| 1243222 | 34960 | 34975 | N/A | N/A | AACTATCTAGTTGGGA | 69 | W | 1790 |
| 1243246 | 22492 | 22507 | N/A | N/A | AGACACCTCCTATAAG | 69 | W | 1791 |
| 1232737 | 5138 | 5153 | 90 | 105 | ATGCCATCTTGACCCA | 43 | X | 22 |
| 1241426 | 5115 | 5130 | 67 | 82 | GAAAATTTTTGTCCCG | 64 | X | 1792 |
| 1241450 | 5219 | 5234 | 171 | 186 | GTTTCCCTATGGAGGG | 81 | X | 1793 |
| 1241474 | 5282 | 5297 | 234 | 249 | TGCTTACCAGAAAGTT | 58 | X | 1794 |
| 1241498 | 5741 | 5756 | 693 | 708 | CTAAGATACCAGGCAG | 57 | X | 1795 |
| 1241522 | 5872 | 5887 | 824 | 839 | GTCCTCTAAGTGCATC | 67 | X | 1796 |
| 1241546 | 5943 | 5958 | 895 | 910 | GCTAGATCCACATGGT | 54 | X | 1797 |
| 1241570 | 6004 | 6019 | 956 | 971 | GAAGATCCACACGGCC | 45 | X | 1798 |
| 1241594 | 6063 | 6078 | 1015 | 1030 | CCCCACTTCGGCTCAT | 62 | X | 1799 |
| 1241618 | 10296 | 10311 | 1096 | 1111 | TCCAGTAAACCCATCC | 48 | X | 1800 |
| 1241642 | 10880 | 10895 | 1183 | 1198 | ATGCACTGGAATCTGC | 58 | X | 1801 |
| 1241666 | 10960 | 10975 | 1263 | 1278 | GGTGCTCCTTGATGAG | 74 | X | 1802 |
| 1241690 | 11238 | 11253 | 1541 | 1556 | CCTCTGTGTCACAAGG | 42 | X | 1803 |
| 1241714 | 11390 | 11405 | 1693 | 1708 | TCAGTGCAGAGCGGTC | 47 | X | 1804 |
| 1241738 | 11593 | 11608 | 1896 | 1911 | CATCAGAGAAGTACTT | 78 | X | 1805 |
| 1241762 | 11912 | 11927 | 2215 | 2230 | TGCAGTCCATGATTCC | 53 | X | 1806 |
| 1241785 | 12091 | 12106 | 2394 | 2409 | TGGGAAGCTTCAAACG | 71 | X | 1807 |
| 1241809 | 12194 | 12209 | 2497 | 2512 | GTCCTCTCCTGGTTTA | 75 | X | 1808 |
| 1241832 | 12300 | 12315 | 2603 | 2618 | CTGGATCTGCAGCTTT | 41 | X | 1809 |
| 1241856 | 12416 | 12431 | 2719 | 2734 | TGGTCCATTCTGGTGG | 61 | X | 1810 |
| 1241880 | 12530 | 12545 | 2833 | 2848 | AGGTGTCGGCCTTCCT | 67 | X | 1811 |
| 1241904 | 12562 | 12577 | 2865 | 2880 | AGGAGCTTGGGAGGAC | 95 | X | 1812 |
| 1241928 | 16668 | 16683 | 2986 | 3001 | GAATTGTCACTGAGGT | 56 | X | 1813 |
| 1241952 | 21136 | 21151 | 3107 | 3122 | CAAGGAGATGTCGAAG | 79 | X | 1814 |
| 1241976 | 22997 | 23012 | 3272 | 3287 | AAGATCCTGACAACAT | 51 | X | 1815 |
| 1242000 | 23036 | 23051 | 3311 | 3326 | GAGTCTGGTCAGGGAA | 35 | X | 1816 |
| 1242024 | 23105 | 23120 | 3380 | 3395 | CTGTGGATTCTTGGCT | 67 | X | 1817 |
| 1242048 | 30993 | 31008 | 3437 | 3452 | TGAACAACAGACTGAC | 50 | X | 1818 |
| 1242072 | 31031 | 31046 | 3475 | 3490 | GTGAGATTCTGATTAG | 38 | X | 1819 |
| 1242096 | 31080 | 31095 | 3524 | 3539 | TAGTTTGATCCCCTTG | 44 | X | 1820 |
| 1242120 | 31687 | 31702 | 3623 | 3638 | AAGTGTGGAAAGATCC | 56 | X | 1821 |
| 1242144 | 35417 | 35432 | 3770 | 3785 | CTCATAATTGAAATAC | 34† | X | 1822 |
| 1242168 | 35622 | 35637 | 3975 | 3990 | TCTTCTCCGACACTCC | 60 | X | 1823 |
| 1242192 | 35752 | 35767 | 4105 | 4120 | GAGGAACATCCTCTAA | 75 | X | 1824 |
| 1242216 | 35894 | 35909 | 4247 | 4262 | AATAGAAAGATAGCGG | 71 | X | 1825 |
| 1242240 | 36028 | 36043 | 4381 | 4396 | TTTCAACCAGCTACAA | 80 | X | 1826 |
| 1242264 | 6549 | 6564 | N/A | N/A | ATTCAATGGGTGAGGA | 79 | X | 1827 |
| 1242288 | 6986 | 7001 | N/A | N/A | CTAAAACAGTAGAGCA | 57 | X | 1828 |
| 1242312 | 8482 | 8497 | N/A | N/A | AGTATATAAGTACTGA | 71 | X | 1829 |
| 1242336 | 9422 | 9437 | N/A | N/A | AGATAACTTCAGGTGG | 51 | X | 1830 |
| 1242360 | 10144 | 10159 | N/A | N/A | ATATTTGGCATGAGAG | 60 | X | 1831 |
| 1242384 | 10599 | 10614 | N/A | N/A | GAATCAATCTTATTCC | 73 | X | 1832 |
| 1242408 | 12897 | 12912 | N/A | N/A | CAAGTAGCTTACAAGA | 56 | X | 1833 |
| 1242432 | 14377 | 14392 | N/A | N/A | CTTAATGAGTAGTTCT | 70 | X | 1834 |
| 1242456 | 14504 | 14519 | N/A | N/A | GGAAGGTGCTATCTAG | 19 | X | 1835 |
| | 14650 | 14665 | | | | | | |
| | 14723 | 14738 | | | | | | |
| | 14796 | 14811 | | | | | | |
| | 14942 | 14957 | | | | | | |
| | 15015 | 15030 | | | | | | |
| | 15088 | 15103 | | | | | | |
| 1242480 | 14578 | 14593 | N/A | N/A | AGGCAGGTGCCATCTA | 40 | X | 1836 |
| | 14870 | 14885 | | | | | | |
| | 15160 | 15175 | | | | | | |
| | 15233 | 15248 | | | | | | |
| 1242504 | 14676 | 14691 | N/A | N/A | CCAATCAGAGTCTGTC | 20 | X | 1837 |
| | 14822 | 14837 | | | | | | |
| | 14968 | 14983 | | | | | | |
| 1242528 | 15406 | 15421 | N/A | N/A | CTAAGATTGGGTCCAC | 47 | X | 1838 |
| 1242552 | 17697 | 17712 | N/A | N/A | ATCTATGAACATGGCC | 42 | X | 1839 |
| 1242576 | 19300 | 19315 | N/A | N/A | AAAGGGTAACATCCAT | 38 | X | 1840 |
| 1242600 | 20253 | 20268 | N/A | N/A | CATCTATATTGAAAGG | 48 | X | 1841 |
| 1242624 | 20632 | 20647 | N/A | N/A | CACACCTGACCAGAGA | 29 | X | 1842 |
| | 20674 | 20689 | | | | | | |
| | 20716 | 20731 | | | | | | |
| | 20758 | 20773 | | | | | | |
| | 20800 | 20815 | | | | | | |
| | 20926 | 20941 | | | | | | |
| | 21052 | 21067 | | | | | | |
| 1242648 | 20932 | 20947 | N/A | N/A | TCAGAACACACCTGAC | 65 | X | 1843 |
| | 21058 | 21073 | | | | | | |
| 1242672 | 22459 | 22474 | N/A | N/A | CCCTTTCGAGTCTAGC | 51 | X | 1844 |
| 1242695 | 22832 | 22847 | N/A | N/A | GCTCTATGTACACTTC | 25 | X | 1845 |
| 1242719 | 23466 | 23481 | N/A | N/A | TAACTTGCATGCTTGC | 108 | X | 1846 |
| 1242743 | 23760 | 23775 | N/A | N/A | CTTATGTAACTGCCAA | 43 | X | 1847 |
| 1242767 | 24124 | 24139 | N/A | N/A | GATGAAGAAGCCCTGT | 64 | X | 1848 |
| 1242791 | 24373 | 24388 | N/A | N/A | GTTAATTATGGATTAG | 72 | X | 1849 |
| 1242815 | 24958 | 24973 | N/A | N/A | GTGGAATTTGGAACCA | 43 | X | 1850 |
| 1242839 | 25829 | 25844 | N/A | N/A | GTACTATGATATGTTT | 65 | X | 1851 |
| 1242863 | 26271 | 26286 | N/A | N/A | GTTTAAGCTGCTCTGT | 55 | X | 1852 |
| 1242887 | 26704 | 26719 | N/A | N/A | TATTATGGTTCTAGTT | 60 | X | 1853 |
| 1242911 | 27103 | 27118 | N/A | N/A | TAACACATAGGCTATA | 91 | X | 1854 |
| | 27121 | 27136 | | | | | | |
| 1242935 | 27499 | 27514 | N/A | N/A | AAGCTAAGATGTCACT | 68 | X | 1855 |
| 1242959 | 28589 | 28604 | N/A | N/A | GTATTAAGTTAATTGG | 73 | X | 1856 |
| 1242983 | 28828 | 28843 | N/A | N/A | GTAGAAATTGCAACAG | 65 | X | 1857 |
| 1243007 | 29094 | 29109 | N/A | N/A | AGCAAGTTTTGAGTGG | 52 | X | 1858 |
| 1243031 | 29430 | 29445 | N/A | N/A | GAGCATAAGAGCTCTA | 98 | X | 1859 |
| 1243055 | 30609 | 30624 | N/A | N/A | CTCAACTAATAATCCC | 70 | X | 1860 |
| 1243079 | 31417 | 31432 | N/A | N/A | GTTCATTGATTTACCA | 43 | X | 1861 |
| 1243103 | 32892 | 32907 | N/A | N/A | ACATATCATGAAGCCA | 80 | X | 1862 |
| 1243127 | 33875 | 33890 | N/A | N/A | GCACAAGGATGCAAGG | 78 | X | 1863 |
| 1243151 | 34112 | 34127 | N/A | N/A | CTAAAGCAGCTTGTTA | 66 | X | 1864 |
| 1243175 | 34382 | 34397 | N/A | N/A | GAAGTAGTTAGAGTTA | 49 | X | 1865 |
| 1243199 | 34534 | 34549 | N/A | N/A | GCAACTTAACTAATAA | 65 | X | 1866 |
| 1243223 | 34963 | 34978 | N/A | N/A | TTTAACTATCTAGTTG | 82 | X | 1867 |
| 1243247 | 13244 | 13259 | N/A | N/A | GGCCTAGAAAGAGGGT | 64 | X | 1868 |
| 1232737 | 5138 | 5153 | 90 | 105 | ATGCCATCTTGACCCA | 60 | Y | 22 |
| 1241427 | 5128 | 5143 | 80 | 95 | GACCCATCAGCAAGAA | 56 | Y | 1869 |
| 1241451 | 5220 | 5235 | 172 | 187 | GGTTTCCCTATGGAGG | 87 | Y | 1870 |
| 1241475 | 5287 | 5302 | 239 | 254 | CCAAATGCTTACCAGA | 42 | Y | 1871 |
| 1241499 | 5743 | 5758 | 695 | 710 | CACTAAGATACCAGGC | 66 | Y | 1872 |
| 1241523 | 5873 | 5888 | 825 | 840 | AGTCCTCTAAGTGCAT | 55 | Y | 1873 |
| 1241547 | 5944 | 5959 | 896 | 911 | GGCTAGATCCACATGG | 71 | Y | 1874 |
| 1241571 | 6005 | 6020 | 957 | 972 | CGAAGATCCACACGGC | 60 | Y | 1875 |
| 1241595 | N/A | N/A | 1020 | 1035 | CTGAACCCCACTTCGG | 117 | Y | 1876 |
| 1241619 | 10297 | 10312 | 1097 | 1112 | CTCCAGTAAACCCATC | 59 | Y | 1877 |
| 1241643 | 10881 | 10896 | 1184 | 1199 | AATGCACTGGAATCTG | 31 | Y | 1878 |
| 1241667 | 11002 | 11017 | 1305 | 1320 | TGCCGATGGCCAGAAG | 64 | Y | 1879 |
| 1241691 | 11258 | 11273 | 1561 | 1576 | ATGATCAGGTCCCCCA | 41 | Y | 1880 |
| 1241715 | 11391 | 11406 | 1694 | 1709 | GTCAGTGCAGAGCGGT | 39 | Y | 1881 |
| 1241739 | 11595 | 11610 | 1898 | 1913 | CTCATCAGAGAAGTAC | 57 | Y | 1882 |
| 1241763 | 11922 | 11937 | 2225 | 2240 | ATCCGCCTTCTGCAGT | 69 | Y | 1883 |
| 1241786 | 12095 | 12110 | 2398 | 2413 | CGGCTGGGAAGCTTCA | 57 | Y | 1884 |
| 1241810 | 12195 | 12210 | 2498 | 2513 | GGTCCTCTCCTGGTTT | 70 | Y | 1885 |
| 1241833 | 12320 | 12335 | 2623 | 2638 | AACAATTCCAGCTGGC | 52 | Y | 1886 |
| 1241857 | 12417 | 12432 | 2720 | 2735 | GTGGTCCATTCTGGTG | 58 | Y | 1887 |
| 1241881 | 12531 | 12546 | 2834 | 2849 | AAGGTGTCGGCCTTCC | 49 | Y | 1888 |
| 1241905 | 12568 | 12583 | 2871 | 2886 | CATGAGAGGAGCTTGG | 83 | Y | 1889 |
| 1241929 | 16669 | 16684 | 2987 | 3002 | AGAATTGTCACTGAGG | 32 | Y | 1890 |
| 1241953 | 21140 | 21155 | 3111 | 3126 | GGACCAAGGAGATGTC | 102 | Y | 1891 |
| 1241977 | 22998 | 23013 | 3273 | 3288 | CAAGATCCTGACAACA | 74 | Y | 1892 |
| 1242001 | 23038 | 23053 | 3313 | 3328 | TAGAGTCTGGTCAGGG | 27 | Y | 1893 |
| 1242025 | 23106 | 23121 | 3381 | 3396 | ACTGTGGATTCTTGGC | 42 | Y | 1894 |
| 1242049 | 30995 | 31010 | 3439 | 3454 | GCTGAACAACAGACTG | 39 | Y | 1895 |
| 1242073 | 31032 | 31047 | 3476 | 3491 | CGTGAGATTCTGATTA | 39 | Y | 1896 |
| 1242097 | 31081 | 31096 | 3525 | 3540 | GTAGTTTGATCCCCTT | 38 | Y | 1897 |
| 1242121 | 31693 | 31708 | 3629 | 3644 | GGTCAGAAGTGTGGAA | 76 | Y | 1898 |
| 1242145 | 35419 | 35434 | 3772 | 3787 | GTCTCATAATTGAAAT | 31† | Y | 1899 |
| 1242169 | 35629 | 35644 | 3982 | 3997 | CAAGCTCTCTTCTCCG | 41 | Y | 1900 |
| 1242193 | 35753 | 35768 | 4106 | 4121 | AGAGGAACATCCTCTA | 100 | Y | 1901 |
| 1242217 | 35895 | 35910 | 4248 | 4263 | TAATAGAAAGATAGCG | 111 | Y | 1902 |
| 1242241 | 36030 | 36045 | 4383 | 4398 | ATTTTCAACCAGCTAC | 48 | Y | 1903 |
| 1242265 | 6553 | 6568 | N/A | N/A | TATCATTCAATGGGTG | 54 | Y | 1904 |
| 1242289 | 6987 | 7002 | N/A | N/A | TCTAAAACAGTAGAGC | 60 | Y | 1905 |
| 1242313 | 8510 | 8525 | N/A | N/A | GATTATGACACTTTCA | 75 | Y | 1906 |
| 1242337 | 9425 | 9440 | N/A | N/A | GACAGATAACTTCAGG | 59 | Y | 1907 |
| 1242361 | 10147 | 10162 | N/A | N/A | CTAATATTTGGCATGA | 68 | Y | 1908 |
| 1242385 | 10613 | 10628 | N/A | N/A | CCCAAAGATCATTTGA | 104 | Y | 1909 |
| 1242409 | 12899 | 12914 | N/A | N/A | TCCAAGTAGCTTACAA | 77 | Y | 1910 |
| 1242433 | 14380 | 14395 | N/A | N/A | ACTCTTAATGAGTAGT | 120 | Y | 1911 |
| 1242457 | 14505 | 14520 | N/A | N/A | AGGAAGGTGCTATCTA | 17 | Y | 1912 |
| | 14651 | 14666 | | | | | | |
| | 14724 | 14739 | | | | | | |
| | 14797 | 14812 | | | | | | |
| | 14943 | 14958 | | | | | | |
| | 15016 | 15031 | | | | | | |
| | 15089 | 15104 | | | | | | |
| 1242481 | 14580 | 14595 | N/A | N/A | TAAGGCAGGTGCCATC | 26 | Y | 1913 |
| | 14872 | 14887 | | | | | | |
| | 15162 | 15177 | | | | | | |
| | 15235 | 15250 | | | | | | |
| 1242505 | 14677 | 14692 | N/A | N/A | TCCAATCAGAGTCTGT | 31 | Y | 1914 |
| | 14823 | 14838 | | | | | | |
| | 14969 | 14984 | | | | | | |
| 1242529 | 15407 | 15422 | N/A | N/A | TCTAAGATTGGGTCCA | 47 | Y | 1915 |
| 1242553 | 17698 | 17713 | N/A | N/A | CATCTATGAACATGGC | 30 | Y | 1916 |
| 1242577 | 19301 | 19316 | N/A | N/A | CAAAGGGTAACATCCA | 39 | Y | 1917 |
| 1242601 | 20274 | 20289 | N/A | N/A | GACGATTTCATCCCAG | 11 | Y | 1918 |
| 1242625 | 20633 | 20648 | N/A | N/A | ACACACCTGACCAGAG | 106 | Y | 1919 |
| | 20675 | 20690 | | | | | | |
| | 20717 | 20732 | | | | | | |
| | 20759 | 20774 | | | | | | |
| | 20801 | 20816 | | | | | | |
| | 20927 | 20942 | | | | | | |
| | 21053 | 21068 | | | | | | |
| 1242649 | 20933 | 20948 | N/A | N/A | ATCAGAACACACCTGA | 89 | Y | 1920 |
| | 21059 | 21074 | | | | | | |
| 1242673 | 22468 | 22483 | N/A | N/A | GAGTATTTGCCCTTTC | 37 | Y | 1921 |
| 1242696 | 22855 | 22870 | N/A | N/A | ACATGGAAAGGGAGTG | 74 | Y | 1922 |
| 1242720 | 23468 | 23483 | N/A | N/A | GATAACTTGCATGCTT | 90 | Y | 1923 |
| 1242744 | 23774 | 23789 | N/A | N/A | CATGAATCAAAAGGCT | 84 | Y | 1924 |
| 1242768 | 24127 | 24142 | N/A | N/A | CCAGATGAAGAAGCCC | 35 | Y | 1925 |
| 1242792 | 24411 | 24426 | N/A | N/A | CCAAGGAAGTATCAGA | 68 | Y | 1926 |
| 1242816 | 24986 | 25001 | N/A | N/A | GCACATTTGTAATTGG | 46 | Y | 1927 |
| 1242840 | 25860 | 25875 | N/A | N/A | TGAGAAGGAGCTTTCA | 63 | Y | 1928 |
| 1242864 | 26325 | 26340 | N/A | N/A | CATCTTAATCTCACAC | 55 | Y | 1929 |
| 1242888 | 26705 | 26720 | N/A | N/A | ATATTATGGTTCTAGT | 72 | Y | 1930 |
| 1242912 | 27104 | 27119 | N/A | N/A | CTAACACATAGGCTAT | 63 | Y | 1931 |
| | 27122 | 27137 | | | | | | |
| 1242936 | 27500 | 27515 | N/A | N/A | CAAGCTAAGATGTCAC | 59 | Y | 1932 |
| 1242960 | 28608 | 28623 | N/A | N/A | GGATATCTTGCCATGT | 39 | Y | 1933 |
| 1242984 | 28830 | 28845 | N/A | N/A | TAGTAGAAATTGCAAC | 93 | Y | 1934 |
| 1243008 | 29144 | 29159 | N/A | N/A | TAAGTAAGACTAATAG | 107 | Y | 1935 |
| 1243032 | 29433 | 29448 | N/A | N/A | GAAGAGCATAAGAGCT | 80 | Y | 1936 |
| 1243056 | 30705 | 30720 | N/A | N/A | GTTACCCCATACTCCT | 40 | Y | 1937 |
| 1243080 | 31432 | 31447 | N/A | N/A | TAGTAACAGTGGTTTG | 74 | Y | 1938 |
| 1243104 | 32916 | 32931 | N/A | N/A | CATATAATCTGGAGGC | 94 | Y | 1939 |
| 1243128 | 33885 | 33900 | N/A | N/A | CATTATGTTGGCACAA | 72 | Y | 1940 |
| 1243152 | 34113 | 34128 | N/A | N/A | TCTAAAGCAGCTTGTT | 59 | Y | 1941 |
| 1243176 | 34383 | 34398 | N/A | N/A | GGAAGTAGTTAGAGTT | 55 | Y | 1942 |
| 1243200 | 34548 | 34563 | N/A | N/A | ATAAAGTTTCCCTTGC | 82 | Y | 1943 |
| 1243224 | 18719 | 18734 | N/A | N/A | TCTAAAGGACCATCTT | 72 | Y | 1944 |
| 1243248 | 19181 | 19196 | N/A | N/A | AAGAGTTACACTAAGG | 40 | Y | 1945 |
| 1232737 | 5138 | 5153 | 90 | 105 | ATGCCATCTTGACCCA | 64 | Z | 22 |
| 1241428 | 5130 | 5145 | 82 | 97 | TTGACCCATCAGCAAG | 59 | Z | 1946 |
| 1241452 | 5221 | 5236 | 173 | 188 | AGGTTTCCCTATGGAG | 85 | Z | 1947 |
| 1241476 | 5290 | 5305 | 242 | 257 | TAGCCAAATGCTTACC | 60 | Z | 1948 |
| 1241500 | 5744 | 5759 | 696 | 711 | ACACTAAGATACCAGG | 46 | Z | 1949 |
| 1241524 | 5874 | 5889 | 826 | 841 | TAGTCCTCTAAGTGCA | 54 | Z | 1950 |
| 1241548 | 5945 | 5960 | 897 | 912 | TGGCTAGATCCACATG | 91 | Z | 1951 |
| 1241572 | 6014 | 6029 | 966 | 981 | TGATCGCAGCGAAGAT | 80 | Z | 1952 |
| 1241596 | N/A | N/A | 1021 | 1036 | TCTGAACCCCACTTCG | 87 | Z | 1953 |
| 1241620 | 10298 | 10313 | 1098 | 1113 | ACTCCAGTAAACCCAT | 46 | Z | 1954 |
| 1241644 | 10882 | 10897 | 1185 | 1200 | CAATGCACTGGAATCT | 64 | Z | 1955 |
| 1241668 | 11006 | 11021 | 1309 | 1324 | GTCTTGCCGATGGCCA | 40 | Z | 1956 |
| 1241692 | 11259 | 11274 | 1562 | 1577 | CATGATCAGGTCCCCC | 50 | Z | 1957 |
| 1241716 | 11393 | 11408 | 1696 | 1711 | CAGTCAGTGCAGAGCG | 63 | Z | 1958 |
| 1241740 | 11625 | 11640 | 1928 | 1943 | AATCAGACTGAAGGCT | 67 | Z | 1959 |
| 1241764 | 11924 | 11939 | 2227 | 2242 | ACATCCGCCTTCTGCA | 66 | Z | 1960 |
| 1241787 | 12097 | 12112 | 2400 | 2415 | CTCGGCTGGGAAGCTT | 58 | Z | 1961 |
| 1241811 | 12196 | 12211 | 2499 | 2514 | AGGTCCTCTCCTGGTT | 83 | Z | 1962 |
| 1241834 | 12321 | 12336 | 2624 | 2639 | GAACAATTCCAGCTGG | 57 | Z | 1963 |
| 1241858 | 12418 | 12433 | 2721 | 2736 | TGTGGTCCATTCTGGT | 64 | Z | 1964 |
| 1241882 | 12532 | 12547 | 2835 | 2850 | CAAGGTGTCGGCCTTC | 66 | Z | 1965 |
| 1241906 | 12570 | 12585 | 2873 | 2888 | AGCATGAGAGGAGCTT | 85 | Z | 1966 |
| 1241930 | 16670 | 16685 | 2988 | 3003 | GAGAATTGTCACTGAG | 55 | Z | 1967 |
| 1241954 | 21141 | 21156 | 3112 | 3127 | AGGACCAAGGAGATGT | 72 | Z | 1968 |
| 1241978 | 22999 | 23014 | 3274 | 3289 | GCAAGATCCTGACAAC | 59 | Z | 1969 |
| 1242002 | 23042 | 23057 | 3317 | 3332 | CACATAGAGTCTGGTC | 61 | Z | 1970 |
| 1242026 | 23108 | 23123 | 3383 | 3398 | ACACTGTGGATTCTTG | 83 | Z | 1971 |
| 1242050 | 30996 | 31011 | 3440 | 3455 | AGCTGAACAACAGACT | 54 | Z | 1972 |
| 1242074 | 31046 | 31061 | 3490 | 3505 | CGCAGGTAAAGGTGCG | 98 | Z | 1973 |
| 1242098 | 31082 | 31097 | 3526 | 3541 | AGTAGTTTGATCCCCT | 32 | Z | 1974 |
| 1242122 | 31705 | 31720 | 3641 | 3656 | GCTCTGGCTGGAGGTC | 47 | Z | 1975 |
| 1242146 | 35479 | 35494 | 3832 | 3847 | GGCTCAAAGACGACGG | 71 | Z | 1976 |
| 1242170 | 35630 | 35645 | 3983 | 3998 | GCAAGCTCTCTTCTCC | 49 | Z | 1977 |
| 1242194 | 35754 | 35769 | 4107 | 4122 | AAGAGGAACATCCTCT | 85 | Z | 1978 |
| 1242218 | 35901 | 35916 | 4254 | 4269 | GTCAGTTAATAGAAAG | 108 | Z | 1979 |
| 1242242 | 36031 | 36046 | 4384 | 4399 | AATTTTCAACCAGCTA | 70 | Z | 1980 |
| 1242266 | 6554 | 6569 | N/A | N/A | TTATCATTCAATGGGT | 75 | Z | 1981 |
| 1242290 | 6988 | 7003 | N/A | N/A | ATCTAAAACAGTAGAG | 74 | Z | 1982 |
| 1242314 | 8511 | 8526 | N/A | N/A | TGATTATGACACTTTC | 67 | Z | 1983 |
| 1242338 | 9453 | 9468 | N/A | N/A | AGAGTTAGTCACTAGG | 58 | Z | 1984 |
| 1242362 | 10148 | 10163 | N/A | N/A | CCTAATATTTGGCATG | 63 | Z | 1985 |
| 1242386 | 10661 | 10676 | N/A | N/A | TAAGATACACAGCTGA | 90 | Z | 1986 |
| 1242410 | 12900 | 12915 | N/A | N/A | CTCCAAGTAGCTTACA | 63 | Z | 1987 |
| 1242434 | 14416 | 14431 | N/A | N/A | TCATAAGGTCTCCCTG | 94 | Z | 1988 |
| 1242458 | 14506 | 14521 | N/A | N/A | AAGGAAGGTGCTATCT | 30 | Z | 1989 |
| | 14652 | 14667 | | | | | | |
| | 14725 | 14740 | | | | | | |
| | 14798 | 14813 | | | | | | |
| | 14944 | 14959 | | | | | | |
| | 15017 | 15032 | | | | | | |
| | 15090 | 15105 | | | | | | |
| 1242482 | 14581 | 14596 | N/A | N/A | GTAAGGCAGGTGCCAT | 34 | Z | 1990 |
| | 14873 | 14888 | | | | | | |
| | 15163 | 15178 | | | | | | |
| | 15236 | 15251 | | | | | | |
| 1242506 | 14678 | 14693 | N/A | N/A | CTCCAATCAGAGTCTG | 40 | Z | 1991 |
| | 14824 | 14839 | | | | | | |
| | 14970 | 14985 | | | | | | |
| 1242530 | 15428 | 15443 | N/A | N/A | TGCAAAGTCTAGCTCT | 47 | Z | 1992 |
| 1242554 | 17716 | 17731 | N/A | N/A | GGCAAATTCTCCAACT | 95 | Z | 1993 |
| 1242578 | 19405 | 19420 | N/A | N/A | ATCTTTGAAGCCACAC | 59 | Z | 1994 |
| 1242602 | 20298 | 20313 | N/A | N/A | CAACATTGTCACCCCA | 46 | Z | 1995 |
| 1242626 | 20634 | 20649 | N/A | N/A | GACACACCTGACCAGA | 36 | Z | 1996 |
| | 20676 | 20691 | | | | | | |
| | 20718 | 20733 | | | | | | |
| | 20760 | 20775 | | | | | | |
| | 20802 | 20817 | | | | | | |
| 1242650 | 20934 | 20949 | N/A | N/A | CATCAGAACACACCTG | 66 | Z | 1997 |
| | 21060 | 21075 | | | | | | |
| 1242674 | 22475 | 22490 | N/A | N/A | ACAATCAGAGTATTTG | 65 | Z | 1998 |
| 1242697 | 22899 | 22914 | N/A | N/A | ACAAGGAAGCACCCGT | 50 | Z | 1999 |
| 1242721 | 23469 | 23484 | N/A | N/A | CGATAACTTGCATGCT | 72 | Z | 2000 |
| 1242745 | 23775 | 23790 | N/A | N/A | ACATGAATCAAAAGGC | 43 | Z | 2001 |
| 1242769 | 24141 | 24156 | N/A | N/A | CCTTAAGCTGAGAGCC | 56 | Z | 2002 |
| 1242793 | 24412 | 24427 | N/A | N/A | TCCAAGGAAGTATCAG | 78 | Z | 2003 |
| 1242817 | 25003 | 25018 | N/A | N/A | TATTACAGATGCATCA | 79 | Z | 2004 |
| 1242841 | 25884 | 25899 | N/A | N/A | TAAATGGCTCTCTCAG | 78 | Z | 2005 |
| 1242865 | 26370 | 26385 | N/A | N/A | CATGAACAGATTCCCC | 91 | Z | 2006 |
| 1242889 | 26706 | 26721 | N/A | N/A | TATATTATGGTTCTAG | 54 | Z | 2007 |
| 1242913 | 27105 | 27120 | N/A | N/A | CCTAACACATAGGCTA | 59 | Z | 2008 |
| | 27123 | 27138 | | | | | | |
| 1242937 | 27547 | 27562 | N/A | N/A | CAAGGTATGGCAGGAG | 50 | Z | 2009 |
| 1242961 | 28609 | 28624 | N/A | N/A | CGGATATCTTGCCATG | 45 | Z | 2010 |
| 1242985 | 28832 | 28847 | N/A | N/A | AGTAGTAGAAATTGCA | 49 | Z | 2011 |
| 1243009 | 29145 | 29160 | N/A | N/A | GTAAGTAAGACTAATA | 89 | Z | 2012 |
| 1243033 | 29459 | 29474 | V/A | N/A | CAAATTAAGCCCATTC | 88 | Z | 2013 |
| 1243057 | 30827 | 30842 | N/A | N/A | TCAGATATGATCTGGG | 83 | Z | 2014 |
| 1243081 | 31433 | 31448 | N/A | N/A | TTAGTAACAGTGGTTT | 63 | Z | 2015 |
| 1243105 | 32917 | 32932 | N/A | N/A | ACATATAATCTGGAGG | 87 | Z | 2016 |
| 1243129 | 33886 | 33901 | N/A | N/A | CCATTATGTTGGCACA | 59 | Z | 2017 |
| 1243153 | 34114 | 34129 | N/A | N/A | GTCTAAAGCAGCTTGT | 71 | Z | 2018 |
| 1243177 | 34384 | 34399 | N/A | N/A | GGGAAGTAGTTAGAGT | 83 | Z | 2019 |
| 1243201 | 34573 | 34588 | N/A | N/A | CGATGTAAGTTCTTCC | 44 | Z | 2020 |
| 1243225 | 22287 | 22302 | N/A | N/A | GTTGCTATGGACATAG | 46 | Z | 2021 |
| 1243249 | 31239 | 31254 | N/A | N/A | AGAAGACACCTCACTT | 87 | Z | 2022 |
| 1232737 | 5138 | 5153 | 90 | 105 | ATGCCATCTTGACCCA | 57 | AA | 22 |
| 1241429 | 5134 | 5149 | 86 | 101 | CATCTTGACCCATCAG | 41 | AA | 2023 |
| 1241453 | 5222 | 5237 | 174 | 189 | AAGGTTTCCCTATGGA | 65 | AA | 2024 |
| 1241477 | 5291 | 5306 | 243 | 258 | TTAGCCAAATGCTTAC | 43 | AA | 2025 |
| 1241501 | 5745 | 5760 | 697 | 712 | CACACTAAGATACCAG | 46 | AA | 2026 |
| 1241525 | 5875 | 5890 | 827 | 842 | ATAGTCCTCTAAGTGC | 64 | AA | 2027 |
| 1241549 | 5946 | 5961 | 898 | 913 | GTGGCTAGATCCACAT | 99 | AA | 2028 |
| 1241573 | 6015 | 6030 | 967 | 982 | TTGATCGCAGCGAAGA | 48 | AA | 2029 |
| 1241597 | N/A | N/A | 1024 | 1039 | TTATCTGAACCCCACT | 95 | AA | 2030 |
| 1241621 | 10300 | 10315 | 1100 | 1115 | GTACTCCAGTAAACCC | 30 | AA | 2031 |
| 1241645 | 10883 | 10898 | 1186 | 1201 | TCAATGCACTGGAATC | 67 | AA | 2032 |
| 1241669 | 11008 | 11023 | 1311 | 1326 | TGGTCTTGCCGATGGC | 27 | AA | 2033 |
| 1241693 | 11260 | 11275 | 1563 | 1578 | TCATGATCAGGTCCCC | 37 | AA | 2034 |
| 1241717 | 11401 | 11416 | 1704 | 1719 | CCTTCTGCCAGTCAGT | 54 | AA | 2035 |
| 1241741 | 11626 | 11641 | 1929 | 1944 | GAATCAGACTGAAGGC | 42 | AA | 2036 |
| 1241765 | 11926 | 11941 | 2229 | 2244 | ACACATCCGCCTTCTG | 64 | AA | 2037 |
| 1241788 | 12116 | 12131 | 2419 | 2434 | TCCAGAAGGACTGTCA | 40 | AA | 2038 |
| 1241812 | 12197 | 12212 | 2500 | 2515 | GAGGTCCTCTCCTGGT | 63 | AA | 2039 |
| 1241835 | 12324 | 12339 | 2627 | 2642 | GTAGAACAATTCCAGC | 42 | AA | 2040 |
| 1241859 | 12451 | 12466 | 2754 | 2769 | GATGACAGTTCTCAAT | 79 | AA | 2041 |
| 1241883 | 12533 | 12548 | 2836 | 2851 | TCAAGGTGTCGGCCTT | 59 | AA | 2042 |
| 1241907 | 12572 | 12587 | 2875 | 2890 | GCAGCATGAGAGGAGC | 81 | AA | 2043 |
| 1241931 | 16677 | 16692 | 2995 | 3010 | TCCCCCAGAGAATTGT | 80 | AA | 2044 |
| 1241955 | 21208 | 21223 | 3179 | 3194 | TCTGATTCCGAAGTCA | 59 | AA | 2045 |
| 1241979 | 23000 | 23015 | 3275 | 3290 | TGCAAGATCCTGACAA | 74 | AA | 2046 |
| 1242003 | 23044 | 23059 | 3319 | 3334 | CCCACATAGAGTCTGG | 58 | AA | 2047 |
| 1242027 | 23109 | 23124 | 3384 | 3399 | TACACTGTGGATTCTT | 77 | AA | 2048 |
| 1242051 | 30997 | 31012 | 3441 | 3456 | AAGCTGAACAACAGAC | 54 | AA | 2049 |
| 1242075 | 31048 | 31063 | 3492 | 3507 | CTCGCAGGTAAAGGTG | 81 | AA | 2050 |
| 1242099 | 31083 | 31098 | 3527 | 3542 | GAGTAGTTTGATCCCC | 34 | AA | 2051 |
| 1242123 | 31719 | 31734 | 3655 | 3670 | CTCAGCTTTCGCAGGC | 51 | AA | 2052 |
| 1242147 | 35482 | 35497 | 3835 | 3850 | GAAGGCTCAAAGACGA | 62 | AA | 2053 |
| 1242171 | 35631 | 35646 | 3984 | 3999 | GGCAAGCTCTCTTCTC | 59 | AA | 2054 |
| 1242195 | 35757 | 35772 | 4110 | 4125 | ACCAAGAGGAACATCC | 53 | AA | 2055 |
| 1242219 | 35904 | 35919 | 4257 | 4272 | ATGGTCAGTTAATAGA | 121 | AA | 2056 |
| 1242243 | 36032 | 36047 | 4385 | 4400 | GAATTTTCAACCAGCT | 51 | AA | 2057 |
| 1242267 | 6558 | 6573 | N/A | N/A | GGAGTTATCATTCAAT | 46 | AA | 2058 |
| 1242291 | 6990 | 7005 | N/A | N/A | GTATCTAAAACAGTAG | 70 | AA | 2059 |
| 1242315 | 8530 | 8545 | N/A | N/A | CAAAGTACTICTACAG | 62 | AA | 2060 |
| 1242339 | 9455 | 9470 | N/A | N/A | CAAGAGTTAGTCACTA | 60 | AA | 2061 |
| 1242363 | 10163 | 10178 | N/A | N/A | GCAATTGAAACCTGGC | 56 | AA | 2062 |
| 1242387 | 10737 | 10752 | N/A | N/A | CGGAATGGAGAAATGG | 48 | AA | 2063 |
| 1242411 | 12917 | 12932 | N/A | N/A | ACTCTTAGGCACTAGT | 73 | AA | 2064 |
| 1242435 | 14418 | 14433 | N/A | N/A | CTTCATAAGGTCTCCC | 54 | AA | 2065 |
| 1242459 | 14507 | 14522 | N/A | N/A | GAAGGAAGGTGCTATC | 26 | AA | 2066 |
| | 14653 | 14668 | | | | | | |
| | 14726 | 14741 | | | | | | |
| | 14799 | 14814 | | | | | | |
| | 14945 | 14960 | | | | | | |
| | 15018 | 15033 | | | | | | |
| | 15091 | 15106 | | | | | | |
| 1242483 | 14582 | 14597 | N/A | N/A | AGTAAGGCAGGTGCCA | 31 | AA | 2067 |
| | 14874 | 14889 | | | | | | |
| | 15164 | 15179 | | | | | | |
| | 15237 | 15252 | | | | | | |
| 1242507 | 14679 | 14694 | N/A | N/A | GCTCCAATCAGAGTCT | 56 | AA | 2068 |
| | 14825 | 14840 | | | | | | |
| | 14971 | 14986 | | | | | | |
| 1242531 | 15622 | 15637 | N/A | N/A | CTCCAACATGCAGTCA | 46 | AA | 2069 |
| 1242555 | 17747 | 17762 | N/A | N/A | GTTTTTAGGTGAGGTT | 34 | AA | 2070 |
| 1242579 | 19474 | 19489 | N/A | N/A | GCAAAGATGAAGAGTG | 26 | AA | 2071 |
| 1242603 | 20421 | 20436 | N/A | N/A | GTAAGTGAAGCGGGCG | 27 | AA | 2072 |
| 1242627 | 20635 | 20650 | N/A | N/A | GGACACACCTGACCAG | 25 | AA | 2073 |
| | 20677 | 20692 | | | | | | |
| | 20719 | 20734 | | | | | | |
| | 20761 | 20776 | | | | | | |
| | 20803 | 20818 | | | | | | |
| 1242651 | 20935 | 20950 | N/A | N/A | GCATCAGAACACACCT | 33 | AA | 2074 |
| | 21061 | 21076 | | | | | | |
| 1242675 | 22483 | 22498 | N/A | N/A | CTATAAGGACAATCAG | 63 | AA | 2075 |
| 1242698 | 22900 | 22915 | N/A | N/A | GACAAGGAAGCACCCG | 27 | AA | 2076 |
| 1242722 | 23470 | 23485 | N/A | N/A | GCGATAACTTGCATGC | 56 | AA | 2077 |
| 1242746 | 23821 | 23836 | N/A | N/A | TGATTATTAGTGGGTA | 46 | AA | 2078 |
| 1242770 | 24158 | 24173 | N/A | N/A | TAAATTGGAGGTCCCA | 46 | AA | 2079 |
| 1242794 | 24449 | 24464 | N/A | N/A | GATATGGTCACATCTG | 33 | AA | 2080 |
| 1242818 | 25004 | 25019 | N/A | N/A | ATATTACAGATGCATC | 60 | AA | 2081 |
| 1242842 | 25885 | 25900 | N/A | N/A | GTAAATGGCTCTCTCA | 62 | AA | 2082 |
| 1242866 | 26403 | 26418 | N/A | N/A | CAAGATGACATTCCAC | 63 | AA | 2083 |
| 1242890 | 26754 | 26769 | N/A | N/A | GGAAATTTATCCATGA | 70 | AA | 2084 |
| 1242914 | 27106 | 27121 | N/A | N/A | ACCTAACACATAGGCT | 50 | AA | 2085 |
| | 27124 | 27139 | | | | | | |
| 1242938 | 27549 | 27564 | N/A | N/A | TGCAAGGTATGGCAGG | 48 | AA | 2086 |
| 1242962 | 28615 | 28630 | N/A | N/A | CAAGCACGGATATCTT | 47 | AA | 2087 |
| 1242986 | 28833 | 28848 | N/A | N/A | TAGTAGTAGAAATTGC | 82 | AA | 2088 |
| 1243010 | 29148 | 29163 | N/A | N/A | ACAGTAAGTAAGACTA | 78 | AA | 2089 |
| 1243034 | 29473 | 29488 | N/A | N/A | TACTATTCTTGAGTCA | 44 | AA | 2090 |
| 1243058 | 30828 | 30843 | N/A | N/A | CTCAGATATGATCTGG | 64 | AA | 2091 |
| 1243082 | 31512 | 31527 | N/A | N/A | AACTAGCTTCAAGCAG | 90 | AA | 2092 |
| 1243106 | 32918 | 32933 | N/A | N/A | GACATATAATCTGGAG | 69 | AA | 2093 |
| 1243130 | 33887 | 33902 | N/A | N/A | TCCATTATGTTGGCAC | 57 | AA | 2094 |
| 1243154 | 34115 | 34130 | N/A | N/A | AGTCTAAAGCAGCTTG | 52 | AA | 2095 |
| 1243178 | 34385 | 34400 | N/A | N/A | TGGGAAGTAGTTAGAG | 89 | AA | 2096 |
| 1243202 | 34574 | 34589 | N/A | N/A | TCGATGTAAGTTCTTC | 37 | AA | 2097 |
| 1243226 | 10520 | 10535 | N/A | N/A | GGGACTGCAAGAGCCA | 85 | AA | 2098 |
| 1243250 | 13394 | 13409 | N/A | N/A | ACTGAACGATAAACCC | 42 | AA | 2099 |
| 1232737 | 5138 | 5153 | 90 | 105 | ATGCCATCTTGACCCA | 73 | AB | 22 |
| 1241431 | 5139 | 5154 | 91 | 106 | GATGCCATCTTGACCC | 59 | AB | 2100 |
| 1241455 | 5244 | 5259 | 196 | 211 | GTGTGTCCTGAGCCAT | 26 | AB | 2101 |
| 1241479 | 5594 | 5609 | 546 | 561 | TTAGCCAAGGCCGGGC | 76 | AB | 2102 |
| 1241503 | 5750 | 5765 | 702 | 717 | CGGTCCACACTAAGAT | 55 | AB | 2103 |
| 1241527 | 5877 | 5892 | 829 | 844 | GGATAGTCCTCTAAGT | 70 | AB | 2104 |
| 1241551 | 5962 | 5977 | 914 | 929 | GAAGTCGATCATTAGC | 42 | AB | 2105 |
| 1241575 | 6017 | 6032 | 969 | 984 | TGTTGATCGCAGCGAA | 53 | AB | 2106 |
| 1241599 | N/A | N/A | 1026 | 1041 | CATTATCTGAACCCCA | 69 | AB | 2107 |
| 1241623 | 10315 | 10330 | 1115 | 1130 | AGAGATTCTCGAAAGG | 46 | AB | 2108 |
| 1241647 | 10885 | 10900 | 1188 | 1203 | CTTCAATGCACTGGAA | 50 | AB | 2109 |
| 1241671 | 11048 | 11063 | 1351 | 1366 | AACTCCATCTTAATGG | 81 | AB | 2110 |
| 1241695 | 11262 | 11277 | 1565 | 1580 | GCTCATGATCAGGTCC | 32 | AB | 2111 |
| 1241719 | 11421 | 11436 | 1724 | 1739 | GAGAATGTCTCCCCGC | 43 | AB | 2112 |
| 1241743 | 11629 | 11644 | 1932 | 1947 | CCTGAATCAGACTGAA | 62 | AB | 2113 |
| 1241767 | 11930 | 11945 | 2233 | 2248 | GCAGACACATCCGCCT | 47 | AB | 2114 |
| 1241790 | 12134 | 12149 | 2437 | 2452 | TCGAATTTGCCATAGT | 44 | AB | 2115 |
| 1241814 | 12200 | 12215 | 2503 | 2518 | TAGGAGGTCCTCTCCT | 104 | AB | 2116 |
| 1241837 | 12326 | 12341 | 2629 | 2644 | CAGTAGAACAATTCCA | 36 | AB | 2117 |
| 1241861 | 12453 | 12468 | 2756 | 2771 | CCGATGACAGTTCTCA | 29 | AB | 2118 |
| 1241885 | 12535 | 12550 | 2838 | 2853 | TATCAAGGTGTCGGCC | 59 | AB | 2119 |
| 1241909 | N/A | N/A | 2892 | 2907 | CCAATCCATGAGAACA | 71 | AB | 2120 |
| 1241933 | 16697 | 16712 | 3015 | 3030 | ACAACACTCTCATCCC | 48 | AB | 2121 |
| 1241957 | 21238 | 21253 | 3209 | 3224 | CAACAGGTGCTTCAGT | 84 | AB | 2122 |
| 1241981 | 23002 | 23017 | 3277 | 3292 | GATGCAAGATCCTGAC | 38 | AB | 2123 |
| 1242005 | 23049 | 23064 | 3324 | 3339 | TCTCCCCCACATAGAG | 75 | AB | 2124 |
| 1242029 | 23111 | 23126 | 3386 | 3401 | GTTACACTGTGGATTC | 54 | AB | 2125 |
| 1242053 | 31005 | 31020 | 3449 | 3464 | CGAGGACAAAGCTGAA | 54 | AB | 2126 |
| 1242077 | 31053 | 31068 | 3497 | 3512 | GTTGCCTCGCAGGTAA | 51 | AB | 2127 |
| 1242101 | 31098 | 31113 | 3542 | 3557 | CAAGAGTCCCTCACAG | 50 | AB | 2128 |
| 1242125 | 31723 | 31738 | 3659 | 3674 | CAGGCTCAGCTTTCGC | 44 | AB | 2129 |
| 1242149 | 35488 | 35503 | 3841 | 3856 | TACCAAGAAGGCTCAA | 82 | AB | 2130 |
| 1242173 | 35633 | 35648 | 3986 | 4001 | TCGGCAAGCTCTCTTC | 52 | AB | 2131 |
| 1242197 | 35759 | 35774 | 4112 | 4127 | TCACCAAGAGGAACAT | 50 | AB | 2132 |
| 1242221 | 35908 | 35923 | 4261 | 4276 | TGTTATGGTCAGTTAA | 84 | AB | 2133 |
| 1242245 | 36036 | 36051 | 4389 | 4404 | TCCTGAATTTTCAACC | 58 | AB | 2134 |
| 1242269 | 6629 | 6644 | N/A | N/A | CCAGAGTAGTAAGATT | 79 | AB | 2135 |
| 1242293 | 7051 | 7066 | N/A | N/A | GTTCTTAGGATATGGA | 70 | AB | 2136 |
| 1242317 | 8533 | 8548 | N/A | N/A | TAGCAAAGTACTTCTA | 76 | AB | 2137 |
| 1242341 | 9476 | 9491 | N/A | N/A | AGAAATTCAGGTTAGG | 44 | AB | 2138 |
| 1242365 | 10181 | 10196 | N/A | N/A | ACTATTATCACAGAGG | 79 | AB | 2139 |
| 1242389 | 12605 | 12620 | N/A | N/A | CTGGAAGCCGAGTTTC | 72 | AB | 2140 |
| 1242413 | 13016 | 13031 | N/A | N/A | TAAGAAACATCCCCCC | 85 | AB | 2141 |
| 1242437 | 14476 | 14491 | N/A | N/A | TGAGCCTCCTACCGGG | 36 | AB | 2142 |
| | 14622 | 14637 | | | | | | |
| | 14768 | 14783 | | | | | | |
| | 14914 | 14929 | | | | | | |
| | 15060 | 15075 | | | | | | |
| 1242461 | 14540 | 14555 | N/A | N/A | TACCAGGGCTCCAGTC | 62 | AB | 2143 |
| | 15268 | 15283 | | | | | | |
| 1242485 | 14584 | 14599 | N/A | N/A | AGAGTAAGGCAGGTGC | 6 | AB | 2144 |
| | 14876 | 14891 | | | | | | |
| | 15166 | 15181 | | | | | | |
| | 15239 | 15254 | | | | | | |
| 1242509 | 14681 | 14696 | N/A | N/A | GGGCTCCAATCAGAGT | 107 | AB | 2145 |
| | 14827 | 14842 | | | | | | |
| | 14973 | 14988 | | | | | | |
| 1242533 | 15876 | 15891 | N/A | N/A | AGAAAATCGCAAGCCC | 67 | AB | 2146 |
| 1242557 | 17752 | 17767 | N/A | N/A | TCATAGTTTTTAGGTG | 62 | AB | 2147 |
| 1242581 | 19532 | 19547 | N/A | N/A | GTTAAATGTCCCAAAC | 96 | AB | 2148 |
| 1242605 | 20484 | 20499 | N/A | N/A | CGTAAACAGTGCCGGG | 68 | AB | 2149 |
| 1242629 | 20637 | 20652 | N/A | N/A | CAGGACACACCTGACC | 49 | AB | 2150 |
| | 20679 | 20694 | | | | | | |
| | 20721 | 20736 | | | | | | |
| | 20763 | 20778 | | | | | | |
| | 20805 | 20820 | | | | | | |
| 1242653 | 20961 | 20976 | N/A | N/A | GATCAGAGAGCTCCGG | 77 | AB | 2151 |
| 1242677 | 22486 | 22501 | N/A | N/A | CTCCTATAAGGACAAT | 64 | AB | 2152 |
| 1242700 | 23151 | 23166 | N/A | N/A | CTGGTAAGACACCCAT | 68 | AB | 2153 |
| 1242724 | 23477 | 23492 | N/A | N/A | GCACTATGCGATAACT | 40 | AB | 2154 |
| 1242748 | 23844 | 23859 | N/A | N/A | GAAAGTGGTACAGGGA | 79 | AB | 2155 |
| 1242772 | 24165 | 24180 | N/A | N/A | GCAAAGCTAAATTGGA | 51 | AB | 2156 |
| 1242796 | 24462 | 24477 | N/A | N/A | CTTCATAGAAAGAGAT | 71 | AB | 2157 |
| 1242820 | 25021 | 25036 | N/A | N/A | TAGTAACTACATAGAG | 83 | AB | 2158 |
| 1242844 | 25890 | 25905 | N/A | N/A | CTATTGTAAATGGCTC | 64 | AB | 2159 |
| 1242868 | 26405 | 26420 | N/A | N/A | AGCAAGATGACATTCC | 65 | AB | 2160 |
| 1242892 | 26767 | 26782 | N/A | N/A | CACAAATCAGCATGGA | 34 | AB | 2161 |
| 1242916 | 27111 | 27126 | N/A | N/A | GCTATACCTAACACAT | 24 | AB | 2162 |
| | 27129 | 27144 | | | | | | |
| 1242940 | 27572 | 27587 | N/A | N/A | ATCCAATATGACTCAA | 44 | AB | 2163 |
| 1242964 | 28620 | 28635 | N/A | N/A | TTTACCAAGCACGGAT | 49 | AB | 2164 |
| 1242988 | 28839 | 28854 | N/A | N/A | ACACAGTAGTAGTAGA | 72 | AB | 2165 |
| 1243012 | 29179 | 29194 | N/A | N/A | CCCAATTAAGGAAGAG | 91 | AB | 2166 |
| 1243036 | 29487 | 29502 | N/A | N/A | TAAGAGTTTTGATGTA | 52 | AB | 2167 |
| 1243060 | 31214 | 31229 | N/A | N/A | AGATTATCTTGTGGCC | 63 | AB | 2168 |
| 1243084 | 31567 | 31582 | N/A | N/A | ATCTTAGCCTGTCTTG | 88 | AB | 2169 |
| 1243108 | 33038 | 33053 | N/A | N/A | GAAGAATCAGGTTTCG | 101 | AB | 2170 |
| 1243132 | 33915 | 33930 | N/A | N/A | ATATTGGCTTGTCCTG | 43 | AB | 2171 |
| 1243156 | 34154 | 34169 | N/A | N/A | CCAGTATTGTGAGATA | 15 | AB | 2172 |
| 1243180 | 34401 | 34416 | N/A | N/A | TTAAAGGTGAGGCCCT | 80 | AB | 2173 |
| 1243204 | 34616 | 34631 | N/A | N/A | CAATTCTACACATTGC | 47 | AB | 2174 |
| 1243228 | 13396 | 13411 | N/A | N/A | GCACTGAACGATAAAC | 64 | AB | 2175 |
| 1243252 | 30624 | 30639 | N/A | N/A | ATCCCAGTGGTTATCC | 54 | AB | 2176 |
| 1232737 | 5138 | 5153 | 90 | 105 | ATGCCATCTTGACCCA | 49 | AC | 22 |
| 1241432 | 5140 | 5155 | 92 | 107 | CGATGCCATCTTGACC | 55 | AC | 2177 |
| 1241456 | 5245 | 5260 | 197 | 212 | AGTGTGTCCTGAGCCA | 23 | AC | 2178 |
| 1241480 | 5595 | 5610 | 547 | 562 | GTTAGCCAAGGCCGGG | 40 | AC | 2179 |
| 1241504 | 5752 | 5767 | 704 | 719 | TTCGGTCCACACTAAG | 46 | AC | 2180 |
| 1241528 | 5878 | 5893 | 830 | 845 | AGGATAGTCCTCTAAG | 44 | AC | 2181 |
| 1241552 | 5963 | 5978 | 915 | 930 | TGAAGTCGATCATTAG | 49 | AC | 2182 |
| 1241576 | 6018 | 6033 | 970 | 985 | CTGTTGATCGCAGCGA | 42 | AC | 2183 |
| 1241600 | N/A | N/A | 1027 | 1042 | GCATTATCTGAACCCC | 56 | AC | 2184 |
| 1241624 | 10320 | 10335 | 1120 | 1135 | CAAATAGAGATTCTCG | 75 | AC | 2185 |
| 1241648 | 10886 | 10901 | 1189 | 1204 | TCTTCAATGCACTGGA | 28 | AC | 2186 |
| 1241672 | 11058 | 11073 | 1361 | 1376 | GTCAAACAGCAACTCC | 38 | AC | 2187 |
| 1241696 | 11296 | 11311 | 1599 | 1614 | TCTTGTGGATGGGTGG | 39 | AC | 2188 |
| 1241720 | 11422 | 11437 | 1725 | 1740 | GGAGAATGTCTCCCCG | 71 | AC | 2189 |
| 1241744 | 11630 | 11645 | 1933 | 1948 | TCCTGAATCAGACTGA | 56 | AC | 2190 |
| 1241768 | 11932 | 11947 | 2235 | 2250 | AAGCAGACACATCCGC | 58 | AC | 2191 |
| 1241791 | 12137 | 12152 | 2440 | 2455 | TTTTCGAATTTGCCAT | 32 | AC | 2192 |
| 1241815 | 12201 | 12216 | 2504 | 2519 | GTAGGAGGTCCTCTCC | 64 | AC | 2193 |
| 1241838 | 12333 | 12348 | 2636 | 2651 | GTACAAACAGTAGAAC | 39 | AC | 2194 |
| 1241862 | 12455 | 12470 | 2758 | 2773 | ACCCGATGACAGTTCT | 42 | AC | 2195 |
| 1241886 | 12537 | 12552 | 2840 | 2855 | CATATCAAGGTGTCGG | 37 | AC | 2196 |
| 1241910 | N/A | N/A | 2893 | 2908 | ACCAATCCATGAGAAC | 78 | AC | 2197 |
| 1241934 | 16701 | 16716 | 3019 | 3034 | TCACACAACACTCTCA | 55 | AC | 2198 |
| 1241958 | 21241 | 21256 | 3212 | 3227 | GCACAACAGGTGCTTC | 98 | AC | 2199 |
| 1241982 | 23003 | 23018 | 3278 | 3293 | TGATGCAAGATCCTGA | 50 | AC | 2200 |
| 1242006 | 23051 | 23066 | 3326 | 3341 | ATTCTCCCCCACATAG | 57 | AC | 2201 |
| 1242030 | 23112 | 23127 | 3387 | 3402 | GGTTACACTGTGGATT | 55 | AC | 2202 |
| 1242054 | 31006 | 31021 | 3450 | 3465 | CCGAGGACAAAGCTGA | 36 | AC | 2203 |
| 1242078 | 31054 | 31069 | 3498 | 3513 | TGTTGCCTCGCAGGTA | 61 | AC | 2204 |
| 1242102 | 31101 | 31116 | 3545 | 3560 | GTGCAAGAGTCCCTCA | 51 | AC | 2205 |
| 1242126 | 31724 | 31739 | 3660 | 3675 | CCAGGCTCAGCTTTCG | 41 | AC | 2206 |
| 1242150 | 35489 | 35504 | 3842 | 3857 | CTACCAAGAAGGCTCA | 59 | AC | 2207 |
| 1242174 | 35649 | 35664 | 4002 | 4017 | GCACAGGAAGGCATCG | 62 | AC | 2208 |
| 1242198 | 35766 | 35781 | 4119 | 4134 | CATGAGGTCACCAAGA | 65 | AC | 2209 |
| 1242222 | 35909 | 35924 | 4262 | 4277 | GTGTTATGGTCAGTTA | 74 | AC | 2210 |
| 1242246 | N/A | N/A | N/A | N/A | AACCCATGAGAACAGG | 75 | AC | 2211 |
| 1242270 | 6679 | 6694 | N/A | N/A | AACTACTCATAAAGGA | 87 | AC | 2212 |
| 1242294 | 7148 | 7163 | N/A | N/A | ATCATTAGGGAAACGG | 38 | AC | 2213 |
| 1242318 | 8535 | 8550 | N/A | N/A | CTTAGCAAAGTACTTC | 41 | AC | 2214 |
| 1242342 | 9477 | 9492 | N/A | N/A | GAGAAATTCAGGTTAG | 58 | AC | 2215 |
| 1242366 | 10182 | 10197 | N/A | N/A | AACTATTATCACAGAG | 73 | AC | 2216 |
| 1242390 | 12637 | 12652 | N/A | N/A | GAAGAAGCTGGCGAGG | 91 | AC | 2217 |
| 1242414 | 13017 | 13032 | N/A | N/A | CTAAGAAACATCCCCC | 50 | AC | 2218 |
| 1242438 | 14477 | 14492 | N/A | N/A | CTGAGCCTCCTACCGG | 29 | AC | 2219 |
| | 14623 | 14638 | | | | | | |
| | 14769 | 14784 | | | | | | |
| | 14915 | 14930 | | | | | | |
| | 15061 | 15076 | | | | | | |
| 1242462 | 14541 | 14556 | N/A | N/A | CTACCAGGGCTCCAGT | 39 | AC | 2220 |
| | 15269 | 15284 | | | | | | |
| 1242486 | 14586 | 14601 | N/A | N/A | TCAGAGTAAGGCAGGT | 12 | AC | 2221 |
| | 14878 | 14893 | | | | | | |
| | 15168 | 15183 | | | | | | |
| | 15241 | 15256 | | | | | | |
| 1242510 | 14682 | 14697 | N/A | N/A | AGGGCTCCAATCAGAG | 87 | AC | 2222 |
| | 14828 | 14843 | | | | | | |
| | 14974 | 14989 | | | | | | |
| 1242534 | 16784 | 16799 | N/A | N/A | CATATGAACTTGCACT | 69 | AC | 2223 |
| 1242558 | 17987 | 18002 | N/A | N/A | CCATACAAAGCATTCT | 27 | AC | 2224 |
| 1242582 | 19537 | 19552 | N/A | N/A | ATAAGGTTAAATGTCC | 76 | AC | 2225 |
| 1242606 | 20552 | 20567 | N/A | N/A | ACATTGGGAGGTCAGC | 81 | AC | 2226 |
| 1242630 | 20638 | 20653 | N/A | N/A | TCAGGACACACCTGAC | 47 | AC | 2227 |
| | 20680 | 20695 | | | | | | |
| | 20722 | 20737 | | | | | | |
| | 20764 | 20779 | | | | | | |
| | 20806 | 20821 | | | | | | |
| | 20848 | 20863 | | | | | | |
| 1242654 | 21551 | 21566 | N/A | N/A | AAGCAATGCATTACAC | 52 | AC | 2228 |
| 1242678 | 22496 | 22511 | N/A | N/A | TTCTAGACACCTCCTA | 59 | AC | 2229 |
| 1242701 | 23209 | 23224 | N/A | N/A | CAAAGTAACCCCCATC | 66 | AC | 2230 |
| 1242725 | 23543 | 23558 | N/A | N/A | TGTAAGAGTCCTCTCC | 48 | AC | 2231 |
| 1242749 | 23849 | 23864 | N/A | N/A | TACAGGAAAGTGGTAC | 72 | AC | 2232 |
| 1242773 | 24166 | 24181 | N/A | N/A | AGCAAAGCTAAATTGG | 56 | AC | 2233 |
| 1242797 | 24490 | 24505 | N/A | N/A | GCGAGGAAATGAAGGC | 42 | AC | 2234 |
| 1242821 | 25026 | 25041 | N/A | N/A | GACAATAGTAACTACA | 36 | AC | 2235 |
| 1242845 | 25892 | 25907 | N/A | N/A | GACTATTGTAAATGGC | 48 | AC | 2236 |
| 1242869 | 26406 | 26421 | N/A | N/A | CAGCAAGATGACATTC | 63 | AC | 2237 |
| 1242893 | 26770 | 26785 | N/A | N/A | CTACACAAATCAGCAT | 61 | AC | 2238 |
| 1242917 | 27130 | 27145 | N/A | N/A | AGCTATACCTAACACA | 71 | AC | 2239 |
| 1242941 | 27613 | 27628 | N/A | N/A | TCATAGCAAGTCCCCA | 80 | AC | 2240 |
| 1242965 | 28622 | 28637 | N/A | N/A | CATTTACCAAGCACGG | 25 | AC | 2241 |
| 1242989 | 28876 | 28891 | N/A | N/A | GTAGAATGGATTATTC | 54 | AC | 2242 |
| 1243013 | 29206 | 29221 | N/A | N/A | GATTATCCATCCACTG | 37 | AC | 2243 |
| 1243037 | 29504 | 29519 | N/A | N/A | ATCAATCTCGATGGAA | 41 | AC | 2244 |
| 1243061 | 31215 | 31230 | N/A | N/A | CAGATTATCTTGTGGC | 52 | AC | 2245 |
| 1243085 | 31576 | 31591 | N/A | N/A | GATTTTAGCATCTTAG | 53 | AC | 2246 |
| 1243109 | 33090 | 33105 | N/A | N/A | GAAATGTTTTGGCCCG | 79 | AC | 2247 |
| 1243133 | 33916 | 33931 | N/A | N/A | AATATTGGCTTGTCCT | 81 | AC | 2248 |
| 1243157 | 34194 | 34209 | N/A | N/A | ACCAAATTCATCAGAG | 57 | AC | 2249 |
| 1243181 | 34402 | 34417 | N/A | N/A | CTTAAAGGTGAGGCCC | 58 | AC | 2250 |
| 1243205 | 34619 | 34634 | N/A | N/A | GATCAATTCTACACAT | 95 | AC | 2251 |
| 1243229 | 24164 | 24179 | N/A | N/A | CAAAGCTAAATTGGAG | 64 | AC | 2252 |
| 1243253 | 13588 | 13603 | N/A | N/A | TAGGAAGGTGGTCCTG | 75 | AC | 2253 |
| 1232737 | 5138 | 5153 | 90 | 105 | ATGCCATCTTGACCCA | 58 | AD | 22 |
| 1241433 | 5141 | 5156 | 93 | 108 | ACGATGCCATCTTGAC | 39 | AD | 2254 |
| 1241457 | 5247 | 5262 | 199 | 214 | GGAGTGTGTCCTGAGC | 53 | AD | 2255 |
| 1241481 | 5625 | 5640 | 577 | 592 | GTAACAAGTCAAAATC | 67 | AD | 2256 |
| 1241505 | 5754 | 5769 | 706 | 721 | GCTTCGGTCCACACTA | 53 | AD | 2257 |
| 1241529 | 5879 | 5894 | 831 | 846 | GAGGATAGTCCTCTAA | 95 | AD | 2258 |
| 1241553 | 5968 | 5983 | 920 | 935 | CCCATTGAAGTCGATC | 51 | AD | 2259 |
| 1241577 | 6019 | 6034 | 971 | 986 | CCTGTTGATCGCAGCG | 39 | AD | 2260 |
| 1241601 | N/A | N/A | 1028 | 1043 | TGCATTATCTGAACCC | 49 | AD | 2261 |
| 1241625 | N/A | N/A | 1141 | 1156 | CGGTAATCTTTCTTCA | 40 | AD | 2262 |
| 1241649 | 10887 | 10902 | 1190 | 1205 | GTCTTCAATGCACTGG | 35 | AD | 2263 |
| 1241673 | 11059 | 11074 | 1362 | 1377 | GGTCAAACAGCAACTC | 40 | AD | 2264 |
| 1241697 | 11297 | 11312 | 1600 | 1615 | ATCTTGTGGATGGGTG | 48 | AD | 2265 |
| 1241721 | 11423 | 11438 | 1726 | 1741 | AGGAGAATGTCTCCCC | 64 | AD | 2266 |
| 1241745 | 11727 | 11742 | 2030 | 2045 | GGCAAGGCTCTTGCCA | 95 | AD | 2267 |
| 1241769 | 11933 | 11948 | 2236 | 2251 | AAAGCAGACACATCCG | 57 | AD | 2268 |
| 1241792 | 12142 | 12157 | 2445 | 2460 | ACCCCTTTTCGAATTT | 38 | AD | 2269 |
| 1241816 | 12202 | 12217 | 2505 | 2520 | AGTAGGAGGTCCTCTC | 47 | AD | 2270 |
| 1241839 | 12359 | 12374 | 2662 | 2677 | TGCACGAAGTCCTCCT | 39 | AD | 2271 |
| 1241863 | 12463 | 12478 | 2766 | 2781 | GTGACTCCACCCGATG | 56 | AD | 2272 |
| 1241887 | 12538 | 12553 | 2841 | 2856 | CCATATCAAGGTGTCG | 36 | AD | 2273 |
| 1241911 | N/A | N/A | 2894 | 2909 | CACCAATCCATGAGAA | 39 | AD | 2274 |
| 1241935 | 16730 | 16745 | 3048 | 3063 | GAATGTTACAGCCAGG | 33 | AD | 2275 |
| 1241959 | 21246 | 21261 | 3217 | 3232 | AGATTGCACAACAGGT | 43 | AD | 2276 |
| 1241983 | 23004 | 23019 | 3279 | 3294 | CTGATGCAAGATCCTG | 26 | AD | 2277 |
| 1242007 | 23052 | 23067 | 3327 | 3342 | CATTCTCCCCCACATA | 55 | AD | 2278 |
| 1242031 | 23113 | 23128 | 3388 | 3403 | AGGTTACACTGTGGAT | 57 | AD | 2279 |
| 1242055 | 31007 | 31022 | 3451 | 3466 | ACCGAGGACAAAGCTG | 33 | AD | 2280 |
| 1242079 | 31055 | 31070 | 3499 | 3514 | GTGTTGCCTCGCAGGT | 26 | AD | 2281 |
| 1242103 | 31102 | 31117 | 3546 | 3561 | GGTGCAAGAGTCCCTC | 37 | AD | 2282 |
| 1242127 | 31736 | 31751 | 3672 | 3687 | GGTCATTGTTGCCCAG | 57 | AD | 2283 |
| 1242151 | 35490 | 35505 | 3843 | 3858 | CCTACCAAGAAGGCTC | 53 | AD | 2284 |
| 1242175 | 35650 | 35665 | 4003 | 4018 | TGCACAGGAAGGCATC | 76 | AD | 2285 |
| 1242199 | 35771 | 35786 | 4124 | 4139 | AATTACATGAGGTCAC | 53 | AD | 2286 |
| 1242223 | 35910 | 35925 | 4263 | 4278 | TGTGTTATGGTCAGTT | 73 | AD | 2287 |
| 1242247 | N/A | N/A | N/A | N/A | CCCAACCCATGAGAAC | 98 | AD | 2288 |
| 1242271 | 6717 | 6732 | N/A | N/A | CATAGTTGAACCCGGA | 35 | AD | 2289 |
| 1242295 | 7395 | 7410 | N/A | N/A | CATACTAACTTCTGTG | 75 | AD | 2290 |
| 1242319 | 8566 | 8581 | N/A | N/A | CTTGAATGTGCCATAA | 57 | AD | 2291 |
| 1242343 | 9483 | 9498 | N/A | N/A | CTATAGGAGAAATTCA | 89 | AD | 2292 |
| 1242367 | 10183 | 10198 | N/A | N/A | GAACTATTATCACAGA | 90 | AD | 2293 |
| 1242391 | 12639 | 12654 | N/A | N/A | AAGAAGAAGCTGGCGA | 81 | AD | 2294 |
| 1242415 | 13018 | 13033 | N/A | N/A | TCTAAGAAACATCCCC | 59 | AD | 2295 |
| 1242439 | 14478 | 14493 | N/A | N/A | GCTGAGCCTCCTACCG | 24 | AD | 2296 |
| | 14624 | 14639 | | | | | | |
| | 14770 | 14785 | | | | | | |
| | 14916 | 14931 | | | | | | |
| | 15062 | 15077 | | | | | | |
| 1242463 | 14542 | 14557 | N/A | N/A | CCTACCAGGGCTCCAG | 29 | AD | 2297 |
| | 15270 | 15285 | | | | | | |
| 1242487 | 14587 | 14602 | N/A | N/A | CTCAGAGTAAGGCAGG | 14 | AD | 2298 |
| | 14879 | 14894 | | | | | | |
| | 15169 | 15184 | | | | | | |
| | 15242 | 15257 | | | | | | |
| 1242511 | 14683 | 14698 | N/A | N/A | CAGGGCTCCAATCAGA | 70 | AD | 2299 |
| | 14829 | 14844 | | | | | | |
| | 14975 | 14990 | | | | | | |
| 1242535 | 16786 | 16801 | N/A | N/A | CTCATATGAACTTGCA | 30 | AD | 2300 |
| 1242559 | 17999 | 18014 | N/A | N/A | TGACATTTCAACCCAT | 74 | AD | 2301 |
| 1242583 | 19595 | 19610 | N/A | N/A | CCAGAATCATACAGGG | 53 | AD | 2302 |
| 1242607 | 20561 | 20576 | N/A | N/A | GCACAAGGCACATTGG | 59 | AD | 2303 |
| 1242631 | 20641 | 20656 | N/A | N/A | GCATCAGGACACACCT | 22 | AD | 2304 |
| | 20683 | 20698 | | | | | | |
| | 20725 | 20740 | | | | | | |
| | 20767 | 20782 | | | | | | |
| | 20809 | 20824 | | | | | | |
| | 20851 | 20866 | | | | | | |
| | 21019 | 21034 | | | | | | |
| 1242655 | 21597 | 21612 | N/A | N/A | CAATATGAGGACCCAG | 31 | AD | 2305 |
| 1242679 | 22576 | 22591 | N/A | N/A | ACCATTAAACAATGTC | 53 | AD | 2306 |
| 1242702 | 23214 | 23229 | N/A | N/A | CTGACCAAAGTAACCC | 95 | AD | 2307 |
| 1242726 | 23545 | 23560 | N/A | N/A | AGTGTAAGAGTCCTCT | 31 | AD | 2308 |
| 1242750 | 23851 | 23866 | N/A | N/A | GATACAGGAAAGTGGT | 59 | AD | 2309 |
| 1242774 | 24172 | 24187 | N/A | N/A | AGGTTTAGCAAAGCTA | 40 | AD | 2310 |
| 1242798 | 24503 | 24518 | N/A | N/A | GTAATACCCTAAGGCG | 47 | AD | 2311 |
| 1242822 | 25033 | 25048 | N/A | N/A | AGGTATTGACAATAGT | 17 | AD | 2312 |
| 1242846 | 25943 | 25958 | N/A | N/A | GATTATATAACAATCT | 107 | AD | 2313 |
| 1242870 | 26417 | 26432 | N/A | N/A | GCAAAATGTGTCAGCA | 68 | AD | 2314 |
| 1242894 | 26794 | 26809 | N/A | N/A | GATATTATGCCCCAAT | 35 | AD | 2315 |
| 1242918 | 27137 | 27152 | N/A | N/A | AAAATGTAGCTATACC | 89 | AD | 2316 |
| 1242942 | 27614 | 27629 | N/A | N/A | ATCATAGCAAGTCCCC | 59 | AD | 2317 |
| 1242966 | 28625 | 28640 | N/A | N/A | CTACATTTACCAAGCA | 42 | AD | 2318 |
| 1242990 | 28878 | 28893 | N/A | N/A | GAGTAGAATGGATTAT | 70 | AD | 2319 |
| 1243014 | 29209 | 29224 | N/A | N/A | AGTGATTATCCATCCA | 34 | AD | 2320 |
| 1243038 | 29505 | 29520 | N/A | N/A | CATCAATCTCGATGGA | 62 | AD | 2321 |
| 1243062 | 31216 | 31231 | N/A | N/A | ACAGATTATCTTGTGG | 71 | AD | 2322 |
| 1243086 | 31580 | 31595 | N/A | N/A | CCAAGATTTTAGCATC | 63 | AD | 2323 |
| 1243110 | 33139 | 33154 | N/A | N/A | ACACATTGGACATGAT | 73 | AD | 2324 |
| 1243134 | 33918 | 33933 | N/A | N/A | GGAATATTGGCTTGTC | 37 | AD | 2325 |
| 1243158 | 34214 | 34229 | N/A | N/A | GAAGAGGACTCTCGCC | 71 | AD | 2326 |
| 1243182 | 34404 | 34419 | N/A | N/A | TACTTAAAGGTGAGGC | 76 | AD | 2327 |
| 1243206 | 34629 | 34644 | N/A | N/A | TCACTAACCTGATCAA | 108 | AD | 2328 |
| 1243230 | 27223 | 27238 | N/A | N/A | GCTCAGCATGTGTGAC | 68 | AD | 2329 |
| 1243254 | 9548 | 9563 | N/A | N/A | CATGCATCTGTGTTGA | 85 | AD | 2330 |
| 871399 | 22659 | 22674 | N/A | N/A | CCTTAAGCACTTCACA | 58 | AE | 2331 |
| 1232737 | 5138 | 5153 | 90 | 105 | ATGCCATCTTGACCCA | 48 | AE | 22 |
| 1241434 | 5143 | 5158 | 95 | 110 | TCACGATGCCATCTTG | 34 | AE | 2332 |
| 1241458 | 5248 | 5263 | 200 | 215 | AGGAGTGTGTCCTGAG | 46 | AE | 2333 |
| 1241482 | 5629 | 5644 | 581 | 596 | GACTGTAACAAGTCAA | 81 | AE | 2334 |
| 1241506 | 5759 | 5774 | 711 | 726 | CTTAGGCTTCGGTCCA | 45 | AE | 2335 |
| 1241530 | 5880 | 5895 | 832 | 847 | GGAGGATAGTCCTCTA | 90 | AE | 2336 |
| 1241554 | 5970 | 5985 | 922 | 937 | TCCCCATTGAAGTCGA | 74 | AE | 2337 |
| 1241578 | 6020 | 6035 | 972 | 987 | TCCTGTTGATCGCAGC | 27 | AE | 2338 |
| 1241602 | 10229 | 10244 | 1029 | 1044 | GTGCATTATCTGAACC | 53 | AE | 2339 |
| 1241626 | N/A | N/A | 1142 | 1157 | ACGGTAATCTTTCTTC | 32 | AE | 2340 |
| 1241650 | 10888 | 10903 | 1191 | 1206 | TGTCTTCAATGCACTG | 45 | AE | 2341 |
| 1241674 | 11080 | 11095 | 1383 | 1398 | GCTCAGAATGCTCATC | 30 | AE | 2342 |
| 1241698 | 11299 | 11314 | 1602 | 1617 | CGATCTTGTGGATGGG | 27 | AE | 2343 |
| 1241722 | 11424 | 11439 | 1727 | 1742 | CAGGAGAATGTCTCCC | 61 | AE | 2344 |
| 1241746 | 11728 | 11743 | 2031 | 2046 | GGGCAAGGCTCTTGCC | 102 | AE | 2345 |
| 1241770 | 11946 | 11961 | 2249 | 2264 | GTTCATCCTCAGGAAA | 81 | AE | 2346 |
| 1241793 | 12144 | 12159 | 2447 | 2462 | ATACCCCTTTTCGAAT | 63 | AE | 2347 |
| 1241817 | 12205 | 12220 | 2508 | 2523 | CCAAGTAGGAGGTCCT | 34 | AE | 2348 |
| 1241840 | 12360 | 12375 | 2663 | 2678 | TTGCACGAAGTCCTCC | 35 | AE | 2349 |
| 1241864 | 12465 | 12480 | 2768 | 2783 | CAGTGACTCCACCCGA | 25 | AE | 2350 |
| 1241888 | 12539 | 12554 | 2842 | 2857 | ACCATATCAAGGTGTC | 81 | AE | 2351 |
| 1241912 | N/A | N/A | 2897 | 2912 | GTTCACCAATCCATGA | 75 | AE | 2352 |
| 1241936 | 16731 | 16746 | 3049 | 3064 | CGAATGTTACAGCCAG | 46 | AE | 2353 |
| 1241960 | 21248 | 21263 | 3219 | 3234 | TCAGATTGCACAACAG | 32 | AE | 2354 |
| 1241984 | 23005 | 23020 | 3280 | 3295 | ACTGATGCAAGATCCT | 18 | AE | 2355 |
| 1242008 | 23053 | 23068 | 3328 | 3343 | GCATTCTCCCCCACAT | 40 | AE | 2356 |
| 1242032 | 23114 | 23129 | 3389 | 3404 | CAGGTTACACTGTGGA | 41 | AE | 2357 |
| 1242056 | 31012 | 31027 | 3456 | 3471 | TGAGTACCGAGGACAA | 40 | AE | 2358 |
| 1242080 | 31056 | 31071 | 3500 | 3515 | AGTGTTGCCTCGCAGG | 32 | AE | 2359 |
| 1242104 | 31122 | 31137 | 3566 | 3581 | CACCTGAAGCTTGCAG | 17 | AE | 2360 |
| 1242128 | 31737 | 31752 | 3673 | 3688 | AGGTCATTGTTGCCCA | 65 | AE | 2361 |
| 1242152 | 35491 | 35506 | 3844 | 3859 | TCCTACCAAGAAGGCT | 74 | AE | 2362 |
| 1242176 | 35662 | 35677 | 4015 | 4030 | ATGCCCAAGCTCTGCA | 82 | AE | 2363 |
| 1242200 | 35773 | 35788 | 4126 | 4141 | CTAATTACATGAGGTC | 15 | AE | 2364 |
| 1242224 | 35911 | 35926 | 4264 | 4279 | CTGTGTTATGGTCAGT | 78 | AE | 2365 |
| 1242248 | N/A | N/A | N/A | N/A | CCCCAACCCATGAGAA | 88 | AE | 2366 |
| 1242272 | 6719 | 6734 | N/A | N/A | AACATAGTTGAACCCG | 51 | AE | 2367 |
| 1242296 | 7397 | 7412 | N/A | N/A | ATCATACTAACTTCTG | 58 | AE | 2368 |
| 1242320 | 8592 | 8607 | N/A | N/A | AATACCAGAAGCTGTT | 52 | AE | 2369 |
| 1242344 | 9497 | 9512 | N/A | N/A | CTTAAGAAGGCATTCT | 72 | AE | 2370 |
| 1242368 | 10227 | 10242 | N/A | N/A | GCATTATCTGAACCTA | 68 | AE | 2371 |
| 1242392 | 12683 | 12698 | N/A | N/A | GTATTTGGAAGATAGT | 73 | AE | 2372 |
| 1242416 | 13043 | 13058 | N/A | N/A | TTAAAGGAGGCTTAGA | 75 | AE | 2373 |
| 1242440 | 14479 | 14494 | N/A | N/A | TGCTGAGCCTCCTACC | 30 | AE | 2374 |
| | 14625 | 14640 | | | | | | |
| | 14771 | 14786 | | | | | | |
| | 14917 | 14932 | | | | | | |
| | 15063 | 15078 | | | | | | |
| 1242464 | 14544 | 14559 | N/A | N/A | CCCCTACCAGGGCTCC | 50 | AE | 2375 |
| | 15199 | 15214 | | | | | | |
| | 15272 | 15287 | | | | | | |
| 1242488 | 14588 | 14603 | N/A | N/A | CCTCAGAGTAAGGCAG | 23 | AE | 2376 |
| | 14880 | 14895 | | | | | | |
| | 15170 | 15185 | | | | | | |
| | 15243 | 15258 | | | | | | |
| | 15316 | 15331 | | | | | | |
| 1242512 | 14685 | 14700 | N/A | N/A | GCCAGGGCTCCAATCA | 68 | AE | 2377 |
| | 14831 | 14846 | | | | | | |
| | 14977 | 14992 | | | | | | |
| 1242536 | 17258 | 17273 | N/A | N/A | GATCTTAGACTTTCGG | 55 | AE | 2378 |
| 1242560 | 18351 | 18366 | N/A | N/A | GACTTTAACACTTGCT | 19 | AE | 2379 |
| 1242584 | 19596 | 19611 | N/A | N/A | TCCAGAATCATACAGG | 37 | AE | 2380 |
| 1242608 | 20567 | 20582 | N/A | N/A | AATACAGCACAAGGCA | 64 | AE | 2381 |
| 1242632 | 20642 | 20657 | N/A | N/A | AGCATCAGGACACACC | 11 | AE | 2382 |
| | 20684 | 20699 | | | | | | |
| | 20726 | 20741 | | | | | | |
| | 20810 | 20825 | | | | | | |
| | 20852 | 20867 | | | | | | |
| | 21020 | 21035 | | | | | | |
| 1242656 | 21598 | 21613 | N/A | N/A | ACAATATGAGGACCCA | 19 | AE | 2383 |
| 1242703 | 23247 | 23262 | N/A | N/A | GTTTTTACCTCCTTAC | 53 | AE | 2384 |
| 1242727 | 23546 | 23561 | N/A | N/A | CAGTGTAAGAGTCCTC | 22 | AE | 2385 |
| 1242751 | 23859 | 23874 | N/A | N/A | GGGAAAGTGATACAGG | 71 | AE | 2386 |
| 1242775 | 24180 | 24195 | N/A | N/A | GTAAGATGAGGTTTAG | 42 | AE | 2387 |
| 1242799 | 24505 | 24520 | N/A | N/A | GGGTAATACCCTAAGG | 89 | AE | 2388 |
| 1242823 | 25074 | 25089 | N/A | N/A | GCTCTGTTTGTGATAC | 23 | AE | 2389 |
| | 26262 | 26277 | | | | | | |
| 1242847 | 25944 | 25959 | N/A | N/A | GGATTATATAACAATC | 74 | AE | 2390 |
| 1242871 | 26418 | 26433 | N/A | N/A | GGCAAAATGTGTCAGC | 31 | AE | 2391 |
| 1242895 | 26795 | 26810 | N/A | N/A | GGATATTATGCCCCAA | 24 | AE | 2392 |
| 1242919 | 27155 | 27170 | N/A | N/A | CCCAAAGTTCGAGAGA | 65 | AE | 2393 |
| 1242943 | 27651 | 27666 | N/A | N/A | CTGTAGATGAACCCAG | 73 | AE | 2394 |
| 1242967 | 28637 | 28652 | N/A | N/A | CCTAACTGAGCTCTAC | 57 | AE | 2395 |
| 1242991 | 28913 | 28928 | N/A | N/A | AAAGTTATGCAGGCTT | 40 | AE | 2396 |
| 1243015 | 29214 | 29229 | N/A | N/A | GAAAAAGTGATTATCC | 98 | AE | 2397 |
| 1243039 | 29526 | 29541 | N/A | N/A | GAGCATAAAACCCCAA | 37 | AE | 2398 |
| 1243063 | 31219 | 31234 | N/A | N/A | GATACAGATTATCTTG | 51 | AE | 2399 |
| 1243087 | 31581 | 31596 | N/A | N/A | CCCAAGATTTTAGCAT | 87 | AE | 2400 |
| 1243111 | 33267 | 33282 | N/A | N/A | TCAGAATGCCACCATG | 92 | AE | 2401 |
| 1243135 | 33919 | 33934 | N/A | N/A | GGGAATATTGGCTTGT | 29 | AE | 2402 |
| 1243159 | 34218 | 34233 | N/A | N/A | TCAGGAAGAGGACTCT | 53 | AE | 2403 |
| 1243183 | 34405 | 34420 | N/A | N/A | GTACTTAAAGGTGAGG | 45 | AE | 2404 |
| 1243207 | 34636 | 34651 | N/A | N/A | CATTTAGTCACTAACC | 67 | AE | 2405 |
| 1243231 | 34205 | 34220 | N/A | N/A | TCTCGCCAGCAACCAA | 52 | AE | 2406 |
| 1243255 | 24313 | 24328 | N/A | N/A | GAATGCTGATCTGGCT | 47 | AE | 2407 |
| 1232737 | 5138 | 5153 | 90 | 105 | ATGCCATCTTGACCCA | 57 | AF | 22 |
| 1241430 | 5137 | 5152 | 89 | 104 | TGCCATCTTGACCCAT | 37 | AF | 2408 |
| 1241454 | 5223 | 5238 | 175 | 190 | AAAGGTTTCCCTATGG | 76 | AF | 2409 |
| 1241478 | 5292 | 5307 | 244 | 259 | GTTAGCCAAATGCTTA | 40 | AF | 2410 |
| 1241502 | 5747 | 5762 | 699 | 714 | TCCACACTAAGATACC | 77 | AF | 2411 |
| 1241526 | 5876 | 5891 | 828 | 843 | GATAGTCCTCTAAGTG | 95 | AF | 2412 |
| 1241550 | 5947 | 5962 | 899 | 914 | CGTGGCTAGATCCACA | 89 | AF | 2413 |
| 1241574 | 6016 | 6031 | 968 | 983 | GTTGATCGCAGCGAAG | 40 | AF | 2414 |
| 1241598 | N/A | N/A | 1025 | 1040 | ATTATCTGAACCCCAC | 84 | AF | 2415 |
| 1241622 | 10314 | 10329 | 1114 | 1129 | GAGATTCTCGAAAGGT | 50 | AF | 2416 |
| 1241646 | 10884 | 10899 | 1187 | 1202 | TTCAATGCACTGGAAT | 47 | AF | 2417 |
| 1241670 | 11012 | 11027 | 1315 | 1330 | GTCTTGGTCTTGCCGA | 37 | AF | 2418 |
| 1241694 | 11261 | 11276 | 1564 | 1579 | CTCATGATCAGGTCCC | 25 | AF | 2419 |
| 1241718 | 11413 | 11428 | 1716 | 1731 | CTCCCCGCTCGGCCTT | 59 | AF | 2420 |
| 1241742 | 11627 | 11642 | 1930 | 1945 | TGAATCAGACTGAAGG | 50 | AF | 2421 |
| 1241766 | 11928 | 11943 | 2231 | 2246 | AGACACATCCGCCTTC | 56 | AF | 2422 |
| 1241789 | 12133 | 12148 | 2436 | 2451 | CGAATTTGCCATAGTT | 26 | AF | 2423 |
| 1241813 | 12198 | 12213 | 2501 | 2516 | GGAGGTCCTCTCCTGG | 73 | AF | 2424 |
| 1241836 | 12325 | 12340 | 2628 | 2643 | AGTAGAACAATTCCAG | 27 | AF | 2425 |
| 1241860 | 12452 | 12467 | 2755 | 2770 | CGATGACAGTTCTCAA | 47 | AF | 2426 |
| 1241884 | 12534 | 12549 | 2837 | 2852 | ATCAAGGTGTCGGCCT | 36 | AF | 2427 |
| 1241908 | N/A | N/A | 2889 | 2904 | ATCCATGAGAACAGGC | 33 | AF | 2428 |
| 1241932 | 16696 | 16711 | 3014 | 3029 | CAACACTCTCATCCCT | 66 | AF | 2429 |
| 1241956 | 21214 | 21229 | 3185 | 3200 | CAGAAGTCTGATTCCG | 46 | AF | 2430 |
| 1241980 | 23001 | 23016 | 3276 | 3291 | ATGCAAGATCCTGACA | 66 | AF | 2431 |
| 1242004 | 23045 | 23060 | 3320 | 3335 | CCCCACATAGAGTCTG | 51 | AF | 2432 |
| 1242028 | 23110 | 23125 | 3385 | 3400 | TTACACTGTGGATTCT | 64 | AF | 2433 |
| 1242052 | 31001 | 31016 | 3445 | 3460 | GACAAAGCTGAACAAC | 54 | AF | 2434 |
| 1242076 | 31049 | 31064 | 3493 | 3508 | CCTCGCAGGTAAAGGT | 77 | AF | 2435 |
| 1242100 | 31097 | 31112 | 3541 | 3556 | AAGAGTCCCTCACAGA | 38 | AF | 2436 |
| 1242124 | 31720 | 31735 | 3656 | 3671 | GCTCAGCTTTCGCAGG | 59 | AF | 2437 |
| 1242148 | 35486 | 35501 | 3839 | 3854 | CCAAGAAGGCTCAAAG | 83 | AF | 2438 |
| 1242172 | 35632 | 35647 | 3985 | 4000 | CGGCAAGCTCTCTTCT | 52 | AF | 2439 |
| 1242196 | 35758 | 35773 | 4111 | 4126 | CACCAAGAGGAACATC | 47 | AF | 2440 |
| 1242220 | 35907 | 35922 | 4260 | 4275 | GTTATGGTCAGTTAAT | 81 | AF | 2441 |
| 1242244 | 36033 | 36048 | 4386 | 4401 | TGAATTTTCAACCAGC | 23 | AF | 2442 |
| 1242268 | 6624 | 6639 | N/A | N/A | GTAGTAAGATTCACAG | 47 | AF | 2443 |
| 1242292 | 7007 | 7022 | N/A | N/A | GGGAAATGGTGCAGAT | 67 | AF | 2444 |
| 1242316 | 8531 | 8546 | N/A | N/A | GCAAAGTACTTCTACA | 64 | AF | 2445 |
| 1242340 | 9475 | 9490 | N/A | N/A | GAAATTCAGGTTAGGT | 64 | AF | 2446 |
| 1242364 | 10164 | 10179 | N/A | N/A | TGCAATTGAAACCTGG | 67 | AF | 2447 |
| 1242388 | 10838 | 10853 | N/A | N/A | CGGTAATCTACGGCAA | 81 | AF | 2448 |
| 1242412 | 12929 | 12944 | N/A | N/A | AGACATTGACTGACTC | 28 | AF | 2449 |
| 1242436 | 14462 | 14477 | N/A | N/A | GGGCTTCAGTGAGAGT | 46 | AF | 2450 |
| | 14608 | 14623 | | | | | | |
| | 14754 | 14769 | | | | | | |
| | 14900 | 14915 | | | | | | |
| | 15046 | 15061 | | | | | | |
| 1242460 | 14508 | 14523 | N/A | N/A | GGAAGGAAGGTGCTAT | 18 | AF | 2451 |
| | 14654 | 14669 | | | | | | |
| | 14727 | 14742 | | | | | | |
| | 14800 | 14815 | | | | | | |
| | 14946 | 14961 | | | | | | |
| | 15019 | 15034 | | | | | | |
| | 15092 | 15107 | | | | | | |
| 1242484 | 14583 | 14598 | N/A | N/A | GAGTAAGGCAGGTGCC | 12 | AF | 2452 |
| | 14875 | 14890 | | | | | | |
| | 15165 | 15180 | | | | | | |
| | 15238 | 15253 | | | | | | |
| 1242508 | 14680 | 14695 | N/A | N/A | GGCTCCAATCAGAGTC | 81 | AF | 2453 |
| | 14826 | 14841 | | | | | | |
| | 14972 | 14987 | | | | | | |
| 1242532 | 15660 | 15675 | N/A | N/A | GCTATATGGATCTTTT | 47 | AF | 2454 |
| 1242556 | 17751 | 17766 | N/A | N/A | CATAGTTTTTAGGTGA | 30 | AF | 2455 |
| 1242580 | 19520 | 19535 | N/A | N/A | AAACAACCAGTCTCCA | 71 | AF | 2456 |
| 1242604 | 20444 | 20459 | N/A | N/A | TGTGAATGGTGCGCTC | 55 | AF | 2457 |
| 1242628 | 20636 | 20651 | N/A | N/A | AGGACACACCTGACCA | 31 | AF | 2458 |
| | 20678 | 20693 | | | | | | |
| | 20720 | 20735 | | | | | | |
| | 20762 | 20777 | | | | | | |
| | 20804 | 20819 | | | | | | |
| 1242652 | 20936 | 20951 | N/A | N/A | AGCATCAGAACACACC | 11 | AF | 2459 |
| | 21062 | 21077 | | | | | | |
| 1242676 | 22484 | 22499 | N/A | N/A | CCTATAAGGACAATCA | 50 | AF | 2460 |
| 1242699 | 23139 | 23154 | N/A | N/A | CCATGAAGACTTACCC | 99 | AF | 2461 |
| 1242723 | 23471 | 23486 | N/A | N/A | TGCGATAACTTGCATG | 100 | AF | 2462 |
| 1242747 | 23822 | 23837 | N/A | N/A | ATGATTATTAGTGGGT | 11 | AF | 2463 |
| 1242771 | 24159 | 24174 | N/A | N/A | CTAAATTGGAGGTCCC | 53 | AF | 2464 |
| 1242795 | 24450 | 24465 | N/A | N/A | AGATATGGTCACATCT | 97 | AF | 2465 |
| 1242819 | 25019 | 25034 | N/A | N/A | GTAACTACATAGAGCA | 18 | AF | 2466 |
| 1242843 | 25886 | 25901 | N/A | N/A | TGTAAATGGCTCTCTC | 71 | AF | 2467 |
| 1242867 | 26404 | 26419 | N/A | N/A | GCAAGATGACATTCCA | 48 | AF | 2468 |
| 1242891 | 26758 | 26773 | N/A | N/A | GCATGGAAATTTATCC | 38 | AF | 2469 |
| 1242915 | 27107 | 27122 | N/A | N/A | TACCTAACACATAGGC | 52 | AF | 2470 |
| | 27125 | 27140 | | | | | | |
| 1242939 | 27563 | 27578 | N/A | N/A | GACTCAAAATGGAGTG | 55 | AF | 2471 |
| 1242963 | 28617 | 28632 | N/A | N/A | ACCAAGCACGGATATC | 55 | AF | 2472 |
| 1242987 | 28834 | 28849 | N/A | N/A | GTAGTAGTAGAAATTG | 72 | AF | 1213 |
| 1243011 | 29178 | 29193 | N/A | N/A | CCAATTAAGGAAGAGT | 105 | AF | 2473 |
| 1243035 | 29474 | 29489 | N/A | N/A | GTACTATTCTTGAGTC | 54 | AF | 2474 |
| 1243059 | 31213 | 31228 | N/A | N/A | GATTATCTTGTGGCCA | 58 | AF | 2475 |
| 1243083 | 31514 | 31529 | N/A | N/A | CTAACTAGCTTCAAGC | 106 | AF | 2476 |
| 1243107 | 33025 | 33040 | N/A | N/A | TCGGGTAGAAGAGAGT | 80 | AF | 2477 |
| 1243131 | 33888 | 33903 | N/A | N/A | CTCCATTATGTTGGCA | 44 | AF | 2478 |
| 1243155 | 34116 | 34131 | N/A | N/A | CAGTCTAAAGCAGCTT | 44 | AF | 2479 |
| 1243179 | 34400 | 34415 | N/A | N/A | TAAAGGTGAGGCCCTT | 59 | AF | 2480 |
| 1243203 | 34609 | 34624 | N/A | N/A | ACACATTGCATTTGCC | 38 | AF | 2481 |
| 1243227 | 32157 | 32172 | N/A | N/A | ACTATGTAAAGACCCT | 42 | AF | 2482 |
| 1243251 | 9827 | 9842 | N/A | N/A | TGGGCTAAGAAAGGTC | 84 | AF | 2483 |
| 1232737 | 5138 | 5153 | 90 | 105 | ATGCCATCTTGACCCA | 60 | AH | 22 |
| 1301167 | 27196 | 27211 | N/A | N/A | ATTGAGACAGGCAGTA | 79 | AH | 2484 |
| 1301198 | 12701 | 12716 | N/A | N/A | GATGTAGCTGTGGCAA | 50 | AH | 2485 |
| 1301203 | 6740 | 6755 | N/A | N/A | GGGAATTCCGACACAT | 57 | AH | 2486 |
| 1301222 | 17664 | 17679 | N/A | N/A | TATCGAGCATCATGGT | 40 | AH | 2487 |
| 1301243 | 23533 | 23548 | N/A | N/A | CTCTCCTAGTGTAGAG | 110 | AH | 2488 |
| 1301273 | 18857 | 18872 | N/A | N/A | GTGCAGTAACATATCC | 51 | AH | 2489 |
| 1301284 | 20419 | 20434 | N/A | N/A | AAGTGAAGCGGGCGGT | 77 | AH | 2490 |
| 1301292 | 20424 | 20439 | N/A | N/A | TTTGTAAGTGAAGCGG | 24 | AH | 2491 |
| 1301298 | 22898 | 22913 | N/A | N/A | CAAGGAAGCACCCGTA | 69 | AH | 2492 |
| 1301301 | 22429 | 22444 | N/A | N/A | ATAATGTCCCCGAGGC | 37 | AH | 2493 |
| 1301314 | 25034 | 25049 | N/A | N/A | TAGGTATTGACAATAG | 26 | AH | 2494 |
| 1301324 | 29463 | 29478 | N/A | N/A | GAGTCAAATTAAGCCC | 29 | AH | 2495 |
| 1301349 | 26596 | 26611 | N/A | N/A | TGGTGATACGGTTACT | 48 | AH | 2496 |
| 1232737 | 5138 | 5153 | 90 | 105 | ATGCCATCTTGACCCA | 54 | AI | 22 |
| 1301174 | 27202 | 27217 | N/A | N/A | AGCAGTATTGAGACAG | 38 | AI | 2497 |
| 1301193 | 11266 | 11281 | 1569 | 1584 | AGCAGCTCATGATCAG | 41 | AI | 2498 |
| 1301194 | 6739 | 6754 | N/A | N/A | GGAATTCCGACACATG | 49 | AI | 2499 |
| 1301202 | 12700 | 12715 | N/A | N/A | ATGTAGCTGTGGCAAC | 66 | AI | 2500 |
| 1301228 | 23491 | 23506 | N/A | N/A | TGTAGAGGCTGCATGC | 30 | AI | 2501 |
| 1301238 | 25162 | 25177 | N/A | N/A | CGACCTATAAGGACCC | 28 | AI | 2502 |
| 1301263 | 18347 | 18362 | N/A | N/A | TTAACACTTGCTGTGC | 42 | AI | 2503 |
| 1301272 | 19269 | 19284 | N/A | N/A | AGACACTAGAGCCTCA | 35 | AI | 2504 |
| 1301347 | 25824 | 25839 | N/A | N/A | ATGATATGTTTACACC | 34 | AI | 2505 |
| 1232737 | 5138 | 5153 | 90 | 105 | ATGCCATCTTGACCCA | 77 | AJ | 22 |
| 1301177 | 27371 | 27386 | N/A | N/A | GCAGAGCATTTAGGAC | 80 | AJ | 2506 |
| 1301220 | 23281 | 23296 | N/A | N/A | TTATATGCCTCCAGTC | 68 | AJ | 2507 |
| 1301224 | 23306 | 23321 | N/A | N/A | ATTATCTTATTAGCAG | 53 | AJ | 2508 |
| 1301259 | 18755 | 18770 | N/A | N/A | TTTAAGCCACCGAACA | 102 | AJ | 2509 |
| 1301262 | 18346 | 18361 | N/A | N/A | TAACACTTGCTGTGCA | 68 | AJ | 2510 |
| 1301274 | 19884 | 19899 | N/A | N/A | TCTTGATTAGTTTCCC | 23 | AJ | 2511 |
| 1301281 | 20275 | 20290 | N/A | N/A | TGACGATTTCATCCCA | 30 | AJ | 2512 |
| 1301337 | 25821 | 25836 | N/A | N/A | ATATGTTTACACCAAG | 63 | AJ | 2513 |
| 1301346 | 26955 | 26970 | N/A | N/A | TGATTAATGCCACTTA | 31 | AJ | 2514 |
| 1232737 | 5138 | 5153 | 90 | 105 | ATGCCATCTTGACCCA | 64 | AK | 22 |
| 1301172 | 27094 | 27109 | N/A | N/A | GGCTATACCTAACACG | 28 | AK | 2515 |
| 1301176 | 10303 | 10318 | 1103 | 1118 | AAGGTACTCCAGTAAA | 60 | AK | 2516 |
| 1301218 | 17663 | 17678 | N/A | N/A | ATCGAGCATCATGGTT | 30 | AK | 2517 |
| 1301226 | 23500 | 23515 | N/A | N/A | CTTTCCTAGTGTAGAG | 65 | AK | 2518 |
| 1301255 | 17992 | 18007 | N/A | N/A | TCAACCCATACAAAGC | 50 | AK | 2519 |
| 1301295 | 22428 | 22443 | N/A | N/A | TAATGTCCCCGAGGCT | 41 | AK | 2520 |
| 1301308 | 24554 | 24569 | N/A | N/A | AGTCAAATTGCCAAGG | 45 | AK | 2521 |
| 1301336 | 25820 | 25835 | N/A | N/A | TATGTTTACACCAAGC | 16 | AK | 2522 |
| 1301344 | 26954 | 26969 | N/A | N/A | GATTAATGCCACTTAT | 56 | AK | 2523 |
| 1232737 | 5138 | 5153 | 90 | 105 | ATGCCATCTTGACCCA | 63 | AL | 22 |
| 1301170 | 27194 | 27209 | N/A | N/A | TGAGACAGGCAGTACA | 52 | AL | 2524 |
| 1301178 | 27370 | 27385 | N/A | N/A | CAGAGCATTTAGGACC | 80 | AL | 2525 |
| 1301184 | 11256 | 11271 | 1559 | 1574 | GATCAGGTCCCCCAGG | 81 | AL | 2526 |
| 1301205 | 16640 | 16655 | 2958 | 2973 | GACTCTGGCTGGTGCT | 71 | AL | 2527 |
| 1301231 | 11046 | 11061 | 1349 | 1364 | CTCCATCTTAATGGGA | 93 | AL | 2528 |
| 1301257 | 18754 | 18769 | N/A | N/A | TTAAGCCACCGAACAG | 69 | AL | 2529 |
| 1301275 | 20418 | 20433 | N/A | N/A | AGTGAAGCGGGCGGTG | 90 | AL | 2530 |
| 1301286 | 20591 | 20606 | N/A | N/A | ATACACCTGACCAGAG | 65 | AL | 2531 |
| 1301310 | 22905 | 22920 | N/A | N/A | CCATGGACAAGGAAGC | 71 | AL | 2532 |
| 1301348 | 26952 | 26967 | N/A | N/A | TTAATGCCACTTATAG | 73 | AL | 2533 |
| 1232737 | 5138 | 5153 | 90 | 105 | ATGCCATCTTGACCCA | 75 | AM | 22 |
| 1301169 | 27193 | 27208 | N/A | N/A | GAGACAGGCAGTACAC | 82 | AM | 2534 |
| 1301191 | 11263 | 11278 | 1566 | 1581 | AGCTCATGATCAGGTC | 49 | AM | 2535 |
| 1301208 | 15314 | 15329 | N/A | N/A | TCAGAGTAAGGCAGCT | 48 | AM | 2536 |
| 1301223 | 23280 | 23295 | N/A | N/A | TATATGCCTCCAGTCC | 60 | AM | 2537 |
| 1301269 | 19877 | 19892 | N/A | N/A | TAGTTTCCCCGTCACA | 64 | AM | 2538 |
| 1301296 | 22897 | 22912 | N/A | N/A | AAGGAAGCACCCGTAC | 114 | AM | 2539 |
| 1301312 | 22903 | 22918 | N/A | N/A | ATGGACAAGGAAGCAC | 68 | AM | 2540 |
| 1301316 | 26783 | 26798 | N/A | N/A | CCAATCGATAGATCTA | 44 | AM | 2541 |
| 1301331 | 27020 | 27035 | N/A | N/A | CTATACCTAACACACA | 98 | AM | 2542 |
| | 27056 | 27071 | | | | | | |
| 1301342 | 25740 | 25755 | N/A | N/A | AATTGAGAGGTATATG | 65 | AM | 2543 |
| 1232737 | 5138 | 5153 | 90 | 105 | ATGCCATCTTGACCCA | 87 | AN | 22 |
| 1301173 | 26961 | 26976 | N/A | N/A | CTCGACTGATTAATGC | 33 | AN | 2544 |
| 1301187 | 11955 | 11970 | 2258 | 2273 | TTGGAACAGGTTCATC | 94 | AN | 2545 |
| 1301188 | 5153 | 5168 | 105 | 120 | AACAACCACTTCACGA | 97 | AN | 2546 |
| 1301204 | 17662 | 17677 | N/A | N/A | TCGAGCATCATGGTTG | 17 | AN | 2547 |
| 1301209 | 15309 | 15324 | N/A | N/A | GTAAGGCAGCTGCCAT | 98 | AN | 2548 |
| 1301227 | 23532 | 23547 | N/A | N/A | TCTCCTAGTGTAGAGG | 80 | AN | 2549 |
| 1301288 | 20423 | 20438 | N/A | N/A | TTGTAAGTGAAGCGGG | 70 | AN | 2550 |
| 1232737 | 5138 | 5153 | 90 | 105 | ATGCCATCTTGACCCA | 55 | AO | 22 |
| 1301166 | 27134 | 27149 | N/A | N/A | ATGTAGCTATACCTAA | 88 | AO | 2551 |
| 1301210 | 15260 | 15275 | N/A | N/A | CTCCAGTCAGAGTCTG | 28 | AO | 2552 |
| 1301219 | 23279 | 23294 | N/A | N/A | ATATGCCTCCAGTCCT | 64 | AO | 2553 |
| 1301229 | 23499 | 23514 | N/A | N/A | TTTCCTAGTGTAGAGG | 49 | AO | 2554 |
| 1301248 | 17991 | 18006 | N/A | N/A | CAACCCATACAAAGCA | 49 | AO | 2555 |
| 1301251 | 17696 | 17711 | N/A | N/A | TCTATGAACATGGCCC | 35 | AO | 2556 |
| 1301279 | 20417 | 20432 | N/A | N/A | GTGAAGCGGGCGGTGT | 71 | AO | 2557 |
| 1232737 | 5138 | 5153 | 90 | 105 | ATGCCATCTTGACCCA | 65 | AP | 22 |
| 1301171 | 26960 | 26975 | N/A | N/A | TCGACTGATTAATGCC | 49 | AP | 2558 |
| 1301197 | 12705 | 12720 | N/A | N/A | TTATGATGTAGCTGTG | 89 | AP | 2559 |
| 1301221 | 21595 | 21610 | N/A | N/A | ATATGAGGACCCAGGG | 61 | AP | 2560 |
| 1301232 | 23531 | 23546 | N/A | N/A | CTCCTAGTGTAGAGGC | 55 | AP | 2561 |
| 1301250 | 17989 | 18004 | N/A | N/A | ACCCATACAAAGCATT | 55 | AP | 2562 |
| 1301260 | 18753 | 18768 | N/A | N/A | TAAGCCACCGAACAGC | 58 | AP | 2563 |
| 1301300 | 22896 | 22911 | N/A | N/A | AGGAAGCACCCGTACC | 89 | AP | 2564 |
| 1301304 | 22902 | 22917 | N/A | N/A | TGGACAAGGAAGCACC | 70 | AP | 2565 |
| 1301327 | 26788 | 26803 | N/A | N/A | ATGCCCCAATCGATAG | 82 | AP | 2566 |
| 1301334 | 35770 | 35785 | 4123 | 4138 | ATTACATGAGGTCACC | 65 | AP | 2567 |
| 1301345 | 26600 | 26615 | N/A | N/A | TATTTGGTGATACGGT | 36 | AP | 2568 |
| 1232737 | 5138 | 5153 | 90 | 105 | ATGCCATCTTGACCCA | 59 | AQ | 22 |
| 1301179 | 27363 | 27378 | N/A | N/A | TTTAGGACCGTCCCCC | 75 | AQ | 2569 |
| 1301211 | 15187 | 15202 | N/A | N/A | CTCCCGTCAGAGTCTG | 56 | AQ | 2570 |
| 1301339 | 25817 | 25832 | N/A | N/A | GTTTACACCAAGCATG | 66 | AQ | 2571 |
| 1232737 | 5138 | 5153 | 90 | 105 | ATGCCATCTTGACCCA | 65 | AR | 22 |
| 1301180 | 27369 | 27384 | N/A | N/A | AGAGCATTTAGGACCG | 53 | AR | 2572 |
| 1301207 | 17661 | 17676 | N/A | N/A | CGAGCATCATGGTTGT | 28 | AR | 2573 |
| 1301215 | 21594 | 21609 | N/A | N/A | TATGAGGACCCAGGGC | 51 | AR | 2574 |
| 1301235 | 25165 | 25180 | N/A | N/A | GTACGACCTATAAGGA | 91 | AR | 2575 |
| 1301246 | 17988 | 18003 | N/A | N/A | CCCATACAAAGCATTC | 64 | AR | 2576 |
| 1301264 | 19881 | 19896 | N/A | N/A | TGATTAGTTTCCCCGT | 57 | AR | 2577 |
| 1301268 | 19469 | 19484 | N/A | N/A | GATGAAGAGTGTGATC | 92 | AR | 2578 |
| 1301282 | 20416 | 20431 | N/A | N/A | TGAAGCGGGCGGTGTG | 84 | AR | 2579 |
| 1301285 | 20422 | 20437 | N/A | N/A | TGTAAGTGAAGCGGGC | 37 | AR | 2580 |
| 1301311 | 23818 | 23833 | N/A | N/A | TTATTAGTGGGTATTC | 74 | AR | 2581 |
| 1232737 | 5138 | 5153 | 90 | 105 | ATGCCATCTTGACCCA | 55 | AS | 22 |
| 1301182 | 27200 | 27215 | N/A | N/A | CAGTATTGAGACAGGC | 35 | AS | 2582 |
| 1301185 | 11982 | 11997 | 2285 | 2300 | GTAGAACTTCTCGCAG | 73 | AS | 2583 |
| 1301186 | 12470 | 12485 | 2773 | 2788 | AGGGACAGTGACTCCA | 90 | AS | 2584 |
| 1301233 | 11044 | 11059 | 1347 | 1362 | CCATCTTAATGGGACT | 56 | AS | 2585 |
| 1301261 | 18354 | 18369 | N/A | N/A | GCGGACTTTAACACTT | 47 | AS | 2586 |
| 1301287 | 20590 | 20605 | N/A | N/A | TACACCTGACCAGAGA | 40 | AS | 2587 |
| 1301338 | 25816 | 25831 | N/A | N/A | TTTACACCAAGCATGT | 94 | AS | 2588 |
| 1301341 | 31084 | 31099 | 3528 | 3543 | AGAGTAGTTTGATCCC | 53 | AS | 2589 |
| 1232737 | 5138 | 5153 | 90 | 105 | ATGCCATCTTGACCCA | 57 | AT | 22 |
| 1301199 | 14487 | 14502 | N/A | N/A | GAAATGGGTGCTGAGC | 25 | AT | 2590 |
| | 14633 | 14648 | | | | | | |
| | 14706 | 14721 | | | | | | |
| | 14779 | 14794 | | | | | | |
| | 14998 | 15013 | | | | | | |
| | 15071 | 15086 | | | | | | |
| 1301230 | 23299 | 23314 | N/A | N/A | TATTAGCAGCAGTATA | 75 | AT | 2591 |
| 1301239 | 25158 | 25173 | N/A | N/A | CTATAAGGACCCAGCC | 59 | AT | 2592 |
| 1301245 | 17444 | 17459 | N/A | N/A | ATTAACATTAGGTCTT | 40 | AT | 2593 |
| 1301283 | 20273 | 20288 | N/A | N/A | ACGATTTCATCCCAGC | 19 | AT | 2594 |
| 1301317 | 25038 | 25053 | N/A | N/A | AGGTTAGGTATTGACA | 32 | AT | 2595 |
| 1301325 | 26787 | 26802 | N/A | N/A | TGCCCCAATCGATAGA | 83 | AT | 2596 |
| 1301330 | 28765 | 28780 | N/A | N/A | CTGGTAACTATTAAGC | 77 | AT | 2597 |
| 1301333 | 27021 | 27036 | N/A | N/A | GCTATACCTAACACAC | 44 | AT | 2598 |
| | 27057 | 27072 | | | | | | |
| 1301335 | 25815 | 25830 | N/A | N/A | TTACACCAAGCATGTG | 60 | AT | 2599 |
| 1232737 | 5138 | 5153 | 90 | 105 | ATGCCATCTTGACCCA | 52 | AU | 22 |
| 1301190 | 11298 | 11313 | 1601 | 1616 | GATCTTGTGGATGGGT | 29 | AU | 2600 |
| 1301206 | 17660 | 17675 | N/A | N/A | GAGCATCATGGTTGTT | 29 | AU | 2601 |
| 1301214 | 23298 | 23313 | N/A | N/A | ATTAGCAGCAGTATAC | 50 | AU | 2602 |
| 1301236 | 25164 | 25179 | N/A | N/A | TACGACCTATAAGGAC | 53 | AU | 2603 |
| 1301249 | 17983 | 17998 | N/A | N/A | ACAAAGCATTCTGACC | 40 | AU | 2604 |
| 1301297 | 22901 | 22916 | N/A | N/A | GGACAAGGAAGCACCC | 55 | AU | 2605 |
| 1301309 | 23825 | 23840 | N/A | N/A | TGCATGATTATTAGTG | 67 | AU | 2606 |
| 1301340 | 25814 | 25829 | N/A | N/A | TACACCAAGCATGTGT | 103 | AU | 2607 |
| 1232737 | 5138 | 5153 | 90 | 105 | ATGCCATCTTGACCCA | 43 | AV | 22 |
| 1301212 | 16643 | 16658 | 2961 | 2976 | TTAGACTCTGGCTGGT | 47 | AV | 2608 |
| 1301217 | 23283 | 23298 | N/A | N/A | CATTATATGCCTCCAG | 34 | AV | 2609 |
| 1301225 | 23492 | 23507 | N/A | N/A | GTGTAGAGGCTGCATG | 43 | AV | 2610 |
| 1301241 | 23502 | 23517 | N/A | N/A | CTCTTTCCTAGTGTAG | 105 | AV | 2611 |
| 1301244 | 17982 | 17997 | N/A | N/A | CAAAGCATTCTGACCA | 42 | AV | 2612 |
| 1301270 | 19273 | 19288 | N/A | N/A | TGATAGACACTAGAGC | 63 | AV | 2613 |
| 1301271 | 12143 | 12158 | 2446 | 2461 | TACCCCTTTTCGAATT | 81 | AV | 2614 |
| 1301277 | 20272 | 20287 | N/A | N/A | CGATTTCATCCCAGCC | 30 | AV | 2615 |
| 1301320 | 25018 | 25033 | N/A | N/A | TAACTACATAGAGCAT | 74 | AV | 2616 |
| 1301321 | 26779 | 26794 | N/A | N/A | TCGATAGATCTACACA | 44 | AV | 2617 |
| 1301323 | 25037 | 25052 | N/A | N/A | GGTTAGGTATTGACAA | 40 | AV | 2618 |
| 1301332 | 26970 | 26985 | N/A | N/A | CATAAAAGTCTCGACT | 85 | AV | 2619 |
| 1232737 | 5138 | 5153 | 90 | 105 | ATGCCATCTTGACCCA | 65 | AW | 22 |
| 1301165 | 26958 | 26973 | N/A | N/A | GACTGATTAATGCCAC | 15 | AW | 2620 |
| 1301175 | 11001 | 11016 | 1304 | 1319 | GCCGATGGCCAGAAGC | 70 | AW | 2621 |
| 1301181 | 27199 | 27214 | N/A | N/A | AGTATTGAGACAGGCA | 45 | AW | 2622 |
| 1301189 | 11981 | 11996 | 2284 | 2299 | TAGAACTTCTCGCAGT | 66 | AW | 2623 |
| 1301192 | 11231 | 11246 | 1534 | 1549 | GTCACAAGGCTCACCT | 69 | AW | 2624 |
| 1301201 | 12703 | 12718 | N/A | N/A | ATGATGTAGCTGTGGC | 102 | AW | 2625 |
| 1301216 | 23282 | 23297 | N/A | N/A | ATTATATGCCTCCAGT | 58 | AW | 2626 |
| 1301247 | 17460 | 17475 | N/A | N/A | TTAAAGGAGTGACTAG | 48 | AW | 2627 |
| 1301278 | 20278 | 20293 | N/A | N/A | GTTTGACGATTTCATC | 33 | AW | 2628 |
| 1301291 | 12450 | 12465 | 2753 | 2768 | ATGACAGTTCTCAATG | 83 | AW | 2629 |
| 1301293 | 20420 | 20435 | N/A | N/A | TAAGTGAAGCGGGCGG | 58 | AW | 2630 |
| 1301294 | 12457 | 12472 | 2760 | 2775 | CCACCCGATGACAGTT | 82 | AW | 2631 |
| 1301299 | 22430 | 22445 | N/A | N/A | CATAATGTCCCCGAGG | 43 | AW | 2632 |
| 1301313 | 23824 | 23839 | N/A | N/A | GCATGATTATTAGTGG | 29 | AW | 2633 |
| 1232737 | 5138 | 5153 | 90 | 105 | ATGCCATCTTGACCCA | 43 | AX | 22 |
| 1301168 | 27131 | 27146 | N/A | N/A | TAGCTATACCTAACAC | 81 | AX | 2634 |
| 1301234 | 23501 | 23516 | N/A | N/A | TCTTTCCTAGTGTAGA | 78 | AX | 2635 |
| 1301240 | 25157 | 25172 | N/A | N/A | TATAAGGACCCAGCCT | 59 | AX | 2636 |
| 1301252 | 17459 | 17474 | N/A | N/A | TAAAGGAGTGACTAGA | 57 | AX | 2637 |
| 1301253 | 17443 | 17458 | N/A | N/A | TTAACATTAGGTCTTG | 43 | AX | 2638 |
| 1301258 | 18353 | 18368 | N/A | N/A | CGGACTTTAACACTTG | 44 | AX | 2639 |
| 1301266 | 19272 | 19287 | N/A | N/A | GATAGACACTAGAGCC | 24 | AX | 2640 |
| 1301303 | 20771 | 20786 | N/A | N/A | GAATGCATCAGGACAC | 71 | AX | 2641 |
| 1301319 | 26786 | 26801 | N/A | N/A | GCCCCAATCGATAGAT | 72 | AX | 2642 |
| 1301328 | 28764 | 28779 | N/A | N/A | TGGTAACTATTAAGCA | 66 | AX | 2643 |
| 1232737 | 5138 | 5153 | 90 | 105 | ATGCCATCTTGACCCA | 69 | AY | 22 |
| 1301183 | 27206 | 27221 | N/A | N/A | CTTGAGCAGTATTGAG | 77 | AY | 2644 |
| 1301242 | 25156 | 25171 | N/A | N/A | ATAAGGACCCAGCCTG | 77 | AY | 2645 |
| 1301254 | 18756 | 18771 | N/A | N/A | ATTTAAGCCACCGAAC | 96 | AY | 2646 |
| 1301267 | 19879 | 19894 | N/A | N/A | ATTAGTTTCCCCGTCA | 44 | AY | 2647 |
| 1301276 | 20271 | 20286 | N/A | N/A | GATTTCATCCCAGCCC | 47 | AY | 2648 |
| 1301302 | 20769 | 20784 | N/A | N/A | ATGCATCAGGACACAC | 61 | AY | 2649 |
| 1301305 | 25017 | 25032 | N/A | N/A | AACTACATAGAGCATA | 58 | AY | 2650 |
| 1301322 | 25036 | 25051 | N/A | N/A | GTTAGGTATTGACAAT | 66 | AY | 2651 |
| 1301326 | 26969 | 26984 | N/A | N/A | ATAAAAGTCTCGACTG | 75 | AY | 2652 |
| 1301329 | 28763 | 28778 | N/A | N/A | GGTAACTATTAAGCAA | 38 | AY | 2653 |
| 1232737 | 5138 | 5153 | 90 | 105 | ATGCCATCTTGACCCA | 63 | AZ | 22 |
| 1301195 | 12702 | 12717 | N/A | N/A | TGATGTAGCTGTGGCA | 44 | AZ | 2654 |
| 1301196 | 5283 | 5298 | 235 | 250 | ATGCTTACCAGAAAGT | 40 | AZ | 2655 |
| 1301213 | 15183 | 15198 | N/A | N/A | CGTCAGAGTCTGTCCT | 24 | AZ | 2656 |
| 1301237 | 25163 | 25178 | N/A | N/A | ACGACCTATAAGGACC | 46 | AZ | 2657 |
| 1301265 | 19270 | 19285 | N/A | N/A | TAGACACTAGAGCCTC | 23 | AZ | 2658 |
| 1301289 | 20426 | 20441 | N/A | N/A | GGTTTGTAAGTGAAGC | 30 | AZ | 2659 |
| 1301306 | 25016 | 25031 | N/A | N/A | ACTACATAGAGCATAT | 65 | AZ | 2660 |
| 1301315 | 26785 | 26800 | N/A | N/A | CCCCAATCGATAGATC | 65 | AZ | 2661 |
| 1301343 | 29466 | 29481 | N/A | N/A | CTTGAGTCAAATTAAG | 99 | AZ | 2662 |

TABLE 3
Reduction of NLRP3 RNA by 3-10-3 cEt modified
oligonucleotides with uniform phosphorothioate
internucleoside linkages at a concentration
of 2000 nM in THP-1 cells

| | SEQ | SEQ | SEQ | SEQ | SEQ | SEQ | | | | |
|---|---|---|---|---|---|---|---|---|---|---|
| | ID | ID | ID | ID | ID | ID | | | | |
| | No: 3 | No: 3 | No: 4 | No: 4 | No: 5 | No: 5 | | NLRP3 | | SEQ |
| Compound | Start | Stop | Start | Stop | Start | Stop | Sequence | (% | | ID |
| Number | Site | Site | Site | Site | Site | Site | (5′ to 3′) | UTC) | AID | NO |
| 1242249 | 3065 | 3080 | 3270 | 3285 | N/A | N/A | CACCAACCAGAGCTTC | 72 | I | 672 |
| 1242250 | 3068 | 3083 | 3273 | 3288 | N/A | N/A | ATTCACCAACCAGAGC | 76 | J | 749 |
| 1242251 | 3069 | 3084 | 3274 | 3289 | N/A | N/A | AATTCACCAACCAGAG | 91 | K | 826 |
| 1242252 | 3070 | 3085 | 3275 | 3290 | N/A | N/A | GAATTCACCAACCAGA | 63 | L | 903 |
| 1242257 | N/A | N/A | 25 | 40 | N/A | N/A | ATCCAGATGCCAGCCT | 75 | Q | 1288 |
| 1242258 | N/A | N/A | 30 | 45 | N/A | N/A | TCCTCATCCAGATGCC | 74 | R | 1365 |
| 1242259 | N/A | N/A | 31 | 46 | N/A | N/A | TTCCTCATCCAGATGC | 63 | S | 1442 |
| 1242260 | N/A | N/A | N/A | N/A | 32 | 47 | AAGGTTTCCCCAGATG | 92 | T | 1519 |
| 1242261 | N/A | N/A | N/A | N/A | 37 | 52 | GAAGAAAGGTTTCCCC | 77 | U | 1596 |
| 1242246 | 2890 | 2905 | N/A | N/A | N/A | N/A | AACCCATGAGAACAGG | 75 | AC | 2211 |
| 1242247 | 2893 | 2908 | N/A | N/A | N/A | N/A | CCCAACCCATGAGAAC | 98 | AD | 2288 |
| 1242248 | 2894 | 2909 | N/A | N/A | N/A | N/A | CCCCAACCCATGAGAA | 88 | AE | 2366 |

### Example 2: Effect of Mixed MOE and cEt, Uniform Phosphorothioate Modified Oligonucleotides on Human NLRP3 RNA In Vitro, Single Dose

Modified oligonucleotides complementary to human NLRP3 nucleic acid were designed and tested for their single dose effects on NLRP3 RNA in vitro. The modified oligonucleotides were tested in a series of experiments that had the same culture conditions.

The modified oligonucleotides in the table below are 16 nucleosides in length, wherein the sugar motif for the modified oligonucleotides is (from 5′ to 3′): kekdddddddddeekk; wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. “N/A” indicates that the modified oligonucleotide is not 100% complementary to that particular target nucleic acid sequence.

Cultured THP-1 cells were treated with modified oligonucleotide at a concentration of 2000 nM by electroporation at a density of 300,000 cells per well. After a treatment period of approximately 24 hours, total RNA was isolated from the cells and NLRP3 RNA levels were measured by quantitative real-time RTPCR. NLRP3 RNA levels were measured by human primer-probe set RTS37509 (described herein above). NLRP3 RNA levels were normalized to total RNA content, as measured by RIBOGREEN®. Reduction of NLRP3 RNA is presented in the tables below as percent NLRP3 RNA relative to the amount of NLRP3 RNA in untreated control cells (% UTC).

Each separate experimental analysis described in this example is identified by a letter ID in the table column below labeled “AID” (Analysis ID). In the table below, Compound No. 1232737 (described herein above) was used as a benchmark on multiple plates.

TABLE 4
Reduction of NLRP3 RNA by modified oligonucleotides
with a mixedMOE/cEt sugar motif and uniform
phosphorothioate internucleosidelinkages at a
concentration of 2000 nM in THP-1 cells

| | SEQ ID | SEQ | SEQ ID | SEQ ID | | | | |
|---|---|---|---|---|---|---|---|---|
| | No: 1 | ID No: | No: 2 | No: 2 | | NLRP3 | | SEQ |
| Compound | Start | 1 Stop | Start | Stop | Sequence | (% | | ID |
| Number | Site | Site | Site | Site | (5′ to 3′) | UTC) | AID | NO |
| 1232737 | 5138 | 5153 | 90 | 105 | ATGCCATCTTGACCCA | 60 | AH | 22 |
| 1299495 | 5249 | 5264 | 201 | 216 | CAGGAGTGTGTCCTGA | 100 | AH | 2663 |
| 1299554 | 10928 | 10943 | 1231 | 1246 | CGTTTGTTGAGGCTCA | 38 | AH | 1417 |
| 1299568 | 11259 | 11274 | 1562 | 1577 | CATGATCAGGTCCCCC | 48 | AH | 1957 |
| 1299584 | 11296 | 11311 | 1599 | 1614 | TCTTGTGGATGGGTGG | 62 | AH | 2188 |
| 1299585 | 11302 | 11317 | 1605 | 1620 | TCACGATCTTGTGGAT | 73 | AH | 803 |
| 1299614 | 12276 | 12291 | 2579 | 2594 | GGCTTTCACTTCAATC | 52 | AH | 2664 |
| 1299642 | 14574 | 14589 | N/A | N/A | AGGTGCCATCTAGAGG | 19 | AH | 1528 |
| | 14866 | 14881 | | | | | | |
| | 15156 | 15171 | | | | | | |
| | 15229 | 15244 | | | | | | |
| 1299648 | 16737 | 16752 | 3055 | 3070 | AATCTCCGAATGTTAC | 94 | AH | 271 |
| 1299651 | 16730 | 16745 | 3048 | 3063 | GAATGTTACAGCCAGG | 55 | AH | 2275 |
| 1299693 | 11041 | 11056 | 1344 | 1359 | TCTTAATGGGACTCAC | 61 | AH | 263 |
| 1299700 | 23281 | 23296 | N/A | N/A | TTATATGCCTCCAGTC | 66 | AH | 2507 |
| 1299713 | 23491 | 23506 | N/A | N/A | TGTAGAGGCTGCATGC | 32 | AH | 2501 |
| 1299724 | 23496 | 23511 | N/A | N/A | CCTAGTGTAGAGGCTG | 36 | AH | 77 |
| | 23529 | 23544 | | | | | | |
| 1299730 | 25162 | 25177 | N/A | N/A | CGACCTATAAGGACCC | 37 | AH | 2502 |
| 1299767 | 18352 | 18367 | N/A | N/A | GGACTTTAACACTTGC | 27 | AH | 2665 |
| 1299795 | 19879 | 19894 | N/A | N/A | ATTAGTTTCCCCGTCA | 45 | AH | 2647 |
| 1299863 | 24554 | 24569 | N/A | N/A | AGTCAAATTGCCAAGG | 44 | AH | 2521 |
| 1299869 | 25079 | 25094 | N/A | N/A | AGGCCGCTCTGTTTGT | 72 | AH | 2666 |
| 1299881 | 26785 | 26800 | N/A | N/A | CCCCAATCGATAGATC | 105 | AH | 2661 |
| 1299889 | 27026 | 27041 | N/A | N/A | CATAGGCTATACCTAA | 69 | AH | 393 |
| | 27062 | 27077 | | | | | | |
| | 27098 | 27113 | | | | | | |
| | 27116 | 27131 | | | | | | |
| 1299898 | 28755 | 28770 | N/A | N/A | TTAAGCAACGGTTACA | 56 | AH | 1472 |
| 1299909 | 28763 | 28778 | N/A | N/A | GGTAACTATTAAGCAA | 58 | AH | 2653 |
| 1299916 | 31081 | 31096 | 3525 | 3540 | GTAGTTTGATCCCCTT | 56 | AH | 1897 |
| 1232737 | 5138 | 5153 | 90 | 105 | ATGCCATCTTGACCCA | 54 | AI | 22 |
| 1299497 | 27094 | 27109 | N/A | N/A | GGCTATACCTAACACG | 41 | AI | 2515 |
| 1299500 | 26962 | 26977 | N/A | N/A | TCTCGACTGATTAATG | 46 | AI | 2667 |
| 1299503 | 26957 | 26972 | N/A | N/A | ACTGATTAATGCCACT | 33 | AI | 1392 |
| 1299556 | 5147 | 5162 | 99 | 114 | CACTTCACGATGCCAT | 56 | AI | 330 |
| 1299588 | 11972 | 11987 | 2275 | 2290 | TCGCAGTCCACTTCCT | 74 | AI | 2668 |
| 1299598 | 11979 | 11994 | 2282 | 2297 | GAACTTCTCGCAGTCC | 40 | AI | 1114 |
| 1299611 | 12463 | 12478 | 2766 | 2781 | GTGACTCCACCCGATG | 51 | AI | 2272 |
| 1299646 | 16727 | 16742 | 3045 | 3060 | TGTTACAGCCAGGATG | 74 | AI | 2669 |
| 1299647 | 16641 | 16656 | 2959 | 2974 | AGACTCTGGCTGGTGC | 66 | AI | 2670 |
| 1299659 | 15111 | 15126 | N/A | N/A | CAGTCAGAGTCTGTCC | 18 | AI | 914 |
| | 15257 | 15272 | | | | | | |
| 1299719 | 23500 | 23515 | N/A | N/A | CTTTCCTAGTGTAGAG | 80 | AI | 2518 |
| 1299745 | 23038 | 23053 | 3313 | 3328 | TAGAGTCTGGTCAGGG | 57 | AI | 1893 |
| 1299764 | 17448 | 17463 | N/A | N/A | CTAGATTAACATTAGG | 57 | AI | 915 |
| 1299769 | 12140 | 12155 | 2443 | 2458 | CCCTTTTCGAATTTGC | 34 | AI | 497 |
| 1299798 | 19887 | 19902 | N/A | N/A | GATTCTTGATTAGTTT | 50 | AI | 2671 |
| 1299809 | 20276 | 20291 | N/A | N/A | TTGACGATTTCATCCC | 39 | AI | 2672 |
| 1299810 | 20419 | 20434 | N/A | N/A | AAGTGAAGCGGGCGGT | 84 | AI | 2490 |
| 1299841 | 22429 | 22444 | N/A | N/A | ATAATGTCCCCGAGGC | 57 | AI | 2493 |
| 1299852 | 23822 | 23837 | N/A | N/A | ATGATTATTAGTGGGT | 79 | AI | 2463 |
| 1299855 | 14500 | 14515 | N/A | N/A | GGTGCTATCTAGAGAA | 37 | AI | 1604 |
| | 14646 | 14661 | | | | | | |
| | 14719 | 14734 | | | | | | |
| | 14792 | 14807 | | | | | | |
| | 14938 | 14953 | | | | | | |
| | 15011 | 15026 | | | | | | |
| | 15084 | 15099 | | | | | | |
| 1299890 | 27022 | 27037 | N/A | N/A | GGCTATACCTAACACA | 42 | AI | 162 |
| | 27058 | 27073 | | | | | | |
| | 27112 | 27127 | | | | | | |
| 1299929 | 25745 | 25760 | N/A | N/A | GTTGAAATTGAGAGGT | 37 | AI | 1235 |
| 1232737 | 5138 | 5153 | 90 | 105 | ATGCCATCTTGACCCA | 77 | AJ | 22 |
| 1299510 | 27195 | 27210 | N/A | N/A | TTGAGACAGGCAGTAC | 81 | AJ | 2673 |
| 1299520 | 27365 | 27380 | N/A | N/A | CATTTAGGACCGTCCC | 49 | AJ | 164 |
| 1299530 | 6018 | 6033 | 970 | 985 | CTGTTGATCGCAGCGA | 65 | AJ | 2183 |
| 1299535 | 6025 | 6040 | 977 | 992 | GTCTCTCCTGTTGATC | 73 | AJ | 2674 |
| 1299537 | 10303 | 10318 | 1103 | 1118 | AAGGTACTCCAGTAAA | 68 | AJ | 2516 |
| 1299551 | 5146 | 5161 | 98 | 113 | ACTTCACGATGCCATC | 50 | AJ | 715 |
| 1299561 | 11007 | 11022 | 1310 | 1325 | GGTCTTGCCGATGGCC | 65 | AJ | 2675 |
| 1299583 | 11264 | 11279 | 1567 | 1582 | CAGCTCATGATCAGGT | 60 | AJ | 2676 |
| 1299601 | 11978 | 11993 | 2281 | 2296 | AACTTCTCGCAGTCCA | 41 | AJ | 1037 |
| 1299613 | 5166 | 5181 | 118 | 133 | ACAGTTTACGGTGAAC | 53 | AJ | 562 |
| 1299640 | 14678 | 14693 | N/A | N/A | CTCCAATCAGAGTCTG | 56 | AJ | 1991 |
| | 14824 | 14839 | | | | | | |
| | 14970 | 14985 | | | | | | |
| 1299677 | 11040 | 11055 | 1343 | 1358 | CTTAATGGGACTCACG | 58 | AJ | 186 |
| 1299681 | 17670 | 17685 | N/A | N/A | GGAATATATCGAGCAT | 36 | AJ | 1377 |
| 1299686 | 20704 | 20719 | N/A | N/A | GAGAGCTCCGGAATAG | 57 | AJ | 842 |
| | 20746 | 20761 | | | | | | |
| | 20914 | 20929 | | | | | | |
| 1299714 | 5137 | 5152 | 89 | 104 | TGCCATCTTGACCCAT | 71 | AJ | 2408 |
| 1299716 | 11046 | 11061 | 1349 | 1364 | CTCCATCTTAATGGGA | 90 | AJ | 2528 |
| 1299759 | 17697 | 17712 | N/A | N/A | ATCTATGAACATGGCC | 43 | AJ | 1839 |
| 1299815 | 20592 | 20607 | N/A | N/A | AATACACCTGACCAGA | 81 | AJ | 1457 |
| 1299828 | 12455 | 12470 | 2758 | 2773 | ACCCGATGACAGTTCT | 43 | AJ | 2195 |
| 1299839 | 22898 | 22913 | N/A | N/A | CAAGGAAGCACCCGTA | 72 | AJ | 2492 |
| 1299870 | 25077 | 25092 | N/A | N/A | GCCGCTCTGTTTGTGA | 60 | AJ | 2677 |
| 1299879 | 26784 | 26799 | N/A | N/A | CCCAATCGATAGATCT | 47 | AJ | 468 |
| 1299899 | 28762 | 28777 | N/A | N/A | GTAACTATTAAGCAAC | 78 | AJ | 1626 |
| 1299912 | 29463 | 29478 | N/A | N/A | GAGTCAAATTAAGCCC | 53 | AJ | 2495 |
| 1299915 | 31018 | 31033 | 3462 | 3477 | TAGTGCTGAGTACCGA | 68 | AJ | 1049 |
| 1299932 | 35773 | 35788 | 4126 | 4141 | CTAATTACATGAGGTC | 49 | AJ | 2364 |
| 1299939 | 5247 | 5262 | 199 | 214 | GGAGTGTGTCCTGAGC | 57 | AJ | 2255 |
| 1232737 | 5138 | 5153 | 90 | 105 | ATGCCATCTTGACCCA | 64 | AK | 22 |
| 1299564 | 11006 | 11021 | 1309 | 1324 | GTCTTGCCGATGGCCA | 47 | AK | 1956 |
| 1299571 | 11258 | 11273 | 1561 | 1576 | ATGATCAGGTCCCCCA | 50 | AK | 1880 |
| 1299591 | 11955 | 11970 | 2258 | 2273 | TTGGAACAGGTTCATC | 71 | AK | 2545 |
| 1299604 | 5153 | 5168 | 105 | 120 | AACAACCACTTCACGA | 75 | AK | 2546 |
| 1299639 | 14679 | 14694 | N/A | N/A | GCTCCAATCAGAGTCT | 64 | AK | 2068 |
| | 14825 | 14840 | | | | | | |
| | 14971 | 14986 | | | | | | |
| 1299649 | 16735 | 16750 | 3053 | 3068 | TCTCCGAATGTTACAG | 69 | AK | 117 |
| 1299664 | 5136 | 5151 | 88 | 103 | GCCATCTTGACCCATC | 38 | AK | 2678 |
| 1299688 | 21597 | 21612 | N/A | N/A | CAATATGAGGACCCAG | 52 | AK | 2305 |
| 1299705 | 23495 | 23510 | N/A | N/A | CTAGTGTAGAGGCTGC | 17 | AK | 615 |
| | 23528 | 23543 | | | | | | |
| 1299717 | 23533 | 23548 | N/A | N/A | CTCTCCTAGTGTAGAG | 95 | AK | 2488 |
| 1299735 | 23000 | 23015 | 3275 | 3290 | TGCAAGATCCTGACAA | 70 | AK | 2046 |
| 1299738 | 12133 | 12148 | 2436 | 2451 | CGAATTTGCCATAGTT | 33 | AK | 2423 |
| 1299766 | 18755 | 18770 | N/A | N/A | TTTAAGCCACCGAACA | 74 | AK | 2509 |
| 1299779 | 18856 | 18871 | N/A | N/A | TGCAGTAACATATCCA | 37 | AK | 1532 |
| 1299803 | 19878 | 19893 | N/A | N/A | TTAGTTTCCCCGTCAC | 63 | AK | 2679 |
| 1299822 | 20424 | 20439 | N/A | N/A | TTTGTAAGTGAAGCGG | 34 | AK | 2491 |
| 1299858 | 25034 | 25049 | N/A | N/A | TAGGTATTGACAATAG | 53 | AK | 2494 |
| 1299891 | 27023 | 27038 | N/A | N/A | AGGCTATACCTAACAC | 36 | AK | 1546 |
| | 27059 | 27074 | | | | | | |
| | 27095 | 27110 | | | | | | |
| | 27113 | 27128 | | | | | | |
| 1299910 | 29462 | 29477 | N/A | N/A | AGTCAAATTAAGCCCA | 39 | AK | 245 |
| 1299927 | 25740 | 25755 | N/A | N/A | AATTGAGAGGTATATG | 79 | AK | 2543 |
| 1299935 | 26602 | 26617 | N/A | N/A | CTTATTTGGTGATACG | 43 | AK | 1237 |
| 1232737 | 5138 | 5153 | 90 | 105 | ATGCCATCTTGACCCA | 63 | AL | 22 |
| 1299501 | 26961 | 26976 | N/A | N/A | CTCGACTGATTAATGC | 62 | AL | 2544 |
| 1299515 | 27202 | 27217 | N/A | N/A | AGCAGTATTGAGACAG | 55 | AL | 2497 |
| 1299521 | 27364 | 27379 | N/A | N/A | ATTTAGGACCGTCCCC | 89 | AL | 87 |
| 1299525 | 6017 | 6032 | 969 | 984 | TGTTGATCGCAGCGAA | 48 | AL | 2106 |
| 1299553 | 10927 | 10942 | 1230 | 1245 | GTTTGTTGAGGCTCAC | 63 | AL | 2680 |
| 1299593 | 11301 | 11316 | 1604 | 1619 | CACGATCTTGTGGATG | 69 | AL | 726 |
| 1299605 | 12275 | 12290 | 2578 | 2593 | GCTTTCACTTCAATCC | 44 | AL | 1347 |
| 1299623 | 12491 | 12506 | 2794 | 2809 | GGCATGTTATGGAGAA | 72 | AL | 1118 |
| 1299638 | 14680 | 14695 | N/A | N/A | GGCTCCAATCAGAGTC | 91 | AL | 2453 |
| | 14826 | 14841 | | | | | | |
| | 14972 | 14987 | | | | | | |
| 1299656 | 15115 | 15130 | N/A | N/A | GTTCCAGTCAGAGTCT | 39 | AL | 2681 |
| 1299698 | 23280 | 23295 | N/A | N/A | TATATGCCTCCAGTCC | 68 | AL | 2537 |
| 1299723 | 23532 | 23547 | N/A | N/A | TCTCCTAGTGTAGAGG | 72 | AL | 2549 |
| 1299763 | 17447 | 17462 | N/A | N/A | TAGATTAACATTAGGT | 90 | AL | 838 |
| 1299785 | 19269 | 19284 | N/A | N/A | AGACACTAGAGCCTCA | 55 | AL | 2504 |
| 1299801 | 19883 | 19898 | N/A | N/A | CTTGATTAGTTTCCCC | 31 | AL | 1764 |
| 1299812 | 20275 | 20290 | N/A | N/A | TGACGATTTCATCCCA | 33 | AL | 2512 |
| 1299823 | 20423 | 20438 | N/A | N/A | TTGTAAGTGAAGCGGG | 88 | AL | 2550 |
| 1299838 | 22897 | 22912 | N/A | N/A | AAGGAAGCACCCGTAC | 102 | AL | 2539 |
| 1299856 | 24553 | 24568 | N/A | N/A | GTCAAATTGCCAAGGA | 55 | AL | 1003 |
| 1299857 | 25032 | 25047 | N/A | N/A | GGTATTGACAATAGTA | 29 | AL | 2682 |
| 1299868 | 25076 | 25091 | N/A | N/A | CCGCTCTGTTTGTGAT | 94 | AL | 2683 |
| 1299893 | 27021 | 27036 | N/A | N/A | GCTATACCTAACACAC | 64 | AL | 2598 |
| | 27057 | 27072 | | | | | | |
| 1299923 | 31080 | 31095 | 3524 | 3539 | TAGTTTGATCCCCTTG | 63 | AL | 1820 |
| 1299938 | 26601 | 26616 | N/A | N/A | TTATTTGGTGATACGG | 36 | AL | 1160 |
| 1299941 | 25820 | 25835 | N/A | N/A | TATGTTTACACCAAGC | 22 | AL | 2522 |
| 1232737 | 5138 | 5153 | 90 | 105 | ATGCCATCTTGACCCA | 75 | AM | 22 |
| 1299532 | 27370 | 27385 | N/A | N/A | CAGAGCATTTAGGACC | 79 | AM | 2525 |
| 1299542 | 6023 | 6038 | 975 | 990 | CTCTCCTGTTGATCGC | 58 | AM | 798 |
| 1299548 | 10925 | 10940 | 1228 | 1243 | TTGTTGAGGCTCACAC | 79 | AM | 2684 |
| 1299552 | 5145 | 5160 | 97 | 112 | CTTCACGATGCCATCT | 56 | AM | 253 |
| 1299596 | 11983 | 11998 | 2286 | 2301 | TGTAGAACTTCTCGCA | 46 | AM | 2685 |
| 1299669 | 17663 | 17678 | N/A | N/A | ATCGAGCATCATGGTT | 31 | AM | 2517 |
| 1299683 | 17669 | 17684 | N/A | N/A | GAATATATCGAGCATC | 30 | AM | 68 |
| 1299689 | 21596 | 21611 | N/A | N/A | AATATGAGGACCCAGG | 51 | AM | 151 |
| 1299712 | 23305 | 23320 | N/A | N/A | TTATCTTATTAGCAGC | 55 | AM | 922 |
| 1299722 | 23499 | 23514 | N/A | N/A | TTTCCTAGTGTAGAGG | 76 | AM | 2554 |
| 1299728 | 25161 | 25176 | N/A | N/A | GACCTATAAGGACCCA | 38 | AM | 81 |
| 1299737 | 23035 | 23050 | 3310 | 3325 | AGTCTGGTCAGGGAAT | 33 | AM | 2686 |
| 1299762 | 17696 | 17711 | N/A | N/A | TCTATGAACATGGCCC | 46 | AM | 2556 |
| 1299773 | 12139 | 12154 | 2442 | 2457 | CCTTTTCGAATTTGCC | 29 | AM | 420 |
| 1299780 | 18760 | 18775 | N/A | N/A | AAGCATTTAAGCCACC | 49 | AM | 1301 |
| 1299811 | 20418 | 20433 | N/A | N/A | AGTGAAGCGGGCGGTG | 92 | AM | 2530 |
| 1299820 | 20591 | 20606 | N/A | N/A | ATACACCTGACCAGAG | 82 | AM | 2531 |
| 1299831 | 12454 | 12469 | 2757 | 2772 | CCCGATGACAGTTCTC | 52 | AM | 268 |
| 1299842 | 22427 | 22442 | N/A | N/A | AATGTCCCCGAGGCTC | 62 | AM | 2687 |
| 1299880 | 26789 | 26804 | N/A | N/A | TATGCCCCAATCGATA | 73 | AM | 2688 |
| 1299896 | 27107 | 27122 | N/A | N/A | TACCTAACACATAGGC | 72 | AM | 2470 |
| | 27125 | 27140 | | | | | | |
| 1299924 | 31017 | 31032 | 3461 | 3476 | AGTGCTGAGTACCGAG | 35 | AM | 972 |
| 1232737 | 5138 | 5153 | 90 | 105 | ATGCCATCTTGACCCA | 87 | AN | 22 |
| 1299508 | 6015 | 6030 | 967 | 982 | TTGATCGCAGCGAAGA | 74 | AN | 2029 |
| 1299523 | 27201 | 27216 | N/A | N/A | GCAGTATTGAGACAGG | 41 | AN | 931 |
| 1299538 | 10302 | 10317 | 1102 | 1117 | AGGTACTCCAGTAAAC | 74 | AN | 2689 |
| 1299557 | 11013 | 11028 | 1316 | 1331 | CGTCTTGGTCTTGCCG | 73 | AN | 2690 |
| 1299562 | 11005 | 11020 | 1308 | 1323 | TCTTGCCGATGGCCAG | 68 | AN | 2691 |
| 1299573 | 11235 | 11250 | 1538 | 1553 | CTGTGTCACAAGGCTC | 48 | AN | 1572 |
| 1299576 | 11263 | 11278 | 1566 | 1581 | AGCTCATGATCAGGTC | 69 | AN | 2535 |
| 1299606 | 12462 | 12477 | 2765 | 2780 | TGACTCCACCCGATGA | 100 | AN | 2692 |
| 1299622 | 12487 | 12502 | 2790 | 2805 | TGTTATGGAGAAACCC | 64 | AN | 576 |
| 1299628 | 5172 | 5187 | 124 | 139 | TGTATTACAGTTTACG | 66 | AN | 255 |
| 1299636 | 14681 | 14696 | N/A | N/A | GGGCTCCAATCAGAGT | 95 | AN | 2145 |
| | 14827 | 14842 | | | | | | |
| | 14973 | 14988 | | | | | | |
| 1299662 | 16647 | 16662 | 2965 | 2980 | TCAGTTAGACTCTGGC | 38 | AN | 1505 |
| 1299707 | 11045 | 11060 | 1348 | 1363 | TCCATCTTAATGGGAC | 73 | AN | 2693 |
| 1299736 | 23033 | 23048 | 3308 | 3323 | TCTGGTCAGGGAATGG | 68 | AN | 1662 |
| 1299768 | 18753 | 18768 | N/A | N/A | TAAGCCACCGAACAGC | 67 | AN | 2563 |
| 1299774 | 17992 | 18007 | N/A | N/A | TCAACCCATACAAAGC | 68 | AN | 2519 |
| 1299800 | 12145 | 12160 | 2448 | 2463 | AATACCCCTTTTCGAA | 96 | AN | 36 |
| 1299805 | 20274 | 20289 | N/A | N/A | GACGATTTCATCCCAG | 15 | AN | 1918 |
| 1299849 | 23821 | 23836 | N/A | N/A | TGATTATTAGTGGGTA | 107 | AN | 2078 |
| 1299882 | 26783 | 26798 | N/A | N/A | CCAATCGATAGATCTA | 62 | AN | 2541 |
| 1299892 | 26973 | 26988 | N/A | N/A | ATGCATAAAAGTCTCG | 39 | AN | 85 |
| 1299895 | 28761 | 28776 | N/A | N/A | TAACTATTAAGCAACG | 86 | AN | 90 |
| 1299900 | 27111 | 27126 | N/A | N/A | GCTATACCTAACACAT | 93 | AN | 2162 |
| | 27129 | 27144 | | | | | | |
| 1299920 | 31079 | 31094 | 3523 | 3538 | AGTTTGATCCCCTTGT | 67 | AN | 1743 |
| 1232737 | 5138 | 5153 | 90 | 105 | ATGCCATCTTGACCCA | 55 | AO | 22 |
| 1299504 | 5133 | 5148 | 85 | 100 | ATCTTGACCCATCAGC | 77 | AO | 21 |
| 1299513 | 27106 | 27121 | N/A | N/A | ACCTAACACATAGGCT | 66 | AO | 2085 |
| | 27124 | 27139 | | | | | | |
| 1299539 | 10301 | 10316 | 1101 | 1116 | GGTACTCCAGTAAACC | 67 | AO | 2694 |
| 1299579 | 11300 | 11315 | 1603 | 1618 | ACGATCTTGTGGATGG | 38 | AO | 649 |
| 1299603 | 11977 | 11992 | 2280 | 2295 | ACTTCTCGCAGTCCAC | 55 | AO | 960 |
| 1299634 | 12709 | 12724 | N/A | N/A | GGCATTATGATGTAGC | 63 | AO | 832 |
| 1299635 | 14580 | 14595 | N/A | N/A | TAAGGCAGGTGCCATC | 38 | AO | 1913 |
| | 14872 | 14887 | | | | | | |
| | 15162 | 15177 | | | | | | |
| | 15235 | 15250 | | | | | | |
| 1299653 | 16734 | 16749 | 3052 | 3067 | CTCCGAATGTTACAGC | 68 | AO | 40 |
| 1299672 | 17662 | 17677 | N/A | N/A | TCGAGCATCATGGTTG | 37 | AO | 2547 |
| 1299687 | 20584 | 20599 | N/A | N/A | TGACCAGAGAGCTCCG | 33 | AO | 150 |
| | 20710 | 20725 | | | | | | |
| | 20752 | 20767 | | | | | | |
| | 20920 | 20935 | | | | | | |
| 1299709 | 23494 | 23509 | N/A | N/A | TAGTGTAGAGGCTGCA | 22 | AO | 538 |
| | 23527 | 23542 | | | | | | |
| 1299729 | 25160 | 25175 | N/A | N/A | ACCTATAAGGACCCAG | 58 | AO | 619 |
| 1299783 | 18759 | 18774 | N/A | N/A | AGCATTTAAGCCACCG | 33 | AO | 1224 |
| 1299786 | 19877 | 19892 | N/A | N/A | TAGTTTCCCCGTCACA | 47 | AO | 2538 |
| 1299791 | 19268 | 19283 | N/A | N/A | GACACTAGAGCCTCAG | 32 | AO | 1686 |
| 1299885 | 26972 | 26987 | N/A | N/A | TGCATAAAAGTCTCGA | 34 | AO | 1469 |
| 1299930 | 35770 | 35785 | 4123 | 4138 | ATTACATGAGGTCACC | 51 | AO | 2567 |
| 1299940 | 26600 | 26615 | N/A | N/A | TATTTGGTGATACGGT | 37 | AO | 2568 |
| 1299948 | 16646 | 16661 | 2964 | 2979 | CAGTTAGACTCTGGCT | 56 | AO | 1428 |
| 1232737 | 5138 | 5153 | 90 | 105 | ATGCCATCTTGACCCA | 65 | AP | 22 |
| 1299518 | 27363 | 27378 | N/A | N/A | TTTAGGACCGTCCCCC | 83 | AP | 2569 |
| 1299522 | 27200 | 27215 | N/A | N/A | CAGTATTGAGACAGGC | 54 | AP | 2582 |
| 1299543 | 5143 | 5158 | 95 | 110 | TCACGATGCCATCTTG | 70 | AP | 2332 |
| 1299575 | 5151 | 5166 | 103 | 118 | CAACCACTTCACGATG | 79 | AP | 946 |
| 1299597 | 11982 | 11997 | 2285 | 2300 | GTAGAACTTCTCGCAG | 63 | AP | 2583 |
| 1299612 | 12461 | 12476 | 2764 | 2779 | GACTCCACCCGATGAC | 57 | AP | 2695 |
| 1299676 | 17668 | 17683 | N/A | N/A | AATATATCGAGCATCA | 41 | AP | 606 |
| 1299680 | 17675 | 17690 | N/A | N/A | GAAATGGAATATATCG | 101 | AP | 1608 |
| 1299701 | 21604 | 21619 | N/A | N/A | TCTTATACAATATGAG | 65 | AP | 305 |
| 1299710 | 23303 | 23318 | N/A | N/A | ATCTTATTAGCAGCAG | 37 | AP | 76 |
| 1299721 | 23498 | 23513 | N/A | N/A | TTCCTAGTGTAGAGGC | 42 | AP | 2696 |
| 1299744 | 25166 | 25181 | N/A | N/A | TGTACGACCTATAAGG | 111 | AP | 2697 |
| 1299747 | 17446 | 17461 | N/A | N/A | AGATTAACATTAGGTC | 60 | AP | 761 |
| 1299750 | 23043 | 23058 | 3318 | 3333 | CCACATAGAGTCTGGT | 88 | AP | 582 |
| 1299765 | 12138 | 12153 | 2441 | 2456 | CTTTTCGAATTTGCCA | 59 | AP | 343 |
| 1299788 | 19469 | 19484 | N/A | N/A | GATGAAGAGTGTGATC | 46 | AP | 2578 |
| 1299824 | 20422 | 20437 | N/A | N/A | TGTAAGTGAAGCGGGC | 75 | AP | 2580 |
| 1299833 | 12453 | 12468 | 2756 | 2771 | CCGATGACAGTTCTCA | 54 | AP | 2118 |
| 1299864 | 23827 | 23842 | N/A | N/A | AGTGCATGATTATTAG | 87 | AP | 2698 |
| 1299873 | 25075 | 25090 | N/A | N/A | CGCTCTGTTTGTGATA | 84 | AP | 2699 |
| 1299901 | 28760 | 28775 | N/A | N/A | AACTATTAAGCAACGG | 44 | AP | 628 |
| 1299902 | 27099 | 27114 | N/A | N/A | ACATAGGCTATACCTA | 69 | AP | 394 |
| | 27117 | 27132 | | | | | | |
| 1299913 | 29461 | 29476 | N/A | N/A | GTCAAATTAAGCCCAT | 84 | AP | 168 |
| 1299921 | 31016 | 31031 | 3460 | 3475 | GTGCTGAGTACCGAGG | 45 | AP | 895 |
| 1232737 | 5138 | 5153 | 90 | 105 | ATGCCATCTTGACCCA | 59 | AQ | 22 |
| 1299511 | 27134 | 27149 | N/A | N/A | ATGTAGCTATACCTAA | 110 | AQ | 2551 |
| 1299549 | 10923 | 10938 | 1226 | 1241 | GTTGAGGCTCACACTC | 72 | AQ | 1340 |
| 1299574 | 11233 | 11248 | 1536 | 1551 | GTGTCACAAGGCTCAC | 61 | AQ | 1418 |
| 1299578 | 11299 | 11314 | 1602 | 1617 | CGATCTTGTGGATGGG | 35 | AQ | 2343 |
| 1299589 | 11952 | 11967 | 2255 | 2270 | GAACAGGTTCATCCTC | 70 | AQ | 34 |
| 1299615 | 12486 | 12501 | 2789 | 2804 | GTTATGGAGAAACCCC | 51 | AQ | 887 |
| 1299633 | 14477 | 14492 | N/A | N/A | CTGAGCCTCCTACCGG | 55 | AQ | 2219 |
| | 14623 | 14638 | | | | | | |
| | 14769 | 14784 | | | | | | |
| | 14915 | 14930 | | | | | | |
| | 15061 | 15076 | | | | | | |
| 1299697 | 23301 | 23316 | N/A | N/A | CTTATTAGCAGCAGTA | 35 | AQ | 845 |
| 1299746 | 17445 | 17460 | N/A | N/A | GATTAACATTAGGTCT | 60 | AQ | 144 |
| 1299772 | 18354 | 18369 | N/A | N/A | GCGGACTTTAACACTT | 63 | AQ | 2586 |
| 1299787 | 12144 | 12159 | 2447 | 2462 | ATACCCCTTTTCGAAT | 72 | AQ | 2347 |
| 1299792 | 18860 | 18875 | N/A | N/A | ACTGTGCAGTAACATA | 61 | AQ | 2700 |
| 1299802 | 19882 | 19897 | N/A | N/A | TTGATTAGTTTCCCCG | 30 | AQ | 1687 |
| 1299830 | 20929 | 20944 | N/A | N/A | GAACACACCTGACCAG | 84 | AQ | 1689 |
| | 21055 | 21070 | | | | | | |
| 1299846 | 22902 | 22917 | N/A | N/A | TGGACAAGGAAGCACC | 72 | AQ | 2565 |
| 1299850 | 23820 | 23835 | N/A | N/A | GATTATTAGTGGGTAT | 69 | AQ | 616 |
| 1299861 | 25020 | 25035 | N/A | N/A | AGTAACTACATAGAGC | 48 | AQ | 465 |
| 1299876 | 26782 | 26797 | N/A | N/A | CAATCGATAGATCTAC | 76 | AQ | 391 |
| 1299911 | 29459 | 29474 | N/A | N/A | CAAATTAAGCCCATTC | 81 | AQ | 2013 |
| 1299934 | 31084 | 31099 | 3528 | 3543 | AGAGTAGTTTGATCCC | 44 | AQ | 2589 |
| 1232737 | 5138 | 5153 | 90 | 105 | ATGCCATCTTGACCCA | 65 | AR | 22 |
| 1299514 | 27132 | 27147 | N/A | N/A | GTAGCTATACCTAACA | 66 | AR | 2701 |
| 1299527 | 6022 | 6037 | 974 | 989 | TCTCCTGTTGATCGCA | 27 | AR | 721 |
| 1299555 | 11011 | 11026 | 1314 | 1329 | TCTTGGTCTTGCCGAT | 55 | AR | 2702 |
| 1299563 | 11004 | 11019 | 1307 | 1322 | CTTGCCGATGGCCAGA | 55 | AR | 2703 |
| 1299577 | 11262 | 11277 | 1565 | 1580 | GCTCATGATCAGGTCC | 43 | AR | 2111 |
| 1299590 | 11948 | 11963 | 2251 | 2266 | AGGTTCATCCTCAGGA | 103 | AR | 729 |
| 1299595 | 11976 | 11991 | 2279 | 2294 | CTTCTCGCAGTCCACT | 70 | AR | 2704 |
| 1299616 | 5171 | 5186 | 123 | 138 | GTATTACAGTTTACGG | 52 | AR | 178 |
| 1299632 | 14572 | 14587 | N/A | N/A | GTGCCATCTAGAGGGA | 83 | AR | 1374 |
| | 14864 | 14879 | | | | | | |
| | 15154 | 15169 | | | | | | |
| | 15227 | 15242 | | | | | | |
| 1299660 | 16645 | 16660 | 2963 | 2978 | AGTTAGACTCTGGCTG | 68 | AR | 1351 |
| 1299671 | 15187 | 15202 | N/A | N/A | CTCCCGTCAGAGTCTG | 66 | AR | 2570 |
| 1299702 | 21603 | 21618 | N/A | N/A | CTTATACAATATGAGG | 48 | AR | 843 |
| 1299706 | 11044 | 11059 | 1347 | 1362 | CCATCTTAATGGGACT | 50 | AR | 2585 |
| 1299740 | 23007 | 23022 | 3282 | 3297 | ATACTGATGCAAGATC | 70 | AR | 738 |
| 1299797 | 20273 | 20288 | N/A | N/A | ACGATTTCATCCCAGC | 29 | AR | 2594 |
| 1299819 | 20590 | 20605 | N/A | N/A | TACACCTGACCAGAGA | 70 | AR | 2587 |
| 1299825 | 12452 | 12467 | 2755 | 2770 | CGATGACAGTTCTCAA | 79 | AR | 2426 |
| 1299865 | 14501 | 14516 | N/A | N/A | AGGTGCTATCTAGAGA | 26 | AR | 1681 |
| | 14647 | 14662 | | | | | | |
| | 14720 | 14735 | | | | | | |
| | 14793 | 14808 | | | | | | |
| | 14939 | 14954 | | | | | | |
| | 15012 | 15027 | | | | | | |
| | 15085 | 15100 | | | | | | |
| 1299894 | 26971 | 26986 | N/A | N/A | GCATAAAAGTCTCGAC | 44 | AR | 623 |
| 1299906 | 31015 | 31030 | 3459 | 3474 | TGCTGAGTACCGAGGA | 49 | AR | 818 |
| 1299918 | 31077 | 31092 | 3521 | 3536 | TTTGATCCCCTTGTCT | 101 | AR | 1589 |
| 1299943 | 25817 | 25832 | N/A | N/A | GTTTACACCAAGCATG | 56 | AR | 2571 |
| 1232737 | 5138 | 5153 | 90 | 105 | ATGCCATCTTGACCCA | 55 | AS | 22 |
| 1299496 | 26960 | 26975 | N/A | N/A | TCGACTGATTAATGCC | 32 | AS | 2558 |
| 1299531 | 27369 | 27384 | N/A | N/A | AGAGCATTTAGGACCG | 52 | AS | 2572 |
| 1299550 | 10883 | 10898 | 1186 | 1201 | TCAATGCACTGGAATC | 94 | AS | 2032 |
| 1299602 | 11975 | 11990 | 2278 | 2293 | TTCTCGCAGTCCACTT | 110 | AS | 2705 |
| 1299607 | 12460 | 12475 | 2763 | 2778 | ACTCCACCCGATGACA | 106 | AS | 2706 |
| 1299618 | 12704 | 12719 | N/A | N/A | TATGATGTAGCTGTGG | 47 | AS | 678 |
| 1299631 | 14478 | 14493 | N/A | N/A | GCTGAGCCTCCTACCG | 40 | AS | 2296 |
| | 14624 | 14639 | | | | | | |
| | 14770 | 14785 | | | | | | |
| | 14916 | 14931 | | | | | | |
| | 15062 | 15077 | | | | | | |
| 1299652 | 16733 | 16748 | 3051 | 3066 | TCCGAATGTTACAGCC | 82 | AS | 578 |
| 1299674 | 5293 | 5308 | 245 | 260 | AGTTAGCCAAATGCTT | 97 | AS | 410 |
| 1299675 | 17667 | 17682 | N/A | N/A | ATATATCGAGCATCAT | 76 | AS | 529 |
| 1299690 | 21594 | 21609 | N/A | N/A | TATGAGGACCCAGGGC | 80 | AS | 2574 |
| 1299720 | 23531 | 23546 | N/A | N/A | CTCCTAGTGTAGAGGC | 67 | AS | 2561 |
| 1299731 | 25159 | 25174 | N/A | N/A | CCTATAAGGACCCAGC | 81 | AS | 542 |
| 1299758 | 12137 | 12152 | 2440 | 2455 | TTTTCGAATTTGCCAT | 99 | AS | 2192 |
| 1299775 | 18859 | 18874 | N/A | N/A | CTGTGCAGTAACATAT | 85 | AS | 2707 |
| 1299782 | 18758 | 18773 | N/A | N/A | GCATTTAAGCCACCGA | 50 | AS | 1147 |
| 1299789 | 12143 | 12158 | 2446 | 2461 | TACCCCTTTTCGAATT | 111 | AS | 2614 |
| 1299799 | 19881 | 19896 | N/A | N/A | TGATTAGTTTCCCCGT | 43 | AS | 2577 |
| 1299807 | 20416 | 20431 | N/A | N/A | TGAAGCGGGCGGTGTG | 79 | AS | 2579 |
| 1299827 | 20698 | 20713 | N/A | N/A | TCCGGAATAGAGAAAG | 86 | AS | 381 |
| | 20740 | 20755 | | | | | | |
| | 20908 | 20923 | | | | | | |
| | 20992 | 21007 | | | | | | |
| 1299836 | 22895 | 22910 | N/A | N/A | GGAAGCACCCGTACCT | 76 | AS | 2708 |
| 1299872 | 25038 | 25053 | N/A | N/A | AGGTTAGGTATTGACA | 57 | AS | 2595 |
| 1299883 | 26787 | 26802 | N/A | N/A | TGCCCCAATCGATAGA | 74 | AS | 2596 |
| 1299942 | 26599 | 26614 | N/A | N/A | ATTTGGTGATACGGTT | 54 | AS | 2709 |
| 1232737 | 5138 | 5153 | 90 | 105 | ATGCCATCTTGACCCA | 57 | AT | 22 |
| 1299526 | 6021 | 6036 | 973 | 988 | CTCCTGTTGATCGCAG | 63 | AT | 644 |
| 1299541 | 10300 | 10315 | 1100 | 1115 | GTACTCCAGTAAACCC | 43 | AT | 2031 |
| 1299566 | 11232 | 11247 | 1535 | 1550 | TGTCACAAGGCTCACC | 74 | AT | 2710 |
| 1299580 | 5150 | 5165 | 102 | 117 | AACCACTTCACGATGC | 80 | AT | 871 |
| 1299600 | 11981 | 11996 | 2284 | 2299 | TAGAACTTCTCGCAGT | 85 | AT | 2623 |
| 1299610 | 12466 | 12481 | 2769 | 2784 | ACAGTGACTCCACCCG | 43 | AT | 656 |
| 1299617 | 12703 | 12718 | N/A | N/A | ATGATGTAGCTGTGGC | 30 | AT | 2625 |
| 1299630 | 14569 | 14584 | N/A | N/A | CCATCTAGAGGGATGG | 95 | AT | 1220 |
| | 14861 | 14876 | | | | | | |
| | 15151 | 15166 | | | | | | |
| | 15224 | 15239 | | | | | | |
| 1299655 | 5290 | 5305 | 242 | 257 | TAGCCAAATGCTTACC | 57 | AT | 1948 |
| 1299663 | 16644 | 16659 | 2962 | 2977 | GTTAGACTCTGGCTGG | 30 | AT | 1274 |
| 1299666 | 15185 | 15200 | N/A | N/A | CCCGTCAGAGTCTGTC | 38 | AT | 2711 |
| 1299670 | 17660 | 17675 | N/A | N/A | GAGCATCATGGTTGTT | 21 | AT | 2601 |
| 1299679 | 17673 | 17688 | N/A | N/A | AATGGAATATATCGAG | 49 | AT | 222 |
| 1299725 | 23502 | 23517 | N/A | N/A | CTCTTTCCTAGTGTAG | 91 | AT | 2611 |
| 1299770 | 17988 | 18003 | N/A | N/A | CCCATACAAAGCATTC | 64 | AT | 2576 |
| 1299790 | 19273 | 19288 | N/A | N/A | TGATAGACACTAGAGC | 70 | AT | 2613 |
| 1299813 | 20279 | 20294 | N/A | N/A | TGTTTGACGATTTCAT | 69 | AT | 2712 |
| 1299826 | 20697 | 20712 | N/A | N/A | CCGGAATAGAGAAAGC | 66 | AT | 304 |
| | 20739 | 20754 | | | | | | |
| | 20907 | 20922 | | | | | | |
| | 20991 | 21006 | | | | | | |
| 1299845 | 23825 | 23840 | N/A | N/A | TGCATGATTATTAGTG | 56 | AT | 2606 |
| 1299851 | 23817 | 23832 | N/A | N/A | TATTAGTGGGTATTCC | 87 | AT | 462 |
| 1299854 | 22901 | 22916 | N/A | N/A | GGACAAGGAAGCACCC | 62 | AT | 2605 |
| 1299860 | 25019 | 25034 | N/A | N/A | GTAACTACATAGAGCA | 61 | AT | 2466 |
| 1299866 | 26781 | 26796 | N/A | N/A | AATCGATAGATCTACA | 67 | AT | 314 |
| 1299904 | 28759 | 28774 | N/A | N/A | ACTATTAAGCAACGGT | 46 | AT | 551 |
| 1299907 | 31014 | 31029 | 3458 | 3473 | GCTGAGTACCGAGGAC | 36 | AT | 741 |
| 1299919 | 31022 | 31037 | 3466 | 3481 | TGATTAGTGCTGAGTA | 56 | AT | 1280 |
| 1232737 | 5138 | 5153 | 90 | 105 | ATGCCATCTTGACCCA | 52 | AU | 22 |
| 1299502 | 26959 | 26974 | N/A | N/A | CGACTGATTAATGCCA | 36 | AU | 2713 |
| 1299517 | 27362 | 27377 | N/A | N/A | TTAGGACCGTCCCCCG | 100 | AU | 2714 |
| 1299558 | 11010 | 11025 | 1313 | 1328 | CTTGGTCTTGCCGATG | 54 | AU | 2715 |
| 1299592 | 11947 | 11962 | 2250 | 2265 | GGTTCATCCTCAGGAA | 72 | AU | 652 |
| 1299619 | 5170 | 5185 | 122 | 137 | TATTACAGTTTACGGT | 45 | AU | 101 |
| 1299629 | 14573 | 14588 | N/A | N/A | GGTGCCATCTAGAGGG | 93 | AU | 1451 |
| | 14865 | 14880 | | | | | | |
| | 15155 | 15170 | | | | | | |
| | 15228 | 15243 | | | | | | |
| 1299678 | 20578 | 20593 | N/A | N/A | GAGAGCTCCGGAATAC | 35 | AU | 841 |
| | 20956 | 20971 | | | | | | |
| 1299692 | 20580 | 20595 | N/A | N/A | CAGAGAGCTCCGGAAT | 64 | AU | 918 |
| | 20706 | 20721 | | | | | | |
| | 20748 | 20763 | | | | | | |
| | 20916 | 20931 | | | | | | |
| | 20958 | 20973 | | | | | | |
| 1299711 | 23493 | 23508 | N/A | N/A | AGTGTAGAGGCTGCAT | 29 | AU | 2716 |
| 1299715 | 23497 | 23512 | N/A | N/A | TCCTAGTGTAGAGGCT | 23 | AU | 154 |
| | 23530 | 23545 | | | | | | |
| 1299732 | 25158 | 25173 | N/A | N/A | CTATAAGGACCCAGCC | 68 | AU | 2592 |
| 1299751 | 23041 | 23056 | 3316 | 3331 | ACATAGAGTCTGGTCA | 63 | AU | 505 |
| 1299816 | 20421 | 20436 | N/A | N/A | GTAAGTGAAGCGGGCG | 66 | AU | 2072 |
| 1299834 | 21014 | 21029 | N/A | N/A | AGGACACACCTGATCA | 59 | AU | 2717 |
| 1299835 | 12457 | 12472 | 2760 | 2775 | CCACCCGATGACAGTT | 78 | AU | 2631 |
| 1299859 | 25018 | 25033 | N/A | N/A | TAACTACATAGAGCAT | 59 | AU | 2616 |
| 1299874 | 25074 | 25089 | N/A | N/A | GCTCTGTTTGTGATAC | 56 | AU | 2389 |
| | 26262 | 26277 | | | | | | |
| 1299884 | 26786 | 26801 | N/A | N/A | GCCCCAATCGATAGAT | 86 | AU | 2642 |
| 1299905 | 5141 | 5156 | 93 | 108 | ACGATGCCATCTTGAC | 50 | AU | 2254 |
| 1232737 | 5138 | 5153 | 90 | 105 | ATGCCATCTTGACCCA | 43 | AV | 22 |
| 1299505 | 27199 | 27214 | N/A | N/A | AGTATTGAGACAGGCA | 40 | AV | 2622 |
| 1299506 | 27100 | 27115 | N/A | N/A | CACATAGGCTATACCT | 55 | AV | 1623 |
| | 27118 | 27133 | | | | | | |
| 1299533 | 27368 | 27383 | N/A | N/A | GAGCATTTAGGACCGT | 38 | AV | 1393 |
| 1299540 | 10297 | 10312 | 1097 | 1112 | CTCCAGTAAACCCATC | 91 | AV | 1877 |
| 1299546 | 11003 | 11018 | 1306 | 1321 | TTGCCGATGGCCAGAA | 76 | AV | 2718 |
| 1299565 | 11261 | 11276 | 1564 | 1579 | CTCATGATCAGGTCCC | 55 | AV | 2419 |
| 1299581 | 11298 | 11313 | 1601 | 1616 | GATCTTGTGGATGGGT | 62 | AV | 2600 |
| 1299650 | 16732 | 16747 | 3050 | 3065 | CCGAATGTTACAGCCA | 55 | AV | 501 |
| 1299667 | 17666 | 17681 | N/A | N/A | TATATCGAGCATCATG | 65 | AV | 452 |
| 1299682 | 17672 | 17687 | N/A | N/A | ATGGAATATATCGAGC | 32 | AV | 145 |
| 1299696 | 11043 | 11058 | 1346 | 1361 | CATCTTAATGGGACTC | 44 | AV | 417 |
| 1299726 | 25164 | 25179 | N/A | N/A | TACGACCTATAAGGAC | 96 | AV | 2603 |
| 1299742 | 23006 | 23021 | 3281 | 3296 | TACTGATGCAAGATCC | 65 | AV | 661 |
| 1299752 | 23040 | 23055 | 3315 | 3330 | CATAGAGTCTGGTCAG | 87 | AV | 428 |
| 1299771 | 18353 | 18368 | N/A | N/A | CGGACTTTAACACTTG | 58 | AV | 2639 |
| 1299817 | 12451 | 12466 | 2754 | 2769 | GATGACAGTTCTCAAT | 95 | AV | 2041 |
| 1299840 | 22431 | 22446 | N/A | N/A | GCATAATGTCCCCGAG | 6 | AV | 536 |
| 1299848 | 23824 | 23839 | N/A | N/A | GCATGATTATTAGTGG | 62 | AV | 2633 |
| 1299877 | 14504 | 14519 | N/A | N/A | GGAAGGTGCTATCTAG | 31 | AV | 1835 |
| | 14650 | 14665 | | | | | | |
| | 14723 | 14738 | | | | | | |
| | 14796 | 14811 | | | | | | |
| | 14942 | 14957 | | | | | | |
| | 15015 | 15030 | | | | | | |
| | 15088 | 15103 | | | | | | |
| 1299903 | 28758 | 28773 | N/A | N/A | CTATTAAGCAACGGTT | 49 | AV | 474 |
| 1299914 | 28765 | 28780 | N/A | N/A | CTGGTAACTATTAAGC | 73 | AV | 2597 |
| 1299925 | 31083 | 31098 | 3527 | 3542 | GAGTAGTTTGATCCCC | 57 | AV | 2051 |
| 1232737 | 5138 | 5153 | 90 | 105 | ATGCCATCTTGACCCA | 65 | AW | 22 |
| 1299498 | 27028 | 27043 | N/A | N/A | CCCATAGGCTATACCT | 40 | AW | 547 |
| | 27064 | 27079 | | | | | | |
| 1299512 | 27131 | 27146 | N/A | N/A | TAGCTATACCTAACAC | 74 | AW | 2634 |
| 1299534 | 27367 | 27382 | N/A | N/A | AGCATTTAGGACCGTC | 37 | AW | 1316 |
| 1299545 | 10880 | 10895 | 1183 | 1198 | ATGCACTGGAATCTGC | 53 | AW | 1801 |
| 1299560 | 11009 | 11024 | 1312 | 1327 | TTGGTCTTGCCGATGG | 78 | AW | 2719 |
| 1299569 | 5149 | 5164 | 101 | 116 | ACCACTTCACGATGCC | 42 | AW | 792 |
| 1299586 | 11974 | 11989 | 2277 | 2292 | TCTCGCAGTCCACTTC | 104 | AW | 2720 |
| 1299621 | 5169 | 5184 | 121 | 136 | ATTACAGTTTACGGTG | 60 | AW | 1331 |
| 1299627 | 14570 | 14585 | N/A | N/A | GCCATCTAGAGGGATG | 74 | AW | 1297 |
| | 14862 | 14877 | | | | | | |
| | 15152 | 15167 | | | | | | |
| | 15225 | 15240 | | | | | | |
| 1299654 | 16731 | 16746 | 3049 | 3064 | CGAATGTTACAGCCAG | 68 | AW | 2353 |
| 1299657 | 15112 | 15127 | N/A | N/A | CCAGTCAGAGTCTGTC | 10 | AW | 991 |
| | 15258 | 15273 | | | | | | |
| 1299673 | 15184 | 15199 | N/A | N/A | CCGTCAGAGTCTGTCC | 23 | AW | 2721 |
| 1299694 | 20583 | 20598 | N/A | N/A | GACCAGAGAGCTCCGG | 48 | AW | 73 |
| | 20709 | 20724 | | | | | | |
| | 20751 | 20766 | | | | | | |
| | 20919 | 20934 | | | | | | |
| 1299695 | 21600 | 21615 | N/A | N/A | ATACAATATGAGGACC | 55 | AW | 689 |
| 1299708 | 23492 | 23507 | N/A | N/A | GTGTAGAGGCTGCATG | 45 | AW | 2610 |
| 1299749 | 17443 | 17458 | N/A | N/A | TTAACATTAGGTCTTG | 54 | AW | 2638 |
| 1299761 | 12136 | 12151 | 2439 | 2454 | TTTCGAATTTGCCATA | 59 | AW | 266 |
| 1299776 | 18757 | 18772 | N/A | N/A | CATTTAAGCCACCGAA | 111 | AW | 224 |
| 1299778 | 18858 | 18873 | N/A | N/A | TGTGCAGTAACATATC | 63 | AW | 2722 |
| 1299796 | 20272 | 20287 | N/A | N/A | CGATTTCATCCCAGCC | 21 | AW | 2615 |
| 1299871 | 25036 | 25051 | N/A | N/A | GTTAGGTATTGACAAT | 58 | AW | 2651 |
| 1299875 | 26970 | 26985 | N/A | N/A | CATAAAAGTCTCGACT | 55 | AW | 2619 |
| 1232737 | 5138 | 5153 | 90 | 105 | ATGCCATCTTGACCCA | 43 | AX | 22 |
| 1299499 | 26958 | 26973 | N/A | N/A | GACTGATTAATGCCAC | 47 | AX | 2620 |
| 1299519 | 27206 | 27221 | N/A | N/A | CTTGAGCAGTATTGAG | 72 | AX | 2644 |
| 1299529 | 6020 | 6035 | 972 | 987 | TCCTGTTGATCGCAGC | 53 | AX | 2338 |
| 1299570 | 11231 | 11246 | 1534 | 1549 | GTCACAAGGCTCACCT | 53 | AX | 2624 |
| 1299572 | 5148 | 5163 | 100 | 115 | CCACTTCACGATGCCA | 33 | AX | 407 |
| 1299582 | 11297 | 11312 | 1600 | 1615 | ATCTTGTGGATGGGTG | 61 | AX | 2265 |
| 1299594 | 11304 | 11319 | 1607 | 1622 | TCTCACGATCTTGTGG | 72 | AX | 882 |
| 1299599 | 11980 | 11995 | 2283 | 2298 | AGAACTTCTCGCAGTC | 52 | AX | 2723 |
| 1299626 | 14571 | 14586 | N/A | N/A | TGCCATCTAGAGGGAT | 47 | AX | 450 |
| | 14863 | 14878 | | | | | | |
| | 15153 | 15168 | | | | | | |
| | 15226 | 15241 | | | | | | |
| 1299685 | 20582 | 20597 | N/A | N/A | ACCAGAGAGCTCCGGA | 41 | AX | 611 |
| | 20708 | 20723 | | | | | | |
| | 20750 | 20765 | | | | | | |
| | 20918 | 20933 | | | | | | |
| 1299727 | 25163 | 25178 | N/A | N/A | ACGACCTATAAGGACC | 47 | AX | 2657 |
| 1299733 | 23535 | 23550 | N/A | N/A | TCCTCTCCTAGTGTAG | 95 | AX | 2724 |
| 1299741 | 23004 | 23019 | 3279 | 3294 | CTGATGCAAGATCCTG | 58 | AX | 2277 |
| 1299757 | 17703 | 17718 | N/A | N/A | ACTCTCATCTATGAAC | 65 | AX | 2725 |
| 1299804 | 19880 | 19895 | N/A | N/A | GATTAGTTTCCCCGTC | 25 | AX | 1610 |
| 1299853 | 22900 | 22915 | N/A | N/A | GACAAGGAAGCACCCG | 49 | AX | 2076 |
| 1299862 | 5140 | 5155 | 92 | 107 | CGATGCCATCTTGACC | 48 | AX | 2177 |
| 1299867 | 26779 | 26794 | N/A | N/A | TCGATAGATCTACACA | 56 | AX | 2617 |
| 1299917 | 31020 | 31035 | 3464 | 3479 | ATTAGTGCTGAGTACC | 61 | AX | 1203 |
| 1299928 | 25814 | 25829 | N/A | N/A | TACACCAAGCATGTGT | 80 | AX | 2607 |
| 1299936 | 26598 | 26613 | N/A | N/A | TTTGGTGATACGGTTA | 48 | AX | 2726 |
| 1232737 | 5138 | 5153 | 90 | 105 | ATGCCATCTTGACCCA | 69 | AY | 22 |
| 1299547 | 11001 | 11016 | 1304 | 1319 | GCCGATGGCCAGAAGC | 41 | AY | 2621 |
| 1299567 | 11260 | 11275 | 1563 | 1578 | TCATGATCAGGTCCCC | 47 | AY | 2034 |
| 1299608 | 12465 | 12480 | 2768 | 2783 | CAGTGACTCCACCCGA | 39 | AY | 2350 |
| 1299624 | 5168 | 5183 | 120 | 135 | TTACAGTTTACGGTGA | 53 | AY | 24 |
| 1299625 | 14476 | 14491 | N/A | N/A | TGAGCCTCCTACCGGG | 52 | AY | 2142 |
| | 14622 | 14637 | | | | | | |
| | 14768 | 14783 | | | | | | |
| | 14914 | 14929 | | | | | | |
| | 15060 | 15075 | | | | | | |
| 1299641 | 5288 | 5303 | 240 | 255 | GCCAAATGCTTACCAG | 52 | AY | 333 |
| 1299661 | 16643 | 16658 | 2961 | 2976 | TTAGACTCTGGCTGGT | 59 | AY | 2608 |
| 1299665 | 17665 | 17680 | N/A | N/A | ATATCGAGCATCATGG | 60 | AY | 375 |
| 1299755 | 12135 | 12150 | 2438 | 2453 | TTCGAATTTGCCATAG | 51 | AY | 189 |
| 1299793 | 19270 | 19285 | N/A | N/A | TAGACACTAGAGCCTC | 48 | AY | 2658 |
| 1299794 | 12142 | 12157 | 2445 | 2460 | ACCCCTTTTCGAATTT | 62 | AY | 2269 |
| 1299814 | 12449 | 12464 | 2752 | 2767 | TGACAGTTCTCAATGC | 73 | AY | 2727 |
| 1299818 | 5139 | 5154 | 91 | 106 | GATGCCATCTTGACCC | 83 | AY | 2100 |
| 1299821 | 20426 | 20441 | N/A | N/A | GGTTTGTAAGTGAAGC | 59 | AY | 2659 |
| 1299829 | 12456 | 12471 | 2759 | 2774 | CACCCGATGACAGTTC | 48 | AY | 345 |
| 1299843 | 20701 | 20716 | N/A | N/A | AGCTCCGGAATAGAGA | 91 | AY | 612 |
| | 20743 | 20758 | | | | | | |
| | 20911 | 20926 | | | | | | |
| | 20995 | 21010 | | | | | | |
| 1299844 | 22430 | 22445 | N/A | N/A | CATAATGTCCCCGAGG | 65 | AY | 2632 |
| 1299886 | 27027 | 27042 | N/A | N/A | CCATAGGCTATACCTA | 61 | AY | 470 |
| | 27063 | 27078 | | | | | | |
| 1299908 | 31012 | 31027 | 3456 | 3471 | TGAGTACCGAGGACAA | 43 | AY | 2358 |
| 1299926 | 25746 | 25761 | N/A | N/A | TGTTGAAATTGAGAGG | 44 | AY | 2728 |
| 1299933 | 31082 | 31097 | 3526 | 3541 | AGTAGTTTGATCCCCT | 56 | AY | 1974 |
| 1232737 | 5138 | 5153 | 90 | 105 | ATGCCATCTTGACCCA | 63 | AZ | 22 |
| 1299507 | 27130 | 27145 | N/A | N/A | AGCTATACCTAACACA | 67 | AZ | 2239 |
| 1299509 | 27198 | 27213 | N/A | N/A | GTATTGAGACAGGCAG | 42 | AZ | 854 |
| 1299516 | 27366 | 27381 | N/A | N/A | GCATTTAGGACCGTCC | 58 | AZ | 241 |
| 1299524 | 27204 | 27219 | N/A | N/A | TGAGCAGTATTGAGAC | 74 | AZ | 2729 |
| 1299528 | 6019 | 6034 | 971 | 986 | CCTGTTGATCGCAGCG | 52 | AZ | 2260 |
| 1299536 | 10305 | 10320 | 1105 | 1120 | GAAAGGTACTCCAGTA | 48 | AZ | 338 |
| 1299544 | 10295 | 10310 | 1095 | 1110 | CCAGTAAACCCATCCA | 48 | AZ | 1723 |
| 1299559 | 11008 | 11023 | 1311 | 1326 | TGGTCTTGCCGATGGC | 35 | AZ | 2033 |
| 1299587 | 11973 | 11988 | 2276 | 2291 | CTCGCAGTCCACTTCC | 86 | AZ | 868 |
| 1299637 | 14577 | 14592 | N/A | N/A | GGCAGGTGCCATCTAG | 72 | AZ | 1759 |
| | 14869 | 14884 | | | | | | |
| | 15159 | 15174 | | | | | | |
| | 15232 | 15247 | | | | | | |
| 1299644 | 14575 | 14590 | N/A | N/A | CAGGTGCCATCTAGAG | 31 | AZ | 1605 |
| | 14867 | 14882 | | | | | | |
| | 15157 | 15172 | | | | | | |
| | 15230 | 15245 | | | | | | |
| 1299645 | 16642 | 16657 | 2960 | 2975 | TAGACTCTGGCTGGTG | 43 | AZ | 2730 |
| 1299668 | 17664 | 17679 | N/A | N/A | TATCGAGCATCATGGT | 71 | AZ | 2487 |
| 1299699 | 23282 | 23297 | N/A | N/A | ATTATATGCCTCCAGT | 62 | AZ | 2626 |
| 1299703 | 21599 | 21614 | N/A | N/A | TACAATATGAGGACCC | 41 | AZ | 228 |
| 1299704 | 11042 | 11057 | 1345 | 1360 | ATCTTAATGGGACTCA | 65 | AZ | 340 |
| 1299777 | 18857 | 18872 | N/A | N/A | GTGCAGTAACATATCC | 66 | AZ | 2489 |
| 1299781 | 12141 | 12156 | 2444 | 2459 | CCCCTTTTCGAATTTG | 44 | AZ | 574 |
| 1299784 | 18756 | 18771 | N/A | N/A | ATTTAAGCCACCGAAC | 95 | AZ | 2646 |
| 1299806 | 20420 | 20435 | N/A | N/A | TAAGTGAAGCGGGCGG | 52 | AZ | 2630 |
| 1299808 | 20277 | 20292 | N/A | N/A | TTTGACGATTTCATCC | 53 | AZ | 2731 |
| 1299832 | 20593 | 20608 | N/A | N/A | GAATACACCTGACCAG | 139 | AZ | 227 |
| 1299837 | 22899 | 22914 | N/A | N/A | ACAAGGAAGCACCCGT | 72 | AZ | 1999 |
| 1299847 | 23823 | 23838 | N/A | N/A | CATGATTATTAGTGGG | 62 | AZ | 78 |
| 1299887 | 27025 | 27040 | N/A | N/A | ATAGGCTATACCTAAC | 65 | AZ | 316 |
| | 27061 | 27076 | | | | | | |
| | 27097 | 27112 | | | | | | |
| | 27115 | 27130 | | | | | | |
| 1299897 | 28757 | 28772 | N/A | N/A | TATTAAGCAACGGTTA | 71 | AZ | 1549 |
| 1299922 | 31019 | 31034 | 3463 | 3478 | TTAGTGCTGAGTACCG | 58 | AZ | 1126 |
| 1299944 | 26596 | 26611 | N/A | N/A | TGGTGATACGGTTACT | 30 | AZ | 2496 |

### Example 3: Effect of Mixed MOE and cEt, Uniform Phosphorothioate Modified Oligonucleotides on Human NLRP3 RNA In Vitro, Single Dose

Modified oligonucleotides complementary to human NLRP3 nucleic acid were designed and tested for their single dose effects on NLRP3 RNA in vitro. The modified oligonucleotides were tested in a series of experiments that had the same culture conditions.

The modified oligonucleotides in the table below are 16 nucleosides in length, wherein the sugar motif for the modified oligonucleotides is (from 5′ to 3′): kkdddddddddkekek; wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. “N/A” indicates that the modified oligonucleotide is not 100% complementary to that particular target nucleic acid sequence.

Cultured THP-1 cells were treated with modified oligonucleotide at a concentration of 2000 nM by electroporation at a density of 300,000 cells per well. After a treatment period of approximately 24 hours, total RNA was isolated from the cells and NLRP3 RNA levels were measured by quantitative real-time RTPCR. NLRP3 RNA levels were measured by human primer-probe set RTS37509 (described herein above). NLRP3 RNA levels were normalized to total RNA content, as measured by RIBOGREEN®. Reduction of NLRP3 RNA is presented in the tables below as percent NLRP3 RNA relative to the amount of NLRP3 RNA in untreated control cells (% UTC).

Each separate experimental analysis described in this example is identified by a letter ID in the table column below labeled “AID” (Analysis ID). In the table below, Compound No. 1232737 (described herein above) was used as a benchmark on multiple plates.

TABLE 5
Reduction of NLRP3 RNA by modified oligonucleotides with a mixed MOE/cEt sugar motif
and uniform phosphorothioate internucleoside linkages at a concentration of 2000 nM
in THP-1 cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | NLRP3 | | |
| Compound | Start | Stop | Start | Stop | | (% | | SEQ ID |
| Number | Site | Site | Site | Site | Sequence (5′ to 3′) | UTC) | AID | NO |
| 1232737 | 5138 | 5153 | 90 | 105 | ATGCCATCTTGACCCA | 60 | AH | 22 |
| 1300733 | 27366 | 27381 | N/A | N/A | GCATTTAGGACCGTCC | 61 | AH | 241 |
| 1300739 | 27204 | 27219 | N/A | N/A | TGAGCAGTATTGAGAC | 60 | AH | 2729 |
| 1300749 | 6018 | 6033 | 970 | 985 | CTGTTGATCGCAGCGA | 70 | AH | 2183 |
| 1300754 | 10303 | 10318 | 1103 | 1118 | AAGGTACTCCAGTAAA | 87 | AH | 2516 |
| 1300781 | 11007 | 11022 | 1310 | 1325 | GGTCTTGCCGATGGCC | 91 | AH | 2675 |
| 1300808 | 11972 | 11987 | 2275 | 2290 | TCGCAGTCCACTTCCT | 68 | AH | 2668 |
| 1300873 | 16642 | 16657 | 2960 | 2975 | TAGACTCTGGCTGGTG | 56 | AH | 2730 |
| 1300883 | 15183 | 15198 | N/A | N/A | CGTCAGAGTCTGTCCT | 25 | AH | 2656 |
| 1300912 | 20583 | 20598 | N/A | N/A | GACCAGAGAGCTCCGG | 55 | AH | 73 |
| | 20709 | 20724 | | | | | | |
| | 20751 | 20766 | | | | | | |
| | 20919 | 20934 | | | | | | |
| 1300914 | 21598 | 21613 | N/A | N/A | ACAATATGAGGACCCA | 51 | AH | 2383 |
| 1300954 | 11048 | 11063 | 1351 | 1366 | AACTCCATCTTAATGG | 62 | AH | 2110 |
| 1300959 | 23002 | 23017 | 3277 | 3292 | GATGCAAGATCCTGAC | 52 | AH | 2123 |
| 1300968 | 12134 | 12149 | 2437 | 2452 | TCGAATTTGCCATAGT | 39 | AH | 2115 |
| 1301007 | 19270 | 19285 | N/A | N/A | TAGACACTAGAGCCTC | 62 | AH | 2658 |
| 1301030 | 20276 | 20291 | N/A | N/A | TTGACGATTTCATCCC | 54 | AH | 2672 |
| 1301045 | 12455 | 12470 | 2758 | 2773 | ACCCGATGACAGTTCT | 80 | AH | 2195 |
| 1301069 | 23823 | 23838 | N/A | N/A | CATGATTATTAGTGGG | 96 | AH | 78 |
| 1301145 | 25745 | 25760 | N/A | N/A | GTTGAAATTGAGAGGT | 33 | AH | 1235 |
| 1301146 | 35776 | 35791 | 4129 | 4144 | GAGCTAATTACATGAG | 58 | AH | 824 |
| 1301163 | 26603 | 26618 | N/A | N/A | CCTTATTTGGTGATAC | 53 | AH | 1314 |
| 1232737 | 5138 | 5153 | 90 | 105 | ATGCCATCTTGACCCA | 54 | AI | 22 |
| 1300728 | 27195 | 27210 | N/A | N/A | TTGAGACAGGCAGTAC | 62 | AI | 2383 |
| 1300737 | 27365 | 27380 | N/A | N/A | CATTTAGGACCGTCCC | 74 | AI | 2110 |
| 1300786 | 11258 | 11273 | 1561 | 1576 | ATGATCAGGTCCCCCA | 65 | AI | 2123 |
| 1300824 | 5166 | 5181 | 118 | 133 | ACAGTTTACGGTGAAC | 51 | AI | 2115 |
| 1300831 | 12276 | 12291 | 2579 | 2594 | GGCTTTCACTTCAATC | 36 | AI | 2658 |
| 1300865 | 16737 | 16752 | 3055 | 3070 | AATCTCCGAATGTTAC | 60 | AI | 2672 |
| 1300885 | 17664 | 17679 | N/A | N/A | TATCGAGCATCATGGT | 38 | AI | 2195 |
| 1300899 | 17670 | 17685 | N/A | N/A | GGAATATATCGAGCAT | 23 | AI | 78 |
| 1300909 | 11041 | 11056 | 1344 | 1359 | TCTTAATGGGACTCAC | 43 | AI | 1235 |
| 1300921 | 23281 | 23296 | N/A | N/A | TTATATGCCTCCAGTC | 49 | AI | 824 |
| 1300932 | 5137 | 5152 | 89 | 104 | TGCCATCTTGACCCAT | 83 | AI | 1314 |
| 1300933 | 23533 | 23548 | N/A | N/A | CTCTCCTAGTGTAGAG | 99 | AI | 2488 |
| 1300960 | 23000 | 23015 | 3275 | 3290 | TGCAAGATCCTGACAA | 72 | AI | 2046 |
| 1300980 | 17697 | 17712 | N/A | N/A | ATCTATGAACATGGCC | 64 | AI | 1839 |
| 1300999 | 18755 | 18770 | N/A | N/A | TTTAAGCCACCGAACA | 85 | AI | 2509 |
| 1301022 | 19878 | 19893 | N/A | N/A | TTAGTTTCCCCGTCAC | 40 | AI | 2679 |
| 1301081 | 24554 | 24569 | N/A | N/A | AGTCAAATTGCCAAGG | 58 | AI | 2521 |
| 1301093 | 26784 | 26799 | N/A | N/A | CCCAATCGATAGATCT | 73 | AI | 468 |
| 1301098 | 14504 | 14519 | N/A | N/A | GGAAGGTGCTATCTAG | 40 | AI | 1835 |
| | 14650 | 14665 | | | | | | |
| | 14723 | 14738 | | | | | | |
| | 14796 | 14811 | | | | | | |
| | 14942 | 14957 | | | | | | |
| | 15015 | 15030 | | | | | | |
| | 15088 | 15103 | | | | | | |
| 1301117 | 28755 | 28770 | N/A | N/A | TTAAGCAACGGTTACA | 65 | AI | 1472 |
| 1301124 | 28762 | 28777 | N/A | N/A | GTAACTATTAAGCAAC | 79 | AI | 1626 |
| 1301126 | 29463 | 29478 | N/A | N/A | GAGTCAAATTAAGCCC | 57 | AI | 2495 |
| 1301137 | 31018 | 31033 | 3462 | 3477 | TAGTGCTGAGTACCGA | 46 | AI | 1049 |
| 1301143 | 31081 | 31096 | 3525 | 3540 | GTAGTTTGATCCCCTT | 59 | AI | 1897 |
| 1301157 | 26602 | 26617 | N/A | N/A | CTTATTTGGTGATACG | 57 | AI | 1237 |
| 1232737 | 5138 | 5153 | 90 | 105 | ATGCCATCTTGACCCA | 77 | AJ | 22 |
| 1300714 | 26962 | 26977 | N/A | N/A | TCTCGACTGATTAATG | 73 | AJ | 2667 |
| 1300715 | 27094 | 27109 | N/A | N/A | GGCTATACCTAACACG | 65 | AJ | 2515 |
| 1300742 | 27202 | 27217 | N/A | N/A | AGCAGTATTGAGACAG | 57 | AJ | 2497 |
| 1300812 | 11301 | 11316 | 1604 | 1619 | CACGATCTTGTGGATG | 57 | AJ | 726 |
| 1300825 | 12463 | 12478 | 2766 | 2781 | GTGACTCCACCCGATG | 42 | AJ | 2272 |
| 1300829 | 12275 | 12290 | 2578 | 2593 | GCTTTCACTTCAATCC | 53 | AJ | 1347 |
| 1300837 | 12491 | 12506 | 2794 | 2809 | GGCATGTTATGGAGAA | 45 | AJ | 1118 |
| 1300854 | 14679 | 14694 | N/A | N/A | GCTCCAATCAGAGTCT | 55 | AJ | 2068 |
| | 14825 | 14840 | | | | | | |
| | 14971 | 14986 | | | | | | |
| 1300863 | 16727 | 16742 | 3045 | 3060 | TGTTACAGCCAGGATG | 67 | AJ | 2669 |
| 1300874 | 15115 | 15130 | N/A | N/A | GTTCCAGTCAGAGTCT | 38 | AJ | 2681 |
| 1300887 | 17663 | 17678 | N/A | N/A | ATCGAGCATCATGGTT | 44 | AJ | 2517 |
| 1300908 | 21597 | 21612 | N/A | N/A | CAATATGAGGACCCAG | 58 | AJ | 2305 |
| 1300934 | 23500 | 23515 | N/A | N/A | CTTTCCTAGTGTAGAG | 54 | AJ | 2518 |
| 1300948 | 25162 | 25177 | N/A | N/A | CGACCTATAAGGACCC | 51 | AJ | 2502 |
| 1300972 | 23038 | 23053 | 3313 | 3328 | TAGAGTCTGGTCAGGG | 67 | AJ | 1893 |
| 1300977 | 17448 | 17463 | N/A | N/A | CTAGATTAACATTAGG | 44 | AJ | 915 |
| 1300996 | 18856 | 18871 | N/A | N/A | TGCAGTAACATATCCA | 58 | AJ | 1532 |
| 1301009 | 19269 | 19284 | N/A | N/A | AGACACTAGAGCCTCA | 73 | AJ | 2504 |
| 1301028 | 20419 | 20434 | N/A | N/A | AAGTGAAGCGGGCGGT | 90 | AJ | 2490 |
| 1301041 | 20424 | 20439 | N/A | N/A | TTTGTAAGTGAAGCGG | 38 | AJ | 2491 |
| 1301068 | 23822 | 23837 | N/A | N/A | ATGATTATTAGTGGGT | 90 | AJ | 2463 |
| 1301085 | 25034 | 25049 | N/A | N/A | TAGGTATTGACAATAG | 30 | AJ | 2494 |
| 1301092 | 14501 | 14516 | N/A | N/A | AGGTGCTATCTAGAGA | 18 | AJ | 1681 |
| | 14647 | 14662 | | | | | | |
| | 14720 | 14735 | | | | | | |
| | 14793 | 14808 | | | | | | |
| | 14939 | 14954 | | | | | | |
| | 15012 | 15027 | | | | | | |
| | 15085 | 15100 | | | | | | |
| 1232737 | 5138 | 5153 | 90 | 105 | ATGCCATCTTGACCCA | 64 | AK | 22 |
| 1300746 | 6017 | 6032 | 969 | 984 | TGTTGATCGCAGCGAA | 67 | AK | 2106 |
| 1300750 | 27371 | 27386 | N/A | N/A | GCAGAGCATTTAGGAC | 61 | AK | 2506 |
| 1300753 | 6025 | 6040 | 977 | 992 | GTCTCTCCTGTTGATC | 48 | AK | 2674 |
| 1300766 | 5146 | 5161 | 98 | 113 | ACTTCACGATGCCATC | 55 | AK | 715 |
| 1300769 | 10927 | 10942 | 1230 | 1245 | GTTTGTTGAGGCTCAC | 48 | AK | 2680 |
| 1300864 | 16641 | 16656 | 2959 | 2974 | AGACTCTGGCTGGTGC | 66 | AK | 2670 |
| 1300898 | 17669 | 17684 | N/A | N/A | GAATATATCGAGCATC | 33 | AK | 68 |
| 1300922 | 23280 | 23295 | N/A | N/A | TATATGCCTCCAGTCC | 52 | AK | 2537 |
| 1300930 | 23305 | 23320 | N/A | N/A | TTATCTTATTAGCAGC | 50 | AK | 922 |
| 1300938 | 11046 | 11061 | 1349 | 1364 | CTCCATCTTAATGGGA | 81 | AK | 2528 |
| 1300984 | 12140 | 12155 | 2443 | 2458 | CCCTTTTCGAATTTGC | 25 | AK | 497 |
| 1301048 | 20592 | 20607 | N/A | N/A | AATACACCTGACCAGA | 72 | AK | 1457 |
| 1301049 | 12454 | 12469 | 2757 | 2772 | CCCGATGACAGTTCTC | 54 | AK | 268 |
| 1301054 | 22898 | 22913 | N/A | N/A | CAAGGAAGCACCCGTA | 62 | AK | 2492 |
| 1301088 | 25077 | 25092 | N/A | N/A | GCCGCTCTGTTTGTGA | 55 | AK | 2677 |
| 1301091 | 25074 | 25089 | N/A | N/A | GCTCTGTTTGTGATAC | 39 | AK | 2389 |
| | 26262 | 26277 | | | | | | |
| 1301136 | 31080 | 31095 | 3524 | 3539 | TAGTTTGATCCCCTTG | 52 | AK | 1820 |
| 1301149 | 35773 | 35788 | 4126 | 4141 | CTAATTACATGAGGTC | 35 | AK | 2364 |
| 1301156 | 5247 | 5262 | 199 | 214 | GGAGTGTGTCCTGAGC | 47 | AK | 2255 |
| 1232737 | 5138 | 5153 | 90 | 105 | ATGCCATCTTGACCCA | 63 | AL | 22 |
| 1300761 | 10302 | 10317 | 1102 | 1117 | AGGTACTCCAGTAAAC | 61 | AL | 2689 |
| 1300782 | 11006 | 11021 | 1309 | 1324 | GTCTTGCCGATGGCCA | 71 | AL | 1956 |
| 1300797 | 11264 | 11279 | 1567 | 1582 | CAGCTCATGATCAGGT | 26 | AL | 2676 |
| 1300820 | 11978 | 11993 | 2281 | 2296 | AACTTCTCGCAGTCCA | 54 | AL | 1037 |
| 1300855 | 14577 | 14592 | N/A | N/A | GGCAGGTGCCATCTAG | 85 | AL | 1759 |
| | 14869 | 14884 | | | | | | |
| | 15159 | 15174 | | | | | | |
| | 15232 | 15247 | | | | | | |
| 1300867 | 16735 | 16750 | 3053 | 3068 | TCTCCGAATGTTACAG | 53 | AL | 117 |
| 1300877 | 16647 | 16662 | 2965 | 2980 | TCAGTTAGACTCTGGC | 49 | AL | 150 |
| 1300882 | 5136 | 5151 | 88 | 103 | GCCATCTTGACCCATC | 63 | AL | 2678 |
| 1300903 | 11040 | 11055 | 1343 | 1358 | CTTAATGGGACTCACG | 69 | AL | 186 |
| 1300925 | 23494 | 23509 | N/A | N/A | TAGTGTAGAGGCTGCA | 37 | AL | 538 |
| | 23527 | 23542 | | | | | | |
| 1300952 | 25161 | 25176 | N/A | N/A | GACCTATAAGGACCCA | 50 | AL | 81 |
| 1300955 | 12133 | 12148 | 2436 | 2451 | CGAATTTGCCATAGTT | 39 | AL | 2423 |
| 1300963 | 23035 | 23050 | 3310 | 3325 | AGTCTGGTCAGGGAAT | 77 | AL | 2686 |
| 1300978 | 17696 | 17711 | N/A | N/A | TCTATGAACATGGCCC | 78 | AL | 2556 |
| 1300987 | 12139 | 12154 | 2442 | 2457 | CCTTTTCGAATTTGCC | 29 | AL | 420 |
| 1301000 | 18760 | 18775 | N/A | N/A | AAGCATTTAAGCCACC | 54 | AL | 1301 |
| 1301064 | 23821 | 23836 | N/A | N/A | TGATTATTAGTGGGTA | 71 | AL | 2078 |
| 1301095 | 26783 | 26798 | N/A | N/A | CCAATCGATAGATCTA | 85 | AL | 2541 |
| 1301120 | 27099 | 27114 | N/A | N/A | ACATAGGCTATACCTA | 87 | AL | 394 |
| | 27117 | 27132 | | | | | | |
| 1301131 | 29462 | 29477 | N/A | N/A | AGTCAAATTAAGCCCA | 107 | AL | 245 |
| 1301138 | 31017 | 31032 | 3461 | 3476 | AGTGCTGAGTACCGAG | 54 | AL | 972 |
| 1301147 | 25740 | 25755 | N/A | N/A | AATTGAGAGGTATATG | 111 | AL | 2543 |
| 1232737 | 5138 | 5153 | 90 | 105 | ATGCCATCTTGACCCA | 75 | AM | 22 |
| 1300717 | 26961 | 26976 | N/A | N/A | CTCGACTGATTAATGC | 45 | AM | 2544 |
| 1300732 | 27106 | 27121 | N/A | N/A | ACCTAACACATAGGCT | 45 | AM | 2085 |
| | 27124 | 27139 | | | | | | |
| 1300740 | 27364 | 27379 | N/A | N/A | ATTTAGGACCGTCCCC | 82 | AM | 87 |
| 1300793 | 11300 | 11315 | 1603 | 1618 | ACGATCTTGTGGATGG | 54 | AM | 649 |
| 1300807 | 11955 | 11970 | 2258 | 2273 | TTGGAACAGGTTCATC | 49 | AM | 2545 |
| 1300819 | 11977 | 11992 | 2280 | 2295 | ACTTCTCGCAGTCCAC | 57 | AM | 960 |
| 1300821 | 5153 | 5168 | 105 | 120 | AACAACCACTTCACGA | 93 | AM | 2546 |
| 1300826 | 12462 | 12477 | 2765 | 2780 | TGACTCCACCCGATGA | 71 | AM | 2692 |
| 1300838 | 12487 | 12502 | 2790 | 2805 | TGTTATGGAGAAACCC | 75 | AM | 576 |
| 1300851 | 12709 | 12724 | N/A | N/A | GGCATTATGATGTAGC | 46 | AM | 832 |
| 1300856 | 14680 | 14695 | N/A | N/A | GGCTCCAATCAGAGTC | 96 | AM | 2453 |
| | 14826 | 14841 | | | | | | |
| | 14972 | 14987 | | | | | | |
| 1300937 | 23532 | 23547 | N/A | N/A | TCTCCTAGTGTAGAGG | 63 | AM | 2549 |
| 1300974 | 17447 | 17462 | N/A | N/A | TAGATTAACATTAGGT | 26 | AM | 838 |
| 1300992 | 17992 | 18007 | N/A | N/A | TCAACCCATACAAAGC | 100 | AM | 2519 |
| 1301014 | 12145 | 12160 | 2448 | 2463 | AATACCCCTTTTCGAA | 99 | AM | 36 |
| 1301026 | 20275 | 20290 | N/A | N/A | TGACGATTTCATCCCA | 69 | AM | 2512 |
| 1301079 | 24553 | 24568 | N/A | N/A | GTCAAATTGCCAAGGA | 84 | AM | 1003 |
| 1301086 | 25076 | 25091 | N/A | N/A | CCGCTCTGTTTGTGAT | 88 | AM | 2683 |
| 1301111 | 26973 | 26988 | N/A | N/A | ATGCATAAAAGTCTCG | 73 | AM | 85 |
| 1301113 | 27107 | 27122 | N/A | N/A | TACCTAACACATAGGC | 27 | AM | 2470 |
| | 27125 | 27140 | | | | | | |
| 1301114 | 28761 | 28776 | N/A | N/A | TAACTATTAAGCAACG | 73 | AM | 90 |
| 1301139 | 31079 | 31094 | 3523 | 3538 | AGTTTGATCCCCTTGT | 75 | AM | 1743 |
| 1232737 | 5138 | 5153 | 90 | 105 | ATGCCATCTTGACCCA | 87 | AN | 22 |
| 1300725 | 27100 | 27115 | N/A | N/A | CACATAGGCTATACCT | 81 | AN | 1623 |
| | 27118 | 27133 | | | | | | |
| 1300726 | 27134 | 27149 | N/A | N/A | ATGTAGCTATACCTAA | 104 | AN | 2551 |
| 1300772 | 5145 | 5160 | 97 | 112 | CTTCACGATGCCATCT | 57 | AN | 253 |
| 1300832 | 11983 | 11998 | 2286 | 2301 | TGTAGAACTTCTCGCA | 62 | AN | 2685 |
| 1300901 | 17668 | 17683 | N/A | N/A | AATATATCGAGCATCA | 58 | AN | 606 |
| 1300927 | 23303 | 23318 | N/A | N/A | ATCTTATTAGCAGCAG | 56 | AN | 76 |
| 1300939 | 23499 | 23514 | N/A | N/A | TTTCCTAGTGTAGAGG | 55 | AN | 2554 |
| 1301012 | 19268 | 19283 | N/A | N/A | GACACTAGAGCCTCAG | 81 | AN | 1686 |
| 1301017 | 19883 | 19898 | N/A | N/A | CTTGATTAGTTTCCCC | 94 | AN | 1764 |
| 1301021 | 19877 | 19892 | N/A | N/A | TAGTTTCCCCGTCACA | 62 | AN | 2538 |
| 1301023 | 20418 | 20433 | N/A | N/A | AGTGAAGCGGGCGGTG | 106 | AN | 2530 |
| 1301050 | 12453 | 12468 | 2756 | 2771 | CCGATGACAGTTCTCA | 52 | AN | 2118 |
| 1301060 | 22427 | 22442 | N/A | N/A | AATGTCCCCGAGGCTC | 89 | AN | 2687 |
| 1301075 | 25032 | 25047 | N/A | N/A | GGTATTGACAATAGTA | 16 | AN | 2682 |
| 1301112 | 27021 | 27036 | N/A | N/A | GCTATACCTAACACAC | 63 | AN | 2598 |
| | 27057 | 27072 | | | | | | |
| 1301141 | 31016 | 31031 | 3460 | 3475 | GTGCTGAGTACCGAGG | 46 | AN | 895 |
| 1301148 | 35770 | 35785 | 4123 | 4138 | ATTACATGAGGTCACC | 99 | AN | 2567 |
| 1301155 | 26601 | 26616 | N/A | N/A | TTATTTGGTGATACGG | 46 | AN | 1160 |
| 1301162 | 25820 | 25835 | N/A | N/A | TATGTTTACACCAAGC | 22 | AN | 2522 |
| 1232737 | 5138 | 5153 | 90 | 105 | ATGCCATCTTGACCCA | 55 | AO | 22 |
| 1300738 | 27201 | 27216 | N/A | N/A | GCAGTATTGAGACAGG | 50 | AO | 931 |
| 1300751 | 27370 | 27385 | N/A | N/A | CAGAGCATTTAGGACC | 71 | AO | 2525 |
| 1300757 | 6023 | 6038 | 975 | 990 | CTCTCCTGTTGATCGC | 40 | AO | 798 |
| 1300768 | 10925 | 10940 | 1228 | 1243 | TTGTTGAGGCTCACAC | 46 | AO | 2684 |
| 1300776 | 11005 | 11020 | 1308 | 1323 | TCTTGCCGATGGCCAG | 65 | AO | 2691 |
| 1300783 | 11013 | 11028 | 1316 | 1331 | CGTCTTGGTCTTGCCG | 59 | AO | 2690 |
| 1300847 | 5172 | 5187 | 124 | 139 | TGTATTACAGTTTACG | 41 | AO | 255 |
| 1300857 | 14678 | 14693 | N/A | N/A | CTCCAATCAGAGTCTG | 47 | AO | 1991 |
| | 14824 | 14839 | | | | | | |
| | 14970 | 14985 | | | | | | |
| 1300907 | 21596 | 21611 | N/A | N/A | AATATGAGGACCCAGG | 70 | AO | 151 |
| 1300916 | 21604 | 21619 | N/A | N/A | TCTTATACAATATGAG | 71 | AO | 305 |
| 1300928 | 11045 | 11060 | 1348 | 1363 | TCCATCTTAATGGGAC | 127 | AO | 2693 |
| 1300981 | 17446 | 17461 | N/A | N/A | AGATTAACATTAGGTC | 18 | AO | 761 |
| 1300986 | 18753 | 18768 | N/A | N/A | TAAGCCACCGAACAGC | 77 | AO | 2563 |
| 1301024 | 20274 | 20289 | N/A | N/A | GACGATTTCATCCCAG | 35 | AO | 1918 |
| 1301036 | 20591 | 20606 | N/A | N/A | ATACACCTGACCAGAG | 72 | AO | 2531 |
| 1301039 | 20423 | 20438 | N/A | N/A | TTGTAAGTGAAGCGGG | 78 | AO | 2550 |
| 1301055 | 22897 | 22912 | N/A | N/A | AAGGAAGCACCCGTAC | 57 | AO | 2539 |
| 1301066 | 22902 | 22917 | N/A | N/A | TGGACAAGGAAGCACC | 53 | AO | 2565 |
| 1301074 | 14500 | 14515 | N/A | N/A | GGTGCTATCTAGAGAA | 19 | AO | 1604 |
| | 14646 | 14661 | | | | | | |
| | 14719 | 14734 | | | | | | |
| | 14792 | 14807 | | | | | | |
| | 14938 | 14953 | | | | | | |
| | 15011 | 15026 | | | | | | |
| | 15084 | 15099 | | | | | | |
| 1301077 | 25020 | 25035 | N/A | N/A | AGTAACTACATAGAGC | 48 | AO | 465 |
| 1301080 | 23827 | 23842 | N/A | N/A | AGTGCATGATTATTAG | 77 | AO | 2698 |
| 1301094 | 26789 | 26804 | N/A | N/A | TATGCCCCAATCGATA | 63 | AO | 2688 |
| 1301128 | 29461 | 29476 | N/A | N/A | GTCAAATTAAGCCCAT | 62 | AO | 168 |
| 1232737 | 5138 | 5153 | 90 | 105 | ATGCCATCTTGACCCA | 65 | AP | 22 |
| 1300721 | 5133 | 5148 | 85 | 100 | ATCTTGACCCATCAGC | 66 | AP | 21 |
| 1300724 | 6015 | 6030 | 967 | 982 | TTGATCGCAGCGAAGA | 68 | AP | 2029 |
| 1300756 | 6022 | 6037 | 974 | 989 | TCTCCTGTTGATCGCA | 83 | AP | 721 |
| 1300770 | 10923 | 10938 | 1226 | 1241 | GTTGAGGCTCACACTC | 64 | AP | 1340 |
| 1300787 | 11235 | 11250 | 1538 | 1553 | CTGTGTCACAAGGCTC | 46 | AP | 1572 |
| 1300796 | 11299 | 11314 | 1602 | 1617 | CGATCTTGTGGATGGG | 52 | AP | 2343 |
| 1300801 | 11263 | 11278 | 1566 | 1581 | AGCTCATGATCAGGTC | 44 | AP | 2535 |
| 1300809 | 11952 | 11967 | 2255 | 2270 | GAACAGGTTCATCCTC | 66 | AP | 34 |
| 1300817 | 11976 | 11991 | 2279 | 2294 | CTTCTCGCAGTCCACT | 76 | AP | 2704 |
| 1300850 | 14478 | 14493 | N/A | N/A | GCTGAGCCTCCTACCG | 45 | AP | 2296 |
| | 14624 | 14639 | | | | | | |
| | 14770 | 14785 | | | | | | |
| | 14916 | 14931 | | | | | | |
| | 15062 | 15077 | | | | | | |
| 1300858 | 14580 | 14595 | N/A | N/A | TAAGGCAGGTGCCATC | 66 | AP | 1913 |
| | 14872 | 14887 | | | | | | |
| | 15162 | 15177 | | | | | | |
| | 15235 | 15250 | | | | | | |
| 1300866 | 16734 | 16749 | 3052 | 3067 | CTCCGAATGTTACAGC | 96 | AP | 40 |
| 1300878 | 16645 | 16660 | 2963 | 2978 | AGTTAGACTCTGGCTG | 83 | AP | 1351 |
| 1300890 | 15187 | 15202 | N/A | N/A | CTCCCGTCAGAGTCTG | 62 | AP | 2570 |
| 1300935 | 23496 | 23511 | N/A | N/A | CCTAGTGTAGAGGCTG | 77 | AP | 77 |
| | 23529 | 23544 | | | | | | |
| 1300947 | 25160 | 25175 | N/A | N/A | ACCTATAAGGACCCAG | 65 | AP | 619 |
| 1300953 | 23033 | 23048 | 3308 | 3323 | TCTGGTCAGGGAATGG | 57 | AP | 1662 |
| 1301001 | 18759 | 18774 | N/A | N/A | AGCATTTAAGCCACCG | 95 | AP | 1224 |
| 1301013 | 19882 | 19897 | N/A | N/A | TTGATTAGTTTCCCCG | 124 | AP | 1687 |
| 1301072 | 23820 | 23835 | N/A | N/A | GATTATTAGTGGGTAT | 52 | AP | 616 |
| 1301102 | 26782 | 26797 | N/A | N/A | CAATCGATAGATCTAC | 57 | AP | 391 |
| 1301104 | 26972 | 26987 | N/A | N/A | TGCATAAAAGTCTCGA | 55 | AP | 1469 |
| 1301108 | 27023 | 27038 | N/A | N/A | AGGCTATACCTAACAC | 33 | AP | 1546 |
| | 27059 | 27074 | | | | | | |
| | 27095 | 27110 | | | | | | |
| | 27113 | 27128 | | | | | | |
| 1301134 | 31077 | 31092 | 3521 | 3536 | TTTGATCCCCTTGTCT | 77 | AP | 1589 |
| 1301161 | 25817 | 25832 | N/A | N/A | GTTTACACCAAGCATG | 68 | AP | 2571 |
| 1232737 | 5138 | 5153 | 90 | 105 | ATGCCATCTTGACCCA | 59 | AQ | 22 |
| 1300734 | 27200 | 27215 | N/A | N/A | CAGTATTGAGACAGGC | 54 | AQ | 2582 |
| 1300752 | 27369 | 27384 | N/A | N/A | AGAGCATTTAGGACCG | 91 | AQ | 2572 |
| 1300758 | 10301 | 10316 | 1101 | 1116 | GGTACTCCAGTAAACC | 72 | AQ | 2694 |
| 1300774 | 11011 | 11026 | 1314 | 1329 | TCTTGGTCTTGCCGAT | 63 | AQ | 2702 |
| 1300800 | 11262 | 11277 | 1565 | 1580 | GCTCATGATCAGGTCC | 64 | AQ | 2111 |
| 1300811 | 5151 | 5166 | 103 | 118 | CAACCACTTCACGATG | 67 | AQ | 946 |
| 1300828 | 12461 | 12476 | 2764 | 2779 | GACTCCACCCGATGAC | 102 | AQ | 2695 |
| 1300843 | 5171 | 5186 | 123 | 138 | GTATTACAGTTTACGG | 61 | AQ | 178 |
| 1300886 | 17662 | 17677 | N/A | N/A | TCGAGCATCATGGTTG | 24 | AQ | 2547 |
| 1300891 | 5293 | 5308 | 245 | 260 | AGTTAGCCAAATGCTT | 81 | AQ | 410 |
| 1300906 | 21594 | 21609 | N/A | N/A | TATGAGGACCCAGGGC | 64 | AQ | 2574 |
| 1300923 | 11044 | 11059 | 1347 | 1362 | CCATCTTAATGGGACT | 73 | AQ | 2585 |
| 1300949 | 25159 | 25174 | N/A | N/A | CCTATAAGGACCCAGC | 72 | AQ | 542 |
| 1300962 | 25166 | 25181 | N/A | N/A | TGTACGACCTATAAGG | 99 | AQ | 2697 |
| 1300965 | 23043 | 23058 | 3318 | 3333 | CCACATAGAGTCTGGT | 72 | AQ | 582 |
| 1300983 | 12138 | 12153 | 2441 | 2456 | CTTTTCGAATTTGCCA | 59 | AQ | 343 |
| 1300991 | 17988 | 18003 | N/A | N/A | CCCATACAAAGCATTC | 63 | AQ | 2576 |
| 1301025 | 20416 | 20431 | N/A | N/A | TGAAGCGGGCGGTGTG | 73 | AQ | 2579 |
| 1301037 | 20590 | 20605 | N/A | N/A | TACACCTGACCAGAGA | 45 | AQ | 2587 |
| 1301053 | 20701 | 20716 | N/A | N/A | AGCTCCGGAATAGAGA | 61 | AQ | 612 |
| | 20743 | 20758 | | | | | | |
| | 20911 | 20926 | | | | | | |
| | 20995 | 21010 | | | | | | |
| 1301056 | 22895 | 22910 | N/A | N/A | GGAAGCACCCGTACCT | 79 | AQ | 2708 |
| 1301089 | 25075 | 25090 | N/A | N/A | CGCTCTGTTTGTGATA | 64 | AQ | 2699 |
| 1301107 | 27022 | 27037 | N/A | N/A | GGCTATACCTAACACA | 56 | AQ | 162 |
| | 27058 | 27073 | | | | | | |
| | 27112 | 27127 | | | | | | |
| 1301116 | 28760 | 28775 | N/A | N/A | AACTATTAAGCAACGG | 58 | AQ | 628 |
| 1301122 | 27027 | 27042 | N/A | N/A | CCATAGGCTATACCTA | 65 | AQ | 470 |
| | 27063 | 27078 | | | | | | |
| 1232737 | 5138 | 5153 | 90 | 105 | ATGCCATCTTGACCCA | 65 | AR | 22 |
| 1300718 | 26960 | 26975 | N/A | N/A | TCGACTGATTAATGCC | 64 | AR | 2558 |
| 1300741 | 27363 | 27378 | N/A | N/A | TTTAGGACCGTCCCCC | 114 | AR | 2569 |
| 1300759 | 5143 | 5158 | 95 | 110 | TCACGATGCCATCTTG | 65 | AR | 2332 |
| 1300760 | 10300 | 10315 | 1100 | 1115 | GTACTCCAGTAAACCC | 65 | AR | 2031 |
| 1300802 | 5150 | 5165 | 102 | 117 | AACCACTTCACGATGC | 68 | AR | 871 |
| 1300813 | 11982 | 11997 | 2285 | 2300 | GTAGAACTTCTCGCAG | 43 | AR | 2583 |
| 1300836 | 12704 | 12719 | N/A | N/A | TATGATGTAGCTGTGG | 82 | AR | 678 |
| 1300840 | 12486 | 12501 | 2789 | 2804 | GTTATGGAGAAACCCC | 68 | AR | 887 |
| 1300859 | 14590 | 14605 | N/A | N/A | GTCCTCAGAGTAAGGC | 37 | AR | 759 |
| | 14882 | 14897 | | | | | | |
| | 15172 | 15187 | | | | | | |
| | 15245 | 15260 | | | | | | |
| 1300869 | 16733 | 16748 | 3051 | 3066 | TCCGAATGTTACAGCC | 77 | AR | 578 |
| 1300893 | 17667 | 17682 | N/A | N/A | ATATATCGAGCATCAT | 56 | AR | 529 |
| 1300895 | 17675 | 17690 | N/A | N/A | GAAATGGAATATATCG | 84 | AR | 1608 |
| 1300904 | 20580 | 20595 | N/A | N/A | CAGAGAGCTCCGGAAT | 40 | AR | 918 |
| | 20706 | 20721 | | | | | | |
| | 20748 | 20763 | | | | | | |
| | 20916 | 20931 | | | | | | |
| | 20958 | 20973 | | | | | | |
| 1300936 | 23531 | 23546 | N/A | N/A | CTCCTAGTGTAGAGGC | 84 | AR | 2561 |
| 1300940 | 23495 | 23510 | N/A | N/A | CTAGTGTAGAGGCTGC | 43 | AR | 615 |
| | 23528 | 23543 | | | | | | |
| 1300942 | 23498 | 23513 | N/A | N/A | TTCCTAGTGTAGAGGC | 68 | AR | 2696 |
| 1300975 | 12137 | 12152 | 2440 | 2455 | TTTTCGAATTTGCCAT | 56 | AR | 2192 |
| 1301002 | 18758 | 18773 | N/A | N/A | GCATTTAAGCCACCGA | 70 | AR | 1147 |
| 1301003 | 18860 | 18875 | N/A | N/A | ACTGTGCAGTAACATA | 47 | AR | 2700 |
| 1301005 | 12144 | 12159 | 2447 | 2462 | ATACCCCTTTTCGAAT | 94 | AR | 2347 |
| 1301078 | 25019 | 25034 | N/A | N/A | GTAACTACATAGAGCA | 49 | AR | 2466 |
| 1301097 | 26787 | 26802 | N/A | N/A | TGCCCCAATCGATAGA | 100 | AR | 2596 |
| 1301106 | 27024 | 27039 | N/A | N/A | TAGGCTATACCTAACA | 100 | AR | 239 |
| | 27060 | 27075 | | | | | | |
| | 27096 | 27111 | | | | | | |
| | 27114 | 27129 | | | | | | |
| 1301115 | 28759 | 28774 | N/A | N/A | ACTATTAAGCAACGGT | 87 | AR | 551 |
| 1301121 | 27111 | 27126 | N/A | N/A | GCTATACCTAACACAT | 57 | AR | 2162 |
| | 27129 | 27144 | | | | | | |
| 1301127 | 29459 | 29474 | N/A | N/A | CAAATTAAGCCCATTC | 58 | AR | 2013 |
| 1301150 | 31084 | 31099 | 3528 | 3543 | AGAGTAGTTTGATCCC | 70 | AR | 2589 |
| 1301158 | 26600 | 26615 | N/A | N/A | TATTTGGTGATACGGT | 36 | AR | 2568 |
| 1232737 | 5138 | 5153 | 90 | 105 | ATGCCATCTTGACCCA | 55 | AS | 22 |
| 1300775 | 11010 | 11025 | 1313 | 1328 | CTTGGTCTTGCCGATG | 83 | AS | 2715 |
| 1300778 | 11004 | 11019 | 1307 | 1322 | CTTGCCGATGGCCAGA | 67 | AS | 2703 |
| 1300788 | 11233 | 11248 | 1536 | 1551 | GTGTCACAAGGCTCAC | 56 | AS | 1418 |
| 1300798 | 11298 | 11313 | 1601 | 1616 | GATCTTGTGGATGGGT | 105 | AS | 2600 |
| 1300835 | 5170 | 5185 | 122 | 137 | TATTACAGTTTACGGT | 61 | AS | 101 |
| 1300860 | 14575 | 14590 | N/A | N/A | CAGGTGCCATCTAGAG | 57 | AS | 1605 |
| | 14867 | 14882 | | | | | | |
| | 15157 | 15172 | | | | | | |
| | 15230 | 15245 | | | | | | |
| 1300888 | 17660 | 17675 | N/A | N/A | GAGCATCATGGTTGTT | 56 | AS | 2601 |
| 1300896 | 17673 | 17688 | N/A | N/A | AATGGAATATATCGAG | 87 | AS | 222 |
| 1300918 | 21603 | 21618 | N/A | N/A | CTTATACAATATGAGG | 68 | AS | 843 |
| 1300926 | 23493 | 23508 | N/A | N/A | AGTGTAGAGGCTGCAT | 51 | AS | 2716 |
| 1300931 | 23301 | 23316 | N/A | N/A | CTTATTAGCAGCAGTA | 41 | AS | 845 |
| 1300941 | 23497 | 23512 | N/A | N/A | TCCTAGTGTAGAGGCT | 42 | AS | 154 |
| | 23530 | 23545 | | | | | | |
| 1300956 | 23007 | 23022 | 3282 | 3297 | ATACTGATGCAAGATC | 57 | AS | 738 |
| 1300964 | 17445 | 17460 | N/A | N/A | GATTAACATTAGGTCT | 76 | AS | 144 |
| 1301004 | 19469 | 19484 | N/A | N/A | GATGAAGAGTGTGATC | 37 | AS | 2578 |
| 1301029 | 20273 | 20288 | N/A | N/A | ACGATTTCATCCCAGC | 53 | AS | 2594 |
| 1301042 | 20422 | 20437 | N/A | N/A | TGTAAGTGAAGCGGGC | 104 | AS | 2580 |
| 1301043 | 12452 | 12467 | 2755 | 2770 | CGATGACAGTTCTCAA | 71 | AS | 2426 |
| 1301065 | 22901 | 22916 | N/A | N/A | GGACAAGGAAGCACCC | 66 | AS | 2605 |
| 1301073 | 23825 | 23840 | N/A | N/A | TGCATGATTATTAGTG | 88 | AS | 2606 |
| 1301105 | 27025 | 27040 | N/A | N/A | ATAGGCTATACCTAAC | 89 | AS | 316 |
| | 27061 | 27076 | | | | | | |
| | 27097 | 27112 | | | | | | |
| | 27115 | 27130 | | | | | | |
| 1301129 | 5141 | 5156 | 93 | 108 | ACGATGCCATCTTGAC | 91 | AS | 2254 |
| 1301133 | 31015 | 31030 | 3459 | 3474 | TGCTGAGTACCGAGGA | 52 | AS | 818 |
| 1301142 | 31022 | 31037 | 3466 | 3481 | TGATTAGTGCTGAGTA | 49 | AS | 1280 |
| 1232737 | 5138 | 5153 | 90 | 105 | ATGCCATCTTGACCCA | 57 | AT | 22 |
| 1300716 | 27028 | 27043 | N/A | N/A | CCCATAGGCTATACCT | 55 | AT | 547 |
| | 27064 | 27079 | | | | | | |
| 1300719 | 26959 | 26974 | N/A | N/A | CGACTGATTAATGCCA | 45 | AT | 2713 |
| 1300729 | 27132 | 27147 | N/A | N/A | GTAGCTATACCTAACA | 79 | AT | 2701 |
| 1300735 | 27362 | 27377 | N/A | N/A | TTAGGACCGTCCCCCG | 94 | AT | 2714 |
| 1300743 | 27368 | 27383 | N/A | N/A | GAGCATTTAGGACCGT | 82 | AT | 1393 |
| 1300771 | 10883 | 10898 | 1186 | 1201 | TCAATGCACTGGAATC | 41 | AT | 2032 |
| 1300773 | 11003 | 11018 | 1306 | 1321 | TTGCCGATGGCCAGAA | 63 | AT | 2718 |
| 1300804 | 11948 | 11963 | 2251 | 2266 | AGGTTCATCCTCAGGA | 73 | AT | 729 |
| 1300822 | 11975 | 11990 | 2278 | 2293 | TTCTCGCAGTCCACTT | 48 | AT | 2705 |
| 1300830 | 12460 | 12475 | 2763 | 2778 | ACTCCACCCGATGACA | 66 | AT | 2706 |
| 1300844 | 14477 | 14492 | N/A | N/A | CTGAGCCTCCTACCGG | 85 | AT | 2219 |
| | 14623 | 14638 | | | | | | |
| | 14769 | 14784 | | | | | | |
| | 14915 | 14930 | | | | | | |
| | 15061 | 15076 | | | | | | |
| 1300861 | 14573 | 14588 | N/A | N/A | GGTGCCATCTAGAGGG | 59 | AT | 1451 |
| | 14865 | 14880 | | | | | | |
| | 15155 | 15170 | | | | | | |
| | 15228 | 15243 | | | | | | |
| 1300868 | 16732 | 16747 | 3050 | 3065 | CCGAATGTTACAGCCA | 67 | AT | 501 |
| 1300894 | 20578 | 20593 | N/A | N/A | GAGAGCTCCGGAATAC | 51 | AT | 841 |
| | 20956 | 20971 | | | | | | |
| 1300902 | 17666 | 17681 | N/A | N/A | TATATCGAGCATCATG | 63 | AT | 452 |
| 1300957 | 23006 | 23021 | 3281 | 3296 | TACTGATGCAAGATCC | 68 | AT | 661 |
| 1300961 | 25164 | 25179 | N/A | N/A | TACGACCTATAAGGAC | 94 | AT | 2603 |
| 1300970 | 23041 | 23056 | 3316 | 3331 | ACATAGAGTCTGGTCA | 92 | AT | 505 |
| 1300988 | 18354 | 18369 | N/A | N/A | GCGGACTTTAACACTT | 55 | AT | 2586 |
| 1301006 | 12143 | 12158 | 2446 | 2461 | TACCCCTTTTCGAATT | 88 | AT | 2614 |
| 1301010 | 18859 | 18874 | N/A | N/A | CTGTGCAGTAACATAT | 64 | AT | 2707 |
| 1301018 | 19881 | 19896 | N/A | N/A | TGATTAGTTTCCCCGT | 32 | AT | 2577 |
| 1301058 | 22431 | 22446 | N/A | N/A | GCATAATGTCCCCGAG | 86 | AT | 536 |
| 1301062 | 20698 | 20713 | N/A | N/A | TCCGGAATAGAGAAAG | 95 | AT | 381 |
| | 20740 | 20755 | | | | | | |
| | 20908 | 20923 | | | | | | |
| | 20992 | 21007 | | | | | | |
| 1301110 | 26971 | 26986 | N/A | N/A | GCATAAAAGTCTCGAC | 63 | AT | 623 |
| 1301153 | 26599 | 26614 | N/A | N/A | ATTTGGTGATACGGTT | 21 | AT | 2709 |
| 1232737 | 5138 | 5153 | 90 | 105 | ATGCCATCTTGACCCA | 52 | AU | 22 |
| 1300723 | 27199 | 27214 | N/A | N/A | AGTATTGAGACAGGCA | 46 | AU | 2622 |
| 1300730 | 27131 | 27146 | N/A | N/A | TAGCTATACCTAACAC | 63 | AU | 2634 |
| 1300744 | 6021 | 6036 | 973 | 988 | CTCCTGTTGATCGCAG | 43 | AU | 644 |
| 1300789 | 5149 | 5164 | 101 | 116 | ACCACTTCACGATGCC | 93 | AU | 792 |
| 1300795 | 11261 | 11276 | 1564 | 1579 | CTCATGATCAGGTCCC | 74 | AU | 2419 |
| 1300834 | 12466 | 12481 | 2769 | 2784 | ACAGTGACTCCACCCG | 55 | AU | 656 |
| 1300845 | 14570 | 14585 | N/A | N/A | GCCATCTAGAGGGATG | 57 | AU | 1297 |
| | 14862 | 14877 | | | | | | |
| | 15152 | 15167 | | | | | | |
| | 15225 | 15240 | | | | | | |
| 1300862 | 14574 | 14589 | N/A | N/A | AGGTGCCATCTAGAGG | 46 | AU | 1528 |
| | 14866 | 14881 | | | | | | |
| | 15156 | 15171 | | | | | | |
| | 15229 | 15244 | | | | | | |
| 1300879 | 16644 | 16659 | 2962 | 2977 | GTTAGACTCTGGCTGG | 68 | AU | 1274 |
| 1300881 | 5290 | 5305 | 242 | 257 | TAGCCAAATGCTTACC | 72 | AU | 1948 |
| 1300889 | 15185 | 15200 | N/A | N/A | CCCGTCAGAGTCTGTC | 28 | AU | 2711 |
| 1300917 | 11043 | 11058 | 1346 | 1361 | CATCTTAATGGGACTC | 60 | AU | 417 |
| 1300951 | 23502 | 23517 | N/A | N/A | CTCTTTCCTAGTGTAG | 40 | AU | 2611 |
| 1300985 | 18353 | 18368 | N/A | N/A | CGGACTTTAACACTTG | 50 | AU | 2639 |
| 1300993 | 18757 | 18772 | N/A | N/A | CATTTAAGCCACCGAA | 71 | AU | 224 |
| 1301027 | 20279 | 20294 | N/A | N/A | TGTTTGACGATTTCAT | 48 | AU | 2712 |
| 1301034 | 12451 | 12466 | 2754 | 2769 | GATGACAGTTCTCAAT | 56 | AU | 2041 |
| 1301046 | 20697 | 20712 | N/A | N/A | CCGGAATAGAGAAAGC | 44 | AU | 304 |
| | 20739 | 20754 | | | | | | |
| | 20907 | 20922 | | | | | | |
| | 20991 | 21006 | | | | | | |
| 1301071 | 23817 | 23832 | N/A | N/A | TATTAGTGGGTATTCC | 80 | AU | 462 |
| 1301087 | 25038 | 25053 | N/A | N/A | AGGTTAGGTATTGACA | 32 | AU | 2595 |
| 1301101 | 26781 | 26796 | N/A | N/A | AATCGATAGATCTACA | 42 | AU | 314 |
| 1301109 | 26970 | 26985 | N/A | N/A | CATAAAAGTCTCGACT | 68 | AU | 2619 |
| 1301118 | 28758 | 28773 | N/A | N/A | CTATTAAGCAACGGTT | 39 | AU | 474 |
| 1301123 | 31014 | 31029 | 3458 | 3473 | GCTGAGTACCGAGGAC | 56 | AU | 741 |
| 1301132 | 28765 | 28780 | N/A | N/A | CTGGTAACTATTAAGC | 89 | AU | 2597 |
| 1301152 | 31083 | 31098 | 3527 | 3542 | GAGTAGTTTGATCCCC | 75 | AU | 2051 |
| 1232737 | 5138 | 5153 | 90 | 105 | ATGCCATCTTGACCCA | 43 | AV | 22 |
| 1300736 | 27206 | 27221 | N/A | N/A | CTTGAGCAGTATTGAG | 75 | AV | 2644 |
| 1300747 | 6020 | 6035 | 972 | 987 | TCCTGTTGATCGCAGC | 63 | AV | 2338 |
| 1300779 | 11009 | 11024 | 1312 | 1327 | TTGGTCTTGCCGATGG | 53 | AV | 2719 |
| 1300790 | 11232 | 11247 | 1535 | 1550 | TGTCACAAGGCTCACC | 59 | AV | 2710 |
| 1300810 | 11947 | 11962 | 2250 | 2265 | GGTTCATCCTCAGGAA | 74 | AV | 652 |
| 1300816 | 11981 | 11996 | 2284 | 2299 | TAGAACTTCTCGCAGT | 12 | AV | 2623 |
| 1300833 | 12703 | 12718 | N/A | N/A | ATGATGTAGCTGTGGC | 63 | AV | 2625 |
| 1300841 | 5169 | 5184 | 121 | 136 | ATTACAGTTTACGGTG | 90 | AV | 1331 |
| 1300846 | 14569 | 14584 | N/A | N/A | CCATCTAGAGGGATGG | 97 | AV | 1220 |
| | 14861 | 14876 | | | | | | |
| | 15151 | 15166 | | | | | | |
| | 15224 | 15239 | | | | | | |
| 1300872 | 14681 | 14696 | N/A | N/A | GGGCTCCAATCAGAGT | 71 | AV | 2145 |
| | 14827 | 14842 | | | | | | |
| | 14973 | 14988 | | | | | | |
| 1300913 | 21600 | 21615 | N/A | N/A | ATACAATATGAGGACC | 88 | AV | 689 |
| 1300966 | 17443 | 17458 | N/A | N/A | TTAACATTAGGTCTTG | 43 | AV | 2638 |
| 1300979 | 12136 | 12151 | 2439 | 2454 | TTTCGAATTTGCCATA | 63 | AV | 266 |
| 1300995 | 18858 | 18873 | N/A | N/A | TGTGCAGTAACATATC | 52 | AV | 2722 |
| 1301019 | 19880 | 19895 | N/A | N/A | GATTAGTTTCCCCGTC | 50 | AV | 1610 |
| 1301033 | 20421 | 20436 | N/A | N/A | GTAAGTGAAGCGGGCG | 113 | AV | 2072 |
| 1301047 | 21014 | 21029 | N/A | N/A | AGGACACACCTGATCA | 88 | AV | 2717 |
| 1301063 | 22900 | 22915 | N/A | N/A | GACAAGGAAGCACCCG | 83 | AV | 2076 |
| 1301082 | 5140 | 5155 | 92 | 107 | CGATGCCATCTTGACC | 48 | AV | 2177 |
| 1301099 | 26786 | 26801 | N/A | N/A | GCCCCAATCGATAGAT | 75 | AV | 2642 |
| 1301103 | 27026 | 27041 | N/A | N/A | CATAGGCTATACCTAA | 107 | AV | 393 |
| | 27062 | 27077 | | | | | | |
| | 27098 | 27113 | | | | | | |
| | 27116 | 27131 | | | | | | |
| 1232737 | 5138 | 5153 | 90 | 105 | ATGCCATCTTGACCCA | 65 | AW | 22 |
| 1300762 | 10297 | 10312 | 1097 | 1112 | CTCCAGTAAACCCATC | 66 | AW | 1877 |
| 1300784 | 11260 | 11275 | 1563 | 1578 | TCATGATCAGGTCCCC | 66 | AW | 2034 |
| 1300823 | 12465 | 12480 | 2768 | 2783 | CAGTGACTCCACCCGA | 61 | AW | 2350 |
| 1300875 | 15111 | 15126 | N/A | N/A | CAGTCAGAGTCTGTCC | 22 | AW | 914 |
| | 15257 | 15272 | | | | | | |
| 1300880 | 16643 | 16658 | 2961 | 2976 | TTAGACTCTGGCTGGT | 80 | AW | 2608 |
| 1300944 | 23535 | 23550 | N/A | N/A | TCCTCTCCTAGTGTAG | 80 | AW | 2724 |
| 1300945 | 25163 | 25178 | N/A | N/A | ACGACCTATAAGGACC | 44 | AW | 2657 |
| 1300946 | 25158 | 25173 | N/A | N/A | CTATAAGGACCCAGCC | 102 | AW | 2592 |
| 1300969 | 23040 | 23055 | 3315 | 3330 | CATAGAGTCTGGTCAG | 69 | AW | 428 |
| 1300989 | 17703 | 17718 | N/A | N/A | ACTCTCATCTATGAAC | 51 | AW | 2725 |
| 1301008 | 19273 | 19288 | N/A | N/A | TGATAGACACTAGAGC | 30 | AW | 2613 |
| 1301011 | 12142 | 12157 | 2445 | 2460 | ACCCCTTTTCGAATTT | 61 | AW | 2269 |
| 1301051 | 20929 | 20944 | N/A | N/A | GAACACACCTGACCAG | 58 | AW | 1689 |
| | 21055 | 21070 | | | | | | |
| 1301076 | 25018 | 25033 | N/A | N/A | TAACTACATAGAGCAT | 63 | AW | 2616 |
| 1301083 | 26779 | 26794 | N/A | N/A | TCGATAGATCTACACA | 62 | AW | 2617 |
| 1301125 | 31012 | 31027 | 3456 | 3471 | TGAGTACCGAGGACAA | 67 | AW | 2358 |
| 1301140 | 31020 | 31035 | 3464 | 3479 | ATTAGTGCTGAGTACC | 123 | AW | 1203 |
| 1301154 | 25814 | 25829 | N/A | N/A | TACACCAAGCATGTGT | 107 | AW | 2607 |
| 1301160 | 26598 | 26613 | N/A | N/A | TTTGGTGATACGGTTA | 31 | AW | 2726 |
| 1232737 | 5138 | 5153 | 90 | 105 | ATGCCATCTTGACCCA | 43 | AX | 22 |
| 1300727 | 27198 | 27213 | N/A | N/A | GTATTGAGACAGGCAG | 73 | AX | 854 |
| 1300748 | 27367 | 27382 | N/A | N/A | AGCATTTAGGACCGTC | 56 | AX | 1316 |
| 1300755 | 10295 | 10310 | 1095 | 1110 | CCAGTAAACCCATCCA | 53 | AX | 1723 |
| 1300763 | 10880 | 10895 | 1183 | 1198 | ATGCACTGGAATCTGC | 41 | AX | 1801 |
| 1300765 | 11001 | 11016 | 1304 | 1319 | GCCGATGGCCAGAAGC | 68 | AX | 2621 |
| 1300814 | 11974 | 11989 | 2277 | 2292 | TCTCGCAGTCCACTTC | 63 | AX | 2720 |
| 1300839 | 12702 | 12717 | N/A | N/A | TGATGTAGCTGTGGCA | 42 | AX | 2654 |
| 1300848 | 14572 | 14587 | N/A | N/A | GTGCCATCTAGAGGGA | 94 | AX | 1374 |
| | 14864 | 14879 | | | | | | |
| | 15154 | 15169 | | | | | | |
| | 15227 | 15242 | | | | | | |
| 1300853 | 5288 | 5303 | 240 | 255 | GCCAAATGCTTACCAG | 47 | AX | 333 |
| 1300876 | 15112 | 15127 | N/A | N/A | CCAGTCAGAGTCTGTC | 11 | AX | 991 |
| | 15258 | 15273 | | | | | | |
| 1300884 | 17665 | 17680 | N/A | N/A | ATATCGAGCATCATGG | 38 | AX | 375 |
| 1300892 | 15184 | 15199 | N/A | N/A | CCGTCAGAGTCTGTCC | 28 | AX | 2721 |
| 1300900 | 17672 | 17687 | N/A | N/A | ATGGAATATATCGAGC | 15 | AX | 145 |
| 1300919 | 11042 | 11057 | 1345 | 1360 | ATCTTAATGGGACTCA | 39 | AX | 340 |
| 1300920 | 23282 | 23297 | N/A | N/A | ATTATATGCCTCCAGT | 73 | AX | 2626 |
| 1300924 | 23492 | 23507 | N/A | N/A | GTGTAGAGGCTGCATG | 35 | AX | 2610 |
| 1300976 | 12135 | 12150 | 2438 | 2453 | TTCGAATTTGCCATAG | 39 | AX | 189 |
| 1300994 | 18756 | 18771 | N/A | N/A | ATTTAAGCCACCGAAC | 80 | AX | 2646 |
| 1301015 | 20272 | 20287 | N/A | N/A | CGATTTCATCCCAGCC | 45 | AX | 2615 |
| 1301038 | 20426 | 20441 | N/A | N/A | GGTTTGTAAGTGAAGC | 34 | AX | 2659 |
| 1301040 | 20420 | 20435 | N/A | N/A | TAAGTGAAGCGGGCGG | 109 | AX | 2630 |
| 1301059 | 22430 | 22445 | N/A | N/A | CATAATGTCCCCGAGG | 57 | AX | 2632 |
| 1301061 | 12457 | 12472 | 2760 | 2775 | CCACCCGATGACAGTT | 64 | AX | 2631 |
| 1301119 | 28757 | 28772 | N/A | N/A | TATTAAGCAACGGTTA | 45 | AX | 1549 |
| 1232737 | 5138 | 5153 | 90 | 105 | ATGCCATCTTGACCCA | 69 | AY | 22 |
| 1300745 | 6019 | 6034 | 971 | 986 | CCTGTTGATCGCAGCG | 69 | AY | 2260 |
| 1300767 | 10305 | 10320 | 1105 | 1120 | GAAAGGTACTCCAGTA | 62 | AY | 338 |
| 1300792 | 11231 | 11246 | 1534 | 1549 | GTCACAAGGCTCACCT | 56 | AY | 2624 |
| 1300794 | 11297 | 11312 | 1600 | 1615 | ATCTTGTGGATGGGTG | 45 | AY | 2265 |
| 1300806 | 11304 | 11319 | 1607 | 1622 | TCTCACGATCTTGTGG | 53 | AY | 882 |
| 1300849 | 14571 | 14586 | N/A | N/A | TGCCATCTAGAGGGAT | 62 | AY | 450 |
| | 14863 | 14878 | | | | | | |
| | 15153 | 15168 | | | | | | |
| | 15226 | 15241 | | | | | | |
| 1300870 | 16731 | 16746 | 3049 | 3064 | CGAATGTTACAGCCAG | 89 | AY | 2353 |
| 1300905 | 20582 | 20597 | N/A | N/A | ACCAGAGAGCTCCGGA | 54 | AY | 611 |
| | 20708 | 20723 | | | | | | |
| | 20750 | 20765 | | | | | | |
| | 20918 | 20933 | | | | | | |
| 1300915 | 21599 | 21614 | N/A | N/A | TACAATATGAGGACCC | 83 | AY | 228 |
| 1300943 | 23501 | 23516 | N/A | N/A | TCTTTCCTAGTGTAGA | 76 | AY | 2635 |
| 1300950 | 23534 | 23549 | N/A | N/A | CCTCTCCTAGTGTAGA | 91 | AY | 2732 |
| 1300958 | 23004 | 23019 | 3279 | 3294 | CTGATGCAAGATCCTG | 47 | AY | 2277 |
| 1300982 | 17449 | 17464 | N/A | N/A | ACTAGATTAACATTAG | 103 | AY | 992 |
| 1300998 | 18857 | 18872 | N/A | N/A | GTGCAGTAACATATCC | 42 | AY | 2489 |
| 1301032 | 20277 | 20292 | N/A | N/A | TTTGACGATTTCATCC | 72 | AY | 2731 |
| 1301067 | 23824 | 23839 | N/A | N/A | GCATGATTATTAGTGG | 47 | AY | 2633 |
| 1301070 | 22899 | 22914 | N/A | N/A | ACAAGGAAGCACCCGT | 58 | AY | 1999 |
| 1301100 | 26785 | 26800 | N/A | N/A | CCCCAATCGATAGATC | 83 | AY | 2661 |
| 1301135 | 31019 | 31034 | 3463 | 3478 | TTAGTGCTGAGTACCG | 77 | AY | 1126 |
| 1301159 | 26596 | 26611 | N/A | N/A | TGGTGATACGGTTACT | 44 | AY | 2496 |
| 1232737 | 5138 | 5153 | 90 | 105 | ATGCCATCTTGACCCA | 63 | AZ | 22 |
| 1300722 | 26958 | 26973 | N/A | N/A | GACTGATTAATGCCAC | 56 | AZ | 2620 |
| 1300785 | 11259 | 11274 | 1562 | 1577 | CATGATCAGGTCCCCC | 77 | AZ | 1957 |
| 1300791 | 5148 | 5163 | 100 | 115 | CCACTTCACGATGCCA | 60 | AZ | 407 |
| 1300815 | 11980 | 11995 | 2283 | 2298 | AGAACTTCTCGCAGTC | 46 | AZ | 2723 |
| 1300827 | 12464 | 12479 | 2767 | 2782 | AGTGACTCCACCCGAT | 42 | AZ | 2733 |
| 1300897 | 17671 | 17686 | N/A | N/A | TGGAATATATCGAGCA | 16 | AZ | 1454 |
| 1300910 | 20704 | 20719 | N/A | N/A | GAGAGCTCCGGAATAG | 82 | AZ | 842 |
| | 20746 | 20761 | | | | | | |
| | 20914 | 20929 | | | | | | |
| 1300929 | 23491 | 23506 | N/A | N/A | TGTAGAGGCTGCATGC | 88 | AZ | 2501 |
| 1300967 | 17441 | 17456 | N/A | N/A | AACATTAGGTCTTGTG | 41 | AZ | 684 |
| 1300971 | 23039 | 23054 | 3314 | 3329 | ATAGAGTCTGGTCAGG | 97 | AZ | 351 |
| 1300973 | 17698 | 17713 | N/A | N/A | CATCTATGAACATGGC | 48 | AZ | 1916 |
| 1300990 | 18352 | 18367 | N/A | N/A | GGACTTTAACACTTGC | 41 | AZ | 2665 |
| 1301016 | 19887 | 19902 | N/A | N/A | GATTCTTGATTAGTTT | 32 | AZ | 2671 |
| 1301031 | 12449 | 12464 | 2752 | 2767 | TGACAGTTCTCAATGC | 69 | AZ | 2727 |
| 1301035 | 5139 | 5154 | 91 | 106 | GATGCCATCTTGACCC | 46 | AZ | 2100 |
| 1301084 | 25079 | 25094 | N/A | N/A | AGGCCGCTCTGTTTGT | 116 | AZ | 2666 |
| 1301090 | 25036 | 25051 | N/A | N/A | GTTAGGTATTGACAAT | 95 | AZ | 2651 |
| 1301096 | 26968 | 26983 | N/A | N/A | TAAAAGTCTCGACTGA | 43 | AZ | 546 |
| 1301130 | 28763 | 28778 | N/A | N/A | GGTAACTATTAAGCAA | 107 | AZ | 2653 |
| 1301151 | 31082 | 31097 | 3526 | 3541 | AGTAGTTTGATCCCCT | 71 | AZ | 1974 |

### Example 4: Effect of Mixed MOE and cEt, Uniform Phosphorothioate Modified Oligonucleotides on Human NLRP3 RNA In Vitro, Single Dose

Modified oligonucleotides complementary to human NLRP3 nucleic acid were designed and tested for their single dose effects on NLRP3 RNA in vitro. The modified oligonucleotides were tested in a series of experiments that had the same culture conditions.

The modified oligonucleotides in the table below are 16 nucleosides in length, wherein the sugar motif for the modified oligonucleotides is (from 5′ to 3′); kkkdddddddddkkke; wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. “N/A” indicates that the modified oligonucleotide is not 100% complementary to that particular target nucleic acid sequence.

Cultured THP-1 cells were treated with modified oligonucleotide at a concentration of 2000 nM by electroporation at a density of 300,000 cells per well. After a treatment period of approximately 24 hours, total RNA was isolated from the cells and NLRP3 RNA levels were measured by quantitative real-time RTPCR. NLRP3 RNA levels were measured by human primer-probe set RTS37509 (described herein above). NLRP3 RNA levels were normalized to total RNA content, as measured by RIBOGREEN®. Reduction of NLRP3 RNA is presented in the tables below as percent NLRP3 RNA relative to the amount of NLRP3 RNA in untreated control cells (% UTC).

Each separate experimental analysis described in this example is identified by a letter ID in the table column below labeled “AID” (Analysis ID). In the table below, Compound No. 1232737 (described herein above) was used as a benchmark on multiple plates.

TABLE 6
Reduction of NLRP3 RNA by modified oligonucleotides with a mixed MOE/cEt sugar motif
and uniform phosphorothioate internucleoside linkages at a concentration of 2000 nM
in THP-1 cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | NLRP3 | | |
| Compound | Start | Stop | Start | Stop | | (% | | SEQ ID |
| Number | Site | Site | Site | Site | Sequence (5′ to 3′) | UTC) | AID | NO |
| 1232737 | 5138 | 5153 | 90 | 105 | ATGCCATCTTGACCCA | 60 | AH | 22 |
| 1300264 | 27094 | 27109 | N/A | N/A | GGCTATACCTAACACG | 35 | AH | 2515 |
| 1300267 | 26957 | 26972 | N/A | N/A | ACTGATTAATGCCACT | 36 | AH | 1392 |
| 1300329 | 5147 | 5162 | 99 | 114 | CACTTCACGATGCCAT | 53 | AH | 330 |
| 1300366 | 11979 | 11994 | 2282 | 2297 | GAACTTCTCGCAGTCC | 61 | AH | 1114 |
| 1300371 | 5166 | 5181 | 118 | 133 | ACAGTTTACGGTGAAC | 58 | AH | 562 |
| 1300375 | 12464 | 12479 | 2767 | 2782 | AGTGACTCCACCCGAT | 66 | AH | 2733 |
| 1300402 | 14680 | 14695 | N/A | N/A | GGCTCCAATCAGAGTC | 89 | AH | 2453 |
| | 14826 | 14841 | | | | | | |
| | 14972 | 14987 | | | | | | |
| 1300409 | 14575 | 14590 | N/A | N/A | CAGGTGCCATCTAGAG | 29 | AH | 1605 |
| | 14867 | 14882 | | | | | | |
| | 15157 | 15172 | | | | | | |
| | 15230 | 15245 | | | | | | |
| 1300450 | 17670 | 17685 | N/A | N/A | GGAATATATCGAGCAT | 22 | AH | 1377 |
| 1300478 | 5137 | 5152 | 89 | 104 | TGCCATCTTGACCCAT | 69 | AH | 2408 |
| 1300484 | 23500 | 23515 | N/A | N/A | CTTTCCTAGTGTAGAG | 66 | AH | 2518 |
| 1300485 | 23496 | 23511 | N/A | N/A | CCTAGTGTAGAGGCTG | 24 | AH | 77 |
| | 23529 | 23544 | | | | | | |
| 1300516 | 23038 | 23053 | 3313 | 3328 | TAGAGTCTGGTCAGGG | 46 | AH | 1893 |
| 1300522 | 17698 | 17713 | N/A | N/A | CATCTATGAACATGGC | 31 | AH | 1916 |
| 1300530 | 17448 | 17463 | N/A | N/A | CTAGATTAACATTAGG | 56 | AH | 915 |
| 1300542 | 18755 | 18770 | N/A | N/A | TTTAAGCCACCGAACA | 88 | AH | 2509 |
| 1300546 | 12141 | 12156 | 2444 | 2459 | CCCCTTTTCGAATTTG | 59 | AH | 574 |
| 1300563 | 19887 | 19902 | N/A | N/A | GATTCTTGATTAGTTT | 53 | AH | 2671 |
| 1300596 | 20593 | 20608 | N/A | N/A | GAATACACCTGACCAG | 75 | AH | 227 |
| 1300660 | 26968 | 26983 | N/A | N/A | TAAAAGTCTCGACTGA | 65 | AH | 546 |
| 1300687 | 31018 | 31033 | 3462 | 3477 | TAGTGCTGAGTACCGA | 38 | AH | 1049 |
| 1232737 | 5138 | 5153 | 90 | 105 | ATGCCATCTTGACCCA | 54 | AI | 22 |
| 1300262 | 27028 | 27043 | N/A | N/A | CCCATAGGCTATACCT | 34 | AI | 547 |
| | 27064 | 27079 | | | | | | |
| 1300269 | 5249 | 5264 | 201 | 216 | CAGGAGTGTGTCCTGA | 91 | AI | 2663 |
| 1300297 | 6018 | 6033 | 970 | 985 | CTGTTGATCGCAGCGA | 57 | AI | 2183 |
| 1300305 | 10303 | 10318 | 1103 | 1118 | AAGGTACTCCAGTAAA | 71 | AI | 2516 |
| 1300316 | 10928 | 10943 | 1231 | 1246 | CGTTTGTTGAGGCTCA | 32 | AI | 1417 |
| 1300328 | 11007 | 11022 | 1310 | 1325 | GGTCTTGCCGATGGCC | 52 | AI | 2675 |
| 1300360 | 11302 | 11317 | 1605 | 1620 | TCACGATCTTGTGGAT | 79 | AI | 803 |
| 1300403 | 14679 | 14694 | N/A | N/A | GCTCCAATCAGAGTCT | 57 | AI | 2068 |
| | 14825 | 14840 | | | | | | |
| | 14971 | 14986 | | | | | | |
| 1300431 | 15183 | 15198 | N/A | N/A | CGTCAGAGTCTGTCCT | 23 | AI | 2656 |
| 1300470 | 21598 | 21613 | N/A | N/A | ACAATATGAGGACCCA | 38 | AI | 2383 |
| 1300510 | 11048 | 11063 | 1351 | 1366 | AACTCCATCTTAATGG | 75 | AI | 2110 |
| 1300517 | 12134 | 12149 | 2437 | 2452 | TCGAATTTGCCATAGT | 49 | AI | 2115 |
| 1300544 | 18856 | 18871 | N/A | N/A | TGCAGTAACATATCCA | 40 | AI | 1532 |
| 1300587 | 20424 | 20439 | N/A | N/A | TTTGTAAGTGAAGCGG | 36 | AI | 2491 |
| 1300594 | 12455 | 12470 | 2758 | 2773 | ACCCGATGACAGTTCT | 57 | AI | 2195 |
| 1300600 | 20592 | 20607 | N/A | N/A | AATACACCTGACCAGA | 58 | AI | 1457 |
| 1300619 | 22898 | 22913 | N/A | N/A | CAAGGAAGCACCCGTA | 54 | AI | 2492 |
| 1300631 | 25077 | 25092 | N/A | N/A | GCCGCTCTGTTTGTGA | 71 | AI | 2677 |
| 1300640 | 25034 | 25049 | N/A | N/A | TAGGTATTGACAATAG | 42 | AI | 2494 |
| 1300657 | 27023 | 27038 | N/A | N/A | AGGCTATACCTAACAC | 22 | AI | 1546 |
| | 27059 | 27074 | | | | | | |
| | 27095 | 27110 | | | | | | |
| | 27113 | 27128 | | | | | | |
| 1300696 | 35776 | 35791 | 4129 | 4144 | GAGCTAATTACATGAG | 57 | AI | 824 |
| 1232737 | 5138 | 5153 | 90 | 105 | ATGCCATCTTGACCCA | 77 | AJ | 22 |
| 1300317 | 10927 | 10942 | 1230 | 1245 | GTTTGTTGAGGCTCAC | 35 | AJ | 2680 |
| 1300332 | 11258 | 11273 | 1561 | 1576 | ATGATCAGGTCCCCCA | 63 | AJ | 1880 |
| 1300353 | 11972 | 11987 | 2275 | 2290 | TCGCAGTCCACTTCCT | 64 | AJ | 2668 |
| 1300396 | 14477 | 14492 | N/A | N/A | CTGAGCCTCCTACCGG | 83 | AJ | 2219 |
| | 14623 | 14638 | | | | | | |
| | 14769 | 14784 | | | | | | |
| | 14915 | 14930 | | | | | | |
| | 15061 | 15076 | | | | | | |
| 1300415 | 16641 | 16656 | 2959 | 2974 | AGACTCTGGCTGGTGC | 64 | AJ | 2670 |
| 1300418 | 16737 | 16752 | 3055 | 3070 | AATCTCCGAATGTTAC | 94 | AJ | 271 |
| 1300482 | 23533 | 23548 | N/A | N/A | CTCTCCTAGTGTAGAG | 96 | AJ | 2488 |
| 1300489 | 23497 | 23512 | N/A | N/A | TCCTAGTGTAGAGGCT | 31 | AJ | 154 |
| | 23530 | 23545 | | | | | | |
| 1300502 | 12133 | 12148 | 2436 | 2451 | CGAATTTGCCATAGTT | 52 | AJ | 2423 |
| 1300509 | 23000 | 23015 | 3275 | 3290 | TGCAAGATCCTGACAA | 83 | AJ | 2046 |
| 1300535 | 12140 | 12155 | 2443 | 2458 | CCCTTTTCGAATTTGC | 39 | AJ | 497 |
| 1300567 | 19878 | 19893 | N/A | N/A | TTAGTTTCCCCGTCAC | 35 | AJ | 2679 |
| 1300606 | 22429 | 22444 | N/A | N/A | ATAATGTCCCCGAGGC | 45 | AJ | 2493 |
| 1300626 | 24554 | 24569 | N/A | N/A | AGTCAAATTGCCAAGG | 78 | AJ | 2521 |
| 1300662 | 27026 | 27041 | N/A | N/A | CATAGGCTATACCTAA | 77 | AJ | 393 |
| | 27062 | 27077 | | | | | | |
| | 27098 | 27113 | | | | | | |
| | 27116 | 27131 | | | | | | |
| 1300691 | 25745 | 25760 | N/A | N/A | GTTGAAATTGAGAGGT | 35 | AJ | 1235 |
| 1300698 | 31080 | 31095 | 3524 | 3539 | TAGTTTGATCCCCTTG | 78 | AJ | 1820 |
| 1300712 | 26602 | 26617 | N/A | N/A | CTTATTTGGTGATACG | 46 | AJ | 1237 |
| 1232737 | 5138 | 5153 | 90 | 105 | ATGCCATCTTGACCCA | 64 | AK | 22 |
| 1300263 | 26962 | 26977 | N/A | N/A | TCTCGACTGATTAATG | 47 | AK | 2667 |
| 1300276 | 27195 | 27210 | N/A | N/A | TTGAGACAGGCAGTAC | 61 | AK | 2673 |
| 1300283 | 27365 | 27380 | N/A | N/A | CATTTAGGACCGTCCC | 81 | AK | 164 |
| 1300285 | 27202 | 27217 | N/A | N/A | AGCAGTATTGAGACAG | 52 | AK | 2497 |
| 1300349 | 11264 | 11279 | 1567 | 1582 | CAGCTCATGATCAGGT | 25 | AK | 2676 |
| 1300359 | 11301 | 11316 | 1604 | 1619 | CACGATCTTGTGGATG | 59 | AK | 726 |
| 1300368 | 11978 | 11993 | 2281 | 2296 | AACTTCTCGCAGTCCA | 37 | AK | 1037 |
| 1300374 | 12463 | 12478 | 2766 | 2781 | GTGACTCCACCCGATG | 36 | AK | 2272 |
| 1300377 | 12275 | 12290 | 2578 | 2593 | GCTTTCACTTCAATCC | 41 | AK | 1347 |
| 1300386 | 12491 | 12506 | 2794 | 2809 | GGCATGTTATGGAGAA | 54 | AK | 1118 |
| 1300395 | 14478 | 14493 | N/A | N/A | GCTGAGCCTCCTACCG | 30 | AK | 2296 |
| | 14624 | 14639 | | | | | | |
| | 14770 | 14785 | | | | | | |
| | 14916 | 14931 | | | | | | |
| | 15062 | 15077 | | | | | | |
| 1300404 | 14678 | 14693 | N/A | N/A | CTCCAATCAGAGTCTG | 51 | AK | 1991 |
| | 14824 | 14839 | | | | | | |
| | 14970 | 14985 | | | | | | |
| 1300416 | 16727 | 16742 | 3045 | 3060 | TGTTACAGCCAGGATG | 78 | AK | 2669 |
| 1300421 | 15115 | 15130 | N/A | N/A | GTTCCAGTCAGAGTCT | 33 | AK | 2681 |
| 1300453 | 11040 | 11055 | 1343 | 1358 | CTTAATGGGACTCACG | 31 | AK | 186 |
| 1300499 | 25161 | 25176 | N/A | N/A | GACCTATAAGGACCCA | 46 | AK | 81 |
| 1300519 | 23035 | 23050 | 3310 | 3325 | AGTCTGGTCAGGGAAT | 44 | AK | 2686 |
| 1300524 | 17697 | 17712 | N/A | N/A | ATCTATGAACATGGCC | 40 | AK | 1839 |
| 1300529 | 17447 | 17462 | N/A | N/A | TAGATTAACATTAGGT | 23 | AK | 838 |
| 1300554 | 19269 | 19284 | N/A | N/A | AGACACTAGAGCCTCA | 49 | AK | 2504 |
| 1300562 | 19884 | 19899 | N/A | N/A | TCTTGATTAGTTTCCC | 20 | AK | 2511 |
| 1300572 | 20275 | 20290 | N/A | N/A | TGACGATTTCATCCCA | 22 | AK | 2512 |
| 1300573 | 20418 | 20433 | N/A | N/A | AGTGAAGCGGGCGGTG | 60 | AK | 2530 |
| 1300615 | 23822 | 23837 | N/A | N/A | ATGATTATTAGTGGGT | 47 | AK | 2463 |
| 1300647 | 26784 | 26799 | N/A | N/A | CCCAATCGATAGATCT | 54 | AK | 468 |
| 1300666 | 27099 | 27114 | N/A | N/A | ACATAGGCTATACCTA | 64 | AK | 394 |
| | 27117 | 27132 | | | | | | |
| 1300672 | 28762 | 28777 | N/A | N/A | GTAACTATTAAGCAAC | 54 | AK | 1626 |
| 1300690 | 31017 | 31032 | 3461 | 3476 | AGTGCTGAGTACCGAG | 35 | AK | 972 |
| 1232737 | 5138 | 5153 | 90 | 105 | ATGCCATCTTGACCCA | 63 | AL | 22 |
| 1300279 | 27100 | 27115 | N/A | N/A | CACATAGGCTATACCT | 40 | AL | 1623 |
| | 27118 | 27133 | | | | | | |
| 1300310 | 6025 | 6040 | 977 | 992 | GTCTCTCCTGTTGATC | 84 | AL | 2674 |
| 1300319 | 5146 | 5161 | 98 | 113 | ACTTCACGATGCCATC | 39 | AL | 715 |
| 1300354 | 11955 | 11970 | 2258 | 2273 | TTGGAACAGGTTCATC | 59 | AL | 2545 |
| 1300362 | 5153 | 5168 | 105 | 120 | AACAACCACTTCACGA | 72 | AL | 2546 |
| 1300378 | 12462 | 12477 | 2765 | 2780 | TGACTCCACCCGATGA | 97 | AL | 2692 |
| 1300393 | 14570 | 14585 | N/A | N/A | GCCATCTAGAGGGATG | 65 | AL | 1297 |
| | 14862 | 14877 | | | | | | |
| | 15152 | 15167 | | | | | | |
| | 15225 | 15240 | | | | | | |
| 1300434 | 17663 | 17678 | N/A | N/A | ATCGAGCATCATGGTT | 50 | AL | 2517 |
| 1300449 | 17669 | 17684 | N/A | N/A | GAATATATCGAGCATC | 32 | AL | 68 |
| 1300452 | 21597 | 21612 | N/A | N/A | CAATATGAGGACCCAG | 55 | AL | 2305 |
| 1300475 | 23305 | 23320 | N/A | N/A | TTATCTTATTAGCAGC | 40 | AL | 922 |
| 1300486 | 23499 | 23514 | N/A | N/A | TTTCCTAGTGTAGAGG | 40 | AL | 2554 |
| 1300533 | 17992 | 18007 | N/A | N/A | TCAACCCATACAAAGC | 61 | AL | 2519 |
| 1300561 | 19877 | 19892 | N/A | N/A | TAGTTTCCCCGTCACA | 49 | AL | 2538 |
| 1300598 | 12454 | 12469 | 2757 | 2772 | CCCGATGACAGTTCTC | 37 | AL | 268 |
| 1300609 | 22427 | 22442 | N/A | N/A | AATGTCCCCGAGGCTC | 57 | AL | 2687 |
| 1300659 | 26973 | 26988 | N/A | N/A | ATGCATAAAAGTCTCG | 44 | AL | 85 |
| 1300680 | 28761 | 28776 | N/A | N/A | TAACTATTAAGCAACG | 64 | AL | 90 |
| 1300695 | 35773 | 35788 | 4126 | 4141 | CTAATTACATGAGGTC | 29 | AL | 2364 |
| 1300704 | 5247 | 5262 | 199 | 214 | GGAGTGTGTCCTGAGC | 49 | AL | 2255 |
| 1232737 | 5138 | 5153 | 90 | 105 | ATGCCATCTTGACCCA | 75 | AM | 22 |
| 1300280 | 27106 | 27121 | N/A | N/A | ACCTAACACATAGGCT | 51 | AM | 2085 |
| | 27124 | 27139 | | | | | | |
| 1300290 | 27201 | 27216 | N/A | N/A | GCAGTATTGAGACAGG | 49 | AM | 931 |
| 1300300 | 6017 | 6032 | 969 | 984 | TGTTGATCGCAGCGAA | 43 | AM | 2106 |
| 1300302 | 10302 | 10317 | 1102 | 1117 | AGGTACTCCAGTAAAC | 62 | AM | 2689 |
| 1300322 | 11013 | 11028 | 1316 | 1331 | CGTCTTGGTCTTGCCG | 88 | AM | 2690 |
| 1300326 | 11006 | 11021 | 1309 | 1324 | GTCTTGCCGATGGCCA | 50 | AM | 1956 |
| 1300335 | 11236 | 11251 | 1539 | 1554 | TCTGTGTCACAAGGCT | 62 | AM | 1649 |
| 1300392 | 14572 | 14587 | N/A | N/A | GTGCCATCTAGAGGGA | 83 | AM | 1374 |
| | 14864 | 14879 | | | | | | |
| | 15154 | 15169 | | | | | | |
| | 15227 | 15242 | | | | | | |
| 1300397 | 5172 | 5187 | 124 | 139 | TGTATTACAGTTTACG | 89 | AM | 255 |
| 1300414 | 16735 | 16750 | 3053 | 3068 | TCTCCGAATGTTACAG | 67 | AM | 117 |
| 1300426 | 16647 | 16662 | 2965 | 2980 | TCAGTTAGACTCTGGC | 60 | AM | 1505 |
| 1300429 | 5136 | 5151 | 88 | 103 | GCCATCTTGACCCATC | 72 | AM | 2678 |
| 1300483 | 11046 | 11061 | 1349 | 1364 | CTCCATCTTAATGGGA | 96 | AM | 2528 |
| 1300534 | 18753 | 18768 | N/A | N/A | TAAGCCACCGAACAGC | 51 | AM | 2563 |
| 1300559 | 19268 | 19283 | N/A | N/A | GACACTAGAGCCTCAG | 44 | AM | 1686 |
| 1300564 | 19883 | 19898 | N/A | N/A | CTTGATTAGTTTCCCC | 33 | AM | 1764 |
| 1300590 | 20423 | 20438 | N/A | N/A | TTGTAAGTGAAGCGGG | 51 | AM | 2550 |
| 1300618 | 23821 | 23836 | N/A | N/A | TGATTATTAGTGGGTA | 46 | AM | 2078 |
| 1300636 | 25032 | 25047 | N/A | N/A | GGTATTGACAATAGTA | 21 | AM | 2682 |
| 1300674 | 29462 | 29477 | N/A | N/A | AGTCAAATTAAGCCCA | 85 | AM | 245 |
| 1300692 | 35770 | 35785 | 4123 | 4138 | ATTACATGAGGTCACC | 37 | AM | 2567 |
| 1300703 | 26601 | 26616 | N/A | N/A | TTATTTGGTGATACGG | 50 | AM | 1160 |
| 1300707 | 25820 | 25835 | N/A | N/A | TATGTTTACACCAAGC | 22 | AM | 2522 |
| 1232737 | 5138 | 5153 | 90 | 105 | ATGCCATCTTGACCCA | 87 | AN | 22 |
| 1300268 | 5133 | 5148 | 85 | 100 | ATCTTGACCCATCAGC | 57 | AN | 21 |
| 1300284 | 27364 | 27379 | N/A | N/A | ATTTAGGACCGTCCCC | 104 | AN | 87 |
| 1300298 | 27370 | 27385 | N/A | N/A | CAGAGCATTTAGGACC | 81 | AN | 2525 |
| 1300304 | 6023 | 6038 | 975 | 990 | CTCTCCTGTTGATCGC | 72 | AN | 798 |
| 1300313 | 10925 | 10940 | 1228 | 1243 | TTGTTGAGGCTCACAC | 75 | AN | 2684 |
| 1300342 | 11300 | 11315 | 1603 | 1618 | ACGATCTTGTGGATGG | 59 | AN | 649 |
| 1300369 | 11977 | 11992 | 2280 | 2295 | ACTTCTCGCAGTCCAC | 46 | AN | 960 |
| 1300391 | 14571 | 14586 | N/A | N/A | TGCCATCTAGAGGGAT | 65 | AN | 450 |
| | 14863 | 14878 | | | | | | |
| | 15153 | 15168 | | | | | | |
| | 15226 | 15241 | | | | | | |
| 1300394 | 12709 | 12724 | N/A | N/A | GGCATTATGATGTAGC | 49 | AN | 832 |
| 1300405 | 14580 | 14595 | N/A | N/A | TAAGGCAGGTGCCATC | 44 | AN | 1913 |
| | 14872 | 14887 | | | | | | |
| | 15162 | 15177 | | | | | | |
| | 15235 | 15250 | | | | | | |
| 1300412 | 16734 | 16749 | 3052 | 3067 | CTCCGAATGTTACAGC | 86 | AN | 40 |
| 1300454 | 21596 | 21611 | N/A | N/A | AATATGAGGACCCAGG | 49 | AN | 151 |
| 1300465 | 23280 | 23295 | N/A | N/A | TATATGCCTCCAGTCC | 72 | AN | 2537 |
| 1300493 | 25160 | 25175 | N/A | N/A | ACCTATAAGGACCCAG | 54 | AN | 619 |
| 1300521 | 17446 | 17461 | N/A | N/A | AGATTAACATTAGGTC | 35 | AN | 761 |
| 1300526 | 17696 | 17711 | N/A | N/A | TCTATGAACATGGCCC | 51 | AN | 2556 |
| 1300539 | 12139 | 12154 | 2442 | 2457 | CCTTTTCGAATTTGCC | 22 | AN | 420 |
| 1300545 | 18760 | 18775 | N/A | N/A | AAGCATTTAAGCCACC | 55 | AN | 1301 |
| 1300584 | 20591 | 20606 | N/A | N/A | ATACACCTGACCAGAG | 47 | AN | 2531 |
| 1300603 | 22897 | 22912 | N/A | N/A | AAGGAAGCACCCGTAC | 101 | AN | 2539 |
| 1300616 | 22902 | 22917 | N/A | N/A | TGGACAAGGAAGCACC | 71 | AN | 2565 |
| 1300629 | 24553 | 24568 | N/A | N/A | GTCAAATTGCCAAGGA | 66 | AN | 1003 |
| 1300633 | 25076 | 25091 | N/A | N/A | CCGCTCTGTTTGTGAT | 88 | AN | 2683 |
| 1300635 | 14501 | 14516 | N/A | N/A | AGGTGCTATCTAGAGA | 19 | AN | 1681 |
| | 14647 | 14662 | | | | | | |
| | 14720 | 14735 | | | | | | |
| | 14793 | 14808 | | | | | | |
| | 14939 | 14954 | | | | | | |
| | 15012 | 15027 | | | | | | |
| | 15085 | 15100 | | | | | | |
| 1300642 | 26789 | 26804 | N/A | N/A | TATGCCCCAATCGATA | 54 | AN | 2688 |
| 1300677 | 29461 | 29476 | N/A | N/A | GTCAAATTAAGCCCAT | 57 | AN | 168 |
| 1300693 | 25740 | 25755 | N/A | N/A | AATTGAGAGGTATATG | 71 | AN | 2543 |
| 1232737 | 5138 | 5153 | 90 | 105 | ATGCCATCTTGACCCA | 55 | AO | 22 |
| 1300265 | 26961 | 26976 | N/A | N/A | CTCGACTGATTAATGC | 71 | AO | 2544 |
| 1300274 | 6015 | 6030 | 967 | 982 | TTGATCGCAGCGAAGA | 75 | AO | 2029 |
| 1300288 | 27363 | 27378 | N/A | N/A | TTTAGGACCGTCCCCC | 67 | AO | 2569 |
| 1300318 | 5145 | 5160 | 97 | 112 | CTTCACGATGCCATCT | 45 | AO | 253 |
| 1300333 | 11235 | 11250 | 1538 | 1553 | CTGTGTCACAAGGCTC | 33 | AO | 1572 |
| 1300341 | 11263 | 11278 | 1566 | 1581 | AGCTCATGATCAGGTC | 63 | AO | 2535 |
| 1300355 | 11952 | 11967 | 2255 | 2270 | GAACAGGTTCATCCTC | 81 | AO | 34 |
| 1300358 | 5151 | 5166 | 103 | 118 | CAACCACTTCACGATG | 57 | AO | 946 |
| 1300372 | 12461 | 12476 | 2764 | 2779 | GACTCCACCCGATGAC | 83 | AO | 2695 |
| 1300376 | 11983 | 11998 | 2286 | 2301 | TGTAGAACTTCTCGCA | 38 | AO | 2685 |
| 1300389 | 12487 | 12502 | 2790 | 2805 | TGTTATGGAGAAACCC | 54 | AO | 576 |
| 1300410 | 14574 | 14589 | N/A | N/A | AGGTGCCATCTAGAGG | 29 | AO | 1528 |
| | 14866 | 14881 | | | | | | |
| | 15156 | 15171 | | | | | | |
| | 15229 | 15244 | | | | | | |
| 1300447 | 17668 | 17683 | N/A | N/A | AATATATCGAGCATCA | 24 | AO | 606 |
| 1300479 | 23303 | 23318 | N/A | N/A | ATCTTATTAGCAGCAG | 36 | AO | 76 |
| 1300490 | 23532 | 23547 | N/A | N/A | TCTCCTAGTGTAGAGG | 87 | AO | 2549 |
| 1300505 | 23033 | 23048 | 3308 | 3323 | TCTGGTCAGGGAATGG | 53 | AO | 1662 |
| 1300506 | 25166 | 25181 | N/A | N/A | TGTACGACCTATAAGG | 91 | AO | 2697 |
| 1300531 | 12138 | 12153 | 2441 | 2456 | CTTTTCGAATTTGCCA | 44 | AO | 343 |
| 1300565 | 19882 | 19897 | N/A | N/A | TTGATTAGTTTCCCCG | 32 | AO | 1687 |
| 1300569 | 12145 | 12160 | 2448 | 2463 | AATACCCCTTTTCGAA | 58 | AO | 36 |
| 1300595 | 12453 | 12468 | 2756 | 2771 | CCGATGACAGTTCTCA | 29 | AO | 2118 |
| 1300620 | 23820 | 23835 | N/A | N/A | GATTATTAGTGGGTAT | 42 | AO | 616 |
| 1300634 | 25075 | 25090 | N/A | N/A | CGCTCTGTTTGTGATA | 40 | AO | 2699 |
| 1300648 | 26783 | 26798 | N/A | N/A | CCAATCGATAGATCTA | 49 | AO | 2541 |
| 1300663 | 28760 | 28775 | N/A | N/A | AACTATTAAGCAACGG | 55 | AO | 628 |
| 1300682 | 31079 | 31094 | 3523 | 3538 | AGTTTGATCCCCTTGT | 56 | AO | 1743 |
| 1300689 | 31016 | 31031 | 3460 | 3475 | GTGCTGAGTACCGAGG | 38 | AO | 895 |
| 1300710 | 25817 | 25832 | N/A | N/A | GTTTACACCAAGCATG | 55 | AO | 2571 |
| 1232737 | 5138 | 5153 | 90 | 105 | ATGCCATCTTGACCCA | 65 | AP | 22 |
| 1300275 | 27134 | 27149 | N/A | N/A | ATGTAGCTATACCTAA | 103 | AP | 2551 |
| 1300294 | 27369 | 27384 | N/A | N/A | AGAGCATTTAGGACCG | 55 | AP | 2572 |
| 1300308 | 10301 | 10316 | 1101 | 1116 | GGTACTCCAGTAAACC | 80 | AP | 2694 |
| 1300323 | 11011 | 11026 | 1314 | 1329 | TCTTGGTCTTGCCGAT | 71 | AP | 2702 |
| 1300330 | 11005 | 11020 | 1308 | 1323 | TCTTGCCGATGGCCAG | 87 | AP | 2691 |
| 1300390 | 12486 | 12501 | 2789 | 2804 | GTTATGGAGAAACCCC | 25 | AP | 887 |
| 1300399 | 5171 | 5186 | 123 | 138 | GTATTACAGTTTACGG | 56 | AP | 178 |
| 1300413 | 14681 | 14696 | N/A | N/A | GGGCTCCAATCAGAGT | 94 | AP | 2145 |
| | 14827 | 14842 | | | | | | |
| | 14973 | 14988 | | | | | | |
| 1300435 | 17662 | 17677 | N/A | N/A | TCGAGCATCATGGTTG | 42 | AP | 2547 |
| 1300476 | 11045 | 11060 | 1348 | 1363 | TCCATCTTAATGGGAC | 84 | AP | 2693 |
| 1300553 | 18860 | 18875 | N/A | N/A | ACTGTGCAGTAACATA | 70 | AP | 2700 |
| 1300556 | 12144 | 12159 | 2447 | 2462 | ATACCCCTTTTCGAAT | 82 | AP | 2347 |
| 1300574 | 20416 | 20431 | N/A | N/A | TGAAGCGGGCGGTGTG | 82 | AP | 2579 |
| 1300578 | 20274 | 20289 | N/A | N/A | GACGATTTCATCCCAG | 20 | AP | 1918 |
| 1300585 | 20590 | 20605 | N/A | N/A | TACACCTGACCAGAGA | 42 | AP | 2587 |
| 1300624 | 25020 | 25035 | N/A | N/A | AGTAACTACATAGAGC | 46 | AP | 465 |
| 1300639 | 25074 | 25089 | N/A | N/A | GCTCTGTTTGTGATAC | 49 | AP | 2389 |
| | 26262 | 26277 | | | | | | |
| 1232737 | 5138 | 5153 | 90 | 105 | ATGCCATCTTGACCCA | 59 | AQ | 22 |
| 1300270 | 26960 | 26975 | N/A | N/A | TCGACTGATTAATGCC | 45 | AQ | 2558 |
| 1300291 | 6022 | 6037 | 974 | 989 | TCTCCTGTTGATCGCA | 40 | AQ | 721 |
| 1300307 | 5143 | 5158 | 95 | 110 | TCACGATGCCATCTTG | 45 | AQ | 2332 |
| 1300321 | 11004 | 11019 | 1307 | 1322 | CTTGCCGATGGCCAGA | 86 | AQ | 2703 |
| 1300363 | 11982 | 11997 | 2285 | 2300 | GTAGAACTTCTCGCAG | 69 | AQ | 2583 |
| 1300370 | 11976 | 11991 | 2279 | 2294 | CTTCTCGCAGTCCACT | 34 | AQ | 2704 |
| 1300381 | 12704 | 12719 | N/A | N/A | TATGATGTAGCTGTGG | 54 | AQ | 678 |
| 1300406 | 14590 | 14605 | N/A | N/A | GTCCTCAGAGTAAGGC | 58 | AQ | 759 |
| | 14882 | 14897 | | | | | | |
| | 15172 | 15187 | | | | | | |
| | 15245 | 15260 | | | | | | |
| 1300420 | 16733 | 16748 | 3051 | 3066 | TCCGAATGTTACAGCC | 59 | AQ | 578 |
| 1300422 | 15112 | 15127 | N/A | N/A | CCAGTCAGAGTCTGTC | 32 | AQ | 991 |
| | 15258 | 15273 | | | | | | |
| 1300427 | 16645 | 16660 | 2963 | 2978 | AGTTAGACTCTGGCTG | 65 | AQ | 1351 |
| 1300442 | 17675 | 17690 | N/A | N/A | GAAATGGAATATATCG | 65 | AQ | 1608 |
| 1300446 | 17667 | 17682 | N/A | N/A | ATATATCGAGCATCAT | 60 | AQ | 529 |
| 1300466 | 21604 | 21619 | N/A | N/A | TCTTATACAATATGAG | 65 | AQ | 305 |
| 1300487 | 23498 | 23513 | N/A | N/A | TTCCTAGTGTAGAGGC | 56 | AQ | 2696 |
| 1300488 | 23531 | 23546 | N/A | N/A | CTCCTAGTGTAGAGGC | 102 | AQ | 2561 |
| 1300507 | 23007 | 23022 | 3282 | 3297 | ATACTGATGCAAGATC | 55 | AQ | 738 |
| 1300547 | 18759 | 18774 | N/A | N/A | AGCATTTAAGCCACCG | 40 | AQ | 1224 |
| 1300557 | 19469 | 19484 | N/A | N/A | GATGAAGAGTGTGATC | 72 | AQ | 2578 |
| 1300576 | 20273 | 20288 | N/A | N/A | ACGATTTCATCCCAGC | 24 | AQ | 2594 |
| 1300588 | 20422 | 20437 | N/A | N/A | TGTAAGTGAAGCGGGC | 52 | AQ | 2580 |
| 1300599 | 12452 | 12467 | 2755 | 2770 | CGATGACAGTTCTCAA | 55 | AQ | 2426 |
| 1300628 | 23827 | 23842 | N/A | N/A | AGTGCATGATTATTAG | 72 | AQ | 2698 |
| 1300641 | 14504 | 14519 | N/A | N/A | GGAAGGTGCTATCTAG | 33 | AQ | 1835 |
| | 14650 | 14665 | | | | | | |
| | 14723 | 14738 | | | | | | |
| | 14796 | 14811 | | | | | | |
| | 14942 | 14957 | | | | | | |
| | 15015 | 15030 | | | | | | |
| | 15088 | 15103 | | | | | | |
| 1300644 | 26787 | 26802 | N/A | N/A | TGCCCCAATCGATAGA | 89 | AQ | 2596 |
| 1300654 | 26972 | 26987 | N/A | N/A | TGCATAAAAGTCTCGA | 58 | AQ | 1469 |
| 1300681 | 31015 | 31030 | 3459 | 3474 | TGCTGAGTACCGAGGA | 51 | AQ | 818 |
| 1300684 | 31077 | 31092 | 3521 | 3536 | TTTGATCCCCTTGTCT | 77 | AQ | 1589 |
| 1300701 | 26600 | 26615 | N/A | N/A | TATTTGGTGATACGGT | 26 | AQ | 2568 |
| 1232737 | 5138 | 5153 | 90 | 105 | ATGCCATCTTGACCCA | 65 | AR | 22 |
| 1300282 | 27200 | 27215 | N/A | N/A | CAGTATTGAGACAGGC | 47 | AR | 2582 |
| 1300312 | 10923 | 10938 | 1226 | 1241 | GTTGAGGCTCACACTC | 73 | AR | 1340 |
| 1300338 | 11233 | 11248 | 1536 | 1551 | GTGTCACAAGGCTCAC | 79 | AR | 1418 |
| 1300343 | 11299 | 11314 | 1602 | 1617 | CGATCTTGTGGATGGG | 41 | AR | 2343 |
| 1300379 | 12460 | 12475 | 2763 | 2778 | ACTCCACCCGATGACA | 60 | AR | 2706 |
| 1300436 | 5293 | 5308 | 245 | 260 | AGTTAGCCAAATGCTT | 72 | AR | 410 |
| 1300456 | 20704 | 20719 | N/A | N/A | GAGAGCTCCGGAATAG | 61 | AR | 842 |
| | 20746 | 20761 | | | | | | |
| | 20914 | 20929 | | | | | | |
| 1300471 | 23301 | 23316 | N/A | N/A | CTTATTAGCAGCAGTA | 39 | AR | 845 |
| 1300496 | 25159 | 25174 | N/A | N/A | CCTATAAGGACCCAGC | 56 | AR | 542 |
| 1300512 | 17445 | 17460 | N/A | N/A | GATTAACATTAGGTCT | 45 | AR | 144 |
| 1300515 | 23043 | 23058 | 3318 | 3333 | CCACATAGAGTCTGGT | 79 | AR | 582 |
| 1300537 | 18354 | 18369 | N/A | N/A | GCGGACTTTAACACTT | 67 | AR | 2586 |
| 1300601 | 22895 | 22910 | N/A | N/A | GGAAGCACCCGTACCT | 96 | AR | 2708 |
| 1300613 | 22901 | 22916 | N/A | N/A | GGACAAGGAAGCACCC | 75 | AR | 2605 |
| 1300622 | 23825 | 23840 | N/A | N/A | TGCATGATTATTAGTG | 69 | AR | 2606 |
| 1300632 | 25038 | 25053 | N/A | N/A | AGGTTAGGTATTGACA | 46 | AR | 2595 |
| 1300650 | 26782 | 26797 | N/A | N/A | CAATCGATAGATCTAC | 46 | AR | 391 |
| 1232737 | 5138 | 5153 | 90 | 105 | ATGCCATCTTGACCCA | 55 | AS | 22 |
| 1300271 | 27132 | 27147 | N/A | N/A | GTAGCTATACCTAACA | 67 | AS | 2701 |
| 1300286 | 27362 | 27377 | N/A | N/A | TTAGGACCGTCCCCCG | 131 | AS | 2714 |
| 1300293 | 6021 | 6036 | 973 | 988 | CTCCTGTTGATCGCAG | 65 | AS | 644 |
| 1300309 | 10300 | 10315 | 1100 | 1115 | GTACTCCAGTAAACCC | 67 | AS | 2031 |
| 1300347 | 5150 | 5165 | 102 | 117 | AACCACTTCACGATGC | 71 | AS | 871 |
| 1300350 | 11262 | 11277 | 1565 | 1580 | GCTCATGATCAGGTCC | 44 | AS | 2111 |
| 1300357 | 11948 | 11963 | 2251 | 2266 | AGGTTCATCCTCAGGA | 94 | AS | 729 |
| 1300424 | 15111 | 15126 | N/A | N/A | CAGTCAGAGTCTGTCC | 12 | AS | 914 |
| | 15257 | 15272 | | | | | | |
| 1300425 | 16644 | 16659 | 2962 | 2977 | GTTAGACTCTGGCTGG | 52 | AS | 1274 |
| 1300439 | 15187 | 15202 | N/A | N/A | CTCCCGTCAGAGTCTG | 87 | AS | 2570 |
| 1300501 | 25164 | 25179 | N/A | N/A | TACGACCTATAAGGAC | 88 | AS | 2603 |
| 1300518 | 23041 | 23056 | 3316 | 3331 | ACATAGAGTCTGGTCA | 97 | AS | 505 |
| 1300538 | 17988 | 18003 | N/A | N/A | CCCATACAAAGCATTC | 66 | AS | 2576 |
| 1300617 | 23817 | 23832 | N/A | N/A | TATTAGTGGGTATTCC | 79 | AS | 462 |
| 1300625 | 25019 | 25034 | N/A | N/A | GTAACTACATAGAGCA | 33 | AS | 2466 |
| 1300643 | 26781 | 26796 | N/A | N/A | AATCGATAGATCTACA | 58 | AS | 314 |
| 1300651 | 27025 | 27040 | N/A | N/A | ATAGGCTATACCTAAC | 105 | AS | 316 |
| | 27061 | 27076 | | | | | | |
| | 27097 | 27112 | | | | | | |
| | 27115 | 27130 | | | | | | |
| 1300658 | 26971 | 26986 | N/A | N/A | GCATAAAAGTCTCGAC | 51 | AS | 623 |
| 1300661 | 28759 | 28774 | N/A | N/A | ACTATTAAGCAACGGT | 49 | AS | 551 |
| 1300669 | 27027 | 27042 | N/A | N/A | CCATAGGCTATACCTA | 47 | AS | 470 |
| | 27063 | 27078 | | | | | | |
| 1300678 | 29459 | 29474 | N/A | N/A | CAAATTAAGCCCATTC | 74 | AS | 2013 |
| 1232737 | 5138 | 5153 | 90 | 105 | ATGCCATCTTGACCCA | 57 | AT | 22 |
| 1300272 | 27199 | 27214 | N/A | N/A | AGTATTGAGACAGGCA | 43 | AT | 2622 |
| 1300324 | 11010 | 11025 | 1313 | 1328 | CTTGGTCTTGCCGATG | 70 | AT | 2715 |
| 1300344 | 11298 | 11313 | 1601 | 1616 | GATCTTGTGGATGGGT | 39 | AT | 2600 |
| 1300348 | 11261 | 11276 | 1564 | 1579 | CTCATGATCAGGTCCC | 59 | AT | 2419 |
| 1300383 | 5170 | 5185 | 122 | 137 | TATTACAGTTTACGGT | 54 | AT | 101 |
| 1300455 | 21594 | 21609 | N/A | N/A | TATGAGGACCCAGGGC | 89 | AT | 2574 |
| 1300467 | 21603 | 21618 | N/A | N/A | CTTATACAATATGAGG | 80 | AT | 843 |
| 1300472 | 23493 | 23508 | N/A | N/A | AGTGTAGAGGCTGCAT | 24 | AT | 2716 |
| 1300473 | 11044 | 11059 | 1347 | 1362 | CCATCTTAATGGGACT | 62 | AT | 2585 |
| 1300523 | 12137 | 12152 | 2440 | 2455 | TTTTCGAATTTGCCAT | 54 | AT | 2192 |
| 1300548 | 18758 | 18773 | N/A | N/A | GCATTTAAGCCACCGA | 47 | AT | 1147 |
| 1300589 | 20421 | 20436 | N/A | N/A | GTAAGTGAAGCGGGCG | 33 | AT | 2072 |
| 1300593 | 12451 | 12466 | 2754 | 2769 | GATGACAGTTCTCAAT | 106 | AT | 2041 |
| 1300676 | 5141 | 5156 | 93 | 108 | ACGATGCCATCTTGAC | 44 | AT | 2254 |
| 1300699 | 31084 | 31099 | 3528 | 3543 | AGAGTAGTTTGATCCC | 62 | AT | 2589 |
| 1232737 | 5138 | 5153 | 90 | 105 | ATGCCATCTTGACCCA | 52 | AU | 22 |
| 1300292 | 27368 | 27383 | N/A | N/A | GAGCATTTAGGACCGT | 29 | AU | 1393 |
| 1300306 | 10297 | 10312 | 1097 | 1112 | CTCCAGTAAACCCATC | 67 | AU | 1877 |
| 1300311 | 10883 | 10898 | 1186 | 1201 | TCAATGCACTGGAATC | 52 | AU | 2032 |
| 1300315 | 11003 | 11018 | 1306 | 1321 | TTGCCGATGGCCAGAA | 73 | AU | 2718 |
| 1300331 | 11232 | 11247 | 1535 | 1550 | TGTCACAAGGCTCACC | 68 | AU | 2710 |
| 1300364 | 11981 | 11996 | 2284 | 2299 | TAGAACTTCTCGCAGT | 60 | AU | 2623 |
| 1300367 | 11975 | 11990 | 2278 | 2293 | TTCTCGCAGTCCACTT | 78 | AU | 2705 |
| 1300385 | 12703 | 12718 | N/A | N/A | ATGATGTAGCTGTGGC | 26 | AU | 2625 |
| 1300419 | 16732 | 16747 | 3050 | 3065 | CCGAATGTTACAGCCA | 69 | AU | 501 |
| 1300445 | 17673 | 17688 | N/A | N/A | AATGGAATATATCGAG | 34 | AU | 222 |
| 1300448 | 17666 | 17681 | N/A | N/A | TATATCGAGCATCATG | 27 | AU | 452 |
| 1300462 | 21600 | 21615 | N/A | N/A | ATACAATATGAGGACC | 55 | AU | 689 |
| 1300504 | 23006 | 23021 | 3281 | 3296 | TACTGATGCAAGATCC | 58 | AU | 661 |
| 1300513 | 17443 | 17458 | N/A | N/A | TTAACATTAGGTCTTG | 16 | AU | 2638 |
| 1300527 | 12136 | 12151 | 2439 | 2454 | TTTCGAATTTGCCATA | 47 | AU | 266 |
| 1300552 | 19273 | 19288 | N/A | N/A | TGATAGACACTAGAGC | 39 | AU | 2613 |
| 1300555 | 12143 | 12158 | 2446 | 2461 | TACCCCTTTTCGAATT | 80 | AU | 2614 |
| 1300558 | 18859 | 18874 | N/A | N/A | CTGTGCAGTAACATAT | 54 | AU | 2707 |
| 1300566 | 19881 | 19896 | N/A | N/A | TGATTAGTTTCCCCGT | 49 | AU | 2577 |
| 1300580 | 20272 | 20287 | N/A | N/A | CGATTTCATCCCAGCC | 22 | AU | 2615 |
| 1300604 | 22431 | 22446 | N/A | N/A | GCATAATGTCCCCGAG | 41 | AU | 536 |
| 1300653 | 27024 | 27039 | N/A | N/A | TAGGCTATACCTAACA | 51 | AU | 239 |
| | 27060 | 27075 | | | | | | |
| | 27096 | 27111 | | | | | | |
| | 27114 | 27129 | | | | | | |
| 1300685 | 31022 | 31037 | 3466 | 3481 | TGATTAGTGCTGAGTA | 27 | AU | 1280 |
| 1300708 | 26599 | 26614 | N/A | N/A | ATTTGGTGATACGGTT | 35 | AU | 2709 |
| 1232737 | 5138 | 5153 | 90 | 105 | ATGCCATCTTGACCCA | 43 | AV | 22 |
| 1300266 | 26959 | 26974 | N/A | N/A | CGACTGATTAATGCCA | 27 | AV | 2713 |
| 1300278 | 27131 | 27146 | N/A | N/A | TAGCTATACCTAACAC | 86 | AV | 2634 |
| 1300320 | 10880 | 10895 | 1183 | 1198 | ATGCACTGGAATCTGC | 63 | AV | 1801 |
| 1300337 | 5149 | 5164 | 101 | 116 | ACCACTTCACGATGCC | 47 | AV | 792 |
| 1300361 | 11974 | 11989 | 2277 | 2292 | TCTCGCAGTCCACTTC | 80 | AV | 2720 |
| 1300382 | 12466 | 12481 | 2769 | 2784 | ACAGTGACTCCACCCG | 28 | AV | 656 |
| 1300400 | 14476 | 14491 | N/A | N/A | TGAGCCTCCTACCGGG | 77 | AV | 2142 |
| | 14622 | 14637 | | | | | | |
| | 14768 | 14783 | | | | | | |
| | 14914 | 14929 | | | | | | |
| | 15060 | 15075 | | | | | | |
| 1300430 | 5290 | 5305 | 242 | 257 | TAGCCAAATGCTTACC | 72 | AV | 1948 |
| 1300437 | 17660 | 17675 | N/A | N/A | GAGCATCATGGTTGTT | 48 | AV | 2601 |
| 1300438 | 15185 | 15200 | N/A | N/A | CCCGTCAGAGTCTGTC | 35 | AV | 2711 |
| 1300443 | 20578 | 20593 | N/A | N/A | GAGAGCTCCGGAATAC | 63 | AV | 841 |
| | 20956 | 20971 | | | | | | |
| 1300458 | 20584 | 20599 | N/A | N/A | TGACCAGAGAGCTCCG | 57 | AV | 150 |
| | 20710 | 20725 | | | | | | |
| | 20752 | 20767 | | | | | | |
| | 20920 | 20935 | | | | | | |
| 1300491 | 23535 | 23550 | N/A | N/A | TCCTCTCCTAGTGTAG | 109 | AV | 2724 |
| 1300497 | 25158 | 25173 | N/A | N/A | CTATAAGGACCCAGCC | 79 | AV | 2592 |
| 1300549 | 18757 | 18772 | N/A | N/A | CATTTAAGCCACCGAA | 69 | AV | 224 |
| 1300577 | 20279 | 20294 | N/A | N/A | TGTTTGACGATTTCAT | 54 | AV | 2712 |
| 1300610 | 12457 | 12472 | 2760 | 2775 | CCACCCGATGACAGTT | 62 | AV | 2631 |
| 1300655 | 27021 | 27036 | N/A | N/A | GCTATACCTAACACAC | 27 | AV | 2598 |
| | 27057 | 27072 | | | | | | |
| 1300683 | 31020 | 31035 | 3464 | 3479 | ATTAGTGCTGAGTACC | 51 | AV | 1203 |
| 1300688 | 31014 | 31029 | 3458 | 3473 | GCTGAGTACCGAGGAC | 53 | AV | 741 |
| 1300702 | 25814 | 25829 | N/A | N/A | TACACCAAGCATGTGT | 112 | AV | 2607 |
| 1300706 | 26598 | 26613 | N/A | N/A | TTTGGTGATACGGTTA | 36 | AV | 2726 |
| 1232737 | 5138 | 5153 | 90 | 105 | ATGCCATCTTGACCCA | 65 | AW | 22 |
| 1300289 | 27206 | 27221 | N/A | N/A | CTTGAGCAGTATTGAG | 77 | AW | 2644 |
| 1300296 | 6020 | 6035 | 972 | 987 | TCCTGTTGATCGCAGC | 66 | AW | 2338 |
| 1300345 | 11297 | 11312 | 1600 | 1615 | ATCTTGTGGATGGGTG | 44 | AW | 2265 |
| 1300356 | 11947 | 11962 | 2250 | 2265 | GGTTCATCCTCAGGAA | 63 | AW | 652 |
| 1300407 | 5288 | 5303 | 240 | 255 | GCCAAATGCTTACCAG | 33 | AW | 333 |
| 1300432 | 17665 | 17680 | N/A | N/A | ATATCGAGCATCATGG | 110 | AW | 375 |
| 1300444 | 17672 | 17687 | N/A | N/A | ATGGAATATATCGAGC | 92 | AW | 145 |
| 1300451 | 20580 | 20595 | N/A | N/A | CAGAGAGCTCCGGAAT | 71 | AW | 918 |
| | 20706 | 20721 | | | | | | |
| | 20748 | 20763 | | | | | | |
| | 20916 | 20931 | | | | | | |
| | 20958 | 20973 | | | | | | |
| 1300461 | 11043 | 11058 | 1346 | 1361 | CATCTTAATGGGACTC | 43 | AW | 417 |
| 1300498 | 23502 | 23517 | N/A | N/A | CTCTTTCCTAGTGTAG | 84 | AW | 2611 |
| 1300503 | 23004 | 23019 | 3279 | 3294 | CTGATGCAAGATCCTG | 23 | AW | 2277 |
| 1300536 | 18353 | 18368 | N/A | N/A | CGGACTTTAACACTTG | 40 | AW | 2639 |
| 1300570 | 19880 | 19895 | N/A | N/A | GATTAGTTTCCCCGTC | 42 | AW | 1610 |
| 1300586 | 20426 | 20441 | N/A | N/A | GGTTTGTAAGTGAAGC | 31 | AW | 2659 |
| 1300591 | 20697 | 20712 | N/A | N/A | CCGGAATAGAGAAAGC | 74 | AW | 304 |
| | 20739 | 20754 | | | | | | |
| | 20907 | 20922 | | | | | | |
| | 20991 | 21006 | | | | | | |
| 1300592 | 21014 | 21029 | N/A | N/A | AGGACACACCTGATCA | 76 | AW | 2717 |
| 1300612 | 22900 | 22915 | N/A | N/A | GACAAGGAAGCACCCG | 90 | AW | 2076 |
| 1300630 | 5140 | 5155 | 92 | 107 | CGATGCCATCTTGACC | 31 | AW | 2177 |
| 1300646 | 26786 | 26801 | N/A | N/A | GCCCCAATCGATAGAT | 111 | AW | 2642 |
| 1300665 | 28758 | 28773 | N/A | N/A | CTATTAAGCAACGGTT | 96 | AW | 474 |
| 1300675 | 28765 | 28780 | N/A | N/A | CTGGTAACTATTAAGC | 66 | AW | 2597 |
| 1300700 | 31083 | 31098 | 3527 | 3542 | GAGTAGTTTGATCCCC | 46 | AW | 2051 |
| 1232737 | 5138 | 5153 | 90 | 105 | ATGCCATCTTGACCCA | 43 | AX | 22 |
| 1300325 | 11009 | 11024 | 1312 | 1327 | TTGGTCTTGCCGATGG | 57 | AX | 2719 |
| 1300334 | 11260 | 11275 | 1563 | 1578 | TCATGATCAGGTCCCC | 55 | AX | 2034 |
| 1300373 | 12465 | 12480 | 2768 | 2783 | CAGTGACTCCACCCGA | 29 | AX | 2350 |
| 1300387 | 5169 | 5184 | 121 | 136 | ATTACAGTTTACGGTG | 63 | AX | 1331 |
| 1300417 | 16731 | 16746 | 3049 | 3064 | CGAATGTTACAGCCAG | 57 | AX | 2353 |
| 1300428 | 16643 | 16658 | 2961 | 2976 | TTAGACTCTGGCTGGT | 44 | AX | 2608 |
| 1300459 | 20583 | 20598 | N/A | N/A | GACCAGAGAGCTCCGG | 51 | AX | 73 |
| | 20709 | 20724 | | | | | | |
| | 20751 | 20766 | | | | | | |
| | 20919 | 20934 | | | | | | |
| 1300468 | 21599 | 21614 | N/A | N/A | TACAATATGAGGACCC | 70 | AX | 228 |
| 1300514 | 23040 | 23055 | 3315 | 3330 | CATAGAGTCTGGTCAG | 49 | AX | 428 |
| 1300543 | 18858 | 18873 | N/A | N/A | TGTGCAGTAACATATC | 59 | AX | 2722 |
| 1300551 | 12142 | 12157 | 2445 | 2460 | ACCCCTTTTCGAATTT | 65 | AX | 2269 |
| 1300571 | 20277 | 20292 | N/A | N/A | TTTGACGATTTCATCC | 37 | AX | 2731 |
| 1300579 | 12449 | 12464 | 2752 | 2767 | TGACAGTTCTCAATGC | 71 | AX | 2727 |
| 1300597 | 20929 | 20944 | N/A | N/A | GAACACACCTGACCAG | 34 | AX | 1689 |
| | 21055 | 21070 | | | | | | |
| 1300621 | 23824 | 23839 | N/A | N/A | GCATGATTATTAGTGG | 53 | AX | 2633 |
| 1300627 | 25018 | 25033 | N/A | N/A | TAACTACATAGAGCAT | 57 | AX | 2616 |
| 1300638 | 25036 | 25051 | N/A | N/A | GTTAGGTATTGACAAT | 94 | AX | 2651 |
| 1300652 | 26970 | 26985 | N/A | N/A | CATAAAAGTCTCGACT | 84 | AX | 2619 |
| 1300656 | 27022 | 27037 | N/A | N/A | GGCTATACCTAACACA | 32 | AX | 162 |
| | 27058 | 27073 | | | | | | |
| | 27112 | 27127 | | | | | | |
| 1300670 | 27107 | 27122 | N/A | N/A | TACCTAACACATAGGC | 63 | AX | 2470 |
| | 27125 | 27140 | | | | | | |
| 1300671 | 31012 | 31027 | 3456 | 3471 | TGAGTACCGAGGACAA | 64 | AX | 2358 |
| 1300694 | 31082 | 31097 | 3526 | 3541 | AGTAGTTTGATCCCCT | 45 | AX | 1974 |
| 1232737 | 5138 | 5153 | 90 | 105 | ATGCCATCTTGACCCA | 69 | AY | 22 |
| 1300261 | 26958 | 26973 | N/A | N/A | GACTGATTAATGCCAC | 34 | AY | 2620 |
| 1300273 | 27198 | 27213 | N/A | N/A | GTATTGAGACAGGCAG | 45 | AY | 854 |
| 1300277 | 27130 | 27145 | N/A | N/A | AGCTATACCTAACACA | 54 | AY | 2239 |
| 1300299 | 27367 | 27382 | N/A | N/A | AGCATTTAGGACCGTC | 34 | AY | 1316 |
| 1300303 | 10295 | 10310 | 1095 | 1110 | CCAGTAAACCCATCCA | 40 | AY | 1723 |
| 1300327 | 11008 | 11023 | 1311 | 1326 | TGGTCTTGCCGATGGC | 61 | AY | 2033 |
| 1300336 | 5148 | 5163 | 100 | 115 | CCACTTCACGATGCCA | 39 | AY | 407 |
| 1300352 | 11973 | 11988 | 2276 | 2291 | CTCGCAGTCCACTTCC | 69 | AY | 868 |
| 1300365 | 11980 | 11995 | 2283 | 2298 | AGAACTTCTCGCAGTC | 57 | AY | 2723 |
| 1300384 | 12702 | 12717 | N/A | N/A | TGATGTAGCTGTGGCA | 41 | AY | 2654 |
| 1300440 | 15184 | 15199 | N/A | N/A | CCGTCAGAGTCTGTCC | 26 | AY | 2721 |
| 1300441 | 17671 | 17686 | N/A | N/A | TGGAATATATCGAGCA | 19 | AY | 1454 |
| 1300460 | 20582 | 20597 | N/A | N/A | ACCAGAGAGCTCCGGA | 40 | AY | 611 |
| | 20708 | 20723 | | | | | | |
| | 20750 | 20765 | | | | | | |
| | 20918 | 20933 | | | | | | |
| 1300463 | 23282 | 23297 | N/A | N/A | ATTATATGCCTCCAGT | 50 | AY | 2626 |
| 1300469 | 11042 | 11057 | 1345 | 1360 | ATCTTAATGGGACTCA | 47 | AY | 340 |
| 1300480 | 23492 | 23507 | N/A | N/A | GTGTAGAGGCTGCATG | 42 | AY | 2610 |
| 1300494 | 25163 | 25178 | N/A | N/A | ACGACCTATAAGGACC | 79 | AY | 2657 |
| 1300511 | 17441 | 17456 | N/A | N/A | AACATTAGGTCTTGTG | 64 | AY | 684 |
| 1300520 | 23039 | 23054 | 3314 | 3329 | ATAGAGTCTGGTCAGG | 60 | AY | 351 |
| 1300532 | 17703 | 17718 | N/A | N/A | ACTCTCATCTATGAAC | 58 | AY | 2725 |
| 1300540 | 18352 | 18367 | N/A | N/A | GGACTTTAACACTTGC | 34 | AY | 2665 |
| 1300581 | 20420 | 20435 | N/A | N/A | TAAGTGAAGCGGGCGG | 81 | AY | 2630 |
| 1300602 | 20698 | 20713 | N/A | N/A | TCCGGAATAGAGAAAG | 97 | AY | 381 |
| | 20740 | 20755 | | | | | | |
| | 20908 | 20923 | | | | | | |
| | 20992 | 21007 | | | | | | |
| 1300649 | 26779 | 26794 | N/A | N/A | TCGATAGATCTACACA | 43 | AY | 2617 |
| 1300664 | 28757 | 28772 | N/A | N/A | TATTAAGCAACGGTTA | 69 | AY | 1549 |
| 1300668 | 27111 | 27126 | N/A | N/A | GCTATACCTAACACAT | 34 | AY | 2162 |
| | 27129 | 27144 | | | | | | |
| 1232737 | 5138 | 5153 | 90 | 105 | ATGCCATCTTGACCCA | 63 | AZ | 22 |
| 1300314 | 11001 | 11016 | 1304 | 1319 | GCCGATGGCCAGAAGC | 105 | AZ | 2621 |
| 1300339 | 11231 | 11246 | 1534 | 1549 | GTCACAAGGCTCACCT | 81 | AZ | 2624 |
| 1300346 | 11296 | 11311 | 1599 | 1614 | TCTTGTGGATGGGTGG | 42 | AZ | 2188 |
| 1300351 | 11304 | 11319 | 1607 | 1622 | TCTCACGATCTTGTGG | 89 | AZ | 882 |
| 1300388 | 5168 | 5183 | 120 | 135 | TTACAGTTTACGGTGA | 90 | AZ | 24 |
| 1300401 | 14573 | 14588 | N/A | N/A | GGTGCCATCTAGAGGG | 87 | AZ | 1451 |
| | 14865 | 14880 | | | | | | |
| | 15155 | 15170 | | | | | | |
| | 15228 | 15243 | | | | | | |
| 1300408 | 14577 | 14592 | N/A | N/A | GGCAGGTGCCATCTAG | 50 | AZ | 1759 |
| | 14869 | 14884 | | | | | | |
| | 15159 | 15174 | | | | | | |
| | 15232 | 15247 | | | | | | |
| 1300411 | 16730 | 16745 | 3048 | 3063 | GAATGTTACAGCCAGG | 30 | AZ | 2275 |
| 1300477 | 23494 | 23509 | N/A | N/A | TAGTGTAGAGGCTGCA | 11 | AZ | 538 |
| | 23527 | 23542 | | | | | | |
| 1300495 | 23534 | 23549 | N/A | N/A | CCTCTCCTAGTGTAGA | 121 | AZ | 2732 |
| 1300500 | 23501 | 23516 | N/A | N/A | TCTTTCCTAGTGTAGA | 117 | AZ | 2635 |
| 1300508 | 23002 | 23017 | 3277 | 3292 | GATGCAAGATCCTGAC | 52 | AZ | 2123 |
| 1300525 | 12135 | 12150 | 2438 | 2453 | TTCGAATTTGCCATAG | 57 | AZ | 189 |
| 1300528 | 17449 | 17464 | N/A | N/A | ACTAGATTAACATTAG | 85 | AZ | 992 |
| 1300568 | 19879 | 19894 | N/A | N/A | ATTAGTTTCCCCGTCA | 25 | AZ | 2647 |
| 1300605 | 12456 | 12471 | 2759 | 2774 | CACCCGATGACAGTTC | 64 | AZ | 345 |
| 1300607 | 22430 | 22445 | N/A | N/A | CATAATGTCCCCGAGG | 68 | AZ | 2632 |
| 1300608 | 20701 | 20716 | N/A | N/A | AGCTCCGGAATAGAGA | 96 | AZ | 612 |
| | 20743 | 20758 | | | | | | |
| | 20911 | 20926 | | | | | | |
| | 20995 | 21010 | | | | | | |
| 1300709 | 25746 | 25761 | N/A | N/A | TGTTGAAATTGAGAGG | 60 | AZ | 2728 |
| 1300711 | 26603 | 26618 | N/A | N/A | CCTTATTTGGTGATAC | 39 | AZ | 1314 |

### Example 5: Effect of Mixed cEt and 2′-OMe, Uniform Phosphorothioate Modified Oligonucleotides on Human NLRP3 RNA In Vitro, Single Dose

Modified oligonucleotides complementary to human NLRP3 nucleic acid were designed and tested for their single dose effects on NLRP3 RNA in vitro. The modified oligonucleotides were tested in a series of experiments that had the same culture conditions.

The modified oligonucleotides in the table below are 16 nucleosides in length, wherein the sugar motif for the modified oligonucleotides is (from 5′ to 3′): kkkdyddddddddkkk; wherein each “d” represents a 2′-β-D-deoxyribosyl sugar moiety, each “y” represents a 2′-O-methylribose (2′-OMe) sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine unless otherwise marked; non-methylated cytosine residues are indicated by a bolded and underlined C.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. “N/A” indicates that the modified oligonucleotide is not 100% complementary to that particular target nucleic acid sequence.

Cultured THP-1 cells were treated with modified oligonucleotide at a concentration of 2000 nM by electroporation at a density of 300,000 cells per well. After a treatment period of approximately 24 hours, total RNA was isolated from the cells and NLRP3 RNA levels were measured by quantitative real-time RTPCR. NLRP3 RNA levels were measured by human primer-probe set RTS37509 (described herein above). NLRP3 RNA levels were normalized to total RNA content, as measured by RIBOGREEN®. Reduction of NLRP3 RNA is presented in the tables below as percent NLRP3 RNA relative to the amount of NLRP3 RNA in untreated control cells (% UTC).

Each separate experimental analysis described in this example is identified by a letter ID in the table column below labeled “AID” (Analysis ID). In the table below, Compound Nos. 1232737 and 1233176 (described herein above) were used as benchmark compounds.

TABLE 7
Reduction of NLRP3 RNA by modified oligonucleotides with a mixed cEt/2′-OMe sugar motif and
uniform phosphorothioate internucleoside linkages at a concentration of 2000 nM in THP-1
cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | NLRP3 | | |
| Compound | Start | Stop | Start | Stop | | (% | | SEQ ID |
| Number | Site | Site | Site | Site | Sequence (5′ to 3′) | UTC) | AID | NO |
| 1232737 | 5138 | 5153 | 90 | 105 | ATGCCATCTTGACCCA | 59 | BA | 22 |
| 1233176 | 23496 | 23511 | N/A | N/A | CCTAGTGTAGAGGCTG | 30 | BA | 77 |
| | 23529 | 23544 | | | | | | |
| 1304679 | 20936 | 20951 | N/A | N/A | AGCAUCAGAACACACC | 35 | BA | 2734 |
| | 21062 | 21077 | | | | | | |
| 1304683 | 25074 | 25089 | N/A | N/A | GCTCUGTTTGTGATAC | 52 | BA | 2735 |
| | 26262 | 26277 | | | | | | |
| 1304684 | 23005 | 23020 | 3280 | 3295 | ACTGATGCAAGATCCT | 41 | BA | 2355 |
| 1304686 | 20641 | 20656 | N/A | N/A | GCATCAGGACACACCT | 26 | BA | 2304 |
| | 20683 | 20698 | | | | | | |
| | 20725 | 20740 | | | | | | |
| | 20767 | 20782 | | | | | | |
| | 20809 | 20824 | | | | | | |
| | 20851 | 20866 | | | | | | |
| | 21019 | 21034 | | | | | | |
| 1304689 | 14587 | 14602 | N/A | N/A | CTCAGAGTAAGGCAGG | 24 | BA | 2298 |
| | 14879 | 14894 | | | | | | |
| | 15169 | 15184 | | | | | | |
| | 15242 | 15257 | | | | | | |
| 1304692 | 5244 | 5259 | 196 | 211 | GTGTGTCCTGAGCCAT | 62 | BA | 2101 |
| 1304694 | 23284 | 23299 | N/A | N/A | ACATUATATGCCTCCA | 45 | BA | 2736 |
| 1304695 | 11008 | 11023 | 1311 | 1326 | TGGTCTTGCCGATGGC | 68 | BA | 2033 |
| 1304697 | 20421 | 20436 | N/A | N/A | GTAAGTGAAGCGGGCG | 45 | BA | 2072 |
| 1304700 | 14576 | 14591 | N/A | N/A | GCAGGTGCCATCTAGA | 82 | BA | 1682 |
| | 14868 | 14883 | | | | | | |
| | 15158 | 15173 | | | | | | |
| | 15231 | 15246 | | | | | | |
| 1304706 | 14575 | 14590 | N/A | N/A | CAGGUGCCATCTAGAG | 57 | BA | 2737 |
| | 14867 | 14882 | | | | | | |
| | 15157 | 15172 | | | | | | |
| | 15230 | 15245 | | | | | | |
| 1304707 | 14503 | 14518 | N/A | N/A | GAAGGTGCTATCTAGA | 29 | BA | 1758 |
| | 14649 | 14664 | | | | | | |
| | 14722 | 14737 | | | | | | |
| | 14795 | 14810 | | | | | | |
| | 14941 | 14956 | | | | | | |
| | 15014 | 15029 | | | | | | |
| | 15087 | 15102 | | | | | | |
| 1304708 | 11237 | 11252 | 1540 | 1555 | CTCTGTGTCACAAGGC | 46 | BA | 1726 |
| 1304709 | 14673 | 14688 | N/A | N/A | ATCAGAGTCTGTCCTC | 11 | BA | 1606 |
| | 14819 | 14834 | | | | | | |
| | 14965 | 14980 | | | | | | |
| 1304710 | 25745 | 25760 | N/A | N/A | GTTGAAATTGAGAGGT | 49 | BA | 1235 |
| 1304712 | 23495 | 23510 | N/A | N/A | CTAGUGTAGAGGCTGC | 46 | BA | 2738 |
| | 23528 | 23543 | | | | | | |
| 1304714 | 14676 | 14691 | N/A | N/A | CCAAUCAGAGTCTGTC | 57 | BA | 2739 |
| | 14822 | 14837 | | | | | | |
| | 14968 | 14983 | | | | | | |
| 1304716 | 12138 | 12153 | 2441 | 2456 | CTTTUCGAATTTGCCA | 38 | BA | 2740 |
| 1304718 | 20587 | 20602 | N/A | N/A | ACCTGACCAGAGAGCT | 43 | BA | 1226 |
| | 20629 | 20644 | | | | | | |
| | 20671 | 20686 | | | | | | |
| | 20713 | 20728 | | | | | | |
| | 20755 | 20770 | | | | | | |
| | 20797 | 20812 | | | | | | |
| | 20923 | 20938 | | | | | | |
| | 21049 | 21064 | | | | | | |
| 1304720 | 11006 | 11021 | 1309 | 1324 | GTCTUGCCGATGGCCA | 61 | BA | 2741 |
| 1304721 | 14505 | 14520 | N/A | N/A | AGGAAGGTGCTATCTA | 30 | BA | 1912 |
| | 14651 | 14666 | | | | | | |
| | 14724 | 14739 | | | | | | |
| | 14797 | 14812 | | | | | | |
| | 14943 | 14958 | | | | | | |
| | 15016 | 15031 | | | | | | |
| | 15089 | 15104 | | | | | | |
| 1304722 | 11978 | 11993 | 2281 | 2296 | AACTUCTCGCAGTCCA | 47 | BA | 2742 |
| 1304723 | 14481 | 14496 | N/A | N/A | GGTGCTGAGCCTCCTA | 36 | BA | 757 |
| | 14627 | 14642 | | | | | | |
| | 14773 | 14788 | | | | | | |
| | 15065 | 15080 | | | | | | |
| 1304724 | 15112 | 15127 | N/A | N/A | CCAGUCAGAGTCTGTC | 38 | BA | 2743 |
| | 15258 | 15273 | | | | | | |
| 1304731 | 14606 | 14621 | N/A | N/A | GCTTCAGTGAGAGTCT | 49 | BA | 1298 |
| | 14752 | 14767 | | | | | | |
| | 14898 | 14913 | | | | | | |
| | 15044 | 15059 | | | | | | |
| 1304732 | 12467 | 12482 | 2770 | 2785 | GACAGTGACTCCACCC | 30 | BA | 733 |
| 1304733 | 20634 | 20649 | N/A | N/A | GACACACCTGACCAGA | 43 | BA | 1996 |
| | 20676 | 20691 | | | | | | |
| | 20718 | 20733 | | | | | | |
| | 20760 | 20775 | | | | | | |
| | 20802 | 20817 | | | | | | |
| 1304734 | 25819 | 25834 | N/A | N/A | ATGTUTACACCAAGCA | 86 | BA | 2744 |
| 1304735 | 14491 | 14506 | N/A | N/A | TAGAGAAATGGGTGCT | 44 | BA | 988 |
| | 14637 | 14652 | | | | | | |
| | 14710 | 14725 | | | | | | |
| | 14783 | 14798 | | | | | | |
| | 15002 | 15017 | | | | | | |
| | 15075 | 15090 | | | | | | |
| 1304736 | 27367 | 27382 | N/A | N/A | AGCAUTTAGGACCGTC | 66 | BA | 2745 |
| 1304738 | 31082 | 31097 | 3526 | 3541 | AGTAGTTTGATCCCCT | 61 | BA | 1974 |
| 1304741 | 15312 | 15327 | N/A | N/A | AGAGUAAGGCAGCTGC | 61 | BA | 2746 |
| 1304743 | 14559 | 14574 | N/A | N/A | GGATGGGTGCTGAGCC | 48 | BA | 1143 |
| | 14851 | 14866 | | | | | | |
| | 15141 | 15156 | | | | | | |
| | 15214 | 15229 | | | | | | |
| 1304745 | 14493 | 14508 | N/A | N/A | TCTAGAGAAATGGGTG | 27 | BA | 1142 |
| | 14639 | 14654 | | | | | | |
| | 14712 | 14727 | | | | | | |
| | 14785 | 14800 | | | | | | |
| | 15004 | 15019 | | | | | | |
| | 15077 | 15092 | | | | | | |
| 1304747 | 25822 | 25837 | N/A | N/A | GATAUGTTTACACCAA | 39 | BA | 2747 |
| 1304749 | 17670 | 17685 | N/A | N/A | GGAAUATATCGAGCAT | 43 | BA | 2748 |
| 1304750 | 27023 | 27038 | N/A | N/A | AGGCUATACCTAACAC | 36 | BA | 2749 |
| | 27059 | 27074 | | | | | | |
| | 27095 | 27110 | | | | | | |
| | 27113 | 27128 | | | | | | |
| 1304751 | 15113 | 15128 | N/A | N/A | TCCAGTCAGAGTCTGT | 22 | BA | 1068 |
| | 15259 | 15274 | | | | | | |
| 1304753 | 27022 | 27037 | N/A | N/A | GGCTATACCTAACACA | 44 | BA | 162 |
| | 27058 | 27073 | | | | | | |
| | 27112 | 27127 | | | | | | |
| 1304757 | 16644 | 16659 | 2962 | 2977 | GTTAGACTCTGGCTGG | 47 | BA | 1274 |
| 1304758 | 14594 | 14609 | N/A | N/A | GTCTGTCCTCAGAGTA | 14 | BA | 990 |
| | 14886 | 14901 | | | | | | |
| | 15176 | 15191 | | | | | | |
| | 15249 | 15264 | | | | | | |
| 1304760 | 27201 | 27216 | N/A | N/A | GCAGUATTGAGACAGG | 55 | BA | 2750 |
| 1304761 | 23305 | 23320 | N/A | N/A | TTATCTTATTAGCAGC | 49 | BA | 922 |
| 1304762 | 11950 | 11965 | 2253 | 2268 | ACAGGTTCATCCTCAG | 53 | BA | 806 |
| 1304765 | 12466 | 12481 | 2769 | 2784 | ACAGUGACTCCACCCG | 55 | BA | 2751 |
| 1304769 | 21596 | 21611 | N/A | N/A | AATAUGAGGACCCAGG | 65 | BA | 2752 |
| 1304770 | 12140 | 12155 | 2443 | 2458 | CCCTUTTCGAATTTGC | 52 | BA | 2753 |
| 1304771 | 5288 | 5303 | 240 | 255 | GCCAAATGCTTACCAG | 48 | BA | 333 |
| 1304776 | 12454 | 12469 | 2757 | 2772 | CCCGATGACAGTTCTC | 43 | BA | 268 |
| 1304777 | 17665 | 17680 | N/A | N/A | ATATCGAGCATCATGG | 64 | BA | 375 |
| 1304778 | 23497 | 23512 | N/A | N/A | TCCTAGTGTAGAGGCT | 31 | BA | 154 |
| | 23530 | 23545 | | | | | | |
| 1304780 | 29461 | 29476 | N/A | N/A | GTCAAATTAAGCCCAT | 31 | BA | 168 |
| 1304782 | 25161 | 25176 | N/A | N/A | GACCUATAAGGACCCA | 62 | BA | 2754 |
| 1304787 | 26959 | 26974 | N/A | N/A | CGACUGATTAATGCCA | 53 | BA | 2755 |
| 1304789 | 26960 | 26975 | N/A | N/A | TCGACTGATTAATGCC | 50 | BA | 2558 |
| 1304792 | 26962 | 26977 | N/A | N/A | TCTCGACTGATTAATG | 86 | BA | 2667 |
| 1304794 | 14583 | 14598 | N/A | N/A | GAGTAAGGCAGGTGCC | 35 | BA | 2452 |
| | 14875 | 14890 | | | | | | |
| | 15165 | 15180 | | | | | | |
| | 15238 | 15253 | | | | | | |
| 1304796 | 18351 | 18366 | N/A | N/A | GACTUTAACACTTGCT | 66 | BA | 2756 |
| 1304798 | 14584 | 14599 | N/A | N/A | AGAGUAAGGCAGGTGC | 24 | BA | 2757 |
| | 14876 | 14891 | | | | | | |
| | 15166 | 15181 | | | | | | |
| | 15239 | 15254 | | | | | | |
| 1304800 | 17987 | 18002 | N/A | N/A | CCATACAAAGCATTCT | 58 | BA | 2224 |
| 1304802 | 17698 | 17713 | N/A | N/A | CATCUATGAACATGGC | 61 | BA | 2758 |
| 1304804 | 19883 | 19898 | N/A | N/A | CTTGATTAGTTTCCCC | 29 | BA | 1764 |
| 1304805 | 19268 | 19283 | N/A | N/A | GACACTAGAGCCTCAG | 71 | BA | 1686 |
| 1304806 | 26957 | 26972 | N/A | N/A | ACTGATTAATGCCACT | 43 | BA | 1392 |
| 1304807 | 17671 | 17686 | N/A | N/A | TGGAATATATCGAGCA | 66 | BA | 1454 |
| 1304808 | 26601 | 26616 | N/A | N/A | TTATUTGGTGATACGG | 71 | BA | 2759 |
| 1304809 | 27368 | 27383 | N/A | N/A | GAGCATTTAGGACCGT | 37 | BA | 1393 |
| 1304811 | 18758 | 18773 | N/A | N/A | GCATUTAAGCCACCGA | 99 | BA | 2760 |
| 1304814 | 6741 | 6756 | N/A | N/A | AGGGAATTCCGACACA | 55 | BA | 673 |
| 1304815 | 17446 | 17461 | N/A | N/A | AGATUAACATTAGGTC | 82 | BA | 2761 |
| 1304817 | 23285 | 23300 | N/A | N/A | TACAUTATATGCCTCC | 47 | BA | 2762 |
| 1304825 | 21599 | 21614 | N/A | N/A | TACAATATGAGGACCC | 67 | BA | 228 |
| 1304826 | 11261 | 11276 | 1564 | 1579 | CTCAUGATCAGGTCCC | 56 | BA | 2763 |
| 1304827 | 25019 | 25034 | N/A | N/A | GTAACTACATAGAGCA | 76 | BA | 2466 |
| 1304828 | 36033 | 36048 | 4386 | 4401 | TGAAUTTTCAACCAGC | 61 | BA | 2764 |
| 1304829 | 14508 | 14523 | N/A | N/A | GGAAGGAAGGTGCTAT | 40 | BA | 2451 |
| | 14654 | 14669 | | | | | | |
| | 14727 | 14742 | | | | | | |
| | 14800 | 14815 | | | | | | |
| | 14946 | 14961 | | | | | | |
| | 15019 | 15034 | | | | | | |
| | 15092 | 15107 | | | | | | |
| 1232737 | 5138 | 5153 | 90 | 105 | ATGCCATCTTGACCCA | 59 | BB | 22 |
| 1304677 | 20583 | 20598 | N/A | N/A | GACCAGAGAGCTCCGG | 30 | BB | 73 |
| | 20709 | 20724 | | | | | | |
| | 20751 | 20766 | | | | | | |
| | 20919 | 20934 | | | | | | |
| 1304678 | 23822 | 23837 | N/A | N/A | ATGAUTATTAGTGGGT | 69 | BB | 2765 |
| 1304680 | 6020 | 6035 | 972 | 987 | TCCTGTTGATCGCAGC | 66 | BB | 2338 |
| 1304681 | 11299 | 11314 | 1602 | 1617 | CGATCTTGTGGATGGG | 27 | BB | 2343 |
| 1304682 | 12465 | 12480 | 2768 | 2783 | CAGTGACTCCACCCGA | 44 | BB | 2350 |
| 1304685 | 20642 | 20657 | N/A | N/A | AGCAUCAGGACACACC | 21 | BB | 2766 |
| | 20684 | 20699 | | | | | | |
| | 20726 | 20741 | | | | | | |
| | 20810 | 20825 | | | | | | |
| | 20852 | 20867 | | | | | | |
| | 21020 | 21035 | | | | | | |
| 1304687 | 5245 | 5260 | 197 | 212 | AGTGUGTCCTGAGCCA | 58 | BB | 2767 |
| 1304688 | 25033 | 25048 | N/A | N/A | AGGTATTGACAATAGT | 29 | BB | 2312 |
| 1304690 | 14586 | 14601 | N/A | N/A | TCAGAGTAAGGCAGGT | 27 | BB | 2221 |
| | 14878 | 14893 | | | | | | |
| | 15168 | 15183 | | | | | | |
| | 15241 | 15256 | | | | | | |
| 1304691 | 10300 | 10315 | 1100 | 1115 | GTACUCCAGTAAACCC | 49 | BB | 2768 |
| 1304693 | 27111 | 27126 | N/A | N/A | GCTAUACCTAACACAT | 27 | BB | 2769 |
| | 27129 | 27144 | | | | | | |
| 1304696 | 14604 | 14619 | N/A | N/A | TTCAGTGAGAGTCTGT | 43 | BB | 1144 |
| | 14750 | 14765 | | | | | | |
| | 14896 | 14911 | | | | | | |
| | 15042 | 15057 | | | | | | |
| 1304698 | 14675 | 14690 | N/A | N/A | CAATCAGAGTCTGTCC | 29 | BB | 1760 |
| | 14821 | 14836 | | | | | | |
| | 14967 | 14982 | | | | | | |
| 1304699 | 20635 | 20650 | N/A | N/A | GGACACACCTGACCAG | 22 | BB | 2073 |
| | 20677 | 20692 | | | | | | |
| | 20719 | 20734 | | | | | | |
| | 20761 | 20776 | | | | | | |
| | 20803 | 20818 | | | | | | |
| 1304701 | 14501 | 14516 | N/A | N/A | AGGTGCTATCTAGAGA | 31 | BB | 1681 |
| | 14647 | 14662 | | | | | | |
| | 14720 | 14735 | | | | | | |
| | 14793 | 14808 | | | | | | |
| | 14939 | 14954 | | | | | | |
| | 15012 | 15027 | | | | | | |
| | 15085 | 15100 | | | | | | |
| 1304702 | 20274 | 20289 | N/A | N/A | GACGATTTCATCCCAG | 26 | BB | 1918 |
| 1304703 | 26956 | 26971 | N/A | N/A | CTGAUTAATGCCACTT | 74 | BB | 2770 |
| 1304704 | 14504 | 14519 | N/A | N/A | GGAAGGTGCTATCTAG | 31 | BB | 1835 |
| | 14650 | 14665 | | | | | | |
| | 14723 | 14738 | | | | | | |
| | 14796 | 14811 | | | | | | |
| | 14942 | 14957 | | | | | | |
| | 15015 | 15030 | | | | | | |
| | 15088 | 15103 | | | | | | |
| 1304705 | 11236 | 11251 | 1539 | 1554 | TCTGUGTCACAAGGCT | 72 | BB | 2771 |
| 1304711 | 14589 | 14604 | N/A | N/A | TCCTCAGAGTAAGGCA | 66 | BB | 682 |
| | 14881 | 14896 | | | | | | |
| | 15171 | 15186 | | | | | | |
| | 15244 | 15259 | | | | | | |
| 1304713 | 20632 | 20647 | N/A | N/A | CACACCTGACCAGAGA | 29 | BB | 1842 |
| | 20674 | 20689 | | | | | | |
| | 20716 | 20731 | | | | | | |
| | 20758 | 20773 | | | | | | |
| | 20800 | 20815 | | | | | | |
| | 20926 | 20941 | | | | | | |
| | 21052 | 21067 | | | | | | |
| 1304715 | 19882 | 19897 | N/A | N/A | TTGAUTAGTTTCCCCG | 61 | BB | 2772 |
| 1304717 | 22432 | 22447 | N/A | N/A | AGCAUAATGTCCCCGA | 56 | BB | 2773 |
| 1304719 | 11235 | 11250 | 1538 | 1553 | CTGTGTCACAAGGCTC | 39 | BB | 1572 |
| 1304725 | 12486 | 12501 | 2789 | 2804 | GTTAUGGAGAAACCCC | 47 | BB | 2774 |
| 1304726 | 18856 | 18871 | N/A | N/A | TGCAGTAACATATCCA | 22 | BB | 1532 |
| 1304727 | 23038 | 23053 | 3313 | 3328 | TAGAGTCTGGTCAGGG | 41 | BB | 1893 |
| 1304728 | 14667 | 14682 | N/A | N/A | GTCTGTCCTCAGAGGA | 15 | BB | 1452 |
| | 14740 | 14755 | | | | | | |
| | 14813 | 14828 | | | | | | |
| | 14959 | 14974 | | | | | | |
| | 15032 | 15047 | | | | | | |
| 1304729 | 14558 | 14573 | N/A | N/A | GATGGGTGCTGAGCCC | 40 | BB | 1066 |
| | 14850 | 14865 | | | | | | |
| | 15140 | 15155 | | | | | | |
| | 15213 | 15228 | | | | | | |
| | 15286 | 15301 | | | | | | |
| 1304730 | 15182 | 15197 | N/A | N/A | GTCAGAGTCTGTCCTC | 3 | BB | 1145 |
| | 15255 | 15270 | | | | | | |
| 1304737 | 14581 | 14596 | N/A | N/A | GTAAGGCAGGTGCCAT | 34 | BB | 1990 |
| | 14873 | 14888 | | | | | | |
| | 15163 | 15178 | | | | | | |
| | 15236 | 15251 | | | | | | |
| 1304739 | 14668 | 14683 | N/A | N/A | AGTCUGTCCTCAGAGG | 7 | BB | 2775 |
| | 14741 | 14756 | | | | | | |
| | 14814 | 14829 | | | | | | |
| | 14960 | 14975 | | | | | | |
| | 15033 | 15048 | | | | | | |
| 1304740 | 10881 | 10896 | 1184 | 1199 | AATGCACTGGAATCTG | 51 | BB | 1878 |
| 1304742 | 12275 | 12290 | 2578 | 2593 | GCTTUCACTTCAATCC | 37 | BB | 2776 |
| 1304744 | 10928 | 10943 | 1231 | 1246 | CGTTUGTTGAGGCTCA | 45 | BB | 2777 |
| 1304746 | 14603 | 14618 | N/A | N/A | TCAGUGAGAGTCTGTC | 32 | BB | 2778 |
| | 14749 | 14764 | | | | | | |
| | 14895 | 14910 | | | | | | |
| | 15041 | 15056 | | | | | | |
| 1304748 | 20588 | 20603 | N/A | N/A | CACCUGACCAGAGAGC | 38 | BB | 2779 |
| | 20630 | 20645 | | | | | | |
| | 20672 | 20687 | | | | | | |
| | 20714 | 20729 | | | | | | |
| | 20756 | 20771 | | | | | | |
| | 20798 | 20813 | | | | | | |
| | 20924 | 20939 | | | | | | |
| | 21050 | 21065 | | | | | | |
| 1304752 | 31017 | 31032 | 3461 | 3476 | AGTGCTGAGTACCGAG | 43 | BB | 972 |
| 1304754 | 14574 | 14589 | N/A | N/A | AGGTGCCATCTAGAGG | 34 | BB | 1528 |
| | 14866 | 14881 | | | | | | |
| | 15156 | 15171 | | | | | | |
| | 15229 | 15244 | | | | | | |
| 1304755 | 18855 | 18870 | N/A | N/A | GCAGUAACATATCCAT | 44 | BB | 2780 |
| 1304756 | 11977 | 11992 | 2280 | 2295 | ACTTCTCGCAGTCCAC | 49 | BB | 960 |
| 1304759 | 24553 | 24568 | N/A | N/A | GTCAAATTGCCAAGGA | 59 | BB | 1003 |
| 1304763 | 14485 | 14500 | N/A | N/A | AATGGGTGCTGAGCCT | 17 | BB | 911 |
| | 14631 | 14646 | | | | | | |
| | 14777 | 14792 | | | | | | |
| | 15069 | 15084 | | | | | | |
| 1304764 | 12704 | 12719 | N/A | N/A | TATGATGTAGCTGTGG | 54 | BB | 678 |
| 1304766 | 14480 | 14495 | N/A | N/A | GTGCUGAGCCTCCTAC | 27 | BB | 2781 |
| | 14626 | 14641 | | | | | | |
| | 14772 | 14787 | | | | | | |
| | 15064 | 15079 | | | | | | |
| 1304767 | 28760 | 28775 | N/A | N/A | AACTATTAAGCAACGG | 54 | BB | 628 |
| 1304768 | 16732 | 16747 | 3050 | 3065 | CCGAATGTTACAGCCA | 60 | BB | 501 |
| 1304772 | 26784 | 26799 | N/A | N/A | CCCAATCGATAGATCT | 38 | BB | 468 |
| 1304773 | 5148 | 5163 | 100 | 115 | CCACUTCACGATGCCA | 56 | BB | 2782 |
| 1304774 | 14502 | 14517 | N/A | N/A | AAGGUGCTATCTAGAG | 18 | BB | 2783 |
| | 14648 | 14663 | | | | | | |
| | 14721 | 14736 | | | | | | |
| | 14794 | 14809 | | | | | | |
| | 14940 | 14955 | | | | | | |
| | 15013 | 15028 | | | | | | |
| | 15086 | 15101 | | | | | | |
| 1304775 | 5171 | 5186 | 123 | 138 | GTATUACAGTTTACGG | 89 | BB | 2784 |
| 1304779 | 26973 | 26988 | N/A | N/A | ATGCATAAAAGTCTCG | 32 | BB | 85 |
| 1304781 | 23496 | 23511 | N/A | N/A | CCTAGTGTAGAGGCTG | 35 | BB | 2801 |
| | 23529 | 23544 | | | | | | |
| 1304783 | 17669 | 17684 | N/A | N/A | GAATATATCGAGCATC | 63 | BB | 68 |
| 1304784 | 27199 | 27214 | N/A | N/A | AGTAUTGAGACAGGCA | 42 | BB | 2785 |
| 1304785 | 26958 | 26973 | N/A | N/A | GACTGATTAATGCCAC | 35 | BB | 2620 |
| 1304786 | 5133 | 5148 | 85 | 100 | ATCTUGACCCATCAGC | 93 | BB | 2786 |
| 1304788 | 26961 | 26976 | N/A | N/A | CTCGACTGATTAATGC | 70 | BB | 2544 |
| 1304790 | 27028 | 27043 | N/A | N/A | CCCAUAGGCTATACCT | 20 | BB | 2787 |
| | 27064 | 27079 | | | | | | |
| 1304791 | 27094 | 27109 | N/A | N/A | GGCTATACCTAACACG | 54 | BB | 2515 |
| 1304793 | 5249 | 5264 | 201 | 216 | CAGGAGTGTGTCCTGA | 39 | BB | 2663 |
| 1304795 | 21598 | 21613 | N/A | N/A | ACAAUATGAGGACCCA | 20 | BB | 2788 |
| 1304797 | 35773 | 35788 | 4126 | 4141 | CTAAUTACATGAGGTC | 72 | BB | 2789 |
| 1304799 | 22900 | 22915 | N/A | N/A | GACAAGGAAGCACCCG | 43 | BB | 2076 |
| 1304801 | 19474 | 19489 | N/A | N/A | GCAAAGATGAAGAGTG | 59 | BB | 2071 |
| 1304803 | 14506 | 14521 | N/A | N/A | AAGGAAGGTGCTATCT | 47 | BB | 1989 |
| | 14652 | 14667 | | | | | | |
| | 14725 | 14740 | | | | | | |
| | 14798 | 14813 | | | | | | |
| | 14944 | 14959 | | | | | | |
| | 15017 | 15032 | | | | | | |
| | 15090 | 15105 | | | | | | |
| 1304810 | 17464 | 17479 | N/A | N/A | GTCTUTAAAGGAGTGA | 50 | BB | 2790 |
| 1304812 | 14492 | 14507 | N/A | N/A | CTAGAGAAATGGGTGC | 28 | BB | 1065 |
| | 14638 | 14653 | | | | | | |
| | 14711 | 14726 | | | | | | |
| | 14784 | 14799 | | | | | | |
| | 15003 | 15018 | | | | | | |
| | 15076 | 15091 | | | | | | |
| 1304813 | 27198 | 27213 | N/A | N/A | GTATUGAGACAGGCAG | 43 | BB | 2791 |
| 1304816 | 35774 | 35789 | 4127 | 4142 | GCTAATTACATGAGGT | 51 | BB | 670 |
| 1304818 | 11043 | 11058 | 1346 | 1361 | CATCUTAATGGGACTC | 59 | BB | 2792 |
| 1304819 | 17672 | 17687 | N/A | N/A | ATGGAATATATCGAGC | 66 | BB | 145 |
| 1304820 | 20696 | 20711 | N/A | N/A | CGGAATAGAGAAAGCA | 46 | BB | 688 |
| | 20738 | 20753 | | | | | | |
| | 20906 | 20921 | | | | | | |
| | 20990 | 21005 | | | | | | |
| 1304821 | 23494 | 23509 | N/A | N/A | TAGTGTAGAGGCTGCA | 41 | BB | 538 |
| | 23527 | 23542 | | | | | | |
| 1304822 | 12139 | 12154 | 2442 | 2457 | CCTTUTCGAATTTGCC | 41 | BB | 2793 |
| 1304823 | 23303 | 23318 | N/A | N/A | ATCTUATTAGCAGCAG | 49 | BB | 2794 |
| 1304824 | 5138 | 5153 | 90 | 105 | ATGCCATCTTGACCCA | 57 | BB | 22 |

### Example 6: Effect of 3-10-3 cEt or Mixed MOE and cEt Uniform Phosphorothioate Modified Oligonucleotides on Human NLRP3 RNA In Vitro, Single Dose

Modified oligonucleotides complementary to human NLRP3 nucleic acid were designed and tested for their single dose effects on NLRP3 RNA in vitro. The modified oligonucleotides were tested in a series of experiments that had the same culture conditions.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), or to both. “N/A” indicates that the modified oligonucleotide is not 100% complementary to that particular target nucleic acid sequence.

Cultured THP-1 cells were treated with modified oligonucleotide at a concentration of 2000 nM by electroporation at a density of 100,000 cells per well. After a treatment period of approximately 24 hours, total RNA was isolated from the cells and NLRP3 RNA levels were measured by quantitative real-time RTPCR. NLRP3 RNA levels were measured by human primer-probe set RTS37509 (described herein above). NLRP3 RNA levels were normalized to total RNA content, as measured by RIBOGREEN®. Reduction of NLRP3 RNA is presented in the tables below as percent NLRP3 RNA relative to the amount of NLRP3 RNA in untreated control cells (% UTC).

Each separate experimental analysis described in this example is identified by a letter ID in the table column below labeled “AID” (Analysis ID). In the tables below, Compound No. 1232737 (described herein above) was used as a benchmark.

The modified oligonucleotides in the tables below are 3-10-3 cEt modified oligonucleotides with uniform phosphorothioate internucleoside linkages. The modified oligonucleotides are 16 nucleosides in length, wherein the central gap segment consists of ten 2′-β-D-deoxynucleosides, and wherein the 5′ and 3′ wing segments each consist of three cEt modified nucleosides. The sugar motif for the modified oligonucleotides is (from 5′ to 3′): kkkddddddddddkkk; wherein each “d” represents a 2′-β-D-deoxyribosyl sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine.

TABLE 8
Reduction of NLRP3 RNA by 3-10-3 cEt modified oligonucleotides with uniform
phosphorothioate internucleoside linkages at a concentration of 2000 nM in THP-1 cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | NLRP3 | | |
| Compound | Start | Stop | Start | Stop | | (% | | SEQ ID |
| Number | Site | Site | Site | Site | Sequence (5′ to 3′) | UTC) | AID | NO |
| 1232737 | 5138 | 5153 | 90 | 105 | ATGCCATCTTGACCCA | 57 | AG | 22 |
| 1301164 | 27197 | 27212 | N/A | N/A | TATTGAGACAGGCAGT | 54 | AG | 2795 |
| 1301200 | 6742 | 6757 | N/A | N/A | AAGGGAATTCCGACAC | 39 | AG | 2796 |
| 1301256 | 18352 | 18367 | N/A | N/A | GGACTTTAACACTTGC | 24 | AG | 2665 |
| 1301280 | 19887 | 19902 | N/A | N/A | GATTCTTGATTAGTTT | 55 | AG | 2671 |
| 1301290 | 20425 | 20440 | N/A | N/A | GTTTGTAAGTGAAGCG | 34 | AG | 2797 |
| 1301307 | 25015 | 25030 | N/A | N/A | CTACATAGAGCATATT | 100 | AG | 2798 |
| 1301318 | 25035 | 25050 | N/A | N/A | TTAGGTATTGACAATA | 58 | AG | 2799 |

The modified oligonucleotides in the table below are 16 nucleosides in length, wherein the sugar motif for the modified oligonucleotides is (from 5′ to 3′): kekdddddddddeekk; wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine.

TABLE 9
Reduction of NLRP3 RNA by modified oligonucleotides with a mixed MOE/cEt sugar motif and
uniform phosphorothioate internucleoside linkages at a concentration of 2000 nM in THP-1
cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | NLRP3 | | |
| Compound | Start | Stop | Start | Stop | | (% | | SEQ ID |
| Number | Site | Site | Site | Site | Sequence (5′ to 3′) | UTC) | AID | NO |
| 1232737 | 5138 | 5153 | 90 | 105 | ATGCCATCTTGACCCA | 57 | AG | 22 |
| 1299609 | 12464 | 12479 | 2767 | 2782 | AGTGACTCCACCCGAT | 41 | AG | 2733 |
| 1299620 | 12702 | 12717 | N/A | N/A | TGATGTAGCTGTGGCA | 42 | AG | 2654 |
| 1299643 | 14590 | 14605 | N/A | N/A | GTCCTCAGAGTAAGGC | 27 | AG | 759 |
| | 14882 | 14897 | | | | | | |
| | 15172 | 15187 | | | | | | |
| | 15245 | 15260 | | | | | | |
| 1299658 | 15183 | 15198 | N/A | N/A | CGTCAGAGTCTGTCCT | 15 | AG | 2656 |
| 1299684 | 17671 | 17686 | N/A | N/A | TGGAATATATCGAGCA | 20 | AG | 1454 |
| 1299691 | 21598 | 21613 | N/A | N/A | ACAATATGAGGACCCA | 49 | AG | 2383 |
| 1299718 | 23501 | 23516 | N/A | N/A | TCTTTCCTAGTGTAGA | 109 | AG | 2635 |
| 1299734 | 23534 | 23549 | N/A | N/A | CCTCTCCTAGTGTAGA | 94 | AG | 2732 |
| 1299739 | 23002 | 23017 | 3277 | 3292 | GATGCAAGATCCTGAC | 49 | AG | 2123 |
| 1299743 | 11048 | 11063 | 1351 | 1366 | AACTCCATCTTAATGG | 97 | AG | 2110 |
| 1299748 | 17441 | 17456 | N/A | N/A | AACATTAGGTCTTGTG | 52 | AG | 684 |
| 1299753 | 12134 | 12149 | 2437 | 2452 | TCGAATTTGCCATAGT | 56 | AG | 2115 |
| 1299754 | 23039 | 23054 | 3314 | 3329 | ATAGAGTCTGGTCAGG | 45 | AG | 351 |
| 1299756 | 17698 | 17713 | N/A | N/A | CATCTATGAACATGGC | 32 | AG | 1916 |
| 1299760 | 17449 | 17464 | N/A | N/A | ACTAGATTAACATTAG | 88 | AG | 992 |
| 1299878 | 26968 | 26983 | N/A | N/A | TAAAAGTCTCGACTGA | 57 | AG | 546 |
| 1299888 | 27024 | 27039 | N/A | N/A | TAGGCTATACCTAACA | 33 | AG | 239 |
| | 27060 | 27075 | | | | | | |
| | 27096 | 27111 | | | | | | |
| | 27114 | 27129 | | | | | | |
| 1299931 | 35776 | 35791 | 4129 | 4144 | GAGCTAATTACATGAG | 54 | AG | 824 |
| 1299937 | 26603 | 26618 | N/A | N/A | CCTTATTTGGTGATAC | 72 | AG | 1314 |

The modified oligonucleotides in the table below are 16 nucleosides in length, wherein the sugar motif for the modified oligonucleotides is (from 5′ to 3′): kkdddddddddkekek; wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine.

TABLE 10
Reduction of NLRP3 RNA by modified oligonucleotides with a mixed MOE/cEt sugar motif and
uniform phosphorothioate internucleoside linkages at a concentration of 2000 nM in THP-1
cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | NLRP3 | | |
| Compound | Start | Stop | Start | Stop | | (% | | SEQ ID |
| Number | Site | Site | Site | Site | Sequence (5′ to 3′) | UTC) | AID | NO |
| 1232737 | 5138 | 5153 | 90 | 105 | ATGCCATCTTGACCCA | 57 | AG | 22 |
| 1300713 | 5249 | 5264 | 201 | 216 | CAGGAGTGTGTCCTGA | 100 | AG | 2663 |
| 1300720 | 26957 | 26972 | N/A | N/A | ACTGATTAATGCCACT | 65 | AG | 1392 |
| 1300731 | 27130 | 27145 | N/A | N/A | AGCTATACCTAACACA | 77 | AG | 2239 |
| 1300764 | 10928 | 10943 | 1231 | 1246 | CGTTTGTTGAGGCTCA | 58 | AG | 1417 |
| 1300777 | 5147 | 5162 | 99 | 114 | CACTTCACGATGCCAT | 45 | AG | 330 |
| 1300780 | 11008 | 11023 | 1311 | 1326 | TGGTCTTGCCGATGGC | 60 | AG | 2033 |
| 1300799 | 11296 | 11311 | 1599 | 1614 | TCTTGTGGATGGGTGG | 95 | AG | 2188 |
| 1300803 | 11302 | 11317 | 1605 | 1620 | TCACGATCTTGTGGAT | 93 | AG | 803 |
| 1300805 | 11973 | 11988 | 2276 | 2291 | CTCGCAGTCCACTTCC | 70 | AG | 868 |
| 1300818 | 11979 | 11994 | 2282 | 2297 | GAACTTCTCGCAGTCC | 67 | AG | 1114 |
| 1300842 | 5168 | 5183 | 120 | 135 | TTACAGTTTACGGTGA | 77 | AG | 24 |
| 1300852 | 14476 | 14491 | N/A | N/A | TGAGCCTCCTACCGGG | 60 | AG | 2142 |
| | 14622 | 14637 | | | | | | |
| | 14768 | 14783 | | | | | | |
| | 14914 | 14929 | | | | | | |
| | 15060 | 15075 | | | | | | |
| 1300871 | 16730 | 16745 | 3048 | 3063 | GAATGTTACAGCCAGG | 77 | AG | 2275 |
| 1300911 | 20584 | 20599 | N/A | N/A | TGACCAGAGAGCTCCG | 74 | AG | 150 |
| | 20710 | 20725 | | | | | | |
| | 20752 | 20767 | | | | | | |
| | 20920 | 20935 | | | | | | |
| 1300997 | 12141 | 12156 | 2444 | 2459 | CCCCTTTTCGAATTTG | 60 | AG | 574 |
| 1301020 | 19879 | 19894 | N/A | N/A | ATTAGTTTCCCCGTCA | 49 | AG | 2647 |
| 1301044 | 12456 | 12471 | 2759 | 2774 | CACCCGATGACAGTTC | 58 | AG | 345 |
| 1301052 | 20593 | 20608 | N/A | N/A | GAATACACCTGACCAG | 79 | AG | 227 |
| 1301057 | 22429 | 22444 | N/A | N/A | ATAATGTCCCCGAGGC | 58 | AG | 2493 |
| 1301144 | 25746 | 25761 | N/A | N/A | TGTTGAAATTGAGAGG | 49 | AG | 2728 |

The modified oligonucleotides in the table below are 16 nucleosides in length, wherein the sugar motif for the modified oligonucleotides is (from 5′ to 3′): kkkdddddddddkkke; wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methyl cytosine.

TABLE 11
Reduction of NLRP3 RNA by modified oligonucleotides with a mixed MOE/cEt sugar motif and
uniform phosphorothioate internucleoside linkages at a concentration of 2000 nM in THP-1
cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | NLRP3 | | |
| Compound | Start | Stop | Start | Stop | | (% | | SEQ ID |
| Number | Site | Site | Site | Site | Sequence (5′ to 3′) | UTC) | AID | NO |
| 1232737 | 5138 | 5153 | 90 | 105 | ATGCCATCTTGACCCA | 57 | AG | 22 |
| 1300281 | 27366 | 27381 | N/A | N/A | GCATTTAGGACCGTCC | 64 | AG | 241 |
| 1300287 | 27204 | 27219 | N/A | N/A | TGAGCAGTATTGAGAC | 74 | AG | 2729 |
| 1300295 | 6019 | 6034 | 971 | 986 | CCTGTTGATCGCAGCG | 57 | AG | 2260 |
| 1300301 | 10305 | 10320 | 1105 | 1120 | GAAAGGTACTCCAGTA | 49 | AG | 338 |
| 1300340 | 11259 | 11274 | 1562 | 1577 | CATGATCAGGTCCCCC | 57 | AG | 1957 |
| 1300380 | 12276 | 12291 | 2579 | 2594 | GGCTTTCACTTCAATC | 58 | AG | 2664 |
| 1300398 | 14569 | 14584 | N/A | N/A | CCATCTAGAGGGATGG | 86 | AG | 1220 |
| | 14861 | 14876 | | | | | | |
| | 15151 | 15166 | | | | | | |
| | 15224 | 15239 | | | | | | |
| 1300423 | 16642 | 16657 | 2960 | 2975 | TAGACTCTGGCTGGTG | 41 | AG | 2730 |
| 1300433 | 17664 | 17679 | N/A | N/A | TATCGAGCATCATGGT | 43 | AG | 2487 |
| 1300457 | 11041 | 11056 | 1344 | 1359 | TCTTAATGGGACTCAC | 45 | AG | 263 |
| 1300464 | 23281 | 23296 | N/A | N/A | TTATATGCCTCCAGTC | 43 | AG | 2507 |
| 1300474 | 23491 | 23506 | N/A | N/A | TGTAGAGGCTGCATGC | 36 | AG | 2501 |
| 1300481 | 23495 | 23510 | N/A | N/A | CTAGTGTAGAGGCTGC | 25 | AG | 615 |
| | 23528 | 23543 | | | | | | |
| 1300492 | 25162 | 25177 | N/A | N/A | CGACCTATAAGGACCC | 47 | AG | 2502 |
| 1300541 | 18857 | 18872 | N/A | N/A | GTGCAGTAACATATCC | 66 | AG | 2489 |
| 1300550 | 18756 | 18771 | N/A | N/A | ATTTAAGCCACCGAAC | 81 | AG | 2646 |
| 1300560 | 19270 | 19285 | N/A | N/A | TAGACACTAGAGCCTC | 56 | AG | 2658 |
| 1300575 | 20276 | 20291 | N/A | N/A | TTGACGATTTCATCCC | 41 | AG | 2672 |
| 1300582 | 20419 | 20434 | N/A | N/A | AAGTGAAGCGGGCGGT | 48 | AG | 2490 |
| 1300583 | 5139 | 5154 | 91 | 106 | GATGCCATCTTGACCC | 62 | AG | 2100 |
| 1300611 | 22899 | 22914 | N/A | N/A | ACAAGGAAGCACCCGT | 47 | AG | 1999 |
| 1300614 | 23823 | 23838 | N/A | N/A | CATGATTATTAGTGGG | 55 | AG | 78 |
| 1300623 | 14500 | 14515 | N/A | N/A | GGTGCTATCTAGAGAA | 25 | AG | 1604 |
| | 14646 | 14661 | | | | | | |
| | 14719 | 14734 | | | | | | |
| | 14792 | 14807 | | | | | | |
| | 14938 | 14953 | | | | | | |
| | 15011 | 15026 | | | | | | |
| | 15084 | 15099 | | | | | | |
| 1300637 | 25079 | 25094 | N/A | N/A | AGGCCGCTCTGTTTGT | 93 | AG | 2666 |
| 1300645 | 26785 | 26800 | N/A | N/A | CCCCAATCGATAGATC | 85 | AG | 2661 |
| 1300667 | 28755 | 28770 | N/A | N/A | TTAAGCAACGGTTACA | 51 | AG | 1472 |
| 1300673 | 29463 | 29478 | N/A | N/A | GAGTCAAATTAAGCCC | 87 | AG | 2495 |
| 1300679 | 28763 | 28778 | N/A | N/A | GGTAACTATTAAGCAA | 75 | AG | 2653 |
| 1300686 | 31019 | 31034 | 3463 | 3478 | TTAGTGCTGAGTACCG | 36 | AG | 1126 |
| 1300697 | 31081 | 31096 | 3525 | 3540 | GTAGTTTGATCCCCTT | 41 | AG | 1897 |
| 1300705 | 26596 | 26611 | N/A | N/A | TGGTGATACGGTTACT | 33 | AG | 2496 |

### Example 7: Dose-Dependent Inhibition of Human NLRP3 in THP-1 Cells by Modified Oligonucleotides

Modified oligonucleotides selected from the examples above were tested at various doses in THP-1 cells. Cultured THP-1 cells at a density of 300,000 cells per well were treated by electroporation with various concentrations of modified oligonucleotide as specified in the tables below. After a treatment period of approximately 24 hours, total RNA was isolated from the cells, and NLRP3 RNA levels were measured by quantitative real-time RTPCR. Human NLRP3 primer-probe set RTS37509 (described herein above) was used to measure RNA levels as described above. NLRP3 RNA levels were normalized to total RNA content, as measured by RIBOGREEN®. Reduction of NLRP3 RNA is presented in the tables below as percent NLRP3 RNA, relative to amount of NLRP3 RNA in untreated control cells (% UTC).

The half maximal inhibitory concentration (IC₅₀) of each modified oligonucleotide was calculated using a linear regression on a log/linear plot of the data in Excel and is also presented in the tables below.

TABLE 12
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 89 | 86 | 70 | 52 | 24 | 1.04 |
| 1233042 | 69 | 102 | 75 | 61 | 26 | 1.95 |
| 1233104 | 72 | 63 | 51 | 26 | 11 | 0.19 |
| 1233105 | 65 | 58 | 36 | 29 | 7 | 0.11 |
| 1233144 | 89 | 78 | 47 | 28 | 7 | 0.31 |
| 1233153 | 79 | 78 | 61 | 30 | 16 | 0.38 |
| 1233154 | 103 | 60 | 58 | 29 | 16 | 0.39 |
| 1233168 | 81 | 75 | 43 | 27 | 11 | 0.26 |
| 1233176 | 73 | 47 | 48 | 20 | 7 | 0.12 |
| 1233177 | 72 | 65 | 37 | 11 | 9 | 0.13 |
| 1233208 | 87 | 73 | 53 | 30 | 12 | 0.34 |
| 1233240 | 105 | 62 | 47 | 24 | 19 | 0.34 |
| 1233241 | 83 | 68 | 47 | 26 | 6 | 0.24 |
| 1233242 | 90 | 90 | 82 | 43 | 17 | 0.89 |
| 1233289 | 99 | 87 | 59 | 32 | 9 | 0.48 |

TABLE 13
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 100 | 90 | 73 | 54 | 24 | 1.21 |
| 1232754 | 70 | 72 | 43 | 40 | 26 | 0.32 |
| 1232764 | 96 | 81 | 56 | 33 | 18 | 0.49 |
| 1232843 | 81 | 75 | 46 | 44 | 27 | 0.49 |
| 1232844 | 83 | 59 | 50 | 34 | 19 | 0.28 |
| 1232859 | 84 | 80 | 46 | 34 | 12 | 0.34 |
| 1233075 | 109 | 78 | 56 | 39 | 14 | 0.55 |
| 1233083 | 84 | 69 | 35 | 16 | 4 | 0.18 |
| 1233100 | 92 | 67 | 41 | 29 | 13 | 0.28 |
| 1233106 | 85 | 76 | 62 | 42 | 20 | 0.57 |
| 1233172 | 84 | 75 | 70 | 36 | 17 | 0.52 |
| 1233220 | 109 | 76 | 61 | 35 | 20 | 0.59 |
| 1233244 | 99 | 76 | 68 | 42 | 11 | 0.58 |
| 1233251 | 85 | 74 | 63 | 40 | 25 | 0.60 |
| 1233290 | 82 | 78 | 67 | 41 | 18 | 0.58 |

TABLE 14
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 87 | 66 | 64 | 48 | 33 | 0.89 |
| 1232741 | 75 | 71 | 52 | 31 | 26 | 0.34 |
| 1232806 | 97 | 85 | 66 | 46 | 25 | 0.88 |
| 1232821 | 89 | 83 | 59 | 33 | 17 | 0.48 |
| 1232845 | 70 | 58 | 41 | 21 | 11 | 0.13 |
| 1232846 | 71 | 45 | 31 | 22 | 14 | 0.08 |
| 1232878 | 79 | 77 | 52 | 33 | 17 | 0.36 |
| 1233117 | 100 | 100 | 76 | 63 | 37 | 2.68 |
| 1233143 | 90 | 79 | 62 | 34 | 33 | 0.72 |
| 1233157 | 91 | 82 | 62 | 45 | 29 | 0.85 |
| 1233174 | 86 | 61 | 35 | 22 | 13 | 0.19 |
| 1233175 | 73 | 57 | 37 | 16 | 9 | 0.12 |
| 1233229 | 70 | 72 | 60 | 37 | 26 | 0.45 |
| 1233246 | 104 | 98 | 70 | 32 | 12 | 0.65 |
| 1233278 | 83 | 70 | 56 | 43 | 21 | 0.48 |

TABLE 15
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 89 | 73 | 75 | 54 | 35 | 1.69 |
| 1233167 | 78 | 70 | 56 | 30 | 17 | 0.32 |
| 1233279 | 80 | 66 | 52 | 22 | 11 | 0.23 |
| 1241459 | 90 | 75 | 51 | 48 | 29 | 0.69 |
| 1241865 | 69 | 60 | 32 | 21 | 10 | 0.11 |
| 1242009 | 83 | 70 | 54 | 42 | 24 | 0.48 |
| 1242081 | 93 | 79 | 65 | 32 | 18 | 0.52 |
| 1242201 | 99 | 72 | 44 | 26 | 21 | 0.37 |
| 1242273 | 101 | 81 | 49 | 39 | 22 | 0.56 |
| 1242393 | 83 | 66 | 53 | 32 | 17 | 0.31 |
| 1242441 | 85 | 72 | 48 | 28 | 21 | 0.34 |
| 1242442 | 71 | 59 | 45 | 25 | 19 | 0.17 |
| 1242489 | 84 | 66 | 46 | 31 | 23 | 0.31 |
| 1242633 | 85 | 69 | 56 | 32 | 9 | 0.31 |
| 1242704 | 78 | 71 | 58 | 27 | 8 | 0.28 |

TABLE 16
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 102 | 87 | 76 | 56 | 37 | 2.05 |
| 1241580 | 110 | 83 | 94 | 64 | 40 | 3.63 |
| 1241773 | 104 | 45 | 63 | 24 | 11 | 0.30 |
| 1241843 | 88 | 107 | 56 | 39 | 22 | 0.74 |
| 1241866 | 100 | 59 | 56 | 21 | 17 | 0.32 |
| 1242274 | 105 | 94 | 66 | 50 | 39 | 1.65 |
| 1242419 | 97 | 72 | 68 | 40 | 24 | 0.70 |
| 1242443 | 102 | 87 | 49 | 21 | 22 | 0.46 |
| 1242491 | 106 | 96 | 68 | 44 | 22 | 0.92 |
| 1242514 | 128 | 117 | 93 | 39 | 16 | 1.17 |
| 1242516 | 84 | 69 | 61 | 38 | 27 | 0.54 |
| 1242538 | 138 | 109 | 108 | 60 | 29 | 2.32 |
| 1242706 | 96 | 79 | 70 | 40 | 31 | 0.93 |
| 1242922 | 66 | 52 | 43 | 16 | 4 | 0.09 |
| 1242969 | 128 | 117 | 91 | 66 | 47 | 4.11 |

TABLE 17
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 90 | 78 | 68 | 49 | 32 | 1.12 |
| 1241703 | 105 | 63 | 68 | 58 | 37 | 1.50 |
| 1241774 | 99 | 75 | 53 | 34 | 18 | 0.45 |
| 1241868 | 85 | 79 | 56 | 32 | 17 | 0.41 |
| 1242061 | 87 | 74 | 54 | 26 | 13 | 0.33 |
| 1242444 | 96 | 65 | 50 | 24 | 17 | 0.32 |
| 1242445 | 81 | 72 | 39 | 16 | 7 | 0.19 |
| 1242470 | 95 | 66 | 43 | 25 | 6 | 0.26 |
| 1242493 | 69 | 64 | 30 | 11 | 11 | 0.11 |
| 1242517 | 70 | 41 | 27 | 18 | 13 | 0.06 |
| 1242518 | 58 | 65 | 35 | 14 | 13 | 0.09 |
| 1242588 | 106 | 99 | 73 | 35 | 12 | 0.72 |
| 1242707 | 80 | 83 | 63 | 30 | 15 | 0.43 |
| 1242804 | 84 | 53 | 47 | 25 | 33 | 0.26 |
| 1242923 | 80 | 74 | 41 | 35 | 20 | 0.31 |

TABLE 18
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 101 | 93 | 81 | 58 | 44 | 3.28 |
| 1241775 | 87 | 70 | 49 | 29 | 13 | 0.30 |
| 1242206 | 112 | 87 | 65 | 29 | 14 | 0.59 |
| 1242446 | 96 | 78 | 56 | 22 | 7 | 0.35 |
| 1242447 | 98 | 63 | 32 | 16 | 12 | 0.22 |
| 1242471 | 103 | 75 | 51 | 24 | 12 | 0.37 |
| 1242494 | 99 | 68 | 44 | 24 | 6 | 0.28 |
| 1242495 | 86 | 78 | 59 | 29 | 8 | 0.36 |
| 1242496 | 103 | 82 | 61 | 48 | 22 | 0.80 |
| 1242519 | 81 | 55 | 22 | 11 | 3 | 0.10 |
| 1242543 | 107 | 74 | 45 | 36 | 15 | 0.44 |
| 1242567 | 93 | 74 | 50 | 36 | 15 | 0.41 |
| 1242614 | 112 | 97 | 64 | 37 | 12 | 0.67 |
| 1242805 | 96 | 98 | 59 | 36 | 18 | 0.65 |
| 1242807 | 103 | 81 | 64 | 45 | 23 | 0.79 |

TABLE 19
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 88 | 69 | 70 | 61 | 42 | 2.87 |
| 1241491 | 83 | 88 | 78 | 45 | 19 | 0.86 |
| 1241586 | 88 | 81 | 53 | 36 | 21 | 0.50 |
| 1241921 | 65 | 58 | 44 | 25 | 13 | 0.13 |
| 1242497 | 91 | 86 | 49 | 25 | 7 | 0.34 |
| 1242546 | 99 | 62 | 30 | 15 | 6 | 0.20 |
| 1242616 | 92 | 77 | 59 | 29 | 10 | 0.40 |
| 1242617 | 105 | 72 | 64 | 29 | 7 | 0.43 |
| 1242618 | 91 | 79 | 69 | 39 | 20 | 0.65 |
| 1242831 | 85 | 67 | 39 | 27 | 13 | 0.24 |
| 1242833 | 95 | 84 | 53 | 37 | 16 | 0.50 |
| 1242904 | 89 | 81 | 45 | 24 | 10 | 0.31 |
| 1242905 | 80 | 68 | 43 | 30 | 14 | 0.24 |
| 1242928 | 97 | 71 | 60 | 27 | 11 | 0.38 |
| 1242929 | 101 | 85 | 64 | 39 | 24 | 0.74 |

TABLE 20
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 84 | 79 | 69 | 50 | 29 | 1.04 |
| 1241661 | 70 | 74 | 61 | 28 | 12 | 0.28 |
| 1241826 | 88 | 57 | 40 | 24 | 10 | 0.20 |
| 1242453 | 95 | 92 | 56 | 40 | 14 | 0.57 |
| 1242476 | 97 | 84 | 50 | 21 | 6 | 0.34 |
| 1242499 | 79 | 56 | 31 | 9 | 3 | 0.11 |
| 1242500 | 74 | 48 | 22 | 8 | 2 | 0.07 |
| 1242501 | 82 | 83 | 50 | 23 | 4 | 0.28 |
| 1242523 | 83 | 71 | 49 | 26 | 9 | 0.26 |
| 1242547 | 74 | 57 | 28 | 10 | 4 | 0.10 |
| 1242571 | 106 | 76 | 39 | 15 | 6 | 0.29 |
| 1242572 | 49 | 54 | 47 | 20 | 8 | 0.06 |
| 1242834 | 100 | 83 | 52 | 21 | 8 | 0.37 |
| 1242835 | 87 | 61 | 60 | 29 | 12 | 0.31 |
| 1242907 | 87 | 71 | 57 | 36 | 6 | 0.34 |

TABLE 21
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 92 | 80 | 63 | 61 | 40 | 2.20 |
| 1241687 | 86 | 53 | 47 | 26 | 16 | 0.22 |
| 1241688 | 87 | 74 | 52 | 34 | 20 | 0.42 |
| 1242071 | 110 | 92 | 58 | 41 | 18 | 0.70 |
| 1242118 | 72 | 89 | 78 | 43 | 32 | 1.25 |
| 1242119 | 110 | 76 | 62 | 25 | 19 | 0.51 |
| 1242454 | 83 | 59 | 45 | 20 | 5 | 0.18 |
| 1242455 | 95 | 68 | 48 | 21 | 7 | 0.27 |
| 1242477 | 95 | 83 | 47 | 20 | 19 | 0.38 |
| 1242478 | 72 | 71 | 62 | 41 | 16 | 0.40 |
| 1242574 | 84 | 74 | 51 | 31 | 15 | 0.34 |
| 1242598 | 100 | 58 | 37 | 20 | 13 | 0.24 |
| 1242599 | 84 | 59 | 41 | 21 | 9 | 0.19 |
| 1242645 | 94 | 106 | 64 | 44 | 28 | 1.08 |
| 1242669 | 62 | 81 | 50 | 31 | 21 | 0.28 |

TABLE 22
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 83 | 78 | 71 | 57 | 37 | 2.02 |
| 1241643 | 92 | 71 | 59 | 30 | 17 | 0.41 |
| 1241689 | 75 | 68 | 40 | 32 | 16 | 0.22 |
| 1242000 | 84 | 76 | 66 | 51 | 29 | 0.99 |
| 1242001 | 77 | 64 | 56 | 37 | 22 | 0.36 |
| 1242072 | 84 | 77 | 61 | 38 | 15 | 0.47 |
| 1242456 | 78 | 70 | 46 | 19 | 6 | 0.20 |
| 1242457 | 79 | 62 | 37 | 18 | 6 | 0.16 |
| 1242481 | 80 | 78 | 64 | 48 | 21 | 0.67 |
| 1242503 | 90 | 81 | 56 | 31 | 13 | 0.41 |
| 1242504 | 69 | 66 | 57 | 29 | 12 | 0.23 |
| 1242553 | 84 | 80 | 50 | 25 | 17 | 0.34 |
| 1242601 | 95 | 40 | 18 | 11 | 6 | 0.11 |
| 1242624 | 75 | 66 | 58 | 26 | 26 | 0.32 |
| 1242695 | 88 | 80 | 72 | 41 | 13 | 0.60 |

TABLE 23
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 89 | 73 | 68 | 53 | 31 | 1.14 |
| 1241621 | 87 | 70 | 56 | 32 | 22 | 0.41 |
| 1241668 | 73 | 82 | 55 | 35 | 24 | 0.44 |
| 1241669 | 76 | 66 | 49 | 33 | 20 | 0.28 |
| 1242098 | 74 | 63 | 59 | 33 | 20 | 0.32 |
| 1242458 | 89 | 83 | 42 | 25 | 7 | 0.30 |
| 1242459 | 94 | 84 | 70 | 42 | 11 | 0.62 |
| 1242482 | 71 | 74 | 58 | 25 | 8 | 0.25 |
| 1242485 | 67 | 58 | 29 | 11 | 3 | 0.09 |
| 1242579 | 85 | 73 | 54 | 29 | 20 | 0.37 |
| 1242603 | 94 | 84 | 56 | 26 | 14 | 0.42 |
| 1242626 | 52 | 74 | 59 | 26 | 9 | 0.17 |
| 1242627 | 80 | 77 | 62 | 32 | 14 | 0.40 |
| 1242698 | 89 | 69 | 59 | 41 | 17 | 0.47 |
| 1242916 | 95 | 76 | 57 | 26 | 12 | 0.39 |

TABLE 24
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 79 | 78 | 73 | 50 | 39 | 1.84 |
| 1241455 | 78 | 79 | 50 | 28 | 25 | 0.37 |
| 1241456 | 77 | 85 | 61 | 31 | 17 | 0.44 |
| 1241648 | 86 | 68 | 58 | 43 | 19 | 0.48 |
| 1241695 | 97 | 98 | 71 | 50 | 25 | 1.15 |
| 1241791 | 90 | 79 | 62 | 46 | 20 | 0.66 |
| 1241861 | 96 | 67 | 63 | 44 | 25 | 0.67 |
| 1242438 | 103 | 91 | 72 | 36 | 29 | 0.95 |
| 1242439 | 83 | 77 | 58 | 30 | 25 | 0.46 |
| 1242486 | 74 | 59 | 39 | 23 | 12 | 0.15 |
| 1242487 | 78 | 73 | 52 | 30 | 17 | 0.31 |
| 1242558 | 75 | 71 | 55 | 28 | 19 | 0.30 |
| 1242631 | 93 | 53 | 43 | 31 | 17 | 0.25 |
| 1242822 | 105 | 71 | 47 | 20 | 15 | 0.34 |
| 1242965 | 81 | 84 | 58 | 32 | 24 | 0.50 |

TABLE 25
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 98 | 73 | 75 | 54 | 42 | 2.15 |
| 1241698 | 80 | 66 | 39 | 21 | 14 | 0.19 |
| 1241864 | 83 | 84 | 59 | 27 | 13 | 0.39 |
| 1241983 | 91 | 95 | 43 | 37 | 13 | 0.45 |
| 1241984 | 80 | 82 | 55 | 27 | 11 | 0.34 |
| 1242079 | 96 | 90 | 55 | 30 | 21 | 0.54 |
| 1242104 | 113 | 103 | 97 | 62 | 36 | 3.06 |
| 1242200 | 83 | 75 | 51 | 25 | 11 | 0.29 |
| 1242488 | 102 | 92 | 69 | 35 | 20 | 0.74 |
| 1242560 | 87 | 79 | 46 | 25 | 8 | 0.29 |
| 1242632 | 106 | 52 | 43 | 19 | 9 | 0.24 |
| 1242656 | 90 | 59 | 49 | 27 | 11 | 0.26 |
| 1242727 | 116 | 96 | 67 | 39 | 14 | 0.74 |
| 1242823 | 89 | 71 | 57 | 30 | 18 | 0.39 |
| 1242895 | 133 | 89 | 59 | 30 | 26 | 0.76 |

TABLE 26
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 20 nM | 78 nM | 313 nM | 1250 nM | 5000 nM | (μM) |
| 1232737 | 88 | 94 | 68 | 54 | 42 | 2.32 |
| 1241578 | 83 | 59 | 42 | 26 | 12 | 0.20 |
| 1241694 | 84 | 69 | 34 | 18 | 8 | 0.19 |
| 1242244 | 93 | 83 | 47 | 21 | 9 | 0.33 |
| 1242460 | 121 | 93 | 52 | 16 | 6 | 0.44 |
| 1242484 | 84 | 70 | 38 | 13 | 6 | 0.19 |
| 1242652 | 118 | 79 | 45 | 12 | 6 | 0.34 |
| 1242747 | 92 | 63 | 40 | 33 | 14 | 0.28 |
| 1242819 | 98 | 73 | 48 | 18 | 10 | 0.30 |

TABLE 27
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 68 | 93 | 77 | 62 | 45 | >4.0 |
| 1299642 | 60 | 66 | 66 | 36 | 10 | 0.19 |
| 1299643 | 95 | 76 | 68 | 41 | 13 | 0.45 |
| 1299658 | 112 | 89 | 52 | 26 | 15 | 0.40 |
| 1299684 | 90 | 83 | 56 | 30 | 15 | 0.34 |
| 1299767 | 87 | 60 | 56 | 38 | 21 | 0.31 |
| 1300450 | 90 | 94 | 58 | 33 | 21 | 0.48 |
| 1300481 | 102 | 92 | 68 | 49 | 12 | 0.62 |
| 1300485 | 76 | 106 | 59 | 39 | 20 | 0.55 |
| 1300623 | 88 | 97 | 73 | 46 | 12 | 0.62 |
| 1300883 | 106 | 85 | 67 | 43 | 28 | 0.74 |
| 1301256 | 132 | 88 | 67 | 44 | 16 | 0.66 |
| 1301292 | 118 | 77 | 61 | 26 | 19 | 0.43 |
| 1301314 | 100 | 70 | 68 | 39 | 16 | 0.45 |
| 1301324 | 86 | 67 | 51 | 49 | 31 | 0.53 |

TABLE 28
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 70 | 83 | 72 | 46 | 39 | 1.34 |
| 1299659 | 85 | 104 | 65 | 27 | 11 | 0.42 |
| 1300409 | 92 | 67 | 81 | 49 | 20 | 0.69 |
| 1300431 | 83 | 58 | 65 | 30 | 11 | 0.24 |
| 1300489 | 91 | 87 | 92 | 52 | 31 | 1.61 |
| 1300567 | 66 | 73 | 78 | 69 | 39 | >4.0 |
| 1300657 | 118 | 98 | 67 | 36 | 13 | 0.57 |
| 1300899 | 83 | 78 | 67 | 41 | 16 | 0.44 |
| 1301085 | 88 | 71 | 70 | 62 | 27 | 1.05 |
| 1301092 | 101 | 89 | 65 | 34 | 14 | 0.48 |
| 1301228 | 73 | 72 | 69 | 46 | 22 | 0.50 |
| 1301238 | 76 | 75 | 68 | 47 | 15 | 0.44 |
| 1301274 | 79 | 63 | 45 | 38 | 20 | 0.22 |
| 1301281 | 103 | 97 | 54 | 39 | 22 | 0.57 |
| 1301346 | 89 | 75 | 64 | 68 | 29 | 1.30 |

TABLE 29
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 91 | 88 | 91 | 71 | 38 | >4.0 |
| 1299527 | 110 | 86 | 35 | 47 | 20 | 0.46 |
| 1299802 | 110 | 66 | 68 | 18 | 15 | 0.34 |
| 1299865 | 83 | 67 | 30 | 32 | 7 | 0.16 |
| 1300390 | 88 | 92 | 47 | 50 | 20 | 0.50 |
| 1300410 | 52 | 66 | 72 | 50 | 16 | 0.31 |
| 1300422 | 49 | 62 | 57 | 26 | 13 | 0.09 |
| 1300447 | 105 | 104 | 75 | 41 | 13 | 0.68 |
| 1300576 | 113 | 79 | 67 | 36 | 15 | 0.50 |
| 1300578 | 87 | 61 | 42 | 24 | 6 | 0.17 |
| 1300595 | 54 | 78 | 77 | 42 | 16 | 0.38 |
| 1300701 | 49 | 101 | 51 | 38 | 12 | 0.25 |
| 1300886 | 96 | 71 | 44 | 26 | 20 | 0.27 |
| 1301108 | 90 | 86 | 35 | 30 | 8 | 0.25 |
| 1301210 | 72 | 60 | 68 | 51 | 22 | 0.45 |

TABLE 30
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 58 | 94 | 74 | 70 | 27 | 2.58 |
| 1299496 | 84 | 63 | 75 | 29 | 28 | 0.44 |
| 1299617 | 58 | 76 | 39 | 51 | 22 | 0.22 |
| 1299663 | 104 | 77 | 39 | 36 | 22 | 0.36 |
| 1299670 | 82 | 94 | 54 | 34 | 17 | 0.40 |
| 1299797 | 95 | 97 | 53 | 35 | 15 | 0.44 |
| 1300424 | 102 | 69 | 50 | 16 | 9 | 0.24 |
| 1300472 | 102 | 44 | 63 | 31 | 16 | 0.27 |
| 1300625 | 102 | 57 | 45 | 51 | 42 | 0.69 |
| 1300859 | 99 | 99 | 62 | 36 | 21 | 0.58 |
| 1301153 | 123 | 85 | 84 | 47 | 22 | 0.91 |
| 1301158 | 67 | 80 | 63 | 42 | 18 | 0.37 |
| 1301199 | 48 | 66 | 57 | 44 | 22 | 0.15 |
| 1301207 | 103 | 68 | 48 | 47 | 20 | 0.42 |
| 1301283 | 135 | 110 | 86 | 44 | 14 | 0.88 |

TABLE 31
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 82 | 68 | 80 | 57 | 42 | 2.84 |
| 1299715 | 80 | 75 | 53 | 34 | 16 | 0.28 |
| 1299840 | 96 | 121 | 103 | 48 | 42 | 2.86 |
| 1300266 | 53 | 74 | 52 | 30 | 21 | 0.15 |
| 1300382 | 52 | 45 | 47 | 42 | 29 | 0.04 |
| 1300385 | 95 | 73 | 60 | 47 | 27 | 0.61 |
| 1300448 | 88 | 85 | 76 | 48 | 27 | 0.93 |
| 1300513 | 100 | 85 | 53 | 29 | 18 | 0.39 |
| 1300580 | 78 | 64 | 55 | 36 | 17 | 0.25 |
| 1300655 | 47 | 65 | 48 | 23 | 20 | 0.06 |
| 1300685 | 91 | 79 | 63 | 40 | 24 | 0.53 |
| 1300816 | 105 | 72 | 83 | 38 | 63 | 3.25 |
| 1300889 | 126 | 109 | 92 | 70 | 31 | 2.14 |
| 1301018 | 112 | 105 | 104 | 76 | 42 | >4.0 |
| 1301277 | 42 | 85 | 69 | 41 | 22 | 0.33 |

TABLE 32
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 122 | 107 | 85 | 70 | 52 | >4.0 |
| 1299657 | 128 | 74 | 52 | 26 | 7 | 0.36 |
| 1299673 | 86 | 72 | 52 | 28 | 9 | 0.24 |
| 1299796 | 89 | 74 | 63 | 44 | 23 | 0.53 |
| 1299804 | 76 | 60 | 67 | 34 | 19 | 0.29 |
| 1300373 | 46 | 80 | 69 | 43 | 24 | 0.37 |
| 1300440 | 46 | 85 | 41 | 32 | 20 | 0.12 |
| 1300441 | 51 | 78 | 45 | 34 | 22 | 0.14 |
| 1300503 | 95 | 81 | 70 | 63 | 16 | 0.82 |
| 1300875 | 89 | 77 | 58 | 40 | 21 | 0.43 |
| 1300876 | 58 | 80 | 48 | 34 | 12 | 0.17 |
| 1300892 | 47 | 62 | 39 | 34 | 13 | 0.05 |
| 1300900 | 157 | 118 | 76 | 50 | 22 | 1.04 |
| 1301165 | 100 | 86 | 58 | 29 | 17 | 0.41 |
| 1301266 | 91 | 127 | 116 | 41 | 36 | 2.43 |

TABLE 33
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 87 | 78 | 78 | 64 | 38 | 2.62 |
| 1299944 | 79 | 79 | 61 | 39 | 22 | 0.43 |
| 1300261 | 83 | 65 | 39 | 36 | 31 | 0.28 |
| 1300299 | 63 | 76 | 49 | 35 | 16 | 0.20 |
| 1300411 | 84 | 81 | 76 | 64 | 35 | 2.12 |
| 1300477 | 73 | 72 | 49 | 21 | 6 | 0.16 |
| 1300540 | 97 | 86 | 69 | 39 | 23 | 0.62 |
| 1300568 | 105 | 89 | 76 | 47 | 19 | 0.76 |
| 1300668 | 74 | 58 | 60 | 37 | 14 | 0.22 |
| 1300897 | 74 | 62 | 34 | 21 | 12 | 0.12 |
| 1301213 | 93 | 83 | 67 | 35 | 14 | 0.44 |
| 1301265 | 70 | 70 | 58 | 44 | 18 | 0.31 |
| 1301289 | 108 | 100 | 74 | 46 | 15 | 0.72 |
| 1304709 | 80 | 63 | 35 | 16 | 9 | 0.12 |
| 1304758 | 66 | 75 | 49 | 31 | 16 | 0.20 |

TABLE 34
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 88 | 87 | 72 | 65 | 48 | >4.0 |
| 1304685 | 81 | 78 | 53 | 35 | 15 | 0.30 |
| 1304686 | 68 | 57 | 55 | 43 | 25 | 0.24 |
| 1304689 | 66 | 61 | 48 | 33 | 11 | 0.14 |
| 1304726 | 87 | 82 | 56 | 34 | 18 | 0.37 |
| 1304728 | 124 | 97 | 77 | 40 | 11 | 0.66 |
| 1304730 | 77 | 37 | 30 | 15 | 7 | 0.06 |
| 1304739 | 87 | 79 | 62 | 21 | 10 | 0.28 |
| 1304745 | 68 | 76 | 60 | 39 | 9 | 0.26 |
| 1304751 | 100 | 92 | 30 | 34 | 19 | 0.34 |
| 1304763 | 81 | 63 | 73 | 46 | 18 | 0.45 |
| 1304774 | 90 | 75 | 55 | 38 | 15 | 0.35 |
| 1304790 | 83 | 68 | 58 | 38 | 18 | 0.32 |
| 1304795 | 81 | 97 | 74 | 37 | 24 | 0.68 |
| 1304798 | 56 | 57 | 55 | 33 | 14 | 0.10 |

TABLE 35
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 95 | 79 | 73 | 57 | 40 | 1.85 |
| 1299705 | 115 | 102 | 75 | 34 | 13 | 0.62 |
| 1299857 | 92 | 73 | 52 | 27 | 16 | 0.29 |
| 1299941 | 85 | 67 | 53 | 25 | 11 | 0.22 |
| 1300317 | 80 | 66 | 57 | 36 | 18 | 0.29 |
| 1300349 | 88 | 69 | 50 | 29 | 15 | 0.25 |
| 1300529 | 87 | 80 | 55 | 33 | 11 | 0.31 |
| 1300562 | 78 | 74 | 51 | 23 | 9 | 0.20 |
| 1300572 | 58 | 71 | 47 | 22 | 10 | 0.11 |
| 1300636 | 83 | 61 | 35 | 22 | 14 | 0.14 |
| 1300691 | 84 | 71 | 60 | 43 | 33 | 0.60 |
| 1300695 | 85 | 100 | 76 | 41 | 24 | 0.78 |
| 1300797 | 77 | 61 | 46 | 30 | 17 | 0.18 |
| 1300984 | 106 | 48 | 50 | 25 | 9 | 0.21 |
| 1301336 | 75 | 66 | 46 | 23 | 11 | 0.16 |

TABLE 36
Dose-dependent reduction of human NLRP3 RNA
in THP-1 cells by modified oligonucleotides

| Compound | NLRP3 RNA (% UTC) | | | | | IC₅₀ |
|---|---|---|---|---|---|---|
| No. | 16 nM | 63 nM | 250 nM | 1000 nM | 4000 nM | (μM) |
| 1232737 | 90 | 101 | 81 | 57 | 41 | 2.68 |
| 1299683 | 88 | 89 | 71 | 39 | 15 | 0.51 |
| 1299709 | 90 | 75 | 50 | 22 | 6 | 0.23 |
| 1299773 | 112 | 92 | 64 | 27 | 17 | 0.49 |
| 1299805 | 87 | 73 | 45 | 22 | 9 | 0.21 |
| 1300539 | 56 | 60 | 32 | 19 | 10 | 0.06 |
| 1300635 | 84 | 69 | 47 | 25 | 12 | 0.21 |
| 1300707 | 91 | 71 | 53 | 24 | 8 | 0.24 |
| 1300974 | 93 | 77 | 55 | 24 | 13 | 0.29 |
| 1300981 | 120 | 96 | 56 | 32 | 13 | 0.49 |
| 1301074 | 86 | 72 | 49 | 22 | 10 | 0.21 |
| 1301075 | 86 | 74 | 47 | 27 | 19 | 0.27 |
| 1301113 | 81 | 78 | 71 | 46 | 23 | 0.62 |
| 1301162 | 91 | 80 | 46 | 25 | 15 | 0.28 |
| 1301204 | 108 | 93 | 63 | 28 | 11 | 0.44 |

### Example 8: Tolerability of Modified Oligonucleotides Complementary to Human NLRP3 in Wildtype Mice

Wildtype BALB/c mice (Jackson Laboratory) were treated with modified oligonucleotides selected from studies described above and evaluated for changes in the levels of various plasma chemistry markers.

### Study 1

### Treatment

Groups of four male BALB/c mice each were injected subcutaneously once a week for five weeks (for a total of six treatments) with 100 mg/kg of modified oligonucleotides. One group of four male BALB/c mice was injected with PBS. The mice were euthanized seventy-two hours post the final administration of modified oligonucleotide.

### Plasma Chemistry Markers

To evaluate the effect of modified oligonucleotides on liver and kidney function, plasma levels of alanine aminotransferase (ALT), aspartate aminotransferase (AST), blood urea nitrogen (BUN), total bilirubin (TBIL), Albumin (ALB), and Creatinine (CREA) were measured on the day the mice were sacrificed (day 37) using an automated clinical chemistry analyzer (Hitachi Olympus AU400c, Melville, NY). The results were averaged for each group of mice and are presented in the tables below. Modified oligonucleotides that caused changes in the levels of any of the liver or kidney function markers outside the expected range for modified oligonucleotides were excluded from further studies.

TABLE 37
Plasma chemistry markers in BALB/c mice

| | Plasma clinical chemistry | | | | | |
|---|---|---|---|---|---|---|
| Compound | ALT | AST | BUN | TBIL | ALB | CREA |
| No. | (U/L) | (U/L) | (mg/dL) | (mg/dL) | (g/dL) | (mg/dL) |
| PBS | 20 | 39 | 26 | 0.14 | 2.8 | 0.11 |
| 1232844 | 87 | 148 | 25 | 0.16 | 2.67 | 0.13 |
| 1233100 | 46 | 58 | 28 | 0.15 | 2.44 | 0.12 |
| 1233104 | 151 | 179 | 22 | 0.12 | 2.38 | 0.09 |
| 1233105 | 36 | 57 | 28 | 0.12 | 2.53 | 0.15 |
| 1233144 | 967 | 732 | 25 | 0.65 | 2.96 | 0.15 |
| 1233279 | 30 | 58 | 21 | 0.14 | 2.57 | 0.1 |
| 1242446 | 55 | 99 | 24 | 0.12 | 2.27 | 0.09 |
| 1242447 | 24 | 46 | 26 | 0.13 | 2.54 | 0.1 |
| 1242484 | 83 | 88 | 24 | 0.11 | 2.59 | 0.1 |
| 1242543 | 134 | 171 | 31 | 0.12 | 2.42 | 0.11 |
| 1242553 | 1085 | 959 | 27 | 0.34 | 2.69 | 0.11 |

### Hematology Assays

Blood obtained from mice at week 5 were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), reticulocytes (RETI), and platelets (PLT) were evaluated. The results are presented in the tables below. Modified oligonucleotides that caused changes in the blood cell count outside the expected range were excluded in further studies.

TABLE 38
Hematology Parameters in BALB/c mice

| Compound | WBC | RBC | HGB | HCT | MCV | MCH |
|---|---|---|---|---|---|---|
| No. | (10\hat{ }3/μL) | (10\hat{ }12/L) | (g/dL) | (%) | (fL) | (pg) |
| PBS | 8 | 10 | 16 | 45 | 45 | 16 |
| 1232844 | 8 | 10 | 16 | 44 | 42 | 15 |
| 1233100 | 4 | 9 | 15 | 44 | 48 | 16 |
| 1233104 | 6 | 9 | 14 | 41 | 45 | 16 |
| 1233105 | 6 | 9 | 14 | 41 | 46 | 16 |
| 1233144 | 13 | 9 | 13 | 37 | 44 | 15 |
| 1233279 | 6 | 9 | 14 | 40 | 45 | 16 |
| 1242446 | 5 | 8 | 12 | 38 | 48 | 15 |
| 1242447 | 6 | 9 | 14 | 42 | 48 | 16 |
| 1242484 | 7 | 9 | 15 | 44 | 47 | 16 |
| 1242543 | 7 | 9 | 14 | 40 | 45 | 16 |
| 1242553 | 6 | 9 | 14 | 41 | 43 | 15 |

TABLE 39
Blood Cell Counts in BALB/c mice

| Compound | NEU | LYM | MON | EOS | BAS | RETI | PLT |
|---|---|---|---|---|---|---|---|
| No. | (10\hat{ }3/μL) | (10\hat{ }3/μL) | (10\hat{ }3/μL) | (10\hat{ }3/μL) | (10\hat{ }3/μL) | (10\hat{ }3/μL) | (10\hat{ }9/L) |
| PBS | 1163 | 6393 | 437 | 106 | 2 | 341 | 1207 |
| 1232844 | 2419 | 4908 | 585 | 208 | 4 | 435 | 525 |
| 1233100 | 1238 | 2431 | 290 | 90 | 2 | 311 | 1120 |
| 1233104 | 3017 | 2627 | 483 | 44 | 5 | 273 | 798 |
| 1233105 | 1001 | 4847 | 450 | 78 | 0 | 306 | 827 |
| 1233144 | 3066 | 8573 | 879 | 382 | 0 | 395 | 869 |
| 1233279 | 1675 | 4183 | 440 | 121 | 7 | 296 | 979 |
| 1242446 | 2347 | 2519 | 237 | 42 | 5 | 345 | 1023 |
| 1242447 | 1522 | 3624 | 436 | 114 | 8 | 598 | 1401 |
| 1242484 | 1515 | 4598 | 330 | 105 | 2 | 354 | 1344 |
| 1242543 | 1815 | 4128 | 508 | 96 | 4 | 272 | 815 |
| 1242553 | 1661 | 3810 | 523 | 128 | 4 | 331 | 722 |

### Body and Organ Weights

Body weights of BALB/c mice were measured on days 1 and 37, and the average body weight for each group is presented in the table below. Liver, kidney, and spleen weights were measured on the day the mice were sacrificed (day 37), and the average organ weights for each group are presented in the tables below. Modified oligonucleotides that caused any changes in organ weights outside the expected range for modified oligonucleotides were excluded from further studies.

TABLE 40
Body and organ weights (in grams)

| Compound | Body weight (g) | | Organ weight (g) | | |
|---|---|---|---|---|---|
| No. | Day 1 | Day 37 | Liver | Kidney | Spleen |
| PBS | 26 | 30 | 1.43 | 0.52 | 0.09 |
| 1232844 | 27 | 29 | 1.79 | 0.51 | 0.26 |
| 1233100 | 25 | 29 | 1.58 | 0.47 | 0.12 |
| 1233104 | 26 | 29 | 1.79 | 0.52 | 0.12 |
| 1233105 | 25 | 28 | 1.41 | 0.44 | 0.11 |
| 1233144 | 26 | 25 | 1.77 | 0.41 | 0.17 |
| 1233279 | 25 | 29 | 1.37 | 0.46 | 0.11 |
| 1242446 | 25 | 27 | 1.75 | 0.47 | 0.17 |
| 1242447 | 26 | 30 | 1.81 | 0.47 | 0.19 |
| 1242484 | 27 | 33 | 1.92 | 0.57 | 0.12 |
| 1242543 | 27 | 31 | 1.76 | 0.48 | 0.14 |
| 1242553 | 27 | 29 | 1.52 | 0.49 | 0.2 |

### Study 2

### Treatment

Groups of four male BALB/c mice each were injected subcutaneously once a week for five weeks (for a total of six treatments) with 100 mg/kg of modified oligonucleotides. One group of four male BALB/c mice was injected with PBS. The mice were euthanized seventy-two hours post the final administration of modified oligonucleotide.

### Plasma Chemistry Markers

To evaluate the effect of modified oligonucleotides on liver and kidney function, plasma levels of alanine aminotransferase (ALT), aspartate aminotransferase (AST), blood urea nitrogen (BUN), total bilirubin (TBIL), Albumin (ALB), and Creatinine (CREA) were measured on the day the mice were sacrificed (day 37) using an automated clinical chemistry analyzer (Hitachi Olympus AU400c, Melville, NY). The results were averaged for each group of mice and are presented in the tables below. Modified oligonucleotides that caused changes in the levels of any of the liver or kidney function markers outside the expected range for modified oligonucleotides were excluded from further studies.

TABLE 41
Plasma chemistry markers in BALB/c mice

| | Plasma clinical chemistry | | | | | |
|---|---|---|---|---|---|---|
| Compound | ALT | AST | BUN | TBIL | ALB | CREA |
| No. | (U/L) | (U/L) | (mg/dL) | (mg/dL) | (g/dL) | (mg/dL) |
| PBS | 20 | 43 | 21 | 0.18 | 2.79 | 0.13 |
| 1232846 | 1391 | 1434 | 25 | 0.13 | 2.7 | 0.16 |
| 1241470 | 1318 | 1292 | 27 | 0.23 | 3.05 | 0.16 |
| 1242482 | 1915 | 1664 | 24 | 0.27 | 3.14 | 0.17 |
| 1242538 | 36 | 67 | 22 | 0.13 | 2.74 | 0.16 |
| 1242546 | 57 | 101 | 23 | 0.11 | 2.59 | 0.13 |
| 1242547 | 192 | 242 | 25 | 0.14 | 2.68 | 0.17 |
| 1242579 | 36 | 57 | 21 | 0.1 | 2.76 | 0.15 |
| 1242601 | 117 | 187 | 25 | 0.12 | 2.97 | 0.17 |
| 1242624 | 1615 | 1452 | 24 | 0.21 | 2.73 | 0.15 |
| 1242633 | 175 | 307 | 21 | 0.12 | 2.79 | 0.15 |

### Hematology Assays

Blood obtained from mice at week 5 were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), reticulocytes (RETI), and platelets (PLT) were evaluated. The results are presented in the tables below. Modified oligonucleotides that caused changes in the blood cell count outside the expected range were excluded in further studies.

TABLE 42
Hematology Parameters in BALB/c mice

| Compound | WBC | RBC | HGB | HCT | MCV | MCH | MCHC |
|---|---|---|---|---|---|---|---|
| No. | (10\hat{ }3/μL) | (10\hat{ }12/L) | (g/dL) | (%) | (fL) | (pg) | (%) |
| PBS | 7 | 10 | 15 | 42 | 44 | 16 | 36 |
| 1232846 | 12 | 10 | 15 | 42 | 42 | 15 | 36 |
| 1241470 | 9 | 8 | 12 | 34 | 42 | 15 | 36 |
| 1242482 | 7 | 9 | 13 | 36 | 43 | 15 | 36 |
| 1242538 | 7 | 10 | 14 | 40 | 42 | 15 | 36 |
| 1242546 | 8 | 10 | 15 | 42 | 43 | 15 | 36 |
| 1242547 | 7 | 9 | 14 | 38 | 41 | 15 | 36 |
| 1242579 | 6 | 9 | 15 | 41 | 43 | 15 | 36 |
| 1242601 | 10 | 10 | 14 | 40 | 42 | 15 | 36 |
| 1242624 | 10 | 8 | 13 | 35 | 42 | 15 | 36 |
| 1242633 | 8 | 10 | 15 | 42 | 42 | 15 | 37 |

TABLE 43
Blood Cell Counts in BALB/c mice

| Compound | NEU | LYM | MON | EOS | BAS | RETI | PLT |
|---|---|---|---|---|---|---|---|
| No. | (10\hat{ }3/μL) | (10\hat{ }3/μL) | (10\hat{ }3/μL) | (10\hat{ }3/μL) | (10\hat{ }3/μL) | (10\hat{ }3/μL) | (10\hat{ }9/L) |
| PBS | 1560 | 5176 | 541 | 94 | 4 | 369 | 1168 |
| 1232846 | 2483 | 8223 | 1359 | 145 | 15 | 268 | 828 |
| 1241470 | 2431 | 5400 | 1097 | 118 | 4 | 256 | 989 |
| 1242482 | 1243 | 4975 | 709 | 118 | 5 | 303 | 875 |
| 1242538 | 1296 | 5167 | 703 | 224 | 10 | 365 | 809 |
| 1242546 | 1366 | 6173 | 766 | 167 | 4 | 350 | 664 |
| 1242547 | 1593 | 4423 | 792 | 87 | 6 | 273 | 508 |
| 1242579 | 1565 | 3454 | 541 | 114 | 2 | 290 | 1071 |
| 1242601 | 2110 | 7204 | 823 | 228 | 11 | 318 | 832 |
| 1242624 | 1691 | 6728 | 940 | 186 | 6 | 279 | 979 |
| 1242633 | 1503 | 5728 | 680 | 235 | 4.25 | 324 | 947 |

### Body and Organ Weights

Body weights of BALB/c mice were measured on days 1 and 37, and the average body weight for each group is presented in the table below. Liver, kidney, and spleen weights were measured on the day the mice were sacrificed (day 37), and the average organ weights for each group are presented in the tables below. Modified oligonucleotides that caused any changes in organ weights outside the expected range for modified oligonucleotides were excluded from further studies.

TABLE 44
Body and organ weights (in grams)

| Compound | Body weight (g) | | Organ weight (g) | | |
|---|---|---|---|---|---|
| No. | Day 1 | Day 37 | Liver | Kidney | Spleen |
| PBS | 29 | 32 | 1.56 | 0.55 | 0.09 |
| 1232846 | 27 | 30 | 2.41 | 0.47 | 0.15 |
| 1241470 | 28 | 31 | 2.26 | 0.51 | 0.10 |
| 1242482 | 26 | 30 | 2.22 | 0.47 | 0.11 |
| 1242538 | 27 | 30 | 1.67 | 0.51 | 0.12 |
| 1242546 | 27 | 31 | 1.56 | 0.46 | 0.11 |
| 1242547 | 24 | 27 | 1.35 | 0.40 | 0.15 |
| 1242579 | 26 | 30 | 1.51 | 0.47 | 0.10 |
| 1242601 | 27 | 29 | 1.31 | 0.43 | 0.11 |
| 1242624 | 27 | 26 | 1.31 | 0.48 | 0.11 |
| 1242633 | 25 | 28 | 1.59 | 0.42 | 0.12 |

### Study 3

### Treatment

Groups of four male BALB/c mice each were injected subcutaneously once a week for five weeks (for a total of six treatments) with 100 mg/kg of modified oligonucleotides. One group of four male BALB/c mice was injected with PBS. The mice were euthanized seventy-two hours post the final administration of modified oligonucleotide.

### Plasma Chemistry Markers

To evaluate the effect of modified oligonucleotides on liver and kidney function, plasma levels of alanine aminotransferase (ALT), aspartate aminotransferase (AST), blood urea nitrogen (BUN), total bilirubin (TBIL), Albumin (ALB), and Creatinine (CREA) were measured on the day the mice were sacrificed (day 39) using an automated clinical chemistry analyzer (Hitachi Olympus AU400c, Melville, NY). The results were averaged for each group of mice and are presented in the tables below. Modified oligonucleotides that caused changes in the levels of any of the liver or kidney function markers outside the expected range for modified oligonucleotides were excluded from further studies.

TABLE 45
Plasma chemistry markers in BALB/c mice

| | Plasma clinical chemistry | | | | | |
|---|---|---|---|---|---|---|
| Compound | ALT | AST | BUN | TBIL | ALB | CREA |
| No. | (U/L) | (U/L) | (mg/dL) | (mg/dL) | (g/dL) | (mg/dL) |
| PBS | 21 | 45 | 22 | 0.15 | 2.64 | 0.11 |
| 1299773 | 66 | 106 | 19 | 0.17 | 2.7 | 0.12 |
| 1299805 | 366 | 650 | 22 | 0.2 | 2.75 | 0.14 |
| 1299941 | 954 | 898 | 17 | 0.16 | 2.34 | 0.1 |
| 1300572 | 3076 | 2593 | 21 | 1.07 | 3.13 | 0.11 |
| 1300578 | 137 | 284 | 21 | 0.16 | 2.88 | 0.1 |
| 1300984 | 295 | 570 | 23 | 0.14 | 2.53 | 0.13 |
| 1301108 | 394 | 588 | 21 | 0.14 | 2.42 | 0.09 |
| 1304745 | 31 | 63 | 22 | 0.14 | 2.53 | 0.1 |
| 1304758 | 4656 | 3068 | 28 | 0.32 | 2.76 | 0.12 |
| 1304790 | 2253 | 3201 | 28 | 0.56 | 2.37 | 0.1 |

### Hematology Assays

Blood obtained from mice at week 5 were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), reticulocytes (RETI), and platelets (PLT) were evaluated. The results are presented in the tables below. Modified oligonucleotides that caused changes in the blood cell count outside the expected range were excluded in further studies.

TABLE 46
Hematology Parameters in BALB/c mice

| Compound | WBC | RBC | HGB | HCT | MCV | MCH | MCHC |
|---|---|---|---|---|---|---|---|
| No. | (10\hat{ }3/μL) | (10\hat{ }12/L) | (g/dL) | (%) | (fL) | (pg) | (%) |
| PBS | 4.5 | 10 | 15 | 43 | 45 | 15 | 34 |
| 1299773 | 7.18 | 10 | 14 | 42 | 43 | 15 | 34 |
| 1299805 | 7.85 | 9 | 14 | 40 | 43 | 15 | 35 |
| 1299941 | 7.35 | 9 | 14 | 39 | 43 | 15 | 35 |
| 1300572 | 12.23 | 10 | 14 | 41 | 43 | 14 | 34 |
| 1300578 | 6.95 | 10 | 15 | 43 | 43 | 15 | 35 |
| 1300984 | 7.1 | 10 | 14 | 42 | 43 | 15 | 35 |
| 1301108 | 6.33 | 10 | 14 | 42 | 43 | 15 | 34 |
| 1304745 | 5.55 | 9 | 14 | 41 | 43 | 15 | 34 |
| 1304758 | 12.15 | 10 | 14 | 42 | 42 | 14 | 34 |
| 1304790 | 6.78 | 9 | 12 | 38 | 41 | 14 | 33 |

TABLE 47
Blood Cell Counts in BALB/c mice

| Compound | NEU | LYM | MON | EOS | BAS | RETI | PLT |
|---|---|---|---|---|---|---|---|
| No. | (10\hat{ }3/μL) | (10\hat{ }3/μL) | (10\hat{ }3/μL) | (10\hat{ }3/μL) | (10\hat{ }3/μL) | (10\hat{ }3/μL) | (10\hat{ }9/L) |
| PBS | 942 | 3224 | 214 | 120 | 0 | 432 | 1017 |
| 1299773 | 1287 | 5084 | 443 | 357 | 4 | 388 | 711 |
| 1299805 | 1399 | 5653 | 489 | 306 | 3 | 426 | 772 |
| 1299941 | 2097 | 4535 | 628 | 89 | 2 | 387 | 783 |
| 1300572 | 2292 | 8468 | 793 | 670 | 3 | 735 | 658 |
| 1300578 | 1348 | 4547 | 418 | 636 | 0 | 423 | 954 |
| 1300984 | 1443 | 4662 | 517 | 472 | 7 | 386 | 774 |
| 1301108 | 1070 | 4332 | 520 | 400 | 4 | 386 | 939 |
| 1304745 | 1137 | 3639 | 358 | 414 | 3 | 419 | 930 |
| 1304758 | 2064 | 8071 | 1118 | 887 | 10 | 465 | 1074 |
| 1304790 | 1904 | 4006 | 617 | 246 | 3 | 502 | 798 |

### Body and Organ Weights

Body weights of BALB/c mice were measured on days 1 and 39, and the average body weight for each group is presented in the table below. Liver, kidney, and spleen weights were measured on the day the mice were sacrificed (day 39), and the average organ weights for each group are presented in the tables below. Modified oligonucleotides that caused any changes in organ weights outside the expected range for modified oligonucleotides were excluded from further studies.

TABLE 48
Body and organ weights (in grams)

| Compound | Body weight (g) | | Organ weight (g) | | |
|---|---|---|---|---|---|
| No. | Day 1 | Day 39 | Liver | Kidney | Spleen |
| PBS | 22 | 28 | 1.35 | 0.46 | 0.09 |
| 1299773 | 20 | 24 | 1.27 | 0.40 | 0.12 |
| 1299805 | 20 | 26 | 1.42 | 0.41 | 0.10 |
| 1299941 | 21 | 26 | 1.47 | 0.42 | 0.14 |
| 1300572 | 19 | 25 | 2.00 | 0.39 | 0.33 |
| 1300578 | 19 | 24 | 1.24 | 0.40 | 0.09 |
| 1300984 | 22 | 27 | 1.60 | 0.35 | 0.20 |
| 1301108 | 18 | 24 | 1.29 | 0.35 | 0.10 |
| 1304745 | 21 | 25 | 1.35 | 0.41 | 0.10 |
| 1304758 | 20 | 25 | 1.95 | 0.39 | 0.15 |
| 1304790 | 21 | 25 | 1.92 | 0.44 | 0.30 |

### Study 4

### Treatment

Groups of four male BALB/c mice each were injected subcutaneously once a week for five weeks (for a total of six treatments) with 100 mg/kg of modified oligonucleotides. One group of four male BALB/c mice was injected with PBS. The mice were euthanized seventy-two hours post the final administration of modified oligonucleotide.

### Plasma Chemistry Markers

To evaluate the effect of modified oligonucleotides on liver and kidney function, plasma levels of alanine aminotransferase (ALT), aspartate aminotransferase (AST), blood urea nitrogen (BUN), total bilirubin (TBIL), Albumin (ALB), and Creatinine (CREA) were measured on the day the mice were sacrificed (day 39) using an automated clinical chemistry analyzer (Hitachi Olympus AU400c, Melville, NY). The results were averaged for each group of mice and are presented in the tables below. Modified oligonucleotides that caused changes in the levels of any of the liver or kidney function markers outside the expected range for modified oligonucleotides were excluded from further studies.

TABLE 49
Plasma chemistry markers in BALB/c mice

| | Plasma clinical chemistry | | | | | |
|---|---|---|---|---|---|---|
| Compound | ALT | AST | BUN | TBIL | ALB | CREA |
| No. | (U/L) | (U/L) | (mg/dL) | (mg/dL) | (g/dL) | (mg/dL) |
| PBS | 21 | 48 | 23 | 0.14 | 2.72 | 0.12 |
| 1232754 | 21 | 59 | 23 | 0.14 | 2.64 | 0.13 |
| 1232845 | 162 | 232 | 24 | 0.16 | 2.89 | 0.18 |
| 1242698 | 906 | 1673 | 19 | 0.18 | 2.94 | 0.15 |
| 1242928 | 94 | 133 | 21 | 0.13 | 2.53 | 0.13 |
| 1242929 | 132 | 149 | 27 | 0.15 | 2.66 | 0.15 |
| 1299684 | 47 | 94 | 24 | 0.11 | 2.61 | 0.12 |
| 1300266 | 5614 | 4448 | 22 | 1 | 3.3 | 0.17 |
| 1300441 | 258 | 356 | 25 | 0.14 | 2.87 | 0.13 |
| 1300539 | 1386 | 1614 | 23 | 0.18 | 2.95 | 0.13 |
| 1300897 | 55 | 100 | 19 | 0.13 | 2.57 | 0.12 |

### Hematology Assays

Blood obtained from mice at week 5 were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), reticulocytes (RETI), and platelets (PLT) were evaluated. The results are presented in the tables below. Modified oligonucleotides that caused changes in the blood cell count outside the expected range were excluded in further studies.

TABLE 50
Hematology Parameters in BALB/c mice

| Compound | WBC | RBC | HGB | HCT | MCV | MCH | MCHC |
|---|---|---|---|---|---|---|---|
| No. | (10\hat{ }3/μL) | (10\hat{ }12/L) | (g/dL) | (%) | (fL) | (pg) | (%) |
| PBS | 3.6 | 9.19 | 14 | 42 | 46 | 15 | 33 |
| 1232754 | 4.85 | 9.36 | 14 | 42 | 45 | 15 | 33 |
| 1232845 | 10.25 | 9.81 | 14 | 44 | 45 | 15 | 32 |
| 1242698 | 6.63 | 9.38 | 14 | 43 | 46 | 15 | 33 |
| 1242928 | 6.4 | 9.65 | 15 | 44 | 46 | 15 | 33 |
| 1242929 | 7.28 | 8.92 | 13 | 40 | 45 | 15 | 33 |
| 1299684 | 6.28 | 9.26 | 14 | 42 | 46 | 15 | 33 |
| 1300266 | 11.63 | 9.4 | 13 | 39 | 42 | 14 | 34 |
| 1300441 | 7.45 | 9.17 | 14 | 42 | 46 | 15 | 32 |
| 1300539 | 12.13 | 9.39 | 14 | 42 | 45 | 15 | 33 |
| 1300897 | 7.15 | 9.49 | 14 | 43 | 46 | 15 | 33 |

TABLE 51
Blood Cell Counts in BALB/c mice

| Compound | NEU | LYM | MON | EOS | BAS | RETI | PLT |
|---|---|---|---|---|---|---|---|
| No. | (10\hat{ }3/μL) | (10\hat{ }3/μL) | (10\hat{ }3/μL) | (10\hat{ }3/μL) | (10\hat{ }3/μL) | (10\hat{ }3/μL) | (10\hat{ }9/L) |
| PBS | 802 | 2656 | 118 | 25 | 0 | 347 | 1225 |
| 1232754 | 1164 | 3283 | 336 | 68 | 0 | 358 | 913 |
| 1232845 | 2712 | 6726 | 786 | 26 | 0 | 267 | 630 |
| 1242698 | 1480 | 4253 | 434 | 35 | 0 | 380 | 984 |
| 1242928 | 1763 | 4136 | 405 | 97 | 0 | 364 | 864 |
| 1242929 | 2070 | 4830 | 230 | 120 | 25 | 301 | 867 |
| 1299684 | 1836 | 3953 | 390 | 80 | 0 | 339 | 834 |
| 1300266 | 3255 | 7285 | 926 | 133 | 27 | 272 | 1650 |
| 1300441 | 2306 | 4800 | 221 | 101 | 21 | 357 | 728 |
| 1300539 | 2223 | 8584 | 1318 | 0 | 14 | 502 | 428 |
| 1300897 | 2793 | 4031 | 278 | 49 | 0 | 348 | 958 |

### Body and Organ Weights

Body weights of BALB/c mice were measured on days 1 and 39, and the average body weight for each group is presented in the table below. Liver, kidney, and spleen weights were measured on the day the mice were sacrificed (day 39), and the average organ weights for each group are presented in the tables below. Modified oligonucleotides that caused any changes in organ weights outside the expected range for modified oligonucleotides were excluded from further studies.

TABLE 52
Body and organ weights (in grams)

| Compound | Body weight (g) | | Organ weight (g) | | |
|---|---|---|---|---|---|
| No. | Day 1 | Day 39 | Liver | Kidney | Spleen |
| PBS | 23 | 26 | 1.28 | 0.45 | 0.09 |
| 1232754 | 23 | 27 | 1.41 | 0.43 | 0.11 |
| 1232845 | 24 | 27 | 1.67 | 0.47 | 0.14 |
| 1242698 | 23 | 27 | 1.65 | 0.44 | 0.11 |
| 1242928 | 24 | 29 | 1.75 | 0.42 | 0.13 |
| 1242929 | 24 | 26 | 1.48 | 0.40 | 0.13 |
| 1299684 | 25 | 28 | 1.40 | 0.41 | 0.15 |
| 1300266 | 24 | 25 | 1.96 | 0.39 | 0.12 |
| 1300441 | 25 | 27 | 1.45 | 0.41 | 0.13 |
| 1300539 | 24 | 26 | 1.85 | 0.48 | 0.30 |
| 1300897 | 24 | 28 | 1.40 | 0.44 | 0.13 |

### Example 9: Tolerability of Modified Oligonucleotides Complementary to Human NLRP3 in Sprague-Dawley Rats

Sprague-Dawley rats are a multipurpose model used for safety and efficacy evaluations. The rats were treated with modified oligonucleotides from the studies described in the Examples above and evaluated for changes in the levels of various plasma chemistry markers.

### Study 1

### Treatment

Groups of 4 Sprague-Dawley rats (Charles River) each were injected subcutaneously once a week for 6 weeks (total 7 treatments) with 50 mg/kg of modified oligonucleotide. The rats were euthanized seventy-two hours post final administration of modified oligonucleotide. Organs, urine, and plasma were harvested for further analysis.

### Plasma Chemistry Markers

To evaluate the effect of modified oligonucleotides on liver and kidney function, plasma levels of alanine aminotransferase (ALT), aspartate aminotransferase (AST), blood urea nitrogen (BUN), albumin (ALB), creatinine (CREA), and total bilirubin (TBIL) were measured on the day the rats were sacrificed (day 35) using an automated clinical chemistry analyzer (Hitachi Olympus AU400c, Melville, NY). The results were averaged for each group of rats and are presented in the tables below. Modified oligonucleotides that caused changes in the levels of any of the liver or kidney function markers outside the expected range for modified oligonucleotides were excluded from further studies.

TABLE 53
Plasma chemistry markers in Sprague-Dawley rats

| Compound | ALT | AST | BUN | ALB | CREA | TBIL |
|---|---|---|---|---|---|---|
| No. | (IU/L) | (IU/L) | (mg/dL) | (g/dL) | (mg/dL) | (mg/dL) |
| PBS | 25 | 85 | 17 | 3.06 | 0.23 | 0.11 |
| 1232844 | 63 | 212 | 35 | 2.62 | 0.36 | 0.19 |
| 1233100 | 33 | 95 | 18 | 2.93 | 0.33 | 0.1 |
| 1233104 | 22 | 68 | 19 | 2.98 | 0.31 | 0.11 |
| 1233105 | 30 | 104 | 20 | 2.61 | 0.33 | 0.13 |
| 1233279 | 23 | 91 | 17 | 2.98 | 0.31 | 0.11 |
| 1242446 | 34 | 113 | 17 | 3.03 | 0.29 | 0.1 |
| 1242447 | 33 | 58 | 16 | 3.08 | 0.26 | 0.1 |
| 1242484 | 70 | 102 | 21 | 3.35 | 0.34 | 0.14 |
| 1242538 | 24 | 59 | 16 | 3.06 | 0.28 | 0.1 |
| 1242543 | 40 | 107 | 19 | 2.98 | 0.28 | 0.11 |
| 1242546 | 26 | 80 | 20 | 2.93 | 0.29 | 0.1 |
| 1242547 | 37 | 110 | 20 | 2.94 | 0.31 | 0.15 |
| 1242579 | 27 | 95 | 16 | 3.11 | 0.27 | 0.1 |
| 1242601 | 41 | 101 | 17 | 2.93 | 0.28 | 0.14 |
| 1242633 | 38 | 78 | 16 | 3.08 | 0.26 | 0.12 |

### Kidney Function

To evaluate the effect of modified oligonucleotides on kidney function, urinary levels of total protein and creatinine were measured using an automated clinical chemistry analyzer (Hitachi Olympus AU400c, Melville, NY). The ratios of total protein to creatinine (P/C ratio) are presented in the table below. Modified oligonucleotides that caused changes in the levels of the ratio outside the expected range for modified oligonucleotides were excluded in further studies.

TABLE 54
Total protein to creatinine ratio in Sprague-Dawley rats

| Compound | P/C |
|---|---|
| No. | Ratio |
| PBS | 1.02 |
| 1232844 | 6.94 |
| 1233100 | 4.34 |
| 1233104 | 5.43 |
| 1233105 | 3.75 |
| 1233279 | 2.46 |
| 1242446 | 3.42 |
| 1242447 | 3.08 |
| 1242484 | 2.42 |
| 1242538 | 3.14 |
| 1242543 | 2.37 |
| 1242546 | 3.85 |
| 1242547 | 3.01 |
| 1242579 | 3.41 |
| 1242601 | 3.9 |
| 1242633 | 2.84 |

### Body and Organ Weights

Body weights of the rats were measured on days 1 and 35 and the average body weight for each group is presented in the table below. Liver, kidney, and spleen weights were measured on the day the rats were sacrificed (day 35), and the average organ weights for each group are presented in the tables below. Modified oligonucleotides that caused any changes in organ weights outside the expected range for modified oligonucleotides were excluded from further studies.

TABLE 55
Body and organ weights (in grams)

| Compound | Body weight (g) | | Organ weight (g) | | |
|---|---|---|---|---|---|
| No. | Day 1 | Day 35 | Liver | Kidney | Spleen |
| PBS | 186 | 424 | 14 | 3.39 | 0.77 |
| 1232844 | 183 | 294 | 11 | 3.02 | 1.61 |
| 1233100 | 178 | 351 | 13 | 3.31 | 1.33 |
| 1233104 | 187 | 348 | 14 | 3.33 | 1.73 |
| 1233105 | 181 | 324 | 17 | 2.84 | 2.19 |
| 1233279 | 185 | 361 | 12 | 3.28 | 1.54 |
| 1242446 | 189 | 385 | 13 | 2.99 | 1.22 |
| 1242447 | 183 | 396 | 13 | 3.29 | 0.82 |
| 1242484 | 194 | 407 | 13 | 3.28 | 0.87 |
| 1242538 | 186 | 365 | 13 | 3.74 | 1.53 |
| 1242543 | 184 | 391 | 13 | 3.13 | 1.06 |
| 1242546 | 182 | 374 | 13 | 3.06 | 2.09 |
| 1242547 | 184 | 341 | 10 | 2.78 | 1.54 |
| 1242579 | 183 | 358 | 13 | 2.97 | 1.02 |
| 1242601 | 184 | 360 | 13 | 3.13 | 1.32 |
| 1242633 | 180 | 385 | 12 | 3.07 | 1.08 |

### Study 2

### Treatment

Groups of 4 Sprague-Dawley rats (Charles River) each were injected subcutaneously once a week for 6 weeks (total 7 treatments) with 50 mg/kg of modified oligonucleotide. The rats were euthanized seventy-two hours post final administration of modified oligonucleotide. Organs, urine, and plasma were harvested for further analysis.

### Plasma Chemistry Markers

To evaluate the effect of modified oligonucleotides on liver and kidney function, plasma levels of alanine aminotransferase (ALT), aspartate aminotransferase (AST), blood urea nitrogen (BUN), albumin (ALB), creatinine (CREA), and total bilirubin (TBIL) were measured on the day the rats were sacrificed (day 33) using an automated clinical chemistry analyzer (Hitachi Olympus AU400c, Melville, NY). The results were averaged for each group of rats and are presented in the tables below. Modified oligonucleotides that caused changes in the levels of any of the liver or kidney function markers outside the expected range for modified oligonucleotides were excluded from further studies.

TABLE 56
Plasma chemistry markers in Sprague-Dawley rats

| Compound | ALT | AST | BUN | ALB | CREA | TBIL |
|---|---|---|---|---|---|---|
| No. | (IU/L) | (IU/L) | (mg/dL) | (g/dL) | (mg/dL) | (mg/dL) |
| PBS | 27 | 88 | 16 | 3.11 | 0.28 | 0.09 |
| 1299773 | 28 | 98 | 21 | 2.78 | 0.38 | 0.13 |
| 1300578 | 94 | 162 | 21 | 3.04 | 0.41 | 0.22 |
| 1304745 | 36 | 100 | 14 | 3.03 | 0.33 | 0.1 |

### Kidney Function

To evaluate the effect of modified oligonucleotides on kidney function, urinary levels of total protein and creatinine were measured using an automated clinical chemistry analyzer (Hitachi Olympus AU400c, Melville, NY). The ratios of total protein to creatinine (P/C ratio) are presented in the table below. Modified oligonucleotides that caused changes in the levels of the ratio outside the expected range for modified oligonucleotides were excluded in further studies.

TABLE 57
Total protein to creatinine ratio in Sprague-Dawley rats

| Compound | P/C |
|---|---|
| No. | Ratio |
| PBS | 1.17 |
| 1299773 | 5.82 |
| 1300578 | 2.87 |
| 1304745 | 3.69 |

### Body and Organ Weights

Body weights of the rats were measured on days 1 and 33 and the average body weight for each group is presented in the table below. Liver, kidney, and spleen weights were measured on the day the rats were sacrificed (day 33), and the average organ weights for each group are presented in the tables below. Modified oligonucleotides that caused any changes in organ weights outside the expected range for modified oligonucleotides were excluded from further studies.

TABLE 58
Body and organ weights (in grams)

| Compound | Body weight (g) | | Organ weight (g) | | |
|---|---|---|---|---|---|
| No. | Day 1 | Day 33 | Liver | Kidney | Spleen |
| PBS | 251 | 447 | 12 | 3.46 | 0.85 |
| 1299773 | 250 | 353 | 12 | 3.75 | 2.78 |
| 1300578 | 255 | 417 | 14 | 3.98 | 1.87 |
| 1304745 | 238 | 390 | 11 | 3.14 | 0.82 |

### Study 3

### Treatment

Groups of 4 Sprague-Dawley rats (Charles River) each were injected subcutaneously once a week for 5 weeks (total 7 treatments) with 100 mg/kg of modified oligonucleotide. The rats were euthanized seventy-two hours post final administration of modified oligonucleotide. Organs, urine and plasma were harvested for further analysis.

### Plasma Chemistry Markers

To evaluate the effect of modified oligonucleotides on liver and kidney function, plasma levels of alanine aminotransferase (ALT), aspartate aminotransferase (AST), blood urea nitrogen (BUN), albumin (ALB), creatinine (CREA), and total bilirubin (TBIL) were measured on the day the rats were sacrificed (day 33) using an automated clinical chemistry analyzer (Hitachi Olympus AU400c, Melville, NY). The results were averaged for each group of rats and are presented in the tables below. Modified oligonucleotides that caused changes in the levels of any of the liver or kidney function markers outside the expected range for modified oligonucleotides were excluded from further studies.

TABLE 59
Plasma chemistry markers in Sprague-Dawley rats

| Compound | ALT | AST | BUN | ALB | CREA | TBIL |
|---|---|---|---|---|---|---|
| No. | (IU/L) | (IU/L) | (mg/dL) | (g/dL) | (mg/dL) | (mg/dL) |
| PBS | 25 | 93 | 0.22 | 3.19 | 16 | 0.12 |
| 1232754 | 25 | 81 | 0.32 | 3.11 | 20 | 0.09 |
| 1232845 | 100 | 176 | 0.49 | 2.09 | 33 | 0.28 |
| 1242928 | 206 | 468 | 0.33 | 2.98 | 19 | 0.22 |
| 1242929 | 45 | 140 | 0.33 | 2.83 | 17 | 0.11 |
| 1299684 | 24 | 79 | 0.34 | 2.86 | 20 | 0.1 |
| 1300441 | 54 | 139 | 0.34 | 3.24 | 19 | 0.12 |
| 1300897 | 42 | 99 | 0.38 | 3.05 | 23 | 0.09 |

### Kidney Function

To evaluate the effect of modified oligonucleotides on kidney function, urinary levels of total protein and creatinine were measured using an automated clinical chemistry analyzer (Hitachi Olympus AU400c, Melville, NY). The ratios of total protein to creatinine (P/C ratio) are presented in the table below. Modified oligonucleotides that caused changes in the levels of the ratio outside the expected range for modified oligonucleotides were excluded in further studies.

TABLE 60
Total protein to creatinine ratio in Sprague-Dawley rats

| Compound | P/C |
|---|---|
| No. | Ratio |
| PBS | 1.28 |
| 1232754 | 3.3 |
| 1232845 | 8.95 |
| 1242928 | 2.95 |
| 1242929 | 2.14 |
| 1299684 | 4.14 |
| 1300441 | 2.9 |
| 1300897 | 3.64 |

### Body and Organ Weights

Body weights of the rats were measured on days 1 and 33 and the average body weight for each group is presented in the table below. Liver, kidney, and spleen weights were measured on the day the rats were sacrificed (day 33), and the average organ weights for each group are presented in the tables below. Modified oligonucleotides that caused any changes in organ weights outside the expected range for modified oligonucleotides were excluded from further studies.

TABLE 61
Body and organ weights (in grams)

| Compound | Body weight (g) | | Organ weight (g) | | |
|---|---|---|---|---|---|
| No. | Day 1 | Day 33 | Liver | Kidney | Spleen |
| PBS | 243 | 434 | 12 | 3.41 | 0.85 |
| 1232754 | 255 | 407 | 13 | 3.33 | 1.1 |
| 1232845 | 242 | 352 | 16 | 4.93 | 3.07 |
| 1242928 | 253 | 361 | 12 | 2.97 | 1.45 |
| 1242929 | 253 | 407 | 13 | 3.75 | 1.9 |
| 1299684 | 250 | 387 | 13 | 3.48 | 2.28 |
| 1300441 | 237 | 354 | 10 | 2.94 | 1.56 |
| 1300897 | 236 | 354 | 10 | 2.79 | 1.38 |

### Example 10: Activity of Modified Oligonucleotides Complementary to Human NLRP3 in Transgenic Mice, Single Dose

A transgenic mouse model was developed using the Fosmid ABC11-49324000P12. The clone was digested at NaeI restriction site to produce a region containing the 33164 base pairs of human NLRP3 gene together with non-genic regions of the fosmid. The gene fragment was introduced into fertilized eggs from C57BL/6NTac strain mice (Taconic Biosciences) by pronuclear injection to produce 4 founder lines. Males from Line 1 were used in the experiments described herein. Human NLRP3 RNA expression is found in the liver, kidney, and heart, in this model.

### Treatment

Transgenic mice were divided into groups of 2 mice each. Each mouse received subcutaneous injections of modified oligonucleotide at a dose of 100 mg/kg once a week for either one or two weeks (2 treatments), as indicated in the tables below. One group of 2-4 mice received subcutaneous injections of PBS or saline twice a week for either one or two weeks (2 treatments), as indicated in the tables below. The PBS or saline-injected group served as the control group to which modified oligonucleotide-treated groups were compared.

### RNA Analysis

72 hours post the final treatment, mice were sacrificed, and RNA was extracted from mouse liver, heart, and/or kidney as indicated for quantitative real-time RTPCR analysis of NLRP3 RNA expression using human NLRP3 primer probe set RTS37509 (described herein below). NLRP3 RNA levels were normalized to total RNA content, as measured by RIBOGREEN®. Results are presented as percent NLRP3 RNA, relative to the amount in tissue from PBS or Saline treated mice (% control).

TABLE 62
Reduction of human NLRP3 in transgenic mice (two-week study)

| Compound | NLRP3 RNA (% control) | | |
|---|---|---|---|
| No. | Liver | Heart | Kidney |
| PBS | 100 | 100 | 100 |
| 1242543 | 66 | 68 | 183 |
| 1232764 | 36 | 31 | 85 |
| 1232844 | 49 | 114 | 97 |
| 1233100 | 49 | 39 | 55 |
| 1233104 | 38 | 38 | 40 |
| 1233105 | 90 | 14 | 53 |
| 1233144 | 52 | 57 | 28 |
| 1233175 | 53 | 32 | 75 |
| 1233279 | 82 | 39 | 95 |
| 1241668 | 74 | 23 | 47 |
| 1241669 | 734 | 58 | 67 |
| 1242447 | 417 | 77 | 109 |

TABLE 63
Reduction of human NLRP3 in transgenic mice (two-week study)

| | Liver |
|---|---|
| Compound | NLRP3 RNA |
| No. | (% control) |
| PBS | 100 |
| 1299773 | 27 |
| 1299805 | 30 |
| 1299941 | 33 |
| 1300422 | 215 |
| 1300572 | 56 |
| 1300578 | 39 |
| 1300636 | 112 |
| 1300655 | 84 |
| 1300668 | 131 |
| 1300984 | 32 |
| 1301108 | 46 |
| 1304745 | 36 |
| 1304758 | 25 |
| 1304790 | 83 |

TABLE 64
Reduction of human NLRP3 in transgenic mice (one-week study)

| Compound | NLRP3 RNA (% control) | | |
|---|---|---|---|
| No. | Liver | Heart | Kidney |
| Saline | 100 | 100 | 100 |
| 1242747 | 37 | 61 | 62 |
| 1242819 | 35 | 23 | 56 |
| 1242907 | 49 | 50 | 71 |
| 1242916 | 41 | 110 | 61 |

### Example 11: Activity of Modified Oligonucleotides Complementary to Human NLRP3 in Transgenic Mice, Multiple Dose

hNLRP3 transgenic mice (described herein above) were used to determine dose response activity of modified oligonucleotides complementary to human NLRP3.

### Treatment

Transgenic mice were divided into groups of 4 mice each. Each mouse received subcutaneous injections of modified oligonucleotide at a dose indicated in the tables below twice a week for one week (2 treatments). One group of 3-4 mice received subcutaneous injections of PBS twice a week for one week (2 treatments). The PBS-injected group served as the control group to which oligonucleotide-treated groups were compared.

### RNA Analysis

72 hours post the final treatment, mice were sacrificed, and RNA was extracted from mouse liver, heart, and kidney for quantitative real-time RTPCR analysis of human NLRP3 RNA expression. Human NLRP3 primer probe set RTS37509 (described herein above) was used to measure human NLRP3 RNA levels. NLRP3 RNA levels were normalized to mouse GAPDH. Mouse GAPDH was amplified using mouse primer probe set mGapdh_LTS00102 (forward sequence GGCAAATTCAACGGCACAGT, designated herein as SEQ ID NO: 13; reverse sequence GGGTCTCGCTCCTGGAAGAT, designated herein as SEQ ID NO: 14; probe sequence AAGGCCGAGAATGGGAAGCTTGTCATC, designated herein as SEQ ID NO: 15). Results are presented as percent NLRP3 RNA, relative to the amount in PBS treated animals (% control). ED50s were calculated in Prism using nonlinear fit with variable slope (four parameter), top constrained to 100% (or 1), bottom constrained to 0. Y=Bottom+(Top−Bottom)/(1+(IC50/X)\hat{ }HillSlope).

TABLE 65
Reduction of human NLRP3 in transgenic mice

| | | Liver | | Kidney | | Heart | |
|---|---|---|---|---|---|---|---|
| | | NLRP3 | | NLRP3 | | NLRP3 | |
| Compound | Dose | RNA | ED50 | RNA | ED50 | RNA | ED50 |
| No. | (mg/kg) | (% control) | (mg/kg) | (% control) | (mg/kg) | (% control) | (mg/kg) |
| PBS | 0 | 100 | — | 100 | 1 | 100 | — |
| 1242484 | 90 | 43 | 44 | 50 | 74 | 40 | 32 |
| | 30 | 60 | | 67 | | 47 | |
| | 10 | 65 | | 87 | | 68 | |
| 1242579 | 90 | 40 | 42 | 55 | 76 | 50 | 72 |
| | 30 | 51 | | 64 | | 64 | |
| | 10 | 81 | | 80 | | 90 | |
| 1299773 | 90 | 29 | 23 | 39 | 39 | 23 | 12 |
| | 30 | 39 | | 53 | | 36 | |
| | 10 | 69 | | 73 | | 45 | |
| 1304745 | 90 | 24 | 29 | 48 | 53 | 39 | 55 |
| | 30 | 57 | | 59 | | 60 | |
| | 10 | 63 | | 74 | | 91 | |

TABLE 66
Reduction of human NLRP3 in transgenic mice

| | | Liver | | Kidney | | Heart | |
|---|---|---|---|---|---|---|---|
| | | NLRP3 | | NLRP3 | | NLRP3 | |
| Compound | Dose | RNA | ED50 | RNA | ED50 | RNA | ED50 |
| No. | (mg/kg) | (% control) | (mg/kg) | (% control) | (mg/kg) | (% control) | (mg/kg) |
| PBS | 0 | 100 | — | 100 | — | 100 | — |
| 1233100 | 90 | 24 | 16 | 43 | 44 | 21 | 15 |
| | 30 | 40 | | 58 | | 38 | |
| | 10 | 50 | | 71 | | 53 | |
| | 3 | 88 | | 90 | | 89 | |
| | 1 | 91 | | 97 | | 78 | |
| 1233279 | 90 | 36 | 21 | 50 | 60 | 23 | 11 |
| | 30 | 55 | | 68 | | 38 | |
| | 10 | 54 | | 75 | | 55 | |
| | 3 | 66 | | 86‡ | | 79‡ | |
| | 1 | 86 | | 97 | | 80 | |
| 1242447 | 90 | 24 | 16 | 51 | 68 | 33 | 20 |
| | 30 | 34 | | 70 | | 56 | |
| | 10 | 59 | | 86 | | 59 | |
| | 3 | 73 | | 63‡ | | 81‡ | |
| | 1 | 120 | | 98 | | 93 | |
| 1242543 | 90 | 34 | 10 | 54 | 97 | 33 | 5 |
| | 30 | 30 | | 90‡ | | 25‡ | |
| | 10 | 45 | | 99 | | 43 | |
| | 3 | 67 | | 107 | | 56 | |
| | 1 | 123 | | 117 | | 70 | |
| 1242547 | 90 | 40 | 7 | 46 | 53 | 60 | 4 |
| | 30 | 34 | | 58 | | 33‡ | |
| | 10 | 31 | | 81 | | 31 | |
| | 3 | 61 | | 89 | | 37 | |
| | 1 | 91 | | 97 | | 82 | |
| 1242601 | 90 | 25 | 14 | 68 | 223 | 28 | 5 |
| | 30 | 42 | | 89 | | 33 | |
| | 10 | 59 | | 102 | | 38 | |
| | 3 | 70 | | 132 | | 62 | |
| | 1 | 68 | | 89 | | 52 | |

‡indicates that fewer than 4 samples were available

1. An oligomeric compound comprising a modified oligonucleotide consisting of 12 to 50, 12 to 45, 12 to 40, 12 to 35, 12 to 30, 12 to 25, or 12 to 20 linked nucleosides, wherein the nucleobase sequence of the modified oligonucleotide is at least 90% complementary to an equal length portion of a NLRP3 nucleic acid, and wherein the modified oligonucleotide comprises at least one modification selected from a modified sugar moiety and a modified internucleoside linkage.

2. An oligomeric compound comprising a modified oligonucleotide consisting of 12 to 30 linked nucleosides and having a nucleobase sequence comprising at least 8, 9, 10, 11, 12, 13, 14, 15, 16, 17, 18, 19, or 20 contiguous nucleobases complementary to an equal length portion of the nucleobase sequence of any one of SEQ ID NOs: 20-2799.

3. An oligomeric compound comprising a modified oligonucleotide, wherein the nucleobase sequence of the modified oligonucleotide is at least 80% complementary, at least 85% complementary, at least 90% complementary, or at least 95% complementary to an equal length portion within any of nucleobases 12138-12155, 14478-14523, 14578-14622, 14667-14693, 15111-15130, 15182-15200, 17668-17686, 20272-20291, 20634-20657, 23475-23512, or 25026-25049 of SEQ ID NO: 1.

4. The oligomeric compound of claim 3, wherein the modified oligonucleotide comprises at least 8, at least 9, at least 10, at least 11, at least 12, at least 13, at least 14, at least 15, at least 16, at least 17, at least 18, at least 19, or at least 20 contiguous nucleobases within any of nucleobases 12138-12155, 14478-14523, 14578-14622, 14667-14693, 15111-15130, 15182-15200, 17668-17686, 20272-20291, 20634-20657, 23475-23512, or 25026-25049 of SEQ ID NO: 1.

5. An oligomeric compound comprising a modified oligonucleotide consisting of 16 linked nucleosides, wherein the modified oligonucleotide has a nucleobase sequence of SEQ ID NO: 1454.

6. An oligomeric compound comprising a modified oligonucleotide consisting of 16 linked nucleosides, wherein the modified oligonucleotide has a nucleobase sequence of SEQ ID NO: 628.

7. An oligomeric compound comprising a modified oligonucleotide consisting of 16 linked nucleosides, wherein the modified oligonucleotide has a nucleobase sequence of SEQ ID NO: 178.

8. An oligomeric compound comprising a modified oligonucleotide consisting of 16 linked nucleosides, wherein the modified oligonucleotide has a nucleobase sequence of SEQ ID NO: 420.

9. The oligomeric compound of any one of claims 1-8, wherein the modified oligonucleotide has a nucleobase sequence that is at least 80%, 85%, 90%, 95%, or 100% complementary to an equal length portion of a nucleobase sequence selected from SEQ ID NO: 1 or SEQ ID NO: 2 when measured across the entire nucleobase sequence of the modified oligonucleotide.

10. The oligomeric compound of any one of claims 1-9, wherein at least one modified nucleoside comprises a modified sugar moiety.

11. The oligomeric compound of claim 10, wherein the modified sugar moiety comprises a bicyclic sugar moiety.

12. The oligomeric compound of claim 11, wherein the bicyclic sugar moiety comprises a 2′-4′ bridge selected from —O—CH₂—; and —O—CH(CH₃)—.

13. The oligomeric compound of claim 10, wherein the modified sugar moiety comprises a non-bicyclic modified sugar moiety.

14. The oligomeric compound of claim 13, wherein the non-bicyclic modified sugar moiety is a 2′-MOE sugar moiety or 2′-OMe sugar moiety.

15. The oligomeric compound of any one of claims 1-9, wherein at least one modified nucleoside comprises a sugar surrogate.

16. The oligomeric compound of claim 15, wherein the sugar surrogate is selected from morpholino and PNA.

17. The oligomeric compound of any of claims 1-16, wherein the modified oligonucleotide has a sugar motif comprising:
a 5′ wing segment consisting of 1-5 linked nucleosides;
a gap segment consisting of 6-10 linked nucleosides; and
a 3′ wing segment consisting of 1-5 linked nucleosides,
wherein each nucleoside of the 5′ wing segment and each nucleoside of the 3′ wing segment comprises a modified sugar moiety, and wherein each nucleoside of the gap segment comprises an unmodified 2′-deoxyribosyl sugar moiety.

18. The oligomeric compound of any of claims 1-16, wherein the modified oligonucleotide has a sugar motif (5′ to 3′) selected from: kkkddddddddddkkk, kekdddddddddeekk, kkdddddddddkekek, kkkdddddddddkkke, kkkdyddddddddkkk, kekdddddddddeekk, kkdddddddddkekek, wherein each “d” represents a 2′-β-D-deoxyribosyl sugar moiety, each “y” represents a 2′-OMe sugar moiety, each “e” represents a 2′-MOE sugar moiety, and each “k” represents a cEt modified sugar moiety.

19. The oligomeric compound of any one of claims 1-18, wherein the modified oligonucleotide comprises at least one modified internucleoside linkage.

20. The oligomeric compound of claim 19, wherein the modified internucleoside linkage is a phosphorothioate internucleoside linkage or a mesyl phosphoramidate internucleoside linkage.

21. The oligomeric compound of claim 19, wherein each internucleoside linkage of the modified oligonucleotide is a phosphorothioate internucleoside linkage.

22. The oligomeric compound of any one of claims 1-20, wherein the modified oligonucleotide comprises at least one phosphodiester internucleoside linkage.

23. The oligomeric compound of claim 21, wherein each internucleoside linkage is independently selected from a phosphodiester internucleoside linkage, a mesyl phosphoramidate internucleoside linkage, or a phosphorothioate internucleoside linkage.

24. The oligomeric compound of any of claims 1-23, wherein the modified oligonucleotide comprises at least one modified nucleobase.

25. The oligomeric compound of claim 24, wherein the modified nucleobase is a 5-methyl cytosine.

26. The oligomeric compound of any of claims 1-25, wherein the modified oligonucleotide consists of 12-30, 12-22, 12-20, 14-20, 15-25, 16-20, 18-22 or 18-20 linked nucleosides.

27. The oligomeric compound of any of claims 1-26, wherein the modified oligonucleotide consists of 16 linked nucleosides.

28. The oligomeric compound of claim 27, wherein each of nucleosides 1-3 and 14-16 (from 5′ to 3′) is a cEt nucleoside and each of nucleosides 4-13 is a 2′-deoxynucleoside.

29. The oligomeric compound of claim 27, wherein each of nucleosides 1, 3, 15, and 16 (from 5′ to 3′) is a cEt nucleoside, nucleosides 2, 13, and 14 is a 2′-MOE nucleoside, and each of nucleosides 4-12 is a 2′-deoxynucleoside.

30. The oligomeric compound of any of claims 1-29, consisting of the modified oligonucleotide.

31. The oligomeric compound of any of claims 1-29, comprising a conjugate group comprising a conjugate moiety and a conjugate linker.

32. The oligomeric compound of claim 31, wherein the conjugate group comprises 1-3 N-Acetylgalactosamine (GalNAc) groups.

33. The oligomeric compound of claim 31 or 32, wherein the conjugate linker consists of a single bond.

34. The oligomeric compound of claim 33, wherein the conjugate linker is cleavable.

35. The oligomeric compound of claim 34, wherein the conjugate linker comprises 1-3 linker-nucleosides.

36. The oligomeric compound of any of claims 31-35, wherein the conjugate group is attached to the modified oligonucleotide at the 5′-end of the modified oligonucleotide.

37. The oligomeric compound of any of claims 31-36, wherein the conjugate group is attached to the modified oligonucleotide at the 3′-end of the modified oligonucleotide.

38. The oligomeric compound of any of claims 1-37 comprising a terminal group.

39. The oligomeric compound of any of claims 1-38 wherein the oligomeric compound is a single-stranded oligomeric compound.

40. The oligomeric compound of any of claim 1-34 or 36-39, wherein the oligomeric compound does not comprise linker-nucleosides.

41. An oligomeric duplex comprising an oligomeric compound of any of claim 1-38 or 40.

42. An antisense compound comprising or consisting of an oligomeric compound of any of claims 1-40 or an oligomeric duplex of claim 41.

43. The antisense agent of claim 42, wherein the antisense agent is:
i. an RNase H agent capable of reducing the amount of NLRP3 nucleic acid through the activation of RNase H; or
ii. an RNAi agent capable of reducing the amount of NLRP3 nucleic acid through the activation of RISC/Ago2.

44. The antisense agent of any of claim 42 or 43, wherein the conjugate group is a cell-targeting moiety.

45. A modified oligonucleotide according to the following chemical structure: 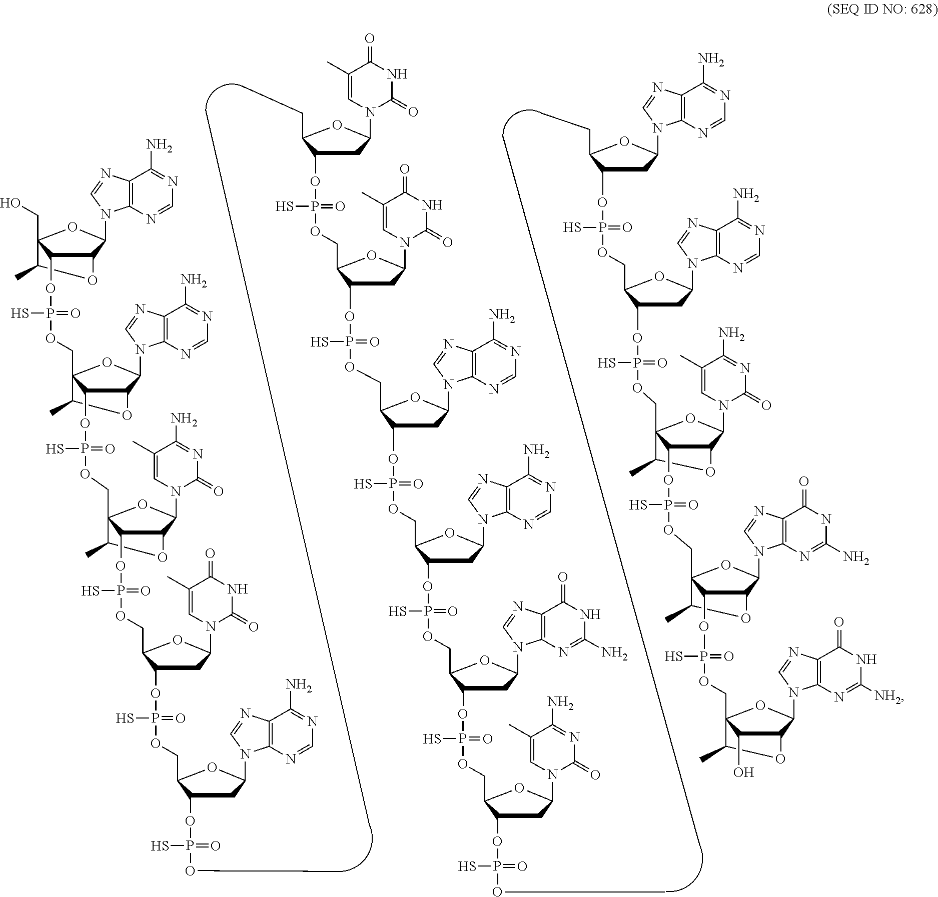
or a salt thereof.

46. The modified oligonucleotide of claim 45, which is a sodium salt or a potassium salt.

47. A modified oligonucleotide according to the following chemical structure:

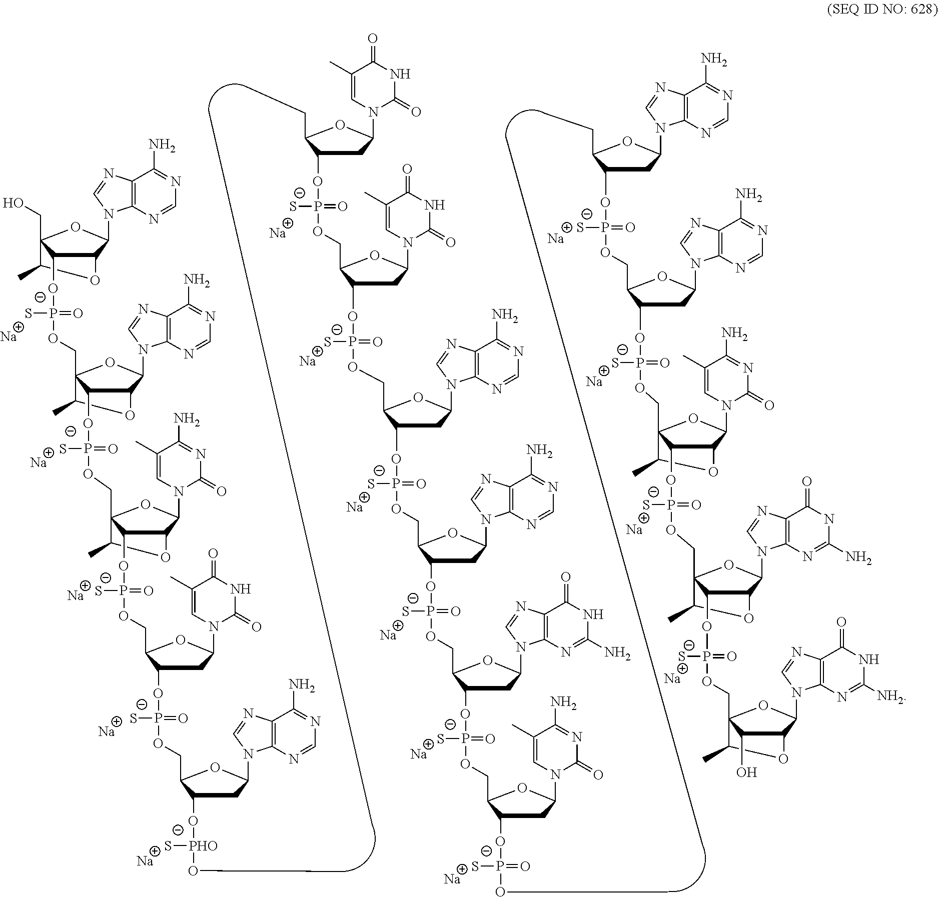

48. A modified oligonucleotide according to the following chemical structure: 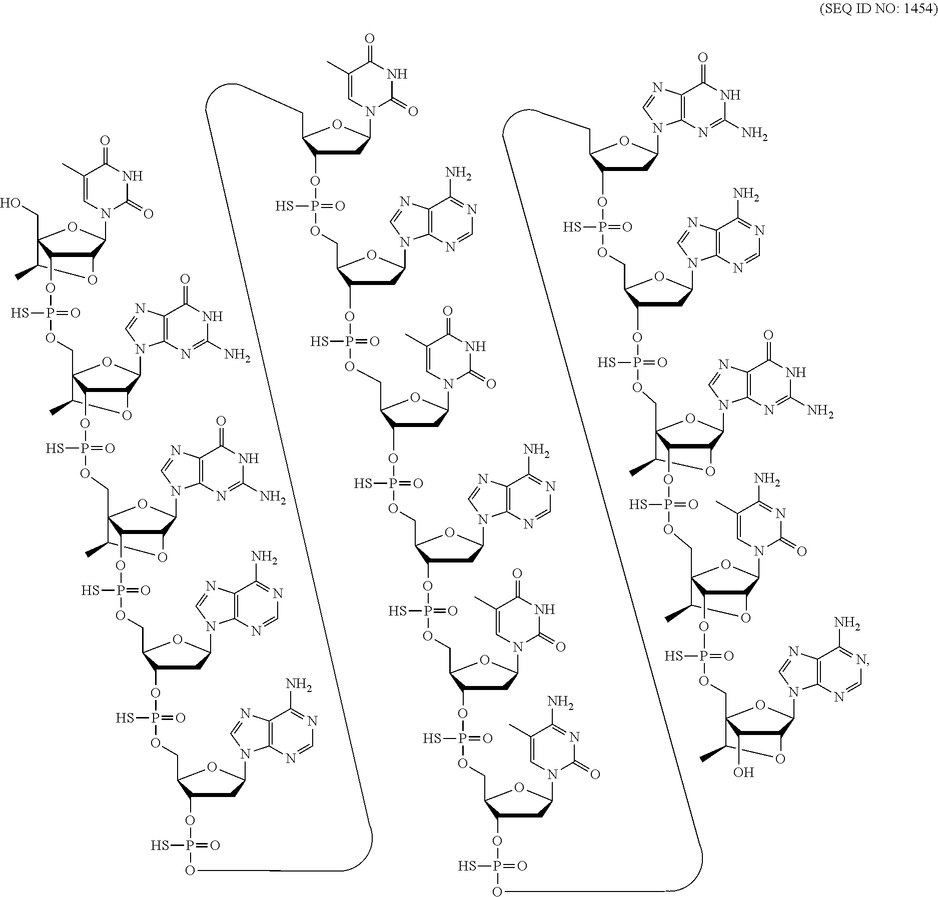
or a salt thereof.

49. The modified oligonucleotide of claim 48, which is a sodium salt or a potassium salt.

50. A modified oligonucleotide according to the following chemical structure:

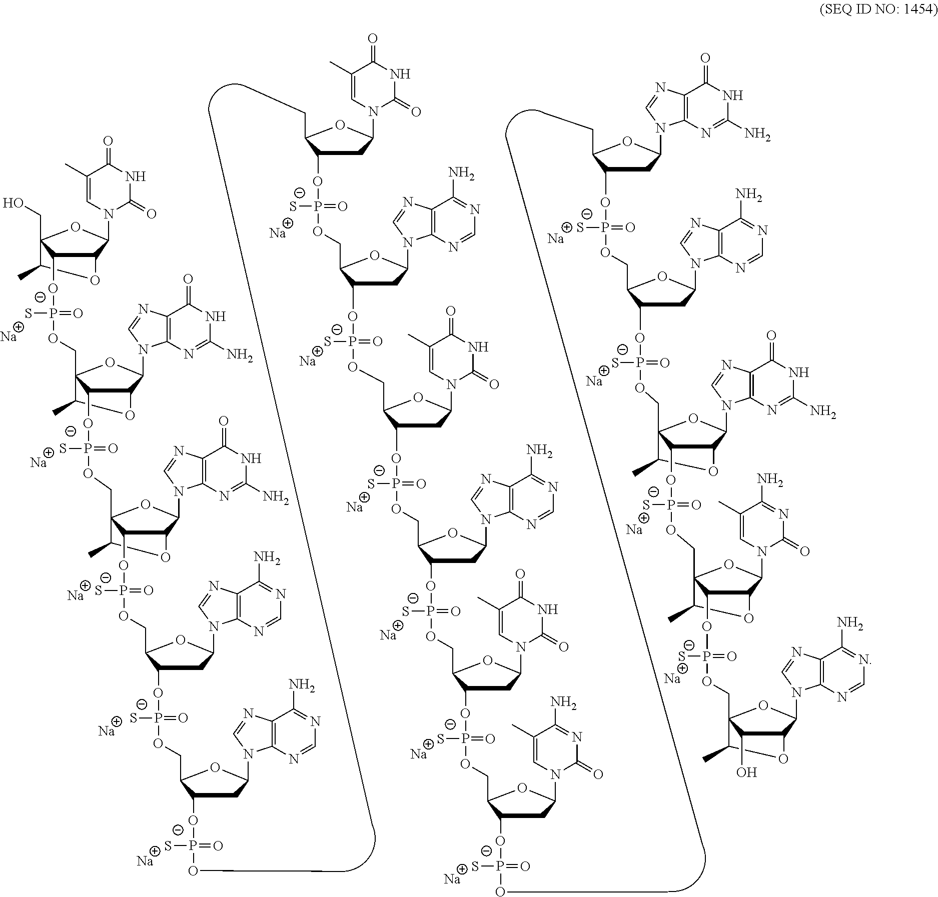

51. A modified oligonucleotide according to the following chemical structure: 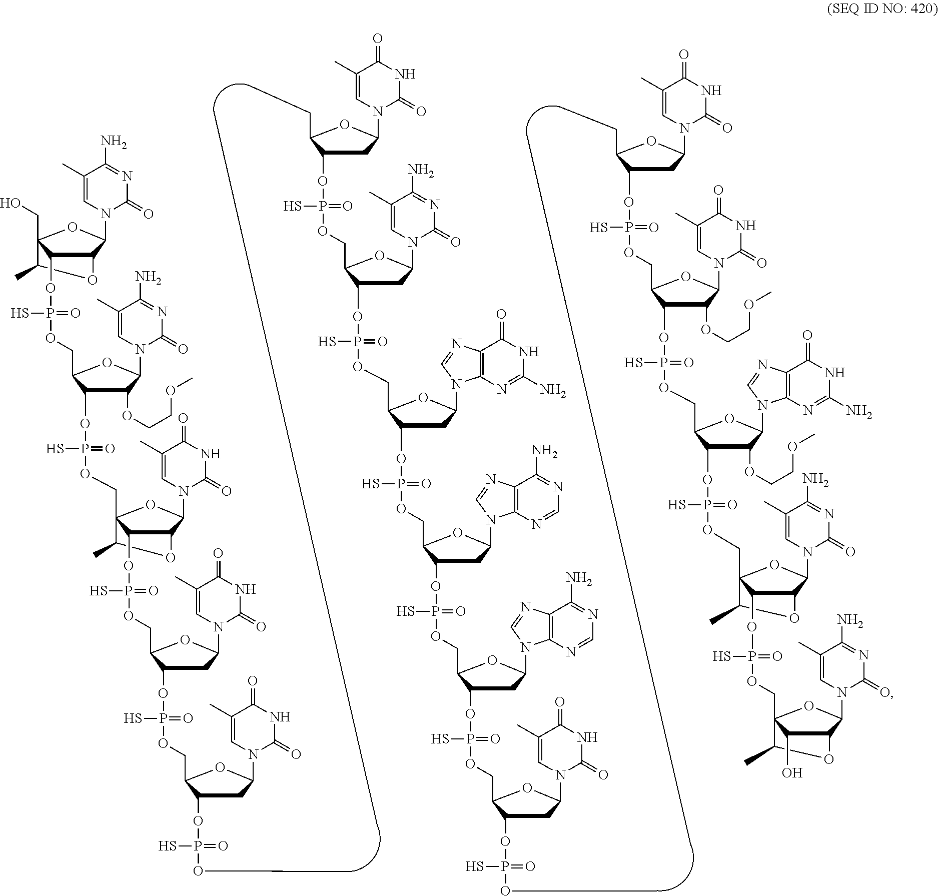
or a salt thereof.

52. The modified oligonucleotide of claim 51, which is a sodium salt or a potassium salt.

53. A modified oligonucleotide according to the following chemical structure:

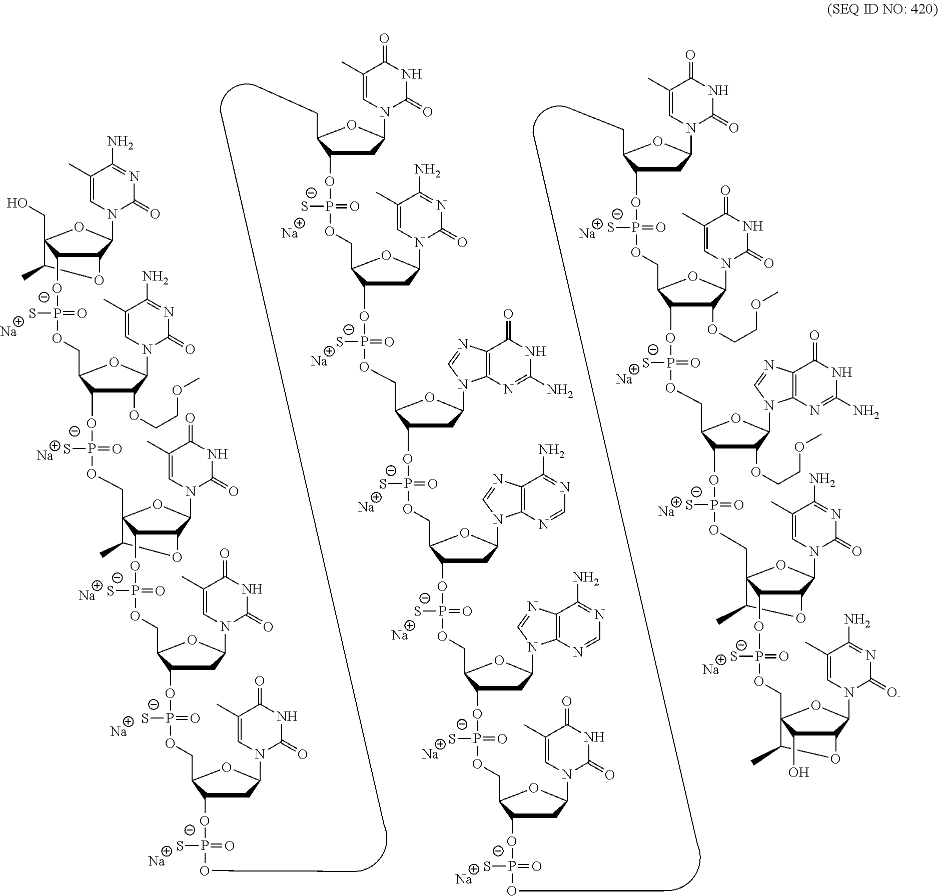

54. A modified oligonucleotide according to the following chemical notation:
AₖₛAₖₛ^{m}CₖₛT_{ds}A_{ds}T_{ds}T_{ds}A_{ds}A_{ds}G_{ds}^{m}C_{ds}A_{ds}A_{ds}^{m}CₖₛGₖₛGₖ (SEQ ID NO: 628); wherein
A=an adenine nucleobase,
mC=a 5-methyl cytosine nucleobase,
G=a guanine nucleobase,
T=a thymine nucleobase,
k=a cEt modified sugar moiety,
d=a 2′43-D-deoxyribosyl sugar moiety, and
s=a phosphorothioate internucleoside linkage.

55. A modified oligonucleotide according to the following chemical notation:
TₖₛGₖₛGₖₛA_{ds}A_{ds}T_{ds}A_{ds}T_{ds}A_{ds}T_{ds}^{m}C_{ds}G_{ds}A_{ds}Gₖₛ^{m}CₖₛAₖ (SEQ ID NO: 1454); wherein
A=an adenine nucleobase,
mC=a 5-methyl cytosine nucleobase,
G=a guanine nucleobase,
T=a thymine nucleobase,
k=a cEt modified sugar moiety,
d=a 2′43-D-deoxyribosyl sugar moiety, and
s=a phosphorothioate internucleoside linkage.

56. A modified oligonucleotide according to the following chemical notation:
^{m}Cₖₛ^{m}CₑₛTₖₛTₐₛTₐₛTₐₛ^{m}C_{ds}GₐₛA_{ds}A_{ds}TₐₛTₐₛTₑₛGₑₛ^{m}Cₖₛ^{m}Cₖ (SEQ ID NO: 420); wherein
A=an adenine nucleobase,
mC=a 5-methyl cytosine nucleobase,
G=a guanine nucleobase,
T=a thymine nucleobase,
k=a cEt modified sugar moiety,
e=a 2′-MOE β-D-ribosyl sugar moiety,
d=a 2′43-D-deoxyribosyl sugar moiety, and
s=a phosphorothioate internucleoside linkage.

57. A chirally enriched population of oligomeric compounds of any of claims 1-40, oligomeric duplexes of claim 41, or modified oligonucleotides of claims 45 to 56, wherein the population is enriched for modified oligonucleotides comprising at least one particular phosphorothioate internucleoside linkage having a particular stereochemical configuration.

58. The chirally enriched population of claim 57, wherein the population is enriched for modified oligonucleotides comprising at least one particular phosphorothioate internucleoside linkage having the (Sp) or (Rp) configuration.

59. The chirally enriched population of claim 57, wherein the population is enriched for modified oligonucleotides having a particular, independently selected stereochemical configuration at each phosphorothioate internucleoside linkage.

60. The chirally enriched population of claim 57, wherein the population is enriched for modified oligonucleotides having the (Rp) configuration at one particular phosphorothioate internucleoside linkage and the (Sp) configuration at each of the remaining phosphorothioate internucleoside linkages.

61. The chirally enriched population of claim 57, wherein the population is enriched for modified oligonucleotides having at least 3 contiguous phosphorothioate internucleoside linkages in the Sp, Sp, and Rp configurations, in the 5′ to 3′ direction.

62. A population of oligomeric compounds comprising modified oligonucleotides of any of claims 1-40, oligomeric duplexes of claim 41, or modified oligonucleotides of claims 45 to 56, wherein the stereochemistry of all internucleoside linkages of the modified oligonucleotide are stereorandom.

63. A pharmaceutical composition comprising the oligomeric compound of any of claims 1-40, the oligomeric duplex of claim 41, the antisense compound of any of claims 42-44, the modified oligonucleotides of any one of claims 45-56, or the population of any of claims 57-62; and a pharmaceutically acceptable carrier or diluent.

64. The pharmaceutical composition of claim 63, wherein the pharmaceutically acceptable carrier or diluent comprises sterile water or sterile saline.

65. The pharmaceutical composition of claim 64, consisting of, or consisting essentially of, the oligomeric compound, antisense compound or oligomeric duplex, and sterile water or sterile saline.

66. A method comprising administering to a subject the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65.

67. A method of treating a kidney disease or kidney injury comprising administering to a subject having or at risk for developing a kidney disease or kidney injury a therapeutically effective amount of the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65.

68. A method of reducing NLRP3 RNA or NLRP3 protein in a kidney, liver or heart of a subject having or at risk for developing a kidney disease or kidney injury comprising administering a therapeutically effective amount of the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65.

69. The method of claim 67 or 68, wherein the kidney disease is chronic kidney disease (CKD).

70. The method of claim 67 or 68, wherein the kidney injury is acute kidney injury (AKI).

71. The method of any one of claims 67-70, wherein at least one symptom of the kidney disease or kidney injury is ameliorated.

72. The method of claim 71, wherein the symptom is selected from nausea, vomiting, loss of appetite, reduced urine output, elevated serum creatinine levels, muscle cramping, swelling, itching, chest pain, shortness of breath, and elevated blood pressure.

73. The method of any of claims 67-72, wherein the method prevents or slows progression of the kidney disease or kidney injury.

74. Use of the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65 for the treatment of a kidney disease or kidney injury.

75. The use of claim 74, wherein the kidney disease is CKD.

76. The use of claim 74, wherein the kidney injury is AKI.

77. A method of treatment comprising administering to a subject having a cardiac disorder or cardiac injury a therapeutically effective amount of the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65.

78. A method of reducing NLRP3 RNA or NLRP3 protein in a kidney, liver, or heart of a subject having or at risk for developing a cardiac disorder or cardiac injury comprising administering a therapeutically effective amount of the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65.

79. The method of claim 77 or 78, wherein the cardiac disorder or cardiac injury is heart failure, hyperkalemia, cardiomyopathy, and/or a cardiac arrhythmia.

80. The method of any of claims 77-79, wherein administering the oligomeric compound, the population, the oligomeric duplex, the antisense agent, or the pharmaceutical composition improves a sign or a symptom selected from cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, and heart rhythm in the subject.

81. Use of the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65 for the treatment of a cardiac disorder or cardiac injury.

82. The use of claim 81, wherein the cardiac disorder or cardiac injury is heart failure, hyperkalemia, cardiomyopathy and/or a cardiac arrhythmia.

83. A method of treatment comprising administering to a subject having a liver disorder a therapeutically effective amount of the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65.

84. The method of claim 83, wherein the liver disorder is non-alcoholic fatty liver disease (NAFLD) or non-alcoholic steatohepatitis (NASH).

85. Use of the oligomeric compound of any of claims 1-40, the oligomeric duplex of claim 41, the antisense compound of any of claims 42-44, the modified oligonucleotides of any one of claims 45-56, or the population of any of claims 57-62, or the pharmaceutical composition of any of claims 63-65 in the manufacture of a medicament for treating a disease associated with NLRP3.
